SEQUENCE LISTING (230 sequences)

SEQ ID NO: 1 - protein, 506 aa, Artificial Sequence
  Flagellin
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLA

SEQ ID NO: 2 - DNA, 1518 bp, Artificial Sequence
  Flagellin
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttagcg

SEQ ID NO: 3 - protein, 277 aa, Artificial Sequence
  Flagellin STF2 delta
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLA

SEQ ID NO: 4 - DNA, 831 bp, Artificial Sequence
  Flagellin STF2 delta
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcatggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttagcg

SEQ ID NO: 5 - protein, 319 aa, Artificial Sequence
  P. falciparum CSP
MRGSSSNTRVLNELNYDNAGTNLYNELEMNYYGKQENWYSLKKNSRSLGENDDGNNNNGDNGREGKDEDKRDGNNEDNEKLRKPKHKKLKQPGDGNPDPNANPNVDPNANPNVDPNANPNVDPNANPNANPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEEYLNKIQNSLSTEWSPCSVTCGNGIQVRIKPGSANKPKDELDYENDIEKKICKMEKCSSVFNVVNSS

SEQ ID NO: 6 - DNA, 957 bp, Artificial Sequence
  P. falciparum CSP
atgagaggatccagcagcaacacccgtgttctgaacgaactgaactacgataacgctggtaccaatctgtacaacgaactggaaatgaactactacggtaaacaggaaaactggtacagcctgaaaaaaaacagcagatctctaggcgaaaacgacgacggcaacaacaacaacggtgataacggtcgcgaaggtaaagacgaagacaaacgcgacggcaacaacgaagacaacgaaaaacttcgcaaaccgaaacacaaaaaacttaagcagccaggggatggtaatccagatccgaacgcgaatccgaacgtagacccgaacgcaaacccgaacgtagacccgaacgcaaacccgaacgtagacccgaacgcgaatccgaacgcgaaccctaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacaaaaacaatcagggtaatggccagggtcacaatatgccaaatgacccaaaccgaaatgtagatgaaaatgctaatgccaacaatgctgtaaaaaataataataacgaagaaccaagtgataagcacatagaagagtatttaaacaaaatacaaaattctctttcaactgaatggtccccatgtagtgtaacttgcggcaacggtattcaggtgcgcatcaagccgggctctgctaacaaacctaaggacgaactggattacgaaaacgatatcgaaaaaaagatctgtaagatggaaaagtgttcctctgtattcaacgtagttaactcttcg

SEQ ID NO: 7 - protein, 824 aa, Artificial Sequence
  STF2. CSP
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLARGSSSNTRVLNELNYDNAGTNLYNELEMNYYGKQENWYSLKKNSRSLGENDDGNNNNGDNGREGKDEDKRDGNNEDNEKLRKPKHKKLKQPGDGNPDPNANPNVDPNANPNVDPNANPNVDPNANPNANPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEEYLNKIQNSLSTEWSPCSVTCGNGIQVRIKPGSANKPKDELDYENDIEKKICKMEKCSSVFNVVNSS

SEQ ID NO: 8 - DNA, 2472 bp, Artificial Sequence
  STF2. CSP
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttagcgagaggatccagcagcaacacccgtgttctgaacgaactgaactacgataacgctggtaccaatctgtacaacgaactggaaatgaactactacggtaaacaggaaaactggtacagcctgaaaaaaaacagcagatccctaggcgaaaacgacgacggcaacaacaacaacggtgataacggtcgcgaaggtaaagacgaagacaaacgcgacggcaacaacgaagacaacgaaaaacttcgcaaaccgaaacacaaaaaacttaagcagccaggggatggtaatccagatccgaacgcgaatccgaacgtagacccgaacgcaaacccgaacgtagacccgaacgcaaacccgaacgtagacccgaacgcgaatccgaacgcgaaccctaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacaaaaacaatcagggtaatggccagggtcacaatatgccaaatgacccaaaccgaaatgtagatgaaaatgctaatgccaacaatgctgtaaaaaataataataacgaagaaccaagtgataagcacatagaagagtatttaaacaaaatacaaaattctctttcaactgaatggtccccatgtagtgtaacttgcggcaacggtattcaggtgcgcatcaagccgggctctgctaacaaacctaaggacgaactggattacgaaaacgatatcgaaaaaaagatctgtaagatggaaaagtgttcctctgtattcaacgtagttaactcttcg

SEQ ID NO: 9 - protein, 554 aa, Artificial Sequence
  STF2.TIBT*
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLADPNANPNVDPNANPNVNANPNANPNANPEYLNKIQNSLSTEWSPCSVT

SEQ ID NO: 10 - DNA, 1662 bp, Artificial Sequence
  STF2.TIBT*
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttagcggacccgaatgcaaatccgaatgttgacccaaatgcgaacccaaatgtcaacgcgaaccctaacgctaacccaaacgctaacccggaatacctgaacaagatccagaacagcctgtccaccgaatggtccccgtgctctgtaacg

SEQ ID NO: 11 - protein, 698 aa, Artificial Sequence
  STF2.4XTIBT*
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLADPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVT

SEQ ID NO: 12 - DNA, 2096 bp, Artificial Sequence
  STF2.4XTIBT*
atatggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttagcggacccaaatgcaaatccgaacgtagatccaaacgctaatccaaacgttaacgcaaacccgaatgcaaatccgaacgcgaacccggaatacctgaacaaaattcagaactctctgtccactgagtggtccccgtgtagcgtcaccgacccgaatgcaaacccaaacgtagatcctaacgcgaatccgaacgtgaacgcgaacccaaacgccaatcctaacgcaaacccagaatacctgaacaaaattcagaatagcctgagcactgaatggtccccatgctccgttaccgatccaaacgcgaacccgaatgttgacccgaatgctaacccgaacgtaaacgctaacccaaatgctaatccgaacgccaacccggagtatctgaacaaaattcaaaacagcctgtctactgaatggagcccatgctctgttacggacccgaacgcgaaccctaacgtcgacccgaatgccaacccaaacgtgaatgcgaaccctaatgcaaaccctaacgctaatccggaatatctgaacaagatccagaatagcctgtccaccgagtggtccccttgctctgtgacc

SEQ ID NO: 13 - protein, 595 aa, Artificial Sequence
  STF2delta.CSP
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLARGSSSNTRVLNELNYDNAGTNLYNELEMNYYGKQENWYSLKKNSRSLGENDDGNNNNGDNGREGKDEDKRDGNNEDNEKLRKPKHKKLKQPGDGNPDPNANPNVDPNANPNVDPNANPNVDPNANPNANPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEEYLNKIQNSLSTEWSPCSVTCGNGIQVRIKPGSANKPKDELDYENDIEKKICKMEKCSSVFNVVNSS

SEQ ID NO: 14 - DNA, 1785 bp, Artificial Sequence
  STF2delta.CSP
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcatggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttagcgagaggatccagcagcaacacccgtgttctgaacgaactgaactacgataacgctggtaccaatctgtacaacgaactggaaatgaactactacggtaaacaggaaaactggtacagcctgaaaaaaaacagcagatccctaggcgaaaacgacgacggcaacaacaacaacggtgataacggtcgcgaaggtaaagacgaagacaaacgcgacggcaacaacgaagacaacgaaaaacttcgcaaaccgaaacacaaaaaacttaagcagccaggggatggtaatccagatccgaacgcgaatccgaacgtagacccgaacgcaaacccgaacgtagacccgaacgcaaacccgaacgtagacccgaacgcgaatccgaacgcgaaccctaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacgcgaacccgaacaaaaacaatcagggtaatggccagggtcacaatatgccaaatgacccaaaccgaaatgtagatgaaaatgctaatgccaacaatgctgtaaaaaataataataacgaagaaccaagtgataagcacatagaagagtatttaaacaaaatacaaaattctctttcaactgaatggtccccatgtagtgtaacttgcggcaacggtattcaggtgcgcatcaagccgggctctgctaacaaacctaaggacgaactggattacgaaaacgatatcgaaaaaaagatctgtaagatggaaaagtgttcctctgtattcaacgtagttaactcttcg

SEQ ID NO: 15 - protein, 325 aa, Artificial Sequence
  STF2delta.TIBT*
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLADPNANPNVDPNANPNVNANPNANPNANPEYLNKIQNSLSTEWSPCSVT

SEQ ID NO: 16 - DNA, 975 bp, Artificial Sequence
  STF2delta.TIBT*
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcatggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttagcggacccgaatgcaaatccgaatgttgacccaaatgcgaacccaaatgtcaacgcgaaccctaacgctaacccaaacgctaacccggaatacctgaacaagatccagaacagcctgtccaccgaatggtccccgtgctctgtaacg

SEQ ID NO: 17 - protein, 469 aa, Artificial Sequence
  STF2delta.4XTIBT*
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLADPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVT

SEQ ID NO: 18 - DNA, 1407 bp, Artificial Sequence
  STF2delta.4XTIBT*
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcatggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttagcggacccaaatgcaaatccgaacgtagatccaaacgctaatccaaacgttaacgcaaacccgaatgcaaatccgaacgcgaacccggaatacctgaacaaaattcagaactctctgtccactgagtggtccccgtgtagcgtcaccgacccgaatgcaaacccaaacgtagatcctaacgcgaatccgaacgtgaacgcgaacccaaacgccaatcctaacgcaaacccagaatacctgaacaaaattcagaatagcctgagcactgaatggtccccatgctccgttaccgatccaaacgcgaacccgaatgttgacccgaatgctaacccgaacgtaaacgctaacccaaatgctaatccgaacgccaacccggagtatctgaacaaaattcaaaacagcctgtctactgaatggagcccatgctctgttacggacccgaacgcgaaccctaacgtcgacccgaatgccaacccaaacgtgaatgcgaaccctaatgcaaaccctaacgctaatccggaatatctgaacaagatccagaatagcctgtccaccgagtggtccccttgctctgtgacc

SEQ ID NO: 19 - DNA, 2982 bp, Artificial Sequence
  STF2.10XTIBT*His6
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttagcggacccaaatgctaatccaaatgtagatccaaacgcaaatccaaatgttaacgccaatccgaatgccaaccctaacgcgaacccggagtacctcaacaagattcagaatagcttgtctaccgaatggagcccatgctctgtgaccgatcctaatgcgaacccgaatgtggacccaaacgcgaatccgaacgttaacgctaacccgaatgcaaacccgaacgccaacccggaatatctgaacaaaattcagaactccctctctaccgagtggtcaccatgctcggtgaccgacccgaatgctaatcctaatgttgatccaaacgcaaatccgaacgttaatgctaacccaaacgctaacccaaatgcgaatccggagtacctgaacaaaattcagaacagcctgtctaccgagtggtccccgtgctctgtaaccgatccgaacgccaacccaaacgtagatccgaacgctaatccgaacgtgaacgctaacccgaacgcaaaccctaatgctaatccggagtatctgaacaaaatccaaaactccctgagcactgaatggtccccttgctctgtgactgatccgaacgcaaatccgaatgttgacccgaatgcaaacccgaatgttaacgcgaacccaaacgcgaaccctaacgcgaacccggaatacctgaataaaatccagaatagcctgtctactgagtggtcgccgtgctctgttaccgatccaaacgcaaacccaaacgtagaccctaatgctaacccaaatgtgaacgcgaatccgaatgcgaatccgaacgctaacccagaatatctcaataagatccaaaattctctcagcactgaatggagcccatgtagcgttactgacccaaatgcaaatccgaatgtggaccctaatgcgaacccaaatgttaatgcgaatccaaatgcgaatccgaacgcgaacccagaatacctcaataagattcagaatagcctctctaccgagtggagcccgtgtagcgttaccgacccaaatgcgaacccaaacgtggacccaaacgcgaatccaaacgtgaatgccaatccgaatgctaatccgaatgctaatccagagtatctcaacaagattcaaaactctctcagcaccgaatggagcccttgtagcgtgactgatccaaatgctaatccgaacgtagatccgaacgctaatccaaacgtcaatgcgaaccctaatgcaaacccaaacgctaatccagagtacttgaataagatccaaaactctctcagcactgagtggagcccatgttctgttactgatccaaacgcgaatccaaatgttgatccgaatgccaacccaaatgtgaatgctaacccgaatgcgaatcctaatgctaatccggagtatctcaataagattcaaaattcgctgtccaccgaatggagcccttgctctgtgacccaccaccatcatcaccattaatag

SEQ ID NO: 20 - protein, 992 aa, Artificial Sequence
  STF2.10XTIBT*His6
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLADPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTHHHHHH

SEQ ID NO: 21 - DNA, 2502 bp, Artificial Sequence
  STF2.10XBT*His6
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttagcgaatgctaaccctaatgcaaaccctaacgctaatcctgagtacctgaacaaaattcagaactctctgagcaccgaatggtctccatgttctgtgactaatgcgaacccgaatgcgaatccgaatgctaatccagagtatctgaacaagattcagaacagcctgtctaccgagtggagcccgtgtagcgttactaatgctaacccaaatgcgaatccaaatgctaacccggaatatctgaataagatccaaaatagcctgagcactgaatggagcccatgtagcgtgactaatgcgaatccaaacgccaatccaaatgcaaacccagaatacctcaataaaatccaaaacagcctgtctactgagtggtccccgtgctctgtgaccaatgcgaaccctaacgcgaacccgaacgctaatcctgaatatctgaacaagatccagaactctttgagcaccgaatggagcccatgtagcgtcactaatgctaacccaaacgcaaatcctaatgcaaatccggaatacctgaacaaaatccagaactccctgagcaccgagtggtccccttgctctgtaaccaacgctaacccgaacgcaaacccgaacgcgaatcctgagtacttgaacaaaattcaaaactcactgtctacggagtggagcccgtgctccgttactaacgcgaatccaaacgctaatccaaatgcgaacccagaatatctgaacaagatccaaaacagcctgtctactgagtggagcccgtgcagcgttaccaacgccaatccaaacgccaaccctaacgcgaacccggaatacctgaataaaatccaaaattctctgagcacggaatggtccccatgctctgtgaccaacgcgaatccgaacgctaacccgaacgcaaatccagagtacctgaacaaaattcagaactctctgtctacggagtggtctccgtgctccgttacccaccaccatcatcaccattaatag

SEQ ID NO: 22 - protein, 833 aa, Artificial Sequence
  STF2.10XBT*His6
HMAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLANANPNANPNANPEYLNKIQNSLSTEWSPCSVTNANPNANPNANPEYLNKIQNSLSTEWSPCSVTNANPNANPNANPEYLNKIQNSLSTEWSPCSVTNANPNANPNANPEYLNKIQNSLSTEWSPCSVTNANPNANPNANPEYLNKIQNSLSTEWSPCSVTNANPNANPNANPEYLNKIQNSLSTEWSPCSVTNANPNANPNANPEYLNKIQNSLSTEWSPCSVTNANPNANPNANPEYLNKIQNSLSTEWSPCSVTNANPNANPNANPEYLNKIQNSLSTEWSPCSVTNANPNANPNANPEYLNKIQNSLSTEWSPCSVTHHHHHH

SEQ ID NO: 23 - DNA, 2622 bp, Artificial Sequence
  STF2.10XTIT*His6
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaaccacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgagcctgttagcggaccctaatgcgaacccaaatgttgacccaaacgctaatccaaacgtagaatatctcaataaaatccaaaactcgctgtccactgaatggagcccgtgctccgtgaccgatccgaacgctaacccgaacgtagatccaaacgcaaatccgaacgtggagtacctgaataagatccagaacagcctgtccaccgaatggtccccgtgttcggttaccgacccaaacgcgaaccctaacgttgacccgaacgccaacccgaatgtggaatacctgaacaagatccaaaattccctgtcgactgaatggtcgccatgcagcgtgactgacccaaacgctaatcctaacgtagatccgaatgcaaaccctaatgttgagtacttgaacaagattcagaatagccttagcaccgagtggtccccatgtagcgttactgacccaaatgccaatccaaatgtggacccgaatgcgaacccaaatgttgaatatctcaacaaaattcaaaattctctcagcactgagtggagcccatgcagcgtgactgatccgaatgctaatccgaatgttgatccaaatgcgaatccaaacgttgagtatctcaataaaatccagaacagcctcagcacggagtggagcccgtgttctgttaccgatcctaacgcaaaccctaacgtagatccgaatgctaacccgaatgtggaatatctgaacaagatccagaatagcctgtcaacggagtggtctccttgcagcgttaccgacccgaacgccaatccgaacgtggaccctaacgctaacccaaacgttgagtacctgaataaaatccagaactctctttctactgaatggtccccttgctccgttaccgatccaaacgcgaacccgaacgttgacccgaacgcgaatccgaatgttgaatacctcaacaaaatccaaaactctctgtcgaccgagtggtccccgtgttcggtgactgacccaaacgcaaacccaaacgtagacccgaacgctaatccgaacgtagagtaccttaacaaaattcagaactcgctgtccacggaatggtctccgtgctccgtgactcaccaccatcatcaccattaatag

SEQ ID NO: 24 - protein, 872 aa, Artificial Sequence
  STF2.10xTIT*His6
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLADPNANPNVDPNANPNVEYLNKIQNSLSTEWSPCSVTDPNANPNVDPNANPNVEYLNKIQNSLSTEWSPCSVTDPNANPNVDPNANPNVEYLNKIQNSLSTEWSPCSVTDPNANPNVDPNANPNVEYLNKIQNSLSTEWSPCSVTDPNANPNVDPNANPNVEYLNKIQNSLSTEWSPCSVTDPNANPNVDPNANPNVEYLNKIQNSLSTEWSPCSVTDPNANPNVDPNANPNVEYLNKIQNSLSTEWSPCSVTDPNANPNVDPNANPNVEYLNKIQNSLSTEWSPCSVTDPNANPNVDPNANPNVEYLNKIQNSLSTEWSPCSVTDPNANPNVDPNANPNVEYLNKIQNSLSTEWSPCSVTHHHHHH

SEQ ID NO: 25 - protein, 405 aa, Artificial Sequence
  P.falciparum CSP
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 26 - protein, 397 aa, Artificial Sequence
  P.falciparum CSP
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 27 - protein, 397 aa, Artificial Sequence; identical to SEQ ID NO: 26
  P.falciparum CSP

SEQ ID NO: 28 - protein, 442 aa, Artificial Sequence
  P.falciparum CSP
MMRKLAILSVSSFLFVEALFQEYQCYGSSSNTRVLNELNYDNAGTNLYNELEMNYYGKQENWYSLKKNSRSLGENDDGDNDNGNNNNGNNNNGDNGREGKDEDKRDGNNEDNEKLRKPKHKKLKQPGDGNPDPNANPNVDPNANPNVDPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEQYLKKIQNSLSTEWSPCSVTCGNGIQVRIKPGSADKPKDQLDYENDIEKKICKMEKCSSVFNVVNSSIGLIMVLSFLFLN

SEQ ID NO: 29 - protein, 424 aa, Artificial Sequence
  P.falciparum CSP
MMRKLAILSVSSFLFVEALFQEYQCYGSSSNTRVLNELNYDNAGTNLYNELEMNYYGKQENWYSLKKNSRSLGENDDGNNNNGDNNREGKDEDKRDGNNEDNETLRKPKHKKLKQPGDGNPDPNANPNVDPNANPNVDPNANPNVDPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEQYLKKIQNSLSTEWSPCSVTCGNGIQVRIKPGSANKPKDELDYENDIEKKICKMEKCSSVFNVVNSSIGLIMVLSFLFLN

SEQ ID NO: 30 - protein, 315 aa, Artificial Sequence
  P.falciparum CSP
EALFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPADGNPDPNANPNVDPNANPNVDPNANPNVDPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNANPNANPNANPNANPNKNNQGNGQGHNMPNDPNRNVDENANGNNAVKNNNNEEPSDQHIEKYL

SEQ ID NO: 31 - protein, 412 aa, Artificial Sequence
  P.falciparum CSP
MMRKLAILSVSSFLFVEALFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPGDGNPDPNANPNVDPNANPNVDPNANPNVDPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEQYLKKIKNSISTEWSPCSVTCGNGIQVRIKPGSANKPKDELDYENDIEKKICKMEKCSSVFNVVNSSIGLIMVLSFLFLN

SEQ ID NO: 32 - protein, 408 aa, Artificial Sequence
  P.falciparum CSP
MMRKLAILSVSSFLFVEALFQEYQCYGSSSNTRVLNELNYDNAGTNLYNELEMNYYGKQENWYSLKKNSRSLGENDDGNNNNGDNGGEGKDEDKRDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNNPNRNVDENANANNAVKNNNNEEPSDKHIEQYLKKIQNSLSTEWSPCSVTCGNGIQVRIKPGSAGKSKDELDYENDIEKKICKMEKCSSVFNVVNSSIGLIMVLSFLFLN

SEQ ID NO: 33 - protein, 418 aa, Artificial Sequence
  Pf CS consensus
MMRKLAILSVSSFLFVEALFQEYQCYGSSSNTRVLNELNYDNAGTNLYNELEMNYYGKQENWYSLKKNSRSLGENDDGNNNNGDNGREGKDEDKRDGNNEDNEKLRKPKHKKLKQPADGNPDPNANPNVDPNANPNVDPNANPNVDPNANPNANPNANPNANPNANPNANPNANPNANPNANPNANPNANPNANPNANPKQILMQTMQTQMQTQTQTQCKSNANPNANPNANPNANPNANPNANPNANPNANPNANPNANPNANPNANPNANPNANPNANPNANPNANPNANPNKNNQGNGQGHNMPNDPNRNVDENANANNAVKNNNNEEPSDKHIEQYLKKIQNSLSTEWSPCSVTCGNGIQVRIKPGSANKPKDELDYENDIEKKICKMEKCSSVFNVVNSSIGLIMVLSFLFLN

SEQ ID NO: 34 - protein, 20 aa, Artificial Sequence
  T*
EYLNKIQNSLSTEWSPCSVT

SEQ ID NO: 35 - protein, 8 aa, Artificial Sequence
  T-cell Repeat
NANPNVDP

SEQ ID NO: 36 - protein, 4 aa, Artificial Sequence
  T-cell Repeat
NANP

SEQ ID NO: 37 - protein, 16 aa, Artificial Sequence
  T1 epitope
DPNANPNVDPNANPNV

SEQ ID NO: 38 - protein, 12 aa, Artificial Sequence
  B cell epitope
NANPNANPNANP

SEQ ID NO: 39 - protein, 378 aa, Artificial Sequence
  P. vivax CSP
MKNFILLAVSSILLVDLFPTHCGHNVDLSKAINLNGVNFNNVDASSLGAAHVGQSASRGRGLGENPDDEEGDAKKKKDGKKAEPKNPRENKLKQPGDRADGQPAGDRADGQPAGDRADGQPAGDRAAGQPAGDRADGQPAGDRADGQPAGDRADGQPAGDRADGQPAGDRAAGQPAGDRAAGQPAGDRADGQPAGDRAAGQPAGDRADGQPAGDRAAGQPAGDRADGQPAGDRAAGQPAGDRAAGQPAGDRAAGQPAGDRAAGQPAGNGAGGQAAGGNAGGGQGQNNEGANAPNEKSVKEYLDKVRATVGTEWTPCSVTCGVGVRVRRRVNAANKKPEDLTLNDLETDVCTMDKCAGIFNVVSNSLGLVILLVLALFN

SEQ ID NO: 40 - protein, 739 aa, Artificial Sequence
  P. vivax CSP
MSISEHTGNEVSIAERERKEEAIGADKNDKDVPPTNGESISLRMEKMKGEANDEGSVEVGNCSPVVENELGSSVLCVGCSGEASNIDGGTTNISGEENPRTEADGDKKAQPEGILHEAKKVKNGVDAETKSNAQVSSNGTYVENAPNVEASPNDNACGEGHSNTHVVDDGPPNAAPTSNGELIANSGGALNVEEDAPLDEGNFSADDRPEENAESTGSFMLEEDLNLSRRAYRNFHICSIFIHGTLLLMVILLMGILFHDFMKPSSVSQKEKIMTYFCGLLLSMLGLHLCLNLYMSLVLLRQAEVSKVLKSVEAKIHVIVLVYFSMCAYIYFFEGKTYPISSIFSFTIILAIIYYFMPIFLYIVLRILFIIVILILIFMKRKSPTPKKILKKLKIMKYMEYRKYCEEEACFRSAYFTNWRELNGEGGSTPQEGVTGTREAMTTTAVEAGTGIAASGGENKGDDIAAVEPTSNCNADGNTISTATSCVRGSSANERPTRSGNSSTRSNLERHLFYDRAGGATGRGGGSSNRGGAQASDREGGNQNGRDNARDNARCNARDNARDNARDNARDNARDNARDNARCNARDNARCNARDNARDNARDNARDNARDNARQHPPPSYDNSREVPNASDEANGEPNKDGKSAAVFEYFQKVLKKKKNALESDNAQVHENYAEENSFHINIESSDYVCSICCVEYLNDDDICILPCNYLHYYHKECIFTWLKRNNDCPLCRKCIGKI

SEQ ID NO: 41 - protein, 235 aa, Artificial Sequence
  Pv VK210 CS Consensus
NGVNFNNVDASSLGAAHVGQSASRGRGLGENPDDEEGDAKKKKDGKKAEPKNPRENKLKQPGDRADGQPAGDRADGQPAGDRADGQPAGDRAGQPAGDRAGQPAGDRADGQPAGDRADGQPAGDRADGQPAGDRAGQPAGDRAAGQPAGDRAGQPAGDRAGQPAGDRAGQPAGDRAGQPAGDRAGQPAGDRAGQPAGDRAAGQPAGDRAAGQPAGDRAAGQAGAAGQAAGGNAGG

SEQ ID NO: 42 - protein, 237 aa, Artificial Sequence
  P. vivax CSP
KLKQPEDGAGNQPGANGAGNQPGANGAGNQPGANGAGNQPGANGAGNQPGANGAGNQPGANGAGNQPGANGAGNQPGANGAGNQPGANGAGNQPGANGAGNQPGANGAGNQPGANGAGNQPGANGAGNQPGANGAGNQPGANGAGNQPGANGAGNQPGANGAGNQPGANGAGNQPGANGAGGQAAGGNAANKKAGDAGAGQGQNNEGANAPNEKSVKEYLDKVRATVGTEWTPCSVT

SEQ ID NO: 43 - protein, 405 aa, Artificial Sequence
  P. vivax CSP
DLSKAINLNGVGFNNVDASSLGAAHVGQSASRGRGLGENPDDEEGDAKKKKDGKKAEPKNPRENKLKQPEDGAGNQPGANGAGNQPGANGAGNQPGANGADDQPGANGAGNQPGANGAGNQPGANGAGNQPGANGAGNQPGANGADDQPGANGAGNQPGANGAGNQPGANGAGNQPGANGAGNQPGANGADDQPGANGAGNQPGANGAGNQPGANGAGNQPGANGAGDQPGANGAGNQPGANGAGNQPGANGAGNQPGANGAGNQPGANGAGNQPGANGAGNQPGANGAGNQPGANGAGGQAAGGNAGGQGQNNEGANAPNEKSVKEYLDKVRATVGTEWTPCSVTCGVGVRVRRRVNAANKKPEDLTLNDLETDVCTMDKCAGIFNVVSNSLGLVILLVLALFN

SEQ ID NO: 44 - protein, 354 aa, Artificial Sequence
  P. vivax CSP
THCGHNVDLSKAINLNGVGFNNVDASSLGAAHVGQSASRGRGLGENPDDEEGDAKKKKDGKKAEPKNPRENKLKQPEDGAGNQPGANGAGNQPGANGAGNQPGANGAGNQPGANGAGNQPGANGAGNQPGANGAGNQPGANGAGNQPGANGADDQPGANGAGNQPGANGAGNQPGANGAGNQPGANGAGDQPGANGAGNQPGANGAGDQPGANGAGNQPGANGAGNQPGANGAGNQPGANGAGNQPGANGAGNQPGANGAGGQAAGGNAANKKAGDAGAGQGQNNEGANAPNEKSVKEYLDKVRATVGTEWTPCSVTCGVGVRVRRRVNAANKKPEDLTLNDLETDVCTMDKCA

SEQ ID NO: 45 - protein, 228 aa, Artificial Sequence
  P. vivax CSP
AEPKNPRENKLKQPEDGAGNQPGANGAGDQPGANGAGNQPGANGAGNQPGANGAGNQPGANGAGNQPGANGAGNQPGANGAGDQPGANGAGNQPGANGAGNQPGANGAGNQPGANGAGNQPGANGAGNQPGANGAGDQPGANGAGNQPGANGAGNQPGANGAGNQPGANGAGNQPGANGAGGQAAGGNAANKKAGDAGAGQGQNNEGANAPNEKSVKEYLDKVRATVG

SEQ ID NO: 46 - protein, 237 aa, Artificial Sequence
  P. vivax CSP
AEPKNPRENKLKQPEDGAGNQPGANGAGDQPGANGAGNQPGANGAGNQPGANGAGNQPGANGAGNQPGANGAGNQPGANGAGDQPGANGAGNQPGANGAGNQPGANGAGNQPGANGAGDQPGANGAGNQPGANGAGDQPGANGAGNQPGANGAGNQPGANGAGNQPGANGAGNQPGANGAGNQPGANGAGGQAAGGNAANKKAGDAGAGQGQNNEGANAPNEKSVKEYLDKVRATVG

SEQ ID NO: 47 - protein, 228 aa, Artificial Sequence
  P. vivax CSP
AEPKNPRENKLKQPEDGAGDQPGANGAGDQPGANGAGNQPGANGAGNQPGANGAGNQPGANGAGNQPGANGAGNQPGANGAGDQPGANGAGNQPGANGAGNQPGANGAGNQPGANGAGDQPGANGAGNQPGANGAGDQPGANGAGNQPGANGAGNQPGANGAGNQPGANGAGNQPGANGAGGQAAGGNAANKKAGDAGAGQGQNNEGANAPNEKSVKEYLDKVRATVG

SEQ ID NO: 48 - protein, 237 aa, Artificial Sequence
  P. vivax CSP
AEPKNPRENKLKQPEDGAGDQPGANGAGNQPGANGAGNQPGANGAGNQPGANGAGNQPGANGAGNQPGANGAGNQPGANGAGDQPGANGAGNQPGANGAGDQPGANGAGNQPGANGAGDQPGANGAGNQPGANGAGDQPGANGAGNQPGANGAGNQPGANGAGNQPGANGAGNQPGANGAGNQPGANGAGGQAAGGNAANKKAGDAGAGQGQNNEGANAPNEKSVKEYLDKVRATVG

SEQ ID NO: 49 - protein, 237 aa, Artificial Sequence
  P. vivax CSP
AEPKNPRENKLKQPEDGAGNQPGANGAGNQPGANGAGNQPGANGAGNQPGANGAGNQPGANGAGNQPGANGAGNQPGANGAGDQPGANGAGNQPGANGAGDQPGANGAGNQPGANGAGDQPGANGAGNQPGANGAGDQPGANGAGNQPGANGAGNQPGANGAGNQPGANGAGNQPGANGAGNQPGANGAGGQAAGGNAANKKAGDAGAGQGQNNEGANAPNEKSVKEYLDKVRATVG

SEQ ID NO: 50 - protein, 228 aa, Artificial Sequence
  P. vivax CSP
AEPKNPRENKLKQPEDGAGDQPGANGAGNQPGANGAGNQPGANGAGNQPGANGAGNQPGANGAGNQPGANGAGNQPGANGADDQPGANGAGNQPGANGAGNQPGANGAGNQPGANGAGDQPGANGAGNQPGANGAGDQPGANGAGNQPGANGAGNQPGANGAGNQPGANGAGNQPGANGAGGQAAGGNAANKKAGDAGAGQGQNNEGANAPNEKSVKEYLDKVRATVG

SEQ ID NO: 51 - protein, 247 aa, Artificial Sequence
  P. vivax CSP
AEPKNPRENKLKQPEDGAGDQPGANGAGNQPGANGAGNQPGANGAGNQPGANGAGNQPGANGAGNQPGANGAGNQPGANGADDQPGANGAGNQPGANGAGNQPGANGAGNQPGANGAGDQPGANGAGNQPGANGAGDQPGANGAGNQPGANGAGNQPGANGAGNQPGANGAGNQPGANGAGGQAAGGNAANKKAGDAGAGGQAAGGNAANKKAGDAGAGQGQNNEGANAPNEKSVKEYLDKVRATVG

SEQ ID NO: 52 - protein, 247 aa, Artificial Sequence
  P. vivax CSP
AEPKNPRENKLKQPEDGAGNQPGANGAGNQPGANGAGNQPGANGAGNQPGANGAGNQPGANGAGNQPGANGAGNQPGANGADDQPGANGAGNQPGANGAGNQPGANGAGNQPGANGAGDQPGANGAGNQPGANGAGDQPGANGAGNQPGANGAGNQPGANGAGNQPGANGAGNQPGANGAGGQAAGGNAANKKAGDAGAGGQAAGGNAANKKAGDAGAGQGQNNEGANAPNEKSVKEYLDKVRATVG

SEQ ID NO: 53 - protein, 239 aa, Artificial Sequence
  P. vivax CSP
KKDGKKAEPKNPRENKLKQPEDGAGNQPGANGAGNQPGANGAGNQPGANGAGNQPGANGAGNQPGANGAGNQPGANGAGNQPGANGAGNQPGANGADDQPGANGAGNQPGANGAGNQPGANGAGNQPGANGAGDQPGANGAGNQPGANGAGDQPGANGAGNQPGANGAGNQPGANEAGNQPGANGAGNQPGANGAGNQPGANGAGGQAAGGNAANKKAGDAGAGQGQNNEGANAPNEKS

SEQ ID NO: 54 - protein, 237 aa, Artificial Sequence
  Pv VK247 CS Consensus
AEPKNPRENKLKQPEDGAGNQPGANGAGNQPGANGAGNQPGANGAGNQPGANGAGNQPGANGAGNQPGANGAGNQPGANGAGDQPGANGAGNQPGANGAGNQPGANGAGNQPGANGAGDQPGANGAGNQPGANGAGDQPGANGAGNQPGANGAGNQPGANGAGNQPGANGAGNQPGANGAGNQPGANGAGGQAAGGNAANKKAGDAGAGQGQNNEGANAPNEKSVKEYLDKVRATVG

SEQ ID NO: 55 - protein, 20 aa, Artificial Sequence
  T-cell epitope T*
EYLDKVRATVGTEWTPCSVT

SEQ ID NO: 56 - protein, 441 aa, Artificial Sequence
  P. malarie CSP
MKKLSVLAISSFLIVDFLFPGYHHNSNSTKSRNLSELCYNNVDTKLFNELEVRYSTNQDHFYNYNKTIRLLNENNNEKDGNVTNERKKKPTKAVENKLKQPPGDDDGAGNDEGNDAGNDAGNAAGNAAGNAAGNAAGNDAGNAAGNDAGNAAGNDAGNAAGNAAGNAAGNAAGNAAGNAAGNAAGNAAGNAAGNDAGNAAGNAAGNAAGNAAGNAAGNAAGNAAGNAAGNAAGNAAGNAAGNDAGNAAGNAAGNAAGNAAGNAAGNAAGNAAGNAAGNAAGNAAGNAAGNAAGNAAGNAAGNAAGNAAGNAAGNAAGNAAGNAAGNEKAKNKDNKVDANTNKKDNQGENNDSSNGPSEEHIKNYLESIRNSITEEWSPCSVTCGSGIRARRKVDAKNKKPAELVLSDLETEICSLDKCSSIFNVVSNSLGIVLVLVLILFH

SEQ ID NO: 57 - protein, 441 aa, Artificial Sequence; identical to SEQ ID NO: 56
  P. malarie CSP

SEQ ID NO: 58 - protein, 429 aa, Artificial Sequence
  P. malarie CSP
MKKLSVLAISSFLIVDFLFPGYHHNSNSTKSRNLSELCYNNVDTKLFNELEVRYSTNQDHFYNYNKTIRLLNENNNEKDGNVTNERKKKPTKAVENKLKQPPGDDDGAGNDAGNDAGNDAGNAAGNAAGNAAGNAAGNAAGNAAGNAAGNAAGNAAGNAAGNDAGNAAGNAAGNAAGNAAGNAAGNDAGNAAGNAAGNAAGNAAGNAAGNAAGNAAGNAAGNAAGNAAGNDAGNAAGNAAGNAAGNAAGNAAGNAAGNAAGNAAGNAAGNAAGNAAGNAAGNAAGNAAGNAAGNAAGNAAGNAAGNAAGNAAGNEKAKNKDNKVDANTNKKDNQEENNDSSNGPSEEHIKNYLESIRNSITEEWSPCSVTCGSGIRARRKVGAKNKKPAELVLSDLETEICSLDKCSSIFNVVSNSLGIVLVLVLILFH

SEQ ID NO: 59 - protein, 429 aa, Artificial Sequence
  P. malarie CSP
MKKLSVLAISSFLIVDFLFPGYHHNSNSTKSRNLSELCYNNVDTKLFNELEVRYSTNQDHFYNYNKTIRLLNENNNEKDGNVTNERKKKPTKAVENKLKQPPGDDDGAGNDAGNDAGNDAGNAAGNAAGNAAGNAAGNAAGNAAGNAAGNAAGNAAGNAAGNDAGNAAGNDAGNAAGNAAGNAAGNAAGNAAGNDAGNAAGNAAGNAAGNAAGNAAGNAAGNAAGNAAGNAAGNAAGNDAGNAAGNAAGNAAGNAAGNAAGNAAGNAAGNAAGNAAGNAAGNAAGNAAGNAAGNAAGNAAGNAAGNAAGNAAGNEKAKNKDNKVDANTNKKDNQEENNDSSNGPSEEHIKNYLESIRNSITEEWSPCSVTCGSGIRARRKVDAKNKKPAELVLSDLETEICSLDKCSSIFNVVSNSLGIVLVLVLILFH

SEQ ID NO: 60 - protein, 421 aa, Artificial Sequence
  P. malarie CSP
MKKLSVLAISSFLIVDFLFPGYHHNSNSTKSRNLSELCYNNVDTKLFNELEVRYSTNQDHFYNYNKTIRLLNENNNEKDGNVTNERKKKPTKAVENKLKQPPGDDDGAGNDAGNDAGNAAGNAAGNAAGNAAGNAAGNAAGNDAGNAAGNAAGNAAGNAAGNAAGNAAGNDAGNAAGNAAGNAAGNAAGNDAGNAAGNAAGNAAGNAAGNAAGNAAGNAAGNAAGNAAGNAAGNDAGNAAGNAAGNAAGNAAGNAAGNAAGNAAGNAAGNAAGNAVGNAAGNAAGNAAGNAAGNAAGNAAGNAAGNEKAKNKDNKVDANTNKKDNQEENNDSSNGPSEEHIKNYLESIRNSITEEWSPCSVTCGSGIRARRKVDAKNKKPAELVLSDLETEICSLDKCSSIFNVVSNSLGIVLVLVLILFH

SEQ ID NO: 61 - protein, 433 aa, Artificial Sequence
  P. malarie CSP
MKKLSVLAISSFLIVDFLFPGYHHNSNSTKSRNLSELCYNNVDTKLFNELEVRYSTNQDHFYNYNKTIRLLNENNNEKDGNVTNERKKKPTKAVENKLKQPPGDDDGAGNDAGNDAGNAAGNAAGNAAGNAAGNAAGNDAGNAAGNAAGNAAGNAAGNAAGNAAGNDAGNAAGNAAGNAAGNAAGNAAGNDAGNAAGNAAGNAAGNAAGNAAGNAAGNAAGNAAGNAAGNAAGNDAGNAAGNAAGNAAGNAAGNAAGNAAGNAAGNAAGNAAGNAAGNAAGNAAGNAAGNAAGNAAGNAAGNAAGNAAGNAAGNAAGNEKAKNKDNKVDANTNKKDNQEENNDSSNGPSEEHIKNYLESIRNSITEEWSPCSVTCGSGIRARRKVDAKNKKPAELVLSDLETEICSLDKCSSTFNVVSNSLGIVLVLVLILFH

SEQ ID NO: 62 - protein, 429 aa, Artificial Sequence; identical to SEQ ID NO: 59
  P. malarie CSP

SEQ ID NO: 63 - protein, 425 aa, Artificial Sequence
  P. malarie CSP
MKKLSVLAISSFLIVDFLFPGYHHNSNSTKSRNLSELCYNNVDTKLFNELEVRYSTNQDHFYNYNKTIRLLNENNNEKDGNVTNERKKKPTKAVENKLKQPPGDDDGAGNDAGNDAGNAAGNAAGNAAGNAAGNAAGNDAGNAAGNAAGNAAGNAAGNAAGNAAGNDAGNAAGNAAGNAAGNAAGNAAGNDAGNAAGNAAGNAAGNAAGNAAGNAAGNAAGNAAGNAAGNAAGNDAGNAAGNAAGNAAGNAAGNAAGNAAGNAAGNAAGNAAGNAAGNAAGNAAGNAAGNAAGNAAGNAAGNAAGNAAGNEKAKNKDNKVDANTNKKDNQGENNDSSNGPSEEHIKNYLESIRNSITEEWSPCSVTCGSGIRARRKVDAKNKKPAELVLSDLETEICSLDKCSSIFNVVSNSLGIVLVLVLILFH

SEQ ID NO: 64 - protein, 425 aa, Artificial Sequence
  P. malarie CSP
MKKLSVLAISSFLIVDFLFPGYHHNSNSTKSRNLSELCYNNVDTKLFNELEVRYSTNQDHFYNYNKTIRLLNENNNEKDGNVTNERKKKPTKAVENKLKQPPGDDDGAGNDAGNDAGNAAGNAAGNAAGNAAGNAAGNAAGNAAGNAAGNAAGNAAGNDAGNAAGNDAGNAAGNAAGNAAGNAAGNAAGNDAGNAAGNAAGNAAGNAAGNAAGNAAGNAAGNAAGNAAGNAAGNDAGNAAGNAAGNAAGNAAGNAAGNAAGNAAGNAAGNAAGNAAGNAAGNAAGNAAGNAAGNAAGNAAGNAAGNAAGNEKAKNKDNKVDANTNKKDNQEENNDSSNGPYEEHIKNYLESIRNSITEEWSPCSVTCGSGIRARRKVDAKNKKPAELVLSDLETEICSLDKCSSIFNVVSNSLGIVLVLVLILFH

SEQ ID NO: 65 - protein, 429 aa, Artificial Sequence; identical to SEQ ID NO: 59
  P. malarie CSP

SEQ ID NO: 66 - protein, 429 aa, Artificial Sequence
  P. malarie CSP
MKKLSVLAISSFLIVDFLFPGYHHNSNSTKSRNLSELCYNNVDTKLFNELEVRYSTNQDHFYNYNKTIRLLNENNNEKDGNVTNERKKKPTKAVENKLKQPPGDDDGAGNDAGNDAGNDAGNAAGNAAGNAAGNAAGNAAGNAAGNAAGNAAGNAAGNAAGNDAGNAAGNDAGNAAGNAAGNAAGNAAGNAAGNDAGNAAGNAAGNAAGNAAGNAAGNAAGNAAGNAAGNAAGNAAGNDAGNAAGNAAGNAAGNAAGNAAGNAAGNAAGNAAGNAAGNAAGNAAGNAAGNAAGNAAGNAAGNAAGNAAGNAAGNEKAKNKDNKVDANTNKKDNQEENNDSSNGPSEEHIKNYLESIRNSITEEWSPCSVTCGSGIRARREVDAKNKKPAELVLSDLETEICSLDKCSSIFNVVSNSLGIVLVLVLILFH

SEQ ID NO: 67 - protein, 429 aa, Artificial Sequence; identical to SEQ ID NO: 66
  P. malarie CSP

SEQ ID NO: 68 - protein, 421 aa, Artificial Sequence
  P. malarie CSP
MKKLSVLAISSFLIVDFLFPGYHHNSNSTKSRNLSELCYNNVDTKLFNELEVRYSTNQDHFYNYNKTIRLLNENNNEKDGNVTNERKKKPTKAVENKLKQPPGDDDGAGNDAGNDAGNAAGNAAGNAAGNDAGNAAGNAAGNAAGNAAGNAAGNAAGNDAGNAAGNAAGNAAGNAAGNAAGNDAGNAAGNAAGNAAGNAAGNAAGNAAGNAAGNAAGNAAGNAAGNDAGNAAGNAAGNAAGNAAGNAAGNAAGNAAGNAAGNAAGNAAGNAAGNAAGNAAGNAAGNAAGNAAGNAAGNAAGNAAGNEKAKNKDNKVDANTNKKDNQEENNDSSNGPSEEHIKNYLESIRNSITEEWSPCSVTCGSGIRARRKVDAKNKKPAELVLSDLETEICSLDKCSSIFNVVSNSLGIVLVLVLILFH

SEQ ID NO: 69 - protein, 421 aa, Artificial Sequence; identical to SEQ ID NO: 68
  P. malarie CSP

SEQ ID NO: 70 - protein, 429 aa, Artificial Sequence
  P. malarie CSP
MKKLSVLAISSFLIVDFLFPGYHHNSNSTKSRNLSELCYNNVDTKLFNELEVRYSTNQDHFYNYNKTIRLLNENNNEKDGNVTNERKKKPTKAVENKLKQPPGDDDGAGNDAGNDAGNAAGNAAGNAAGNAAGNAAGNDAGNAAGNAAGNAAGNAAGNAAGNDAGNAAGNDAGNAAGNAAGNAAGNAAGNAAGNDAGNAAGNAAGNAAGNAAGNAAGNAAGNAAGNAAGNAAGNAAGNDAGNAAGNAAGNAAGNAAGNAAGNAAGNAAGNAAGNAAGNAAGNAAGNAAGNAAGNAAGNAAGNAAGNAAGNAAGNEKAKNKDNKVDANTNKKDNQEENNDSSNGPSEEHIKNYLESIRNSITEEWSPCSVTCGSGIRTRRKVDAKNKKPAELVLSDLETEICSLDKCSSIFNVVSNSLGIVLVLVLILFH

SEQ ID NO: 71 - protein, 425 aa, Artificial Sequence
  P. malarie CSP
MKKLSVLAISSFLIVDFLFPGYHHNSNSTKSRNLSELCYNNVDTKLFNELEVRYSTNQDHFYNYNKTIRLLNENNNEKDGNVTNERKKKPTKAVENKLKQPPGDDDGAGNDAGNDAGNAAGNAAGNAAGNAAGNAAGNAAGNAAGNAAGNAAGNAAGNDAGNAAGNDAGNAAGNAAGNAAGNAAGNAAGNDAGNAAGNAAGNAAGNAAGNAAGNAAGNAAGNAAGNAAGNAAGNDAGNAAGNAAGNAAGNAAGNAAGNAAGNAAGNAAGNAAGNAAGNAAGNAAGNAAGNAAGNAAGNAAGNAAGNAAGNEKAKNKDNKVDANTNKKDNQEENNDSSNGPSEEHIKNYLESIRNSITEEWSPCSVTCGGGIRARRKVDAKNKKPAELVLSDLETEICSLDKCSSIFNVVSNSLGIVLVLVLILFH

SEQ ID NO: 72 - protein, 429 aa, Artificial Sequence; identical to SEQ ID NO: 59
  Pm CS consensus

SEQ ID NO: 73 - protein, 20 aa, Artificial Sequence
  T-cell T*
NYLESIRNSITEEWSPCSVT

SEQ ID NO: 74 - DNA, 1218 bp, Artificial Sequence
  P. falciparum CSP
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaaccccaatgcaaatcctaatgcaaacccaaatgcaaacccaaacgtagatcctaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacagtgctgtaaaaaataataataacgaagaaccaagtgataagcacataaaagaatatttaaacaaaatacaaaattctctttcaactgaatggtccccatgtagtgtaacttgtggaaatggtattcaagttagaataaagcctggctctgctaataaacctaaagacgaattagattatgcaaatgatattgaaaaaaaaatttgtaaaatggaaaaatgttccagtgtgtttaatgtcgtaaatagttcaataggattaataatggtattatccttcttgttccttaattag

SEQ ID NO: 75 - DNA, 1194 bp, Artificial Sequence
  P. falciparum CSP
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataacgaagacaacgagaaattaaggaaaccaaaacataaaaaattaaagcaaccagcggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaaccccaatgcaaatcctaatgcaaacccaaatgcaaacccaaacgtagatcctaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacagtgctgtaaaaaataataataacgaagaaccaagtgataagcacataaaagaatatttaaacaaaatacaaaattctctttcaactgaatggtccccatgtagtgtaacttgtggaaatggtattcaagttagaataaagcctggctctgctaataaacctaaagacgaattagattatgcaaatgatattgaaaaaaaaatttgtaaaatggaaaaatgttccagtgtgtttaatgtcgtaaatagttcaataggattaataatggtattatccttcttgttccttaattag

SEQ ID NO: 76 - DNA, 1387 bp, Artificial Sequence
  P. falciparum CSP
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggagataatgataatggaaataataataatggaaataataataatggagataatggtcgtgaaggtaaagatgaagataaaagagatggaaataacgaagacaacgagaaattaaggaaaccaaaacataaaaaattaaagcaaccaggggatggtaatcctgatccaaatgccaacccaaatgtagatccaaatgccaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaacgtagatcctaatgcaaatccaaatgcaaacccaaatgcaaacccaaacgcaaacccaaatgcaaatcctaatgcaaatcctaatgcaaatcctaatgccaatccaaatgcaaatccaaatgcaaacccaaacgcaaaccccaatgcaaatcctaatgccaatccaaatgcaaatccaaatgcaaacccaaacgcaaaccccaatgcaaatcctaatgccaatccaaatgcaaatccaaatgcaaaccccaatgcaaatcctaatgccaatccaaatgcaaatccaaatgcaaacccaaatgcaaacccaaatgcaaatcctaataaaaacaatcaaggtaatggacaaggtcacaatatgccaaatgacccaaaccgaaatgtagatgaaaatgctaatgccaacaatgctgtaaaaaataataataacgaagaaccaagtgataagcacatagaacaatatttaaagaaaatacaaaattctctttcaactgaatggtccccatgtagtgtaacttgtggaaatggtattcaagttagaataaagcctggctctgctgataaacctaaagaccaattagattatgaaaatgatattgaaaaaaaaatttgtaaaatggaaaaatgttccagtgtgtttaatgtcgtaaatagttcaataggattaataatggtattatccttcttgttccttaattagataaagaacacatcttagtttgagttgtacaatatttataaaaatatatactactttt

SEQ ID NO: 77 - DNA, 1275 bp, Artificial Sequence
  P. falciparum CSP
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataataataatggagataataatcgtgaaggtaaagatgaagataaaagagatggaaataacgaagacaacgagacattaaggaaaccaaaacataaaaaattaaagcaaccaggggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaaccccaatgcaaacccaaatgcaaaccccaatgcaaatcctaatgcaaacccaaatgcaaacccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacaatgctgtaaaaaataataataacgaagaaccaagtgataagcacatagaacaatatttaaagaaaatacaaaattctctttcaactgaatggtccccatgtagtgtaacttgtggaaatggtattcaagttagaataaagcctggctctgctaataaacctaaagacgaattagattatgaaaatgatattgaaaaaaaaatttgtaaaatggaaaaatgttccagtgtgtttaatgtcgtaaatagttcaataggattaataatggtattatccttcttgttccttaattag

SEQ ID NO: 78 - DNA, 947 bp, Artificial Sequence
  P. falciparum CSP
gaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataataataatggagataatggtcgtgaaggtaaagatgaagataaaagagatggaaataacgaagacaacgagaaattaaggaaaccaaaacataaaaaattaaagcaaccagcggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaatcctaatgcaaacccaaatgcaaacccaaacgtagatcctaatgcaaacccaaatgcaaacccaaacgcaaaccccaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatggcaacaatgctgtaaaaaataataataacgaagaaccaagtgatcagcacatagaaaaatatttaaa

SEQ ID NO: 79 - DNA, 1239 bp, Artificial Sequence
  P. falciparum CSP
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataataataatggagataatggtcgtgaaggtaaagatgaagataaaagagatggaaataacgaagacaacgagaaattaaggaaaccaaaacataaaaaattaaagcaaccaggggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaaccccaatgcaaatcctaatgcaaatcctaatgcaaacccaaatgcaaatcctaatgcaaacccaaatgcaaacccaaacgtagatcctaatgcaaatccaaatgcaaacccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacaatgctgtaaaaaataataataacgaagaaccaagtgataagcacatagaacaatatttaaagaaaataaaaaattctatttcaactgaatggtccccatgtagtgtaacttgtggaaatggtattcaagttagaataaagcctggctctgctaataaacctaaagacgaattagattatgaaaatgatattgaaaaaaaaatttgtaaaatggaaaaatgttccagtgtgtttaatgtcgtaaatagttcaataggattaataatggtattatccttcttgttccttaattag

SEQ ID NO: 80 - DNA, 1227 bp, Artificial Sequence
  P. falciparum CSP
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataataataatggagataatggtggtgaaggtaaagatgaagataaaagagatggaaataacgaagacaacgagaaattaaggaaaccaaaacataaaaaattaaagcaaccagcggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaaccccaatgcaaatcctaatgcaaacccaaatgcaaacccaaacgtagatcctaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaataacccaaaccgaaatgtagatgaaaatgctaatgccaacaatgctgtaaaaaataataataacgaagaaccaagtgataagcacatagaacaatatttaaagaaaatacaaaattctctttcaactgaatggtccccatgtagtgtaacttgtggaaatggtattcaagttagaataaagcctggctctgctggtaaatctaaagacgaattagattatgaaaatgatattgaaaaaaaaatttgtaaaatggaaaaatgttccagtgtgtttaatgtcgtaaatagttcaataggattaataatggtattatccttcttgttccttaattag

SEQ ID NO: 81 - DNA, 1257 bp, Artificial Sequence
  Pf CS consensus
atgatgagaaaattagctattttatctgtttcttcctttttatttgttgaggccttattccaggaataccagtgctatggaagttcgtcaaacacaagggttctaaatgaattaaattatgataatgcaggcactaatttatataatgaattagaaatgaattattatgggaaacaggaaaattggtatagtcttaaaaaaaatagtagatcacttggagaaaatgatgatggaaataataataatggagataatggtcgtgaaggtaaagatgaagataaaagagatggaaataacgaagacaacgagaaattaaggaaaccaaaacataaaaaattaaagcaaccagcggatggtaatcctgatccaaatgcaaacccaaatgtagatcccaatgccaacccaaatgtagatccaaatgcaaacccaaatgtagatccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaatgcaaacccaaagcaaatcctaatgcaaacaatgcaaacccaaatgcaaacccaaacgcaaacccaatgcaaatccaatgcaaatccaaatgcaaacccaaacgcaaaccccaatgcaaatcctaatgcaaaccccaatgcaaatcctaatgcaaatcctaatgccaatccaaatgcaaatccaaatgcaaacccaaacgcaaaccccaatgcaaatcctaatgccaatccaaatgcaaatccaaatgcaaacccaaatgcaaacccaaatgcaaaccccaatgcaaatcctaataaaaacaatcaaggtaatggacaaggtcacaatatgccaaatgacccaaaccgaaatgtagatgaaaatgctaatgccaacaatgctgtaaaaaataataataacgaagaaccaagtgataagcacatagaacaatatttaaagaaaatacaaaattctctttcaactgaatggtccccatgtagtgtaacttgtggaaatggtattcaagttagaataaagcctggctctgctaataaacctaaagacgaattagattatgaaaatgatattgaaaaaaaaatttgtaaaatggaaaaatgttccagtgtgtttaatgtcgtaaatagttcaataggattaataatggtattatccttcttgttccttaattag

SEQ ID NO: 82 - DNA, 1137 bp, Artificial Sequence
  P. vivax CSP
atgaagaacttcattctcttggctgtttcttccatcctgttggtggacttgttccccacgcactgcgggcacaatgtagatctgtccaaggccataaatttaaatggagtaaacttcaataatgtagacgccagttcacttggcgcggcacacgtaggacaaagtgctagccgaggcagaggacttggtgagaacccagatgacgaggaaggagatgctaaaaaaaaaaaggatggaaagaaagcagaaccaaaaaatccacgtgaaaataagctgaaacaaccaggagacagagcagatggacagccagcaggagacagagcagatggacagccagcaggtgatagagcagatggacaaccagcaggagatagagcagctggacaaccagcaggagatagagcagatggacagccagcaggagacagagcagatggacagccagcaggagacagagcagatggacaaccagcaggagacagagcagatggacaaccagcaggtgatagagcagctggacaaccagcaggtgatagagcagctggacaaccagcaggagatagagcagatggacagccagcaggagatagagcagctggacagccagcaggagatagagcagatggacagccagcaggagatagagcagctggacagccagcaggagatagagcagatggacagccagcaggagatagagcagctggacagccagcaggagatagagcagctggacagccagcaggagatagagcagctggacagccagcaggagatagagcagctggacagccagcaggaaatggtgcaggtggacaggcagcaggaggaaacgcaggaggaggacagggacaaaataatgaaggtgcgaatgccccaaatgaaaagtctgtgaaagaatacctagataaagttagagctaccgttggcaccgaatggactccatgcagtgtaacctgtggagtgggtgtaagagtcagaagaagagttaatgcagctaacaaaaaaccagaggatcttactttgaatgaccttgagactgatgtttgtacaatggataagtgtgctggcatatttaacgttgtgagtaattcattagggctagtcatattgttagtcctagcattattcaattaa

SEQ ID NO: 83 - DNA, 2220 bp, Artificial Sequence
  P. vivax CSP
atgagcattagcgaacatacaggaaatgaggtcagcatagcagaacgagaaaggaaggaagaagccatcggcgcagataaaaatgacaaagatgtgccacctacaaatggtgagagtatttcccttcgaatggagaaaatgaaaggcgaggcaaatgatgagggttctgtagaggtgggaaattgctctccagttgtggaaaacgagttgggaagtagcgttttgtgtgtcggctgcagtggcgaggcatccaacattgacggaggaacgaccaacatttctggtgaggaaaatccccgaacagaggcggacggtgataagaaggcgcaacctgaaggcatcctccatgaggcaaaaaaggttaaaaatggagtcgacgcggagactaaatcgaacgcccaagttagtagcaacggtacgtatgtagagaacgccccgaatgtagaagcctccccgaatgataacgcatgtggagaggggcatagcaacactcacgtggttgatgatgggccccccaatgctgcaccgacgagcaatggtgagctcattgcaaacagcggtggcgcgctaaatgtggaggaggacgcaccgctggatgaaggaaatttctcagcggatgacagacccgaagagaacgcagagagtacgggcagcttcatgttagaagaagacctaaatttatcccgaagagcatatcgaaatttccacatatgttcaatttttatacatgggactttgttattgatggttatattactgatgggaattttattccacgattttatgaagccatcttctgtttcgcagaaggagaagataatgacctacttctgtgggttgctcctaagcatgctagggttacatctttgcctaaatttatatatgtcgttggtgttactgagacaagcagaagtttcaaaagtgttaaaatcggtggaggccaaaattcatgttattgttttggtgtacttttccatgtgcgcttatatttacttttttgaaggcaaaacgtaccccataagctccatcttttctttcaccatcattttagctatcatatattattttatgccaatatttttatacatcgttttgaggattctgttcataattgtcattttgattttaatttttatgaagaggaagagtcccacgccgaagaagattttgaagaaattgaaaattatgaaatatatggagtataggaagtactgtgaggaggaggcttgctttcgtagcgcctattttaccaactggagggagctcaacggggagggggggtcgactccacaggagggagtgacaggtacgagggaggccatgacgaccaccgcggtggaggcaggcacagggattgctgcgtccggtggtgagaacaagggggacgacattgcggcagtggaacccacgtcaaactgcaatgcggacgggaatacgatatctacggcgacgtcctgcgtgcggggcagcagcgcgaatgagcggccaacccgcagcggcaacagcagcacgaggagcaacctggagcggcaccttttctatgatcgcgctgggggagccactggcaggggaggggggtcctccaatcgcgggggggcgcaagcaagcgacagggagggcggaaatcaaaatggtagggataacgccagggataacgctaggtgtaacgccagggataacgctagggataacgccagggataacgctagggataacgccagggataacgccagggataacgctaggtgtaacgccagggataacgctaggtgtaacgccagggataacgctagggataacgccagggataacgctagggataacgccagggataatgccaggcaacacccccccccgagctacgacaacagccgcgaagttccaaacgccagcgacgaagccaatggtgagccaaataaagacggcaagtcagcagccgtatttgagtacttccagaaagttttgaagaagaaaaaaaatgccctggaaagtgacaacgcgcaagtgcatgaaaattacgcggaggaaaattcctttcacataaacatcgagagctccgattatgtttgttccatctgctgcgtcgaatacctaaatgacgatgacatttgcattttgccttgcaactacctgcactactatcacaaggagtgtattttcacgtggctgaagaggaacaacgactgccccctctgcaggaagtgcattgggaagatctga

SEQ ID NO: 84 - DNA, 523 bp, Artificial Sequence
  Pv VX210 CS consensus
atgaagaacttcattctcttggctgtttcttccatcctgttggtggacttgttccccacgcactgcgggcacaatgtagatctgtccaaggccataaatttaaatggagtaaacttcaataatgtagacgccagttcacttggcgcggcacacgtaggacaaagtgctagccgaggcagaggacttggtgagaacccagatgacgaggaaggagatgctaaaaaaaaaaaggatggaaagaaagcagaaccaaaaaatccacgtgaaaataagctgaaacaaccaggagacagagcagatggacagccagcaggagacagagcagatggacagccagcagggaagagcagatggacaccagcagggatagagcagtggacaaccagcaggagatagagcagtggacagccagcaggagaagagcagatggacagccagcaggagacgagcagatggacaccagcaggagacagagcagatggacaccagcagggaagagcagtggacaccagcaggtgataga

SEQ ID NO: 85 - DNA, 711 bp, Artificial Sequence
  P. vivax CSP
aagctgaaacaaccagaagatggggcaggcaatcaaccaggagcaaatggagcaggcaatcaaccaggagcaaatggggcaggcaatcaaccaggagcaaatggggcaggcaatcaaccaggagcaaatggggctggcaatcaaccaggagcaaatggggctggcaatcaaccaggagcaaatggggctggcaatcaaccaggagcaaatggggctggcaatcaaccaggagcaaatggagcaggcaatcaaccaggagcaaatggggcaggcaatcaaccaggagcaaatggggctggcaatcaaccaggagcaaatggagcaggcaatcaaccaggagcaaatggggctggcaatcaaccaggagcaaatggagcaggcaatcaaccaggagcaaatggggcgggcaatcaaccaggagcaaatggggccggcaatcaaccaggagcaaatggggcaggcaatcaaccaggagcaaatggggctggcaatcaaccaggagcaaatggggcaggtaatcaaccaggagcaaatggtgcaggtggacaggcagcaggaggaaatgctgcaaacaaaaaggcaggagacgcaggagcaggacagggacaaaataatgaaggtgcgaatgccccaaatgaaaagtctgtgaaagaatacctagataaagttagagctaccgttggcaccgaatggactccatgcagtgtaacc

SEQ ID NO: 86 - DNA, 1215 bp, Artificial Sequence
  P. vivax CSP
gatctgtccaaggccataaatttaaatggagtaggcttcaataatgtagacgccagttcacttggcgcggcacacgtaggacaaagtgctagccgaggcagaggacttggtgagaacccagatgacgaggaaggagatgctaaaaaaaaaaaggatggaaagaaagcagaaccaaaaaatccacgtgaaaataagctgaaacaaccagaagatggggcaggcaatcaaccaggagcaaatggggctggcaatcaaccaggagcaaatggggctggcaatcaaccaggagcaaatggagcagatgatcaaccaggagcaaatggggcaggcaatcaaccaggagcaaatggggctggcaatcaaccaggagcaaatggggctggcaatcaaccaggagcaaatggggctggcaatcaaccaggagcaaatggagcagatgatcaaccaggagcaaatggggcaggcaatcaaccaggagcaaatggggctggcaatcaaccaggagcaaatggggctggcaatcaaccaggagcaaatggggctggcaatcaaccaggagcaaatggagcagatgatcaaccaggagcaaatggggcaggcaatcaaccaggagcaaatggggctggcaatcaaccaggagcaaatggggcaggtaatcaaccaggagcaaatggagcaggtgatcaaccaggagcaaatggggccggcaatcaaccaggagcaaatggggccggcaatcaaccaggagcaaatggggccggcaatcaaccaggagcaaatggggccggcaatcaaccaggagcaaatggggcaggtaatcaaccaggagcaaatggggctggcaatcaaccaggagcaaatggggcaggtaatcaaccaggagcaaatggagcaggtggacaggcagcaggaggaaacgcaggaggacagggacaaaataatgaaggtgcgaatgccccaaatgaaaagtctgtgaaagaatacctagataaagttagagctaccgttggcaccgaatggactccatgcagtgtaacctgtggagtgggtgtaagagtcagaagaagagttaatgcagctaacaaaaaaccagaggatcttactttgaatgaccttgagactgatgtttgtacaatggataagtgtgctggcatatttaacgttgtgagtaattcattagggctagtcatattgttagtcctagcattattcaat

SEQ ID NO: 87 - DNA, 1063 bp, Artificial Sequence
  P. vivax CSP
acgcactgcgggcacaatgtagatctgtccaaggccataaatttaaatggagtaggcttcaataatgtagacgccagttcacttggcgcggcacacgtaggacaaagtgctagccgaggcagaggacttggtgagaacccagatgacgaggaaggagatgctaaaaaaaaaaaggatggaaagaaagcagaaccaaaaaatccacgtgaaaataagctgaaacaaccagaagatggggcaggcaatcaaccaggagcaaatggagcaggcaatcaaccaggagcaaatggggcaggcaatcaaccaggagcaaatggggcaggcaatcaaccaggagcaaatggggctggcaatcaaccaggagcaaatggggctggcaatcaaccaggagcaaatggggctggcaatcaaccaggagcaaatggggctggcaatcaaccaggagcaaatggagcagatgatcaaccaggagcaaatggggcaggcaatcaaccaggagcaaatggggctggcaatcaaccaggagcaaatggggcaggtaatcaaccaggagcaaatggagcaggtgatcaaccaggagcaaatggggctggcaatcaaccaggagcaaatggagcaggtgatcaaccaggagcaaatggggccggcaatcaaccaggagcaaatggggccggcaatcaaccaggagcaaatggggcaggtaatcaaccaggagcaaatggggctggcaatcaaccaggagcaaatggggcaggtaatcaaccaggagcaaatggagcaggtggacaggcagcaggaggaaatgctgcaaacaaaaaggcaggagacgcaggagcaggacagggacaaaataatgaaggtgcgaatgccccaaatgaaaagtctgtgaaagaatacctagataaagttagagctaccgttggcaccgaatggactccatgcagtgtaacctgtggagtgggtgtaagagtcagaagaagagttaatgcagctaacaaaaaaccagaggatcttactttgaatgaccttgagactgatgtttgtacaatggataagtgtgctg

SEQ ID NO: 88 - DNA, 683 bp, Artificial Sequence
  P. vivax CSP
gcagaaccaaaaaatccacgtgaaaataagctgaaacaaccagaagatggggcaggcaatcaaccaggagcaaatggagcaggtgatcaaccaggagcaaatggggcaggcaatcaaccaggagcaaatggggcaggcaatcaaccaggagcaaatggggctggcaatcaaccaggagcaaatggggctggcaatcaaccaggagcaaatggggctggcaatcaaccaggagcaaatggagcaggtgatcaaccaggagcaaatggggcaggcaatcaaccaggagcaaatggggctggcaatcaaccaggagcaaatggggcaggtaatcaaccaggagcaaatggagcaggtaatcaaccaggagcaaatggggctggcaatcaaccaggagcaaatggagcaggtgatcaaccaggagcaaatggggccggcaatcaaccaggagcaaatggggctggtaatcaaccaggagcaaatggggctggcaatcaaccaggagcaaatggggcaggtaatcaaccaggagcaaatggggcaggtggacaggcagcaggaggaaatgctgcaaacaaaaaggcaggagacgcaggagcaggacagggacaaaataatgaaggtgcgaatgccccaaatgaaaagtctgtgaaagaatacctagataaagttagagctaccgttgg

SEQ ID NO: 89 - DNA, 710 bp, Artificial Sequence
  P. vivax CSP
gcagaaccaaaaaatccacgtgaaaataagctgaaacaaccagaagatggggcaggcaatcaaccaggagcaaatggagcaggtgatcaaccaggagcaaatggggcaggcaatcaaccaggagcaaatggggcaggcaatcaaccaggagcaaatggggctggcaatcaaccaggagcaaatggggctggcaatcaaccaggagcaaatggggctggcaatcaaccaggagcaaatggagcaggtgatcaaccaggagcaaatggggcaggcaatcaaccaggagcaaatggggctggcaatcaaccaggagcaaatggggcaggtaatcaaccaggagcaaatggagcaggtgatcaaccaggagcaaatggggctggcaatcaaccaggagcaaatggagcaggtgatcaaccaggagcaaatggggccggcaatcaaccaggagcaaatggggccggcaatcaaccaggagcaaatggggcaggtaatcaaccaggagcaaatggggctggcaatcaaccaggagcaaatggggcaggtaatcaaccaggagcaaatggagcaggtggacaggcagcaggaggaaatgctgcaaacaaaaaggcaggagacgcaggagcaggacagggacaaaataatgaaggtgcgaatgccccaaatgaaaagtctgtgaaagaatacctagataaagttagagctaccgttgg

SEQ ID NO: 90 - DNA, 683 bp, Artificial Sequence
  P. vivax CSP
gcagaaccaaaaaatccacgtgaaaataagctgaaacaaccagaagatggggcaggtgatcaaccaggagcaaatggagcaggtgatcaaccaggagcaaatggggcaggcaatcaaccaggagcaaatggggcaggcaatcaaccaggagcaaatggggctggcaatcaaccaggagcaaatggggctggcaatcaaccaggagcaaatggggctggcaatcaaccaggagcaaatggagcaggtgatcaaccaggagcaaatggggcaggcaatcaaccaggagcaaatggggctggcaatcaaccaggagcaaatggggcaggtaatcaaccaggagcaaatggagcaggtgatcaaccaggagcaaatggggctggcaatcaaccaggagcaaatggagcaggtgatcaaccaggagcaaatggggccggcaatcaaccaggagcaaatggggcaggtaatcaaccaggagcaaatggggctggcaatcaaccaggagcaaatggggcaggtaatcaaccaggagcaaatggagcaggtggacaggcagcaggaggaaatgctgcaaacaaaaaggcaggagacgcaggagcaggacagggacaaaataatgaaggtgcgaatgccccaaatgaaaagtctgtgaaagaatacctagataaagttagagctaccgttgg

SEQ ID NO: 91 - DNA, 710 bp, Artificial Sequence
  P. vivax CSP
gcagaaccaaaaaatccacgtgaaaataagctgaaacaaccagaagatggagcaggtgatcaaccaggagcaaatggggcaggcaatcaaccaggagcaaatggggcaggcaatcaaccaggagcaaatggggcaggcaatcaaccaggagcaaatggggcaggcaatcaaccaggagcaaatggggctggcaatcaaccaggagcaaatggggcaggtaatcaaccaggagcaaatggagcaggtgatcaaccaggagcaaatggggctggcaatcaaccaggagcaaatggagcaggtgatcaaccaggagcaaatggggctggcaatcaaccaggagcaaatggagcaggtgatcaaccaggagcaaatggggctggcaatcaaccaggagcaaatggagcaggtgatcaaccaggagcaaatggggccggcaatcaaccaggagcaaatggggcaggtaatcaaccaggagcaaatggggctggcaatcaaccaggagcaaatggggctggcaatcaaccaggagcaaatggggcaggtaatcaaccaggagcaaatggagcaggtggacaggcagcaggaggaaatgctgcaaacaaaaaggcaggagacgcaggagcaggacagggacaaaataatgaaggtgcgaatgccccaaatgaaaagtctgtgaaagaatacctagataaagttagagctaccgttgg

SEQ ID NO: 92 - DNA, 710 bp, Artificial Sequence
  P. vivax CSP
gcagaaccaaaaaatccacgtgaaaataagctgaaacaaccagaagatggagcaggcaatcaaccaggagcaaatggggcaggcaatcaaccaggagcaaatggggcaggcaatcaaccaggagcaaatggggcaggcaatcaaccaggagcaaatggggcaggcaatcaaccaggagcaaatggggctggcaatcaaccaggagcaaatggggcaggtaatcaaccaggagcaaatggagcaggtgatcaaccaggagcaaatggggctggcaatcaaccaggagcaaatggagcaggtgatcaaccaggagcaaatggggctggcaatcaaccaggagcaaatggagcaggtgatcaaccaggagcaaatggggctggcaatcaaccaggagcaaatggagcaggtgatcaaccaggagcaaatggggctggcaatcaaccaggagcaaatggggccggcaatcaaccaggagcaaatggggctggtaatcaaccaggagcaaatggggctggtaatcaaccaggagcaaatggggcaggtaatcaaccaggagcaaatggggcaggtggacaggcagcaggaggaaatgctgcaaacaaaaaggcaggagacgcaggagcaggacagggacaaaataatgaaggtgcgaatgccccaaatgaaaagtctgtgaaagaatacctagataaagttagagctaccgttgg

SEQ ID NO: 93 - DNA, 683 bp, Artificial Sequence
  P. vivax CSP
gcagaaccaaaaaatccacgtgaaaataagctgaaacaaccagaagatggggcaggtgatcaaccaggagcaaatggggcaggcaatcaaccaggagcaaatggggcaggcaatcaaccaggagcaaatggggcaggcaatcaaccaggagcaaatggggctggcaatcaaccaggagcaaatggggctggcaatcaaccaggagcaaatggggctggcaatcaaccaggagcaaatggagcagatgatcaaccaggagcaaatggggcaggcaatcaaccaggagcaaatggggctggcaatcaaccaggagcaaatggggcaggtaatcaaccaggagcaaatggagcaggtgatcaaccaggagcaaatggggctggcaatcaaccaggagcaaatggagcaggtgatcaaccaggagcaaatggggccggtaatcaaccaggagcaaatggggcaggtaatcaaccaggagcaaatggggctggcaatcaaccaggagcaaatggggcaggtaatcaaccaggagcaaatggagcaggtggacaggcagcaggaggaaatgctgcaaacaaaaaggcaggagacgcaggagcaggacagggacaaaataatgaaggtgcgaatgccccaaatgaaaagtctgtgaaagaatacctagataaagttagagctaccgttgg

SEQ ID NO: 94 - DNA, 740 bp, Artificial Sequence
  P. vivax CSP
gcagaaccaaaaaatccacgtgaaaataagctgaaacaaccagaagatggggcaggtgatcaaccaggagcaaatggggcaggcaatcaaccaggagcaaatggggcaggcaatcaaccaggagcaaatggggcaggcaatcaaccaggagcaaatggggctggcaatcaaccaggagcaaatggggctggcaatcaaccaggagcaaatggggctggcaatcaaccaggagcaaatggagcagatgatcaaccaggagcaaatggggcaggcaatcaaccaggagcaaatggggctggcaatcaaccaggagcaaatggggcaggtaatcaaccaggagcaaatggagcaggtgatcaaccaggagcaaatggggctggcaatcaaccaggagcaaatggagcaggtgatcaaccaggagcaaatggggccggcaatcaaccaggagcaaatggggcaggtaatcaaccaggagcaaatggggctggcaatcaaccaggagcaaatggggcaggtaatcaaccaggagcaaatggagcaggtggacaggcagcaggaggaaatgctgcaaacaaaaaggcaggagacgcaggagcaggtggacaggcagcaggaggaaatgctgcaaacaaaaaggcaggagacgcaggagcaggacagggacaaaataatgaaggtgcgaatgccccaaatgaaaagtctgtgaaagaatacctagataaagttagagctaccgttgg

SEQ ID NO: 95 - DNA, 740 bp, Artificial Sequence
  P. vivax CSP
gcagaaccaaaaaatccacgtgaaaataagctgaaacaaccagaagatggggcaggcaatcaaccaggagcaaatggggcaggcaatcaaccaggagcaaatggggcaggcaatcaaccaggagcaaatggggcaggcaatcaaccaggagcaaatggggctggcaatcaaccaggagcaaatggggctggcaatcaaccaggagcaaatggggctggcaatcaaccaggagcaaatggagcagatgatcaaccaggagcaaatggggcaggcaatcaaccaggagcaaatggggctggcaatcaaccaggagcaaatggggcaggtaatcaaccaggagcaaatggagcaggtgatcaaccaggagcaaatggggctggcaatcaaccaggagcaaatggagcaggtgatcaaccaggagcaaatggggccggcaatcaaccaggagcaaatggggcaggtaatcaaccaggagcaaatggggctggcaatcaaccaggagcaaatggggcaggtaatcaaccaggagcaaatggagcaggtggtcaggcagcaggaggaaatgctgcaaacaaaaaggcaggagacgcaggagcaggtggacaggcagcaggaggaaatgctgcaaacaaaaaggcaggagacgcaggagcaggacagggacaaaataatgaaggtgcgaatgccccaaatgaaaagtctgtgaaagaatacctagataaagttagagctaccgttgg

SEQ ID NO: 96 - DNA, 717 bp, Artificial Sequence
  P. vivax CSP
aaaaaggatggaaagaaagcagaaccaaaaaatccacgtgaaaataagctgaaacaaccagaagatggggcaggcaatcaaccaggagcaaatggagcaggcaatcaaccaggagcaaatggggcaggcaatcaaccaggagcaaatggggcaggcaatcaaccaggagcaaatggggcaggcaatcaaccaggagcaaatggggctggcaatcaaccaggagcaaatggggctggcaatcaaccaggagcaaatggggctggcaatcaaccaggagcaaatggagcagatgatcaaccaggagcaaatggggcaggcaatcaaccaggagcaaatggggctggcaatcaaccaggagcaaatggggcaggtaatcaaccaggagcaaatggagcaggtgatcaaccaggagcaaatggggctggcaatcaaccaggagcaaatggagcaggtgatcaaccaggagcaaatggagccggcaatcaaccaggagcaaatggggccggcaatcaaccaggtgcaaatgaggcaggtaatcaaccaggagcaaatggggctggcaatcaaccaggagcaaatggggcaggtaatcaaccaggagcaaatggagcaggtggacaggcagcaggaggaaatgctgcaaacaaaaaggcaggagacgcaggagcaggacagggacaaaataatgaaggtgcgaatgccccaaatgaaaagtca

SEQ ID NO: 97 - DNA, 710 bp, Artificial Sequence
  Pv VK247 CS consensus
gcagaaccaaaaaatccacgtgaaaataagctgaaacaaccagaagatggggcaggcaatcaaccaggagcaaatggggcaggcaatcaaccaggagcaaatggggcaggcaatcaaccaggagcaaatggggcaggcaatcaaccaggagcaaatggggctggcaatcaaccaggagcaaatggggctggcaatcaaccaggagcaaatggggctggcaatcaaccaggagcaaatggagcaggtgatcaaccaggagcaaatggggcaggcaatcaaccaggagcaaatggggctggcaatcaaccaggagcaaatggggcaggtaatcaaccaggagcaaatggagcaggtgatcaaccaggagcaaatggggctggcaatcaaccaggagcaaatggagcaggtgatcaaccaggagcaaatggggccggcaatcaaccaggagcaaatggggccggtaatcaaccaggagcaaatggggctggcaatcaaccaggagcaaatggggctggtaatcaaccaggagcaaatggggcaggtaatcaaccaggagcaaatggagcaggtggacaggcagcaggaggaaatgctgcaaacaaaaaggcaggagacgcaggagcaggacagggacaaaataatgaaggtgcgaatgccccaaatgaaaagtctgtgaaagaatacctagataaagttagagctaccgttgg

SEQ ID NO: 98 - DNA, 1326 bp, Artificial Sequence
  P. malariae CSP
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgaaggaaatgatgcaggaaatgatgcaggaaatgcagcaggaaatgcagcaggtaacgcagcaggaaatgcagcaggaaatgatgcaggaaatgcagcaggaaatgatgcaggaaatgcagcaggaaatgatgcaggaaatgcagcaggtaacgcagcaggaaatgcagcaggaaatgcagcagggaatgcagcaggtaacgcagcaggtaacgcagcaggaaatgcagcaggaaatgcagcaggaaatgatgcaggaaatgcagcaggtaacgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgatgcaggaaatgcagcaggtaacgcagcaggaaatgcagcaggaaatgcagcaggaaatgcagcaggaaatgcagcaggaaatgcagcaggaaatgcagcaggtaacgcagcaggaaatgcagcaggaaatgcagcaggtaacgcagcaggaaatgcagcaggaaatgcagcaggtaacgcagcaggaaatgcagcaggaaatgcagcaggtaacgcagcaggaaatgcagcaggaaatgcagcaggaaatgaaaaagcgaaaaataaggataataaagtggatgcaaatacgaataaaaaggacaatcagggagaaaataatgattcgtctaatggtccatctgaagaacatataaagaattatttagaaagtattcgtaatagtattacggaggaatggtcaccatgtagtgtaacttgtggaagtggtataagggctagaagaaaggttgatgcaaaaaataagaaacctgcagaattagttttaagtgaccttgaaactgaaatttgttcactagataaatgctccagtatatttaatgtcgtaagtaattcgttaggaatagtattagttttagtcttaatactctttcactaa

SEQ ID NO: 99 - DNA, 1326 bp, Artificial Sequence; identical to SEQ ID NO: 98
  P. malariae CSP

SEQ ID NO: 100 - DNA, 1290 bp, Artificial Sequence
  P. malariae CSP
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgcaggaaatgatgcaggaaatgatgcaggaaatgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgatgcaggaaatgcagcaggtaacgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggtaacgcagcaggtaacgcagcaggaaatgcagcaggaaatgcagcaggaaatgatgcaggaaatgcagcaggtaacgcagcaggaaatgcagcaggaaatgcagcaggtaacgcagcaggtaacgcagcaggaaatgcagcaggaaatgcagcaggtaacgcagcaggaaatgcagcaggaaatgcagcaggtaacgcagcaggtaacgcagcaggaaatgcagcaggaaatgcagcaggtaacgcagcaggaaatgcagcaggaaatgcagcaggtaacgcagcaggaaatgcagcaggaaatgaaaaagcgaaaaataaggataataaagtggatgcaaatacgaataaaaaggacaaccaggaagaaaataatgattcgtctaatggtccatctgaagaacatataaagaattatttagaaagtattcgtaatagtattacggaggaatggtcaccatgtagtgtaacttgtggaagtggtataagggctagaagaaaggttggtgcaaaaaataagaaacctgcagaattagttttaagtgaccttgaaactgaaatttgttcactagataaatgctccagtatatttaatgtcgtaagtaattcgttaggaatagtattagttttagtcttaatactctttcactaa

SEQ ID NO: 101 - DNA, 1290 bp, Artificial Sequence
  P. malariae CSP
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgcaggaaatgatgcaggaaatgatgcaggaaatgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgatgcaggaaatgcagcaggaaatgatgcaggaaatgcagcaggtaacgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggtaacgcagcaggtaacgcagcaggaaatgcagcaggaaatgcagcaggaaatgatgcaggaaatgcagcaggtaacgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgcagcaggtaacgcagcaggaaatgcagcaggaaatgcagcaggtaacgcagcaggaaatgcagcaggaaatgcagcaggaaatgaaaaagcgaaaaataaggataataaagtggatgcaaatacgaataaaaaggacaaccaggaagaaaataatgattcgtctaatggtccatctgaagaacatataaagaattatttagaaagtattcgtaatagtattacggaggaatggtcaccatgtagtgtaacctgtggaagtggtataagggctagaagaaaggttggtgcaaaaaataagaaacctgcagaattagttttaagtgaccttgaaactgaaatttgttcactagataaatgctccagtatatttaatgtcgtaagtaattcgttaggaatagtattagttttagtcttaatactctttcactaa

SEQ ID NO: 102 - DNA, 1290 bp, Artificial Sequence
  P. malariae CSP
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgcaggaaatgatgcaggaaatgatgcaggaaatgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgatgcaggaaatgcagcaggaaatgatgcaggaaatgcagcaggtaacgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggtaacgcagcaggtaacgcagcaggaaatgcagcaggaaatgcagcaggaaatgatgcaggaaatgcagcaggtaacgcagcaggaaatgcagcaggaaatgcagcaggaaatgcagcaggtaacgcagcaggtaacgcagcaggaaatgcagcaggtaacgcagcaggaaatgcagcaggaaatgcagcaggtaacgcagcaggaaatgcagcaggaaatgcagcaggtaatgcagcaggtaacgcagcaggaaatgcagcaggaaatgcagcaggaaatgaaaaagcgaaaaataaggataataaagtggatgcaaatacgaataaaaaggacaaccaggaagaaaataatgattcgtctaatggtccatctgaagaacatataaagaattatttagaaagtattcgtaatagtattacggaggaatggtcaccatgtagtgtaacttgtggaagtggtataagggctagaagaaaggttgatgcaaaaaataagaaacctgcagaattagttttaagtgaccttgaaactgaaatttgttcactagataaatgctccagtatatttaatgtcgtaagtaattcgttaggaatagtattagttttagtcttaatactctttcactaa

SEQ ID NO: 103 - DNA, 1266 bp, Artificial Sequence
  P. malariae CSP
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgcaggaaatgatgcaggaaatgcagcaggaaatgcagcaggaaatgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggaaatgatgcaggaaatgcagcaggtaacgcagcaggaaatgcagcaggaaatgcagcaggaaatgatgcaggaaatgcagcaggaaatgcagcaggaaatgcagcaggtaacgcagcaggaaatgcagcaggaaatgcagcaggtaacgcagcaggtaacgcagcaggaaatgcagcaggaaatgcagcaggaaatgatgcaggaaatgcagcaggtaacgcagcaggaaatgcagcaggaaatgcagcaggaaatgcagcaggtaacgcagcaggtaacgcagcaggaaatgcagcaggaaatgcagcaggaaatgcagtaggaaatgcagcaggtaacgcagcaggaaatgcagcaggaaatgcagcaggtaacgcagcaggaaatgcagcaggaaatgcagcaggaaatgaaaaagcgaaaaataaggataataaagtggatgcaaatacgaataaaaaggacaaccaggaagaaaataatgattcgtctaatggtccatctgaagaacatataaagaattatttagaaagtattcgtaatagtattacggaggaatggtcaccatgtagtgtaacttgtggaagtggtataagggctagaagaaaggttgatgcaaaaaataagaaacctgcagaattagttttaagtgaccttgaaactgaaatttgttcactagataaatgctccagtatatttaatgtcgtaagtaattcgttaggaatagtattagttttagtcttaatactctttcactaa

SEQ ID NO: 104 - DNA, 1302 bp, Artificial Sequence
  P. malariae CSP
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgcaggaaatgatgcaggaaatgcagcaggaaatgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggaaatgatgcaggaaatgcagcaggtaacgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggtaacgcagcaggtaacgcagcaggaaatgcagcaggaaatgcagcaggaaatgatgcaggaaatgcagcaggtaacgcagcaggaaatgcagcaggaaatgcagcaggaaatgcagcaggtaacgcagcaggtaacgcagcaggaaatgcagcaggaaatgcagcaggaaatgcagcaggaaatgcagcaggtaacgcagcaggaaatgcagcaggaaatgcagcaggtaacgcagcaggaaatgcagcaggaaatgcagcaggtaacgcagcaggaaatgcagcaggaaatgcagcaggaaatgaaaaagcgaaaaataaggataataaagtggatgcaaatacgaataaaaaggacaaccaggaagaaaataatgattcgtctaatggtccatctgaagaacatataaagaattatttagaaagtattcgtaatagtattacggaggaatggtcaccatgtagtgtaacttgtggaagtggtataagggctagaagaaaggttgatgcaaaaaataagaaacctgcagaattagttttaagtgaccttgaaactgaaatttgttcactagataaatgctccagtacatttaatgtcgtaagtaattcgttaggaatagtattagttttagtcttaatactctttcactaa

SEQ ID NO: 105 - DNA, 1290 bp, Artificial Sequence
  P. malariae CSP
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgcaggaaatgatgcaggaaatgatgcaggaaatgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgatgcaggaaatgcagcaggaaatgatgcaggaaatgcagcaggtaacgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggtaacgcagcaggtaacgcagcaggaaatgcagcaggaaatgcagcaggaaatgatgcaggaaatgcagcaggtaacgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgcagcaggtaacgcagcaggaaatgcagcaggaaatgcagcaggtaacgcagcaggaaatgcagcaggaaatgcagcaggaaatgaaaaagcgaaaaataaggataataaagtggatgcaaatacgaataaaaaggacaaccaggaagaaaataatgattcgtctaatggtccatctgaagaacatataaagaattatttagaaagtattcgtaatagtattacggaggaatggtcaccatgtagtgtaacttgtggaagtggtataagggctagaagaaaggttgatgcaaaaaataagaaacctgcagaattagttttaagtgaccttgaaactgaaatttgttcactagataaatgctccagtatatttaatgtcgtaagtaattcgttaggaatagtattagttttagtcttaatactctttcactaa

SEQ ID NO: 106 - DNA, 1278 bp, Artificial Sequence
  P. malariae CSP
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgcaggaaatgatgcaggaaatgcagcaggaaatgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggaaatgatgcaggaaatgcagcaggtaacgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggtaacgcagcaggtaacgcagcaggaaatgcagcaggaaatgcagcaggaaatgatgcaggaaatgcagcaggtaacgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgcagcaggtaacgcagcaggaaatgcagcaggaaatgcagcaggtaacgcagcaggaaatgcagcaggaaatgcagcaggaaatgaaaaagcgaaaaataaggataataaagtggatgcaaatacgaataaaaaggacaatcagggagaaaataatgattcgtctaatggtccatctgaagaacatataaagaattatttagaaagtattcgtaatagtattacggaggaatggtcaccatgtagtgtaacttgtggaagtggtataagggctagaagaaaggttgatgcaaaaaataagaaacctgcagaattagttttaagtgaccttgaaactgaaatttgttcactagataaatgctccagtatatttaatgtcgtaagtaattcgttaggaatagtattagttttagtcttaatactctttcactaa

SEQ ID NO: 107 - DNA, 1278 bp, Artificial Sequence
  P. malariae CSP
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgcaggaaatgatgcaggaaatgcagcaggaaatgcagcaggaaatgcagcaggaaatgcagcaggtaacgcagcaggtaacgcagcaggaaatgcagcaggaaatgcagcaggaaacgcagcaggaaatgcagcaggaaatgatgcaggaaatgcagcaggaaatgatgcaggaaatgcagcaggtaacgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggtaacgcagcaggtaacgcagcaggaaatgcagcaggaaatgcagcaggaaatgatgcaggaaatgcagcaggtaacgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgcagcaggtaacgcagcaggaaatgcagcaggaaatgcagcaggtaacgcagcaggaaatgcagcaggaaatgcagcaggaaatgaaaaagcgaaaaataaggataataaagtggatgcaaatacgaataaaaaggacaaccaggaagaaaataatgattcgtctaatggtccatatgaagaacatataaagaattatttagaaagtattcgtaatagtattacggaggaatggtcaccatgtagtgtaacttgtggaagtggtataagggctagaagaaaggttgatgcaaaaaataagaaacctgcagaattagttttaagtgaccttgaaactgaaatttgttcactagataaatgctccagtatatttaatgtcgtaagtaattcgttaggaatagtattagttttagtcttaatactctttcactaa

SEQ ID NO: 108 - DNA, 1290 bp, Artificial Sequence
  P. malariae CSP
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgcaggaaatgatgcaggaaatgatgcaggaaatgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgatgcaggaaatgcagcaggaaatgatgcaggaaatgcagcaggtaacgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggtaacgcagcaggtaacgcagcaggaaatgcagcaggaaatgcagcaggtaatgatgcaggaaatgcagcaggtaacgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgcagcaggtaacgcagcaggaaatgcagcaggaaatgcagcaggtaacgcagcaggaaatgcagcaggaaatgcagcaggaaatgaaaaagcgaaaaataaggataataaagtggatgcaaatacgaataaaaaggacaaccaggaagaaaataatgattcgtctaatggtccatctgaagaacatataaagaattatttagaaagtattcgtaatagtattacggaggaatggtcaccatgtagtgtaacttgtggaagtggtataagggctagaagaaaggttgatgcaaaaaataagaaacctgcagaattagttttaagtgaccttgaaactgaaatttgttcactagataaatgctccagtatatttaatgtcgtaagtaattcgttaggaatagtattagttttagtcttaatactctttcactaa

SEQ ID NO: 109 - DNA, 1290 bp, Artificial Sequence
  P. malariae CSP
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgcaggaaatgatgcaggaaatgatgcaggaaatgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgatgcaggaaatgcagcaggaaatgatgcaggaaatgcagcaggtaacgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggtaacgcagcaggtaacgcagcaggaaatgcagcaggaaatgcagcaggaaatgatgcaggaaatgcagcaggtaacgcagcaggaaatgcagcaggaaatgcagcaggaaatgcagcaggtaacgcagcaggtaacgcagcaggaaatgcagcaggaaatgcagcaggaaatgcagcaggaaatgcagcaggtaacgcagcaggaaatgcagcaggaaatgcagcaggtaacgcagcaggaaatgcagcagggaatgcagcaggtaacgcagcaggaaatgaaaaagcgaaaaataaggataataaagtggatgcaaatacgaataaaaaggacaaccaggaagaaaataatgattcgtctaatggtccatctgaagaacatataaagaattatttagaaagtattcgtaatagtattacggaggaatggtcaccatgtagtgtaacttgtggaagtggtataagggctagaagagaggttgatgcaaaaaataagaaacctgcagaattagttttaagtgaccttgaaactgaaatttgttcactagataaatgctccagtatatttaatgtcgtaagtaattcgttaggaatagtattagttttagtcttaatactctttcactaa

SEQ ID NO: 110 - DNA, 1290 bp, Artificial Sequence
  P. malariae CSP
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgcaggaaatgatgcaggaaatgatgcaggaaatgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgatgcaggaaatgcagcaggaaatgatgcaggaaatgcagcaggtaacgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggtaacgcagcaggtaacgcagcaggaaatgcagcaggaaatgcagcaggaaatgatgcaggaaatgcagcaggaaacgcagcaggaaatgcagcaggaaatgcagcaggaaatgcagcaggaaacgcagcaggtaacgcagcaggaaatgcagcaggaaatgcagcaggtaacgcagcaggaaatgcagcaggaaatgcagcaggtaacgcagcaggaaatgcagcaggaaatgcagcaggtaacgcagcaggaaatgcagcaggaaatgcagcaggaaatgaaaaagcgaaaaataaggataataaagtggatgcaaatacgaataaaaaggacaaccaggaagaaaataatgattcgtctaatggtccatctgaagaacatataaagaattatttagaaagtattcgtaatagtattacggaggaatggtcaccatgtagtgtaacttgtggaagtggtataagggctagaagaaaggttgatgcaaaaaataagaaacctgcagaattagttttaagtgaccttgaaactgaaatttgttcactagataaatgctccagtatatttaatgtcgtaagtaattcgttaggaatagtattagttttagtcttaatactctttcactaa

SEQ ID NO: 111 - DNA, 1266 bp, Artificial Sequence
  P. malariae CSP
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgcaggaaatgatgcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggaaatgatgcaggaaatgcagcaggtaacgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggtaacgcagcaggtaacgcagcaggaaatgcagcaggaaatgcagcaggaaatgatgcaggaaatgcagcaggtaacgcagcaggaaatgcagcaggaaatgcagcaggaaatgcagcaggtaacgcagcaggtaacgcagcaggaaatgcagcaggaaatgcagcaggaaatgcagcaggtaacgcagcaggaaatgcagcaggaaatgcagcaggtaacgcagcaggaaatgcagcaggaaatgcagcaggtaacgcagcaggaaatgcagcaggaaatgcagcaggaaatgaaaaagcgaaaaataaggataataaagtggatgcaaatacgaataaaaaggacaaccaggaagaaaataatgattcgtctaatggtccatctgaagaacatataaagaattatttagaaagtattcgtaatagtattacggaggaatggtcaccatgtagtgtaacttgtggaagtggtataagggctagaagaaaggttgatgcaaaaaataagaaacctgcagaattagttttaagtgaccttgaaactgaaatttgttcactagataaatgctccagtatatttaatgtcgtaagtaattcgttaggaatagtattagttttagtcttaatactctttcactaa

SEQ ID NO: 112 - DNA, 1290 bp, Artificial Sequence
  P. malariae CSP
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgcaggaaatgatgcaggaaatgcagcaggaaatgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgatgcaggaaatgcagcaggaaatgatgcaggaaatgcagcaggtaacgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggtaacgcagcaggtaacgcagcaggaaatgcagcaggaaatgcagcaggaaatgatgcaggaaatgcagcaggtaacgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgcagcaggtaacgcagcaggaaatgcagcaggaaatgcagcaggtaacgcagcaggaaatgcagcaggaaatgcagcaggaaatgaaaaagcgaaaaataaggataataaagtggatgcaaatacgaataaaaaggacaaccaggaagaaaataatgattcgtctaatggtccatctgaagaacatataaagaattatttagaaagtattcgtaatagtattacggaggaatggtcaccatgtagtgtaacttgtggaagtggtataaggactagaagaaaggttgatgcaaaaaataagaaacctgcagaattagttttaagtgaccttgaaactgaaatttgttcactagataaatgctccagtatatttaatgtcgtaagtaattcgttaggaatagtattagttttagtcttaatactctttcactaa

SEQ ID NO: 113 - DNA, 1278 bp, Artificial Sequence
  P. malariae CSP
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgcaggaaatgatgcaggaaatgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgatgcaggaaatgcagcaggaaatgatgcaggaaatgcagcaggtaacgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggtaacgcagcaggtaacgcagcaggaaatgcagcaggaaatgcagcaggaaatgatgcaggaaatgcagcaggtaacgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgcagcaggtaacgcagcaggaaatgcagcaggaaatgcagcaggtaacgcagcaggaaatgcagcaggaaatgcagcaggaaatgaaaaagcgaaaaataaggataataaagtggatgcaaatacgaataaaaaggacaaccaggaagaaaataatgattcgtctaatggtccatctgaagaacatataaagaattatttagaaagtattcgtaatagtattacggaggaatggtcaccatgtagtgtaacttgtggaggtggtataagggctagaagaaaggttgatgcaaaaaataagaaacctgcagaattagttttaagtgaccttgaaactgaaatttgttcactagataaatgctccagtatatttaatgtcgtaagtaattcgttaggaatagtattagttttagtcttaatactctttcactaa

SEQ ID NO: 114 - DNA, 1290 bp, Artificial Sequence
  Pm CS consensus
atgaagaagttatctgtcttagcaatatcctcttttttaattgttgatttcctcttccctggatatcatcacaactcaaattccaccaagtcaagaaatttaagtgagttgtgttacaataatgtggacactaaattatttaatgagttagaagtcagatatagcacgaatcaagatcatttctataactataataagacaatcagattacttaatgaaaataacaatgaaaaagatggaaatgtgaccaatgaaagaaaaaaaaaacccacaaaagctgttgaaaataaattgaaacaaccccccggagatgatgatggcgcaggaaatgatgcaggaaatgatgcaggaaatgatgcaggaaatgcagcaggaaatgcagcaggaaatgcagcaggaaatgcagcaggtaacgcagcaggtaatgcagcaggaaatgcagcaggaaatgcagcaggtaacgcagcaggaaatgcagcaggaaatgatgcaggaaatgcagcaggaaatgatgcaggaaatgcagcaggtaacgcagcaggaaatgcagcaggaaatgcagcaggaaatgcagcaggaaatgatgcaggaaatgcagcaggaaatgcagcaggaaatgcagcaggtaacgcagcaggaaatgcagcaggaaatgcagcaggtaacgcagcaggtaacgcagcaggaaatgcagcaggaaatgcagcaggaaatgatgcaggaaatgcagcaggtaacgcagcaggaaatgcagcaggaaatgcagcaggaaatgcagcaggtaacgcagcaggtaacgcagcaggaaatgcagcaggaaatgcagcaggtaacgcagcaggaaatgcagcaggaaatgcagcaggtaacgcagcaggaaatgcagcaggaaatgcagcaggtaacgcagcaggaaatgcagcaggaaatgcagcaggaaatgaaaaagcgaaaaataaggataataaagtggatgcaaatacgaataaaaaggacaaccaggaagaaaataatgattcgtctaatggtccatctgaagaacatataaagaattatttagaaagtattcgtaatagtattacggaggaatggtcaccatgtagtgtaacttgtggaagtggtataagggctagaagaaaggttgatgcaaaaaataagaaacctgcagaattagttttaagtgaccttgaaactgaaatttgttcactagataaatgctccagtatatttaatgtcgtaagtaattcgttaggaatagtattagttttagtcttaatactctttcactaa

SEQ ID NO: 115 - protein, 506 aa, Artificial Sequence
  Flagellin STF2
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 116 - protein, 277 aa, Artificial Sequence
  Flagellin STF2delta
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVHGAPVDPASPWTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 117 - DNA, 1518 bp, Artificial Sequence
  Flagellin STF2
atggcccaggttatcaataccaactccctgtcgttgctcacccaaaataaccttaataaaagccagagcgcactgggaaccgccatagaacgcctctcaagcggcctccggatcaattctgcaaaagacgacgccgccggtcaggccatcgcaaaccgctttaccgccaatatcaagggactgacgcaggcttcgaggaatgctaacgatggaataagcatcgctcaaaccacggagggcgccctgaacgagatcaacaacaacctacagcgcgtcagggagctcgcagtgcagtccgccaattcgaccaactcgcagtcggacctggactcgatccaagccgaaatcacccagcgcctgaatgagattgaccgggtgagcggtcagacacagtttaacggcgtgaaggtacttgcacaggataacacacttacgatacaggtgggcgccaacgatggtgaaaccatagacattgatctcaaacagattaacagccagacgctcgggttggatagcctgaatgtgcaaaaggcgtacgacgtgaaagacacggcggtcactaccaaagcctacgctaacaatggcactaccttggatgtgagcggattggatgatgcagcaatcaaggctgctaccggcggtacgaacggaaccgcgtccgtgaccggcggtgccgtgaagttcgatgctgacaacaataagtatttcgtcaccattggaggctttactggcgccgacgcagcaaagaacggcgactatgaagtgaacgtggcaaccgatggaaccgtgacgctggccgctggtgccaccaagaccaccatgccagccggcgccacaactaagaccgaggtgcaggagttaaaggacacccccgcggtggttagcgcagatgccaaaaacgcgttgatcgccggcggagtggatgcaactgatgctaatggtgcggagctggttaaaatgtcgtatacagacaagaatggtaagacgatcgagggcggttatgcccttaaggcaggagataagtattacgctgctgattacgatgaggcgacgggagctattaaggccaagacaacgtcatacacggcggcggacggaacgactaagacggctgccaatcagttgggaggggttgacgggaagacagaggtcgttacgatcgatggcaagacatacaacgcctccaaggccgctggccacgatttcaaagctcaacccgaactggccgaggccgcggcgaaaacaactgagaacccgttgcagaagattgatgcggccctggcgcaagtagatgccctgcgctcagacctgggcgccgttcaaaatcgattcaattccgcgattacaaacctgggcaatacagtaaacaatctatccgaggccagatcccgcattgaagactccgactacgcgacagaagtaagtaacatgagtcgtgcccagattctgcagcaggccggcactagtgtcctggcccaggccaatcaagtcccgcagaatgtgctgagcctactacga

SEQ ID NO: 118 - protein, 506 aa, Artificial Sequence; identical to SEQ ID NO: 115
  Flagellin STF2

SEQ ID NO: 119 - DNA, 1518 bp, Artificial Sequence
  Flagellin STF2
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcagaaagcgtatgatgtgaaagatacagcagtaacaacgaaagcttatgccaataatggtactacactggatgtatcgggtcttgatgatgcagctattaaagcggctacgggtggtacgaatggtacggcttctgtaaccggtggtgcggttaaatttgacgcagataataacaagtactttgttactattggtggctttactggtgctgatgccgccaaaaatggcgattatgaagttaacgttgctactgacggtacagtaacccttgcggctggcgcaactaaaaccacaatgcctgctggtgcgacaactaaaacagaagtacaggagttaaaagatacaccggcagttgtttcagcagatgctaaaaatgccttaattgctggcggcgttgacgctaccgatgctaatggcgctgagttggtcaaaatgtcttataccgataaaaatggtaagacaattgaaggcggttatgcgcttaaagctggcgataagtattacgccgcagattacgatgaagcgacaggagcaattaaagctaaaactacaagttatactgctgctgacggcactaccaaaacagcggctaaccaactgggtggcgtagacggtaaaaccgaagtcgttactatcgacggtaaaacctacaatgccagcaaagccgctggtcatgatttcaaagcacaaccagagctggcggaagcagccgctaaaaccaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattctgcagcaggccggtacttccgttctggcgcaggctaaccaggtcccgcagaacgtgctgtctctgttacgt

SEQ ID NO: 120 - protein, 277 aa, Artificial Sequence; identical to SEQ ID NO: 116
  Flagellin

SEQ ID NO: 121 - DNA, 832 bp, Artificial Sequence
  Flagellin
atggcacaagtaatcaacactaacagtctgtcgctgctgacccagaataacctgaacaaatcccagtccgcactgggcaccgctatcgagcgtctgtcttctggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggcgccaacgacggtgaaactatcgatatcgatctgaagcagatcaactctcagaccctgggtctggactcactgaacgtgcatggagcgccggtggatcctgctagcccatggaccgaaaacccgctgcagaaaattgatgccgcgctggcgcaggtggatgcgctgcgctctgatctgggtgcggtacaaaaccgtttcaactctgctatcaccaacctgggcaataccgtaaacaatctgtctgaagcgcgtagccgtatcgaagattccgactacgcgaccgaagtttccaacatgtctcgcgcgcagattttgcagcaggccggtacttccgttctggcgcaggctaaccaggtcccgcagaacgtgctgtctctgttacgtg

SEQ ID NO: 122 - protein, 595 aa, Artificial Sequence
  Flagellin
MAQVINTNSLSLITQNNINKNQSALSSSIERLSSGLRINSAKDDAAGQAIANRFTSNIKGLTQAARNANDGISVAQTTEGALSEINNNLQRIRELTVQASTGTNSDSDLDSIQDEIKSRLDEIDRVSGQTQFNGVNVLAKDGSMKIQVGANDGQTITIDLKKIDSDTLGLNGFNVNGSGTIANKAATISDLTAAKMDAATNTITTTNNALTASKALDQLKDGDTVTIKADAAQTATVYTYNASAGNFSLSNVSNNTSEKAGDVAASLLPPAGQTASGVYKAASGEVNFDVDANGKITIGGQKAYLTSDGNLTTNDAGGATAATLDGLFKKAGDGQSIGFKKTASVTMGGTTYNFKTGADADAATANAGVSFTDTASKETVLNKVATAKQGKAAAADGDTSATITYKSGVQTYQAVFAAGDGTASAKYADKADVSNATATYTDADGEMTTIGSYTTKYSIDANNGKVTVDSGTGTGKYAPKVGAEVYVSANGTLTTDATSEGTVTKDPLKALDEAISSIDKFRSSLGAIQNRLDSAVTNLNNTTTNLSEAQSRIQDADYATEVSNMSKAQIIQQAGNSVLAKANQVPQQVLSLLQG

SEQ ID NO: 123 - DNA, 1788 bp, Artificial Sequence
  Flagellin
atggcacaagtcattaataccaacagcctctcgctgatcactcaaaataatatcaacaagaaccagtctgcgctgtcgagttctatcgagcgtctgtcttctggcttgcgtattaacagcgcgaaggatgacgccgcaggtcaggcgattgctaaccgttttacttctaacattaaaggcctgactcaggctgcacgtaacgccaacgacggtatttccgttgcgcagaccaccgaaggcgcgctgtccgaaatcaacaacaacttacagcgtatccgtgaactgacggttcaggcttctaccgggactaactccgattcagatctggactccattcaggacgaaatcaaatcccgtctggacgaaattgaccgcgtatctggccagacccagttcaacggcgtgaacgtactggcgaaagacggttcaatgaaaattcaggttggtgcgaatgacggccagactatcacgattgatctgaagaaaattgactcagatacgctggggctgaatggttttaacgtgaatggttccggtacgatagccaataaagcggcgaccattagcgacctgacagcagcgaaaatggatgctgcaactaatactataactacaacaaataatgcgctgactgcatcaaaggcgcttgatcaactgaaagatggtgacactgttactatcaaagcagatgctgctcaaactgccacggtttatacatacaatgcatcagctggtaacttctcactcagtaatgtatcgaataatacttcagaaaaagcaggtgatgtagcagctagccttctcccgccggctgggcaaactgctagtggtgtttataaagcagcaagcggtgaagtgaactttgatgttgatgcgaatggtaaaatcacaatcggaggacagaaagcatatttaactagtgatggtaacttaactacaaacgatgctggtggtgcgactgcggctacgcttgatggtttattcaagaaagctggtgatggtcaatcaatcgggtttaagaagactgcatcagtcacgatggggggaacaacttataactttaaaacgggtgctgatgctgatgctgcaactgctaacgcaggggtatcgttcactgatacagctagcaaagaaaccgttttaaataaagtggctacagctaaacaaggcaaagcagctgcagctgacggtgatacatccgcaacaattacctataaatctggcgttcagacgtatcaggctgtatttgccgcaggtgacggtactgctagcgcaaaatatgccgataaagctgacgtttctaatgcaacagcaacatacactgatgctgatggtgaaatgactacaattggttcatacaccacgaagtattcaatcgatgctaacaacggcaaggtaactgttgattctggaactggtacgggtaaatatgcgccgaaagtaggggctgaagtatatgttagtgctaatggtactttaacaacagatgcaactagcgaaggcacagtaacaaaagatccactgaaagctctggatgaagctatcagctccatcgacaaattccgttcttccctgggtgctatccagaaccgtctggattccgcagtcaccaacctgaacaacaccactaccaacctgtccgaagcgcagtcccgtattcaggacgccgactatgcgaccgaagtgtccaacatgtcgaaagcgcagatcattcagcaggccggtaactccgtgctggcaaaagccaaccaggtaccgcagcaggttctgtctctgctgcagggttag

SEQ ID NO: 124 - protein, 506 aa, Artificial Sequence
  Flagellin
MAQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDTITAQSNTDIQTAIGGGATGVTGADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEKTGTITAKTTTYTDGAGVAQTGAVKFGGANGKSEVVTATDGKTYLASDLDKHNFRTGGELKEVNTDKTENPLQKIDAALAQVDTLRSDLGAVQNRFNSAITNLGNTVNNLSSARSRIEDSDYATEVSNMSRAQILQQAGTSVLAQANQVPQNVLSLLR

SEQ ID NO: 125 - DNA, 1522 bp, Artificial Sequence
  Flagellin
aatggcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtcaggcgattgctaaccgtttcaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacggtactaactcccagtctgaccttgactctatccaggctgaaatcacccagcgtctgaacgaaatcgaccgtgtatccggtcagactcagttcaacggcgtgaaagtcctggcgcaggacaacaccctgaccatccaggttggtgccaacgacggtgaaactattgatattgatttaaaagaaattagctctaaaacactgggacttgataagcttaatgtccaggatgcctacaccccgaaagaaactgctgtaaccgttgataaaactacctataaaaatggtacagatactattacagcccagagcaatactgatatccaaactgcaattggcggtggtgcaacgggggttactggggctgatatcaaatttaaagatggtcaatactatttagatgttaaaggcggtgcttctgctggtgtttataaagccacttatgatgaaactacaaagaaagttaatattgatacgactgataaaactccgttagcaactgcggaagctacagctattcggggaacggccactataacccacaaccaaattgctgaagtaacaaaagagggtgttgatacgaccacagttgcggctcaacttgctgctgcaggggttactggtgccgataaggacaatactagccttgtaaaactatcgtttgaggataaaaacggtaaggttattgatggtggctatgcagtgaaaatgggcgacgatttctatgccgctacatatgatgagaaaacaggtacaattactgctaaaacaaccacttatacagatggtgctggcgttgctcaaactggagctgtgaaatttggtggcgcaaatggtaaatctgaagttgttactgctaccgatggtaaaacttacttagcaagcgaccttgacaaacataacttcagaacaggcggtgagcttaaagaggttaatacagataagactgaaaacccactgcagaaaattgatgctgccttggcacaggttgatacacttcgttctgacctgggtgcggtacagaaccgtttcaactccgctatcaccaacctgggcaataccgtaaataacctgtcttctgcccgtagccgtatcgaagattccgactacgcgaccgaagtctccaacatgtctcgcgcgcagattctgcagcaggccggtacctccgttctggcgcaggctaaccaggttccgcaaaacgtcctctctttactgcgttaa

SEQ ID NO: 126 - protein, 494 aa, Artificial Sequence
  Flagellin
AQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 127 - protein, 504 aa, Artificial Sequence
  Flagellin
AQVINTNSLSLLTQNNLNKSQSALGTAIERLSSGLRINSAKDDAAGQAIANRFTANIKGLTQASRNANDGISIAQTTEGALNEINNNLQRVRELAVQSANGTNSQSDLDSIQAEITQRLNEIDRVSGQTQFNGVKVLAQDNTLTIQVGANDGETIDIDLKEISSKTLGLDKLNVQDAYTPKETAVTVDKTTYKNGTDTITAQSNTDIQTAIGGGATGVTGADIKFKDGQYYLDVKGGASAGVYKATYDETTKKVNIDTTDKTPLATAEATAIRGTATITHNQIAEVTKEGVDTTTVAAQLAAAGVTGADKDNTSLVKLSFEDKNGKVIDGGYAVKMGDDFYAATYDEKQVQLLLNNHYTDGAGVLQTGAVKFGGANGKSEVVTATVGKTYLASDLDKHNFRTGGELKEVNTDKTENPLQKIDAALAQVDTLRSDLGAVQNRFNSAITNLGNTVNNLSSARSRIEDSDYATEVSNMSRAQILQQAGTSVLAQANQVPQNVLSLLR

SEQ ID NO: 128 - protein, 497 aa, Artificial Sequence
  Flagellin
AQVINTNSLSLITQNNINKNQSALSSSIERLSSGLRINSAKDDAAGQAIANRFTSNIKGLTQAARNANDGISVAQTTEGALSEINNNLQRVRELTVQATTGTNSESDLSSIQDEIKSRLDEIDRVSGQTQFNGVNVLAKNGSMKIQVGANDNQTITIDLKQIDAKTLGLDGFSVKNNDTVTTSAPVTAFGATTTNNIKLTGITLSTEAATDTGGTNPASIEGVYTDNGNDYYAKITGGDNDGKYYAVTVANDGTVTMATGATANATVTDANTTKATTITSGGTPVQIDNTAGSATANLGAVSLVKLQDSKGNDTDTYALKDTNGNLYAADVNETTGAVSVKTITYTDSSGAASSPTAVKLGGDDGKTEVVDIDGKTYDSADLNGGNLQTGLTAGGEALTAVANGKTTDPLKALDDAIASVDKFRSSLGAVQNRLDSAVTNLNNTTTNLSEAQSRIQDADYATEVSNMSKAQIIQQAGNSVLAKANQVPQQVLSLLQG

SEQ ID NO: 129 - protein, 393 aa, Artificial Sequence
  Flagellin
ALTVNTNIASLNTQRNLNNSSASLNTSLQRLSTGSRINSAKDDAAGLQIANRLTSQVNGLNVATKNANDGISLAQTAEGALQQSTNILQRMRDLSLQSANGSNSDSERTALNGEVKQLQKELDRISNTTTFGGRKLLDGSFGVASFQVGSAANEIISVGIDEMSAESLNGTYFKADGGGAVTAATASGTVDIAIGITGGSAVNVKVDMKGNETAEQAAAKIAAAVNDANVGIGAFSDGDTISYVSKAGKDGSGAITSAVSGVVIADTGSTGVGTAAGVTPSATAFAKTNDTVAKIDISTAKGAQSAVLVIDEAIKQIDAQRADLGAVQNRFDNTINNLKNIGENVSAARGRIEDTDFAAETANLTKNQVLQQAGTAILAQANQLPQSVLSLLR

SEQ ID NO: 130 - protein, 506 aa, Artificial Sequence; identical to SEQ ID NO: 115
  Flagellin

SEQ ID NO: 131 - protein, 287 aa, Artificial Sequence
  Flagellin
MKVNTNIISLKTQEYLRKNNEGMTQAQERLASGKRINSSLDDAAGLAVVTRMNVKSTGLDAASKNSSMGIDLLQTADSALSSMSSILQRMRQLAVQSSNGSFSDEDRKQYTAEFGSLIKELDHVADTTNYNNIKLLDQTATNAATQVSIQASDKANDLINIDLFNAKGLSAGTITLGSGSTVAGYSALSVADADSSQEATEAIDELINNISNGRALLGAGMSRLSYNVSNVNNQSIATKASASSIEDADMAAEMSEMTKYKILTQTSISMLSQANQTPQMLTQLINS

SEQ ID NO: 132 - protein, 505 aa, Artificial Sequence
  Flagellin
AQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 133 - protein, 20 aa, Artificial Sequence
  T-cell epitopes
KYLKRIKNSISTEWSPCSVT

SEQ ID NO: 134 - protein, 20 aa, Artificial Sequence
  T-cell epitopes
QYLQTIRNSLSTEWSPCSVT

SEQ ID NO: 135 - protein, 20 aa, Artificial Sequence
  T-cell epitopes
EFLKQIQNSLSTEWSPCSVT

SEQ ID NO: 136 - protein, 20 aa, Artificial Sequence
  T-cell epitopes
EFVKQISSQLTEEWSQCNVT

SEQ ID NO: 137 - protein, 20 aa, Artificial Sequence
  T-cell epitopes
EFVKQIRDSITEEWSQCSVT

SEQ ID NO: 138 - protein, 9 aa, Artificial Sequence
  B-cell epitopes
DRADGQPAG

SEQ ID NO: 139 - protein, 18 aa, Artificial Sequence
  B-cell epitopes
DRADGQPAGDRADGQPAG

SEQ ID NO: 140 - protein, 9 aa, Artificial Sequence
  B-cell epitopes
DRAAGQPAG

SEQ ID NO: 141 - protein, 18 aa, Artificial Sequence
  B-cell epitopes
DRAAGQPAGDRAAGQPAG

SEQ ID NO: 142 - protein, 18 aa, Artificial Sequence
  B-cell epitopes
DRADGQPAGDRAAGQPAG

SEQ ID NO: 143 - protein, 36 aa, Artificial Sequence
  B-cell epitopes
ANGAGNQPGANGAGNQPGANGAGNQPGANGAGNQPG

SEQ ID NO: 144 - protein, 16 aa, Artificial Sequence
  B-cell epitopes
NAAGNAAGNAAGNAAG

SEQ ID NO: 145 - protein, 16 aa, Artificial Sequence
  B-cell epitopes
PPPPNPNDPPPPNPND

SEQ ID NO: 146 - protein, 319 aa, Artificial Sequence; identical to SEQ ID NO: 5
  P. falciparum CSP

SEQ ID NO: 147 - protein, 48 aa, Artificial Sequence
  TIBT*
DPNANPNVDPNANPNVNANPNANPNANPEYLNKIQNSLSTEWSPCSVT

SEQ ID NO: 148 - protein, 192 aa, Artificial Sequence
  4xTIBT*
DPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVT

SEQ ID NO: 149 - protein, 480 aa, Artificial Sequence
  10xTIBT*
DPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVTDPNANPNVDPNANPNVNANPNANPNANPEYLNKIQNSLSTEWSPCSVT

SEQ ID NO: 150 - protein, 360 aa, Artificial Sequence
  10xTIT*
DPNANPNVDPNANPNVEYLNKIQNSLSTEWSPCSVTDPNANPNVDPNANPNVEYLNKIQNSLSTEWSPCSVTDPNANPNVDPNANPNVEYLNKIQNSLSTEWSPCSVTDPNANPNVDPNANPNVEYLNKIQNSLSTEWSPCSVTDPNANPNVDPNANPNVEYLNKIQNSLSTEWSPCSVTDPNANPNVDPNANPNVEYLNKIQNSLSTEWSPCSVTDPNANPNVDPNANPNVEYLNKIQNSLSTEWSPCSVTDPNANPNVDPNANPNVEYLNKIQNSLSTEWSPCSVTDPNANPNVDPNANPNVEYLNKIQNSLSTEWSPCSVTDPNANPNVDPNANPNVEYLNKIQNSLSTEWSPCSVT

SEQ ID NO: 151 - protein, 320 aa, Artificial Sequence
  10xBT*
NANPNANPNANPEYLNKIQNSLSTEWSPCSVTNANPNANPNANPEYLNKIQNSLSTEWSPCSVTNANPNANPNANPEYLNKIQNSLSTEWSPCSVTNANPNANPNANPEYLNKIQNSLSTEWSPCSVTNANPNANPNANPEYLNKIQNSLSTEWSPCSVTNANPNANPNANPEYLNKIQNSLSTEWSPCSVTNANPNANPNANPEYLNKIQNSLSTEWSPCSVTNANPNANPNANPEYLNKIQNSLSTEWSPCSVTNANPNANPNANPEYLNKIQNSLSTEWSPCSVTNANPNANPNANPEYLNKIQNSLSTEWSPCSVT

SEQ ID NO: 152 - protein, 16 aa, Artificial Sequence
  T1 epitope
DPNANPNVDPNANPNV

SEQ ID NO: 153 - protein, 4 aa, Artificial Sequence
  Flagellin
TIAL

SEQ ID NO: 154 - protein, 4 aa, Artificial Sequence
  Flagellin
TTLD

SEQ ID NO: 155 - protein, 7 aa, Artificial Sequence
  Flagellin
GTDQKID

SEQ ID NO: 156 - protein, 6 aa, Artificial Sequence
  Flagellin
NGEVTL

SEQ ID NO: 157 - protein, 5 aa, Artificial Sequence
  Flagellin
GADAA

SEQ ID NO: 158 - protein, 4 aa, Artificial Sequence
  Flagellin
AGGA

SEQ ID NO: 159 - protein, 4 aa, Artificial Sequence
  Flagellin
PATA

SEQ ID NO: 160 - protein, 4 aa, Artificial Sequence
  Flagellin
AAGA

SEQ ID NO: 161 - protein, 4 aa, Artificial Sequence
  Flagellin
ATTK

SEQ ID NO: 162 - protein, 12 aa, Artificial Sequence
  Flagellin
AGATKTTMPAGA

SEQ ID NO: 163 - protein, 5 aa, Artificial Sequence
  Flagellin
GVTGT

SEQ ID NO: 164 - protein, 10 aa, Artificial Sequence
  Flagellin
TEAKAALTAA

SEQ ID NO: 165 - protein, 11 aa, Artificial Sequence
  Flagellin
ASVVKMSYTDN

SEQ ID NO: 166 - protein, 9 aa, Artificial Sequence
  Flagellin
VDATDANGA

SEQ ID NO: 167 - protein, 9 aa, Artificial Sequence
  CSP Repeat
ANGAGNQPG

SEQ ID NO: 168 - protein, 36 aa, Artificial Sequence; identical to SEQ ID NO: 143
  CSP repeat

SEQ ID NO: 169 - protein, 26 aa, Artificial Sequence
  T-cell epitope
DPNANPNVDPNANPNVDPNANPNVDP

SEQ ID NO: 170 - protein, 20 aa, Artificial Sequence
  T-cell epitope
QYLKKIQNSLSTEWSPCSVT

SEQ ID NO: 171 - protein, 20 aa, Artificial Sequence
  T-cell epitope
QYLKKIKNSISTEWSPCSVT

SEQ ID NO: 172 - protein, 8 aa, Artificial Sequence
  Repeat
NANPNANP

SEQ ID NO: 173 - protein, 16 aa, Artificial Sequence
  Repeat
NANPNANPNANPNANP

SEQ ID NO: 174 - protein, 20 aa, Artificial Sequence
  Repeat
NANPNANPNANPNANPNANP

SEQ ID NO: 175 - protein, 8 aa, Artificial Sequence
  TLR4 agonist
GGKSGRTG

SEQ ID NO: 176 - protein, 9 aa, Artificial Sequence
  TLR4 agonist
KGYDWLVVG

SEQ ID NO: 177 - protein, 10 aa, Artificial Sequence
  TLR4 agonist
EDMVYRIGVP

SEQ ID NO: 178 - protein, 6 aa, Artificial Sequence
  TLR4 agonist
VKLSGS

SEQ ID NO: 179 - protein, 8 aa, Artificial Sequence
  TLR4 agonist
GMLSLALF

SEQ ID NO: 180 - protein, 7 aa, Artificial Sequence
  TLR4 agonist
CVVGSVR

SEQ ID NO: 181 - protein, 8 aa, Artificial Sequence
  TLR4 agonist
IVRGCLGW

SEQ ID NO: 182 - protein, 5 aa, Artificial Sequence
  TLR2 agonist
NPPTT

SEQ ID NO: 183 - protein, 5 aa, Artificial Sequence
  TLR2 agonis
MRRIL

SEQ ID NO: 184 - protein, 4 aa, Artificial Sequence
  TLR2 agonis
MISS

SEQ ID NO: 185 - protein, 5 aa, Artificial Sequence
  TLR2 agonis
RGGSK

SEQ ID NO: 186 - protein, 4 aa, Artificial Sequence
  TLR2 agonis
RGGF

SEQ ID NO: 187 - protein, 4 aa, Artificial Sequence
  N-glycosylation site
NXST

SEQ ID NO: 188 - protein, 535 aa, Artificial Sequence
  CRM 197
GADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 189 - protein, 164 aa, Artificial Sequence
  TMV coat protein
MMAYSIPTPSQLVYFTENYADYIPFVNRLINARSNSFQTQSGRDELREILIKSQVSVVSPISRFPAEPAYYIYLRDPSISTVYTALLQSTDTRNRVIEVENSTNVTTAEQLNAVRRTDDASTAIHNNLEQLLSLLTNGTGVFNRTSFESASGLTWLVTTTPRTA

SEQ ID NO: 190 - protein, 221 aa, Artificial Sequence
  coat protein AMV
MSSSQKKAGGKAGKPTKRSQNYAALRKAQLPKPPALKVPVAKPTNTILPQTGCVWQSLGTPLSLSSSNGLGARFLYSFLKDFAAPRILEEDLIFRMVFSITPSHAGSFCLTDDVTTEDGRAVAHGNPMQEFPHGAFHANEKFGFELVFTAPTHAGMQNQNFKHSYAVALCLDFDALPEGSRNPSYRFNEVWVERKAFPRAGPLRSLITVGLFDDADDLDRQ

SEQ ID NO: 191 - protein, 237 aa, Artificial Sequence
  coat protein potato virus x
MTTPANTTQATGSTTSTTTKTAGATPATTSGLFTIPDGEFFSTARAIVASNAVATNEDLSKIEAIWKDMKVPTDTMAQAAWDLVRHCADVGSSAQTEMIDTGPYSNGISRARLAAAIKEVCTLRQFCMKYAPVVWNWMLTNNSPPANWQAQGFKPEHKFAAFDFFNGVTNPAAIMPKEGLIRPPSEAEMNAAQTAAFVKITKARAQSNDFASLDAAVTRGRITGTTTAEAVVTLPPP

SEQ ID NO: 192 - protein, 395 aa, Artificial Sequence
  Porin
MRKKLTALVLSALPLAAVADVSLYGEIKAGVEGRNYQLQLTEAQAANGGASGQVKVTKVTKAKSRIRTKISDFGSFIGFKGSEDLGEGLKAVWQLEQDVSVAGGGATQWGNRESFIGLAGEFGTLRAGRVANQFDDASQAIDPWDSNNDVASQLGIFKRHDDMPVSVRYDSPEFSGFSGSVQFVPAQNSKSAYKPAYWTTVNTGSATTTTFVPAVVGKPGSDVYYAGLNYKNGGFAGNYAFKYARHANVGRDAFELFLLGSGSDQAKGTDPLKNHQVHRLTGGYEEGGLNLALAAQLDLSENGDKTKNSTTEIAATASYRFGNAVPRISYAHGFDFIERGKKGENTSYDQIIAGVDYDFSKRTSAIVSGAWLKRNTGIGNYTQINAASVGLRHKF

SEQ ID NO: 193 - protein, 347 aa, Artificial Sequence
  Fimbrillin
MVLKTSNSNRAFGVGDDESKVAKLTVMVYNGEQQEAIKSAENATKVEDIKCSAGQRTLVVMANTGAMELVGKTLAEVKALTTELTAENQEAAGLIMTAEPKTIVLKAGKNYIGYSGTGEGNHIENDPLKIKRVHARMAFTEIKVQMSAAYDNIYTFVPEKIYGLIAKKQSNLFGATLVNADANYLTGSLTTFNGAYTPANYANVPWLSRNYVAPAADAPQGFYVLENDYSANGGTIHPTILCVYGKLQKNGADLAGADLAAAQAANWVDAEGKTYYPVLVNFNSNNYTYDSNYTPKNKIERNHKYDIKLTITGPGTNNPENPITESAHLNVQCTVAEWVLVGQNATW

SEQ ID NO: 194 - protein, 428 aa, Artificial Sequence
  MALP-2
MKKSKKILLGLSPIAAVLPAVAVSCGNNDESNISFKEKDISKYTTTNANGKQVVKNAELLKLKPVLITDEGKIDDKSFNQSAFEALKAINKQTGIEINSVEPSSNFESAYNSALSAGHKIWVLNGFKHQQSIKQYIDAHREELERNQIKIIGIDFDIETEYKWFYSLQFNIKESAFTTGYAIASWLSEQDESKRVVASFGVGAFPGVTTFNEGFAKGILYYNQKHKSSKIYHTSPVKLDSGFTAGEKMNTVINNVLSSTPADVKYNPHVILSVAGPATFETVRLANKGQYVIGVDSDQGMIQDKDRILTSVLKHIKQAVYETLLDLILEKEEGYKPYVVKDKKADKKWSHFGTQKEKWIGVAENHFSNTEEQAKINNKIKEAIKMFKELPEDFVKYINSDKALKDGNKIDNVSERLEAIISAINKAAK

SEQ ID NO: 195 - protein, 143 aa, Artificial Sequence
  p19 protein
ATTLPVQRHPRSLFPEFSELFAAFPSFAGLRPTFDTRLMRLEDEMKEGRYEVRAELPGVDPDKDVDIMVRDGQLTIKAERTEQKDFDGRSEFAYGSFVRTVSLPVGADEDDIKATYDKGILTVSVAVSEGKPTEKHIQIRSTN

SEQ ID NO: 196 - protein, 16 aa, Artificial Sequence
  T-cell epitope
NVDPNVDPNVDPNVDP

SEQ ID NO: 197 - protein, 8 aa, Artificial Sequence
  T-cell epitope
NVDPNANP

SEQ ID NO: 198 - protein, 24 aa, Artificial Sequence
  T-cell epitope
NVDPNANPNVDPNANPNVDPNANP

SEQ ID NO: 199 - protein, 16 aa, Artificial Sequence
  T-cell epitope
NVDPNVDPNVDPNVDP

SEQ ID NO: 200 - protein, 8 aa, Artificial Sequence
  T1 epitope
DPNANPNV

SEQ ID NO: 201 - DNA, 525 bp, Artificial Sequence
  CSP
gaagaaccaaagaagccaaatgaaaataagctgaaacaaccgaatgaaggacaaccacaagcacagggtgatggagcaaatgcaggacaaccacaagcacaaggagatggagcaaatgcaggacaaccacaagcacagggtgatggagcaaatgcaggacaaccacaagcacagggtgatggagcaaatgcaggacaaccacaagcacaaggagatggagcaaatgcagggcaaccacaagcacagggtgatggagcaaatgcaggacaaccacaagcacagggtgatggagcaaatgcaggacaaccacaagcacaaggagatggagcaaatgcaggacaaccacaagcacaaggagatggagcaaatgcaggacaaccacaagcacagggtgatggagcaaatgcaggacaaccacaagcacagggtgatggagccaatgcaggacaaccacaagcacaaggagatggggcaaatgtaccacgacaaggaagaaacgggggaggtgcaccagcaggaggaaat

SEQ ID NO: 202 - protein, 363 aa, Artificial Sequence
  CSP
MKNFILLAVSSILLVDLLPTHFEHNVDLSRAINVNGVSFNNVDTSSLGAAQVRQSASRGRGLGEKPKEGADKEKKKEKGKEKEEEPKKPNENKLKQPNEGQPQAQGDGANAGQPQAQGDGANAGQPQAQGDGANAGQPQAQGDGANAGQPQAQGDGANAGQPQAQGDGANAGQPQAQGDGANAGQPQAQGDGANAGQPQAQGDGANAGQPQAQGDGANAGQPQAQGDRANAGQPQAQGDGANVPRQGRNGGGAPAGGNEGNKQAGKGQGQNNQGANAPNEKVVNDYLHKIRSSVTTEWTPCSVTCGNGVRIRRKAHAGNKKAEDLTMDDLEVEACVMDKCAGIFNVVSNSLGLVILLVLALFN

SEQ ID NO: 203 - DNA, 1104 bp, Artificial Sequence
  CSP
atgaagaacttcattctcttggccgtctcctccatcctgctggtggacttgctccccacacacttcgaacataatgtagatctctccagggccataaatgtaaatggagtaagcttcaataatgtagacaccagttcacttggcgcagcacaggtgagacaaagtgctagccgaggcagaggacttggtgagaagccaaaagaaggagctgataaagaaaagaaaaaagaaaaagaaaaagaagaagaaccaaagaagccaaatgaaaataagctgaaacaacctaatgcagaaggtgatggagcaaatgcacgacaacctaatgcagaaggtgatggagcaaatgcacgacaaccgaatgcagaaggtgatggagcaaatgcacgacaacctaatgcagaaggtgatggagcaaatgcacgacaaccgaatgcagaaggtgatggagcaaatgcacgacaacctaatgcagaaggtgatggagcaaatgcacgacaacctaatgcagaaggtgatggagcaaatgcacgacaacctaatgcagaaggtgatggagcaaatgcacgacaaccgaatgcagaaggtgatggagcaaatgcacgacaacctaatgcagaaggtgatggagcaaatgcacgacaaccgaatgcagaaggtggtggagcaaatgcacgacagccacaggcagaaggtgatggagcaaatgcacgacaaccacaagcacaaggagatggaggaaatgcacgacaaggaggaaacgggggaggtgcaccagcaggaggaaatgaggggaataaacaagcaggaaaaggacagggacaaaacaatcagggtgcgaatgccccaaatgaaaaagttgtaaatgattacctacagaaaattagatctagcgttaccaccgagtggactccatgcagtgtaacctgtggaaatggtgtaagaattagaagaagagctcatgcagataagaaaaaggcagaggaccttactatggatgaccttgaagtggaagcttgtgtaatggataagtgtgctggcatatttaacgttgtgagtaattcattagggttcgtcatattgttagtcctagcattattcaattaa

SEQ ID NO: 204 - protein, 367 aa, Artificial Sequence
  CSP
MKNFILLAVSSILLVDLLPTHFEHNVDLSRAINVNGVSFNNVDTSSLGAAQVRQSASRGRGLGEKPKEGADKEKKKEKEKEEEPKKPNENKLKQPNAEGDGANARQPNAEGDGANARQPNAEGDGANARQPNAEGDGANARQPNAEGDGANARQPNAEGDGANARQPNAEGDGANARQPNAEGDGANARQPNAEGDGANARQPNAEGDGANARQPNAEGGGANARQPQAEGDGANARQPQAQGDGGNARQGGNGGGAPAGGNEGNKQAGKGQGQNNQGANAPNEKVVNDYLQKIRSSVTTEWTPCSVTCGNGVRIRRRAHADKKKAEDLTMDDLEVEACVMDKCAGIFNVVSNSLGFVILLVLALFN

SEQ ID NO: 205 - DNA, 1089 bp, Artificial Sequence
  CSP
atgaagaacttcattctcttggccgtctcctccatcctgctggtggacttgctccccacatacttcgaacataatgtagatctctccagggccataaatgtaaatggagtaagcttcaataatgtagacaccagttcacttggcgcagcacaggtaagacaaagtgctagccgaggcagaggacttggtgagaagccaaaagaaggagctgataaagaaaagaaaaaagaaaaagaaaaagaaaaagaagaagaaccaaagaagccaaatgaaaataagctgaaacaaccagggggcgaacaagcaggaccagggggcgaacaagcaggaccaggaggcgaacaagcaggaccaggaggcgaacaagcaggaccaagaccagggggcgaacaagcaggaccaggaggcgaacaagcaggaccaagaccagggggcgaacaagcaggaccaggaggcgaacaagcaggaccaagaccagggggcgaacaagcaggaccaggaggcgaacaagcaggaccaagaccagggggcgaacaagcaggaccaggaggcgaacaagcaggaccaagaccagggggcgaacaagcaggaccaggaggcgaacaagcaggaccaagaccagggggcgaacaagcaggaccagggggcgaacaaccagcaccaagaccagggggagaacaaccagcaccagcaccaaggagggaacaaccagcaccaggaccagggggcgaacaaccagcaccaggagcaggtgcgggagatggagcacgaggaggaaacgcaggggcaggtaaaggacagggacaaaacaatcagggtgcgaatgtcccaaatgaaaaagttgtgaatgattacctacacaaaattagatctagcgttaccaccgagtggactccatgcagtgtaacctgtggaaatggtgtaagaattagaagaagacagaatgctggtaataaaaaggcagaggaccttactatggatgaccttgaggtggaaacttgtgtaatggataagtgcgctggcatatttaacgttgtgagtaattcattagggttagtcatattgttagtcctagcattattcaattaa

SEQ ID NO: 206 - protein, 362 aa, Artificial Sequence
  CSP
MKNFILLAVSSILLVDLLPTYFEHNVDLSRAINVNGVSFNNVDTSSLGAAQVRQSASRGRGLGEKPKEGADKEKKKEKEKEKEEEPKKPNENKLKQPGGEQAGPGGEQAGPGGEQAGPGGEQAGPRPGGEQAGPGGEQAGPRPGGEQAGPGGEQAGPRPGGEQAGPGGEQAGPRPGGEQAGPGGEQAGPRPGGEQAGPGGEQAGPRPGGEQAGPGGEQPAPRPGGEQPAPAPRREQPAPGPGGEQPAPGAGAGDGARGGNAGAGKGQGQNNQGANVPNEKVVNDYLHKIRSSVTTEWTPCSVTCGNGVRIRRRQNAGNKKAEDLTMDDLEVETCVMDKCAGIFNVVSNSLGLVILLVLALFN

SEQ ID NO: 207 - DNA, 1089 bp, Artificial Sequence; identical to SEQ ID NO: 205
  CSP

SEQ ID NO: 208 - protein, 362 aa, Artificial Sequence; identical to SEQ ID NO: 206
  CSP

SEQ ID NO: 209 - DNA, 1128 bp, Artificial Sequence
  CSP
atgaagaacttcattctcttggccgtctcctccatcctgctggtggacttgctccccacacacttcgaacataatgtagatctctccagggccataaatgtaaatggagtaagcttcaataatgtagacaccagttcacttggcgcagcacaggtgagacaaagtgctagccgaggcagaggacttggtgagaagccaaaagaaggagctgataaagaaaagaaaaaagaaaaaggaaaagaaaaagaagaagaaccaaagaagccaaatgaaaataagctgaaacaaccgaatgaaggacaaccacaagcacagggtgatggagcaaatgcagggcaaccacaagcacagggtgatggagcaaatgcaggacaaccacaagcacaaggagatggagcaaatgcaggacaaccacaagcacagggtgatggagcaaatgcaggacaaccacaagcacagggtgatggagcaaatgcaggacaaccacaagcacaaggagatggagcaaatgcaggacaaccacaagcacagggtgatggagcaaatgcagggcaaccacaagcacagggtgatggagcaaatgcaggacaaccacaagcacaaggagatggagcaaatgcaggacaaccacaagcacaaggagatggagcaaatgcaggacaaccacaagcacagggtgatggagcaaatgcaggacaaccacaagcacagggtgatagggcgaatgcaggacaaccacaagcacaaggagatggggcaaatgtaccacgacaaggaagaaacgggggaggtgcaccagcaggaggaaatgaggggaataaacaagcaggaaaaggacagggacaaaacaatcagggtgcgaatgccccaaatgaaaaagttgtgaatgattacctacacaaaattagatctagcgttaccaccgagtggactccatgcagtgtaacctgtggaaatggtgtaagaattagaagaaaagctcatgcaggtaataaaaaggcagaggaccttactatggatgaccttgagttggaagcttgtgtaatggataagtgcgctggcatatttaacgttgtgagtaattcattaggcttagtcatattgttagtcctagcattattcaattaa

SEQ ID NO: 210 - protein, 375 aa, Artificial Sequence
  CSP
MKNFILLAVSSILLVDLLPTHFEHNVDLSRAINVNGVSFNNVDTSSLGAAQVRQSASRGRGLGEKPKEGADKEKKKEKGKEKEEEPKKPNENKLKQPNEGQPQAQGDGANAGQPQAQGDGANAGQPQAQGDGANAGQPQAQGDGANAGQPQAQGDGANAGQPQAQGDGANAGQPQAQGDGANAGQPQAQGDGANAGQPQAQGDGANAGQPQAQGDGANAGQPQAQGDGANAGQPQAQGDRANAGQPQAQGDGANVPRQGRNGGGAPAGGNEGNKQAGKGQGQNNQGANAPNEKVVNDYLHKIRSSVTTEWTPCSVTCGNGVRIRRKAHAGNKKAEDLTMDDLELEACVMDKCAGIFNVVSNSLGLVILLVLALFN

SEQ ID NO: 211 - DNA, 1056 bp, Artificial Sequence
  CSP
atgaagaacttcattctcttggccgtctcctccatcctgctggtggacttgctccccacacacttcgaacataatgtagatctctccagggccataaatgtaaatggagtaagcttcaataatgtagacaccagttcacttggcgcagcacgggtaagacaaagtgctagccgaggcagaggacttggtgagaagccaaaagaaggagctgataaagaaaagaaaaaagaaaaagaaaaagaaaaagaagaagaaccaaagaagccaaatgaaaataagctgaaacaaccggaacaaccagcagcaggagcagggggcgaacaaccagcagcaggagcaggaggcgaacaaccagcagcaggagcaggaggcgaacaaccagcagcaggagcaggaggcgaacaaccagcagcaggagcaagaggcgaacaaccagcagcaggagcaggaggcgaacaaccagcagcaggagcaggaggcgaacaaccagcagcaggagcaggaggcgaacaaccagcagcaggagcaagaggcgaacaaccagcagcaggagcaggaggcgaacaaccagcagcaggagcaagaggcgaacaaccagcagcaggagcaggaggcgaacaaccagcagcaggagcaggaggcgaacaaccagcagcaggagcaagaggcgaacaaccagcaccagcaccaaggagggaacaaccagcaccaggagcaggtgcgggagatggagcacgaggaggaaacgcaggggcaggtaaaggacagggacaaaacaatcagggtgcgaatgtcccaaatgaaaaagttgtgaatgattacctacacaaaattagatctagcgttaccaccgagtggactccatgcagtgtaacctgtggaaatggtgtaagaattagaagaaaaggtcatgcaggtaataaaaaggcagaggaccttactatggatgaccttgaggtggaagcttgtgtaatggataagtgcgctggcatatttaacgttgtgagtaattcattaggcttagtcatattgttagtcctagcattattcaattaa

SEQ ID NO: 212 - protein, 351 aa, Artificial Sequence
  CSP
MKNFILLAVSSILLVDLLPTHFEHNVDLSRAINVNGVSFNNVDTSSLGAARVRQSASRGRGLGEKPKEGADKEKKKEKEKEKEEEPKKPNENKLKQPEQPAAGAGGEQPAAGAGGEQPAAGAGGEQPAAGAGGEQPAAGARGEQPAAGAGGEQPAAGAGGEQPAAGAGGEQPAAGARGEQPAAGAGGEQPAAGARGEQPAAGAGGEQPAAGAGGEQPAAGARGEQPAPAPRREQPAPGAGAGDGARGGNAGAGKGQGQNNQGANVPNEKVVNDYLHKIRSSVTTEWTPCSVTCGNGVRIRRKGHAGNKKAEDLTMDDLEVEACVMDKCAGIFNVVSNSLGLVILLVLALFN

SEQ ID NO: 213 - DNA, 1056 bp, Artificial Sequence
  CSP
atgaagaacttcattctcttggccgtctcctccatcctgctggtggacttgctccccacacacttcgaacataatgtagatctctccagggcaataaatgtaaatggagtaagcttcaataatgtagacaccagttcacttggcgcagcacaggtaagacaaagtgctagccgaggcagaggacttggtgagaagccaaaagaaggagctgataaagaaaagaaaaaagaaaaagaaaaagaaaaagaagaagaaccaaagaagccaaatgaaaataagctgaaacaaccggaacaaccagcagcaggagcaggaggcgaacaaccagcagcaggagcaggaggcgaacaaccagcagcaggagcaggaggcgaacaaccagcagcaggagcaagaggcgaacaaccagcagcaggagcaggaggcgaacaaccagcagcaggagcaggaggcgaacaaccagcagcaggagcaggaggcgaacaaccagcagcaggagcaggaggcgaacaaccagcagcaggagcaggaggcgaacaaccagcagcaggagcaagaggcgaacaaccagcagcaggagcaggaggcgaacaaccagcagcaggagcaggaggcgaacaaccagcagcaggagcaagaggcgaacaaccagcagcaggagcaggaggcgaacaaccagcaccagcaccaaggagggaacaaccagcaccaggagcagtcgccggagatggagcacgaggaggaaacgcaggggcaggtaaaggacagggacaaaacaatcagggtgcgaatgtcccaaatgaaaaagttgtgaatgattacctacacaaaattagatctagcgttaccaccgagtggactccatgcagtgtaacctgtggaaatggtgtaagaataagaagaaaaggtcatgcaggtaataaaaaggcagaggaccttactatggatgaccttgaggtggaagcttgtgtaatggataagtgcgctggcatatttaacgttgtgagtaattcattaggcttagtcatattgttagtcctagcattattcaattaa

SEQ ID NO: 214 - protein, 351 aa, Artificial Sequence
  CSP
MKNFILLAVSSILLVDLLPTHFEHNVDLSRAINVNGVSFNNVDTSSLGAAQVRQSASRGRGLGEKPKEGADKEKKKEKEKEKEEEPKKPNENKLKQPEQPAAGAGGEQPAAGAGGEQPAAGAGGEQPAAGARGEQPAAGAGGEQPAAGAGGEQPAAGAGGEQPAAGAGGEQPAAGAGGEQPAAGARGEQPAAGAGGEQPAAGAGGEQPAAGARGEQPAAGAGGEQPAPAPRREQPAPGAVAGDGARGGNAGAGKGQGQNNQGANVPNEKVVNDYLHKIRSSVTTEWTPCSVTCGNGVRIRRKGHAGNKKAEDLTMDDLEVEACVMDKCAGIFNVVSNSLGLVILLVLALFN

SEQ ID NO: 215 - DNA, 5063 bp, Artificial Sequence
  MSP1
atgaagatcatattctttttatgttcatttcttttttttattataaatacacaatgtgtaacacatgaaagttatcaagaacttgtcaaaaaactagaagctttagaagatgcagtattgacaggttatagtttatttcaaaaggaaaaaatggtattaaatgaagaagaaattactacaaaaggtgcaagtgctcaaagtggtgcaagtgctcaaagtggtgcaagtgctcaaagtggtgcaagtgctcaaagtggtgcaagtgctcaaagtggtgcaagtgctcaaagtggtacaagtggtccaagtggtccaagtggtacaagtccatcatctcgttcaaacactttacctcgttcaaatacttcatctggtgcaagccctccagctgatgcaagcgattcagatgctaaatcttacgctgatttaaaacacagagtacgaaattacttgttcactattaaagaactcaaatatcccgaactctttgatttaaccaatcatatgttaactttgtgtgataatattcatggtttcaaatatttaattgatggatatgaagaaattaatgaattattatataaattaaacttttattttgatttattaagagcaaaattaaatgatgtatgtgctaatgattattgtcaaatacctttcaatcttaaaattcgtgcaaatgaattagacgtacttaaaaaacttgtgttcggatatagaaaaccattagacaatattaaagataatgtaggaaaaatggaagattacattaaaaaaaataaaacaaccatagcaaatataaatgaattaattgaaggaagtaagaaaacaattgatcaaaataagaatgcagataatgaagaagggaaaaaaaaattataccaagctcaatatgatctttctatttacaataaacaattagaagaagcacataatttaataagcgttttagaaaaacgtattgacactttaaaaaaaaatgaaaacataaagaaattacttgataagataaatgaaattaaaaatcccccaccggccaattctggaaatacaccaaatactctccttgataagaacaaaaaaatcgaggaacacgaagaaaaaataaaagaaattgccaaaactattaaatttaacattgatagtttatttactgatccacttgaattagaatattatttaagagaaaaaaataaaaaagttgatgtaacacctaaatcacaagatcctacgaaatctgttcaaataccaaaagttccttatccaaatggtattgtatatcctttaccactcactgatattcataattcattagctgcagataatgataaaaattcatatggtgatttaatgaatcctcatactaaagaaaaaattaatgaaaaaattattacagataataaggaaagaaaaatattcattaataacattaaaaaaaaaattgatttagaagaaaaaaacattaatcacacaaaagaacaaaataaaaaattacttgaagattatgaaaagtcaaaaaaggattatgaagaattacttgaaaaattttatgaaatgaaatttaataataattttaacaaagatgtcgtagataaaatattcagtgcaagatatacatataatgttgaaaaacaaagatataataataaattttcatcctctaataattctgtatataatgttcaaaaattaaaaaaggctctttcatatcttgaagattattctttaagaaaaggaatttctgaaaaagattttaatcattattatactttgaaaactggcctcgaagctgatataaaaaaattaacagaagaaataaagagtagtgaaaacaaaattctagaaaaaaattttaaaggactaacacattcagcaaatggttccttagaagtatctgatattgtaaaattacaagtacaaaaagttttattaattaaaaaaatagaagacttaagaaagatagaattatttttaaaaaatgcacaactaaaagatagtattcatgtaccaaatatttataaaccacaaaataaaccagaaccatattatttaattgtattaaaaaaagaagtagataaattaaaagaatttataccaaaagtaaaagacatgttaaagaaagaacaagctgtcttatcaagtattacacaacctttagttgcagcaagcgaaacaactgaagatgggggtcactccacacacacattatcccaatcaggagaaacagaagtaacagaagaaacagaagaaacagaagaaacagtaggacacacaacaacggtaacaataacattaccaccaacacaaccatcaccaccaaaagaagtaaaagttgttgaaaattcaatagaacataagagtaatgacaattcacaagccttgacaaaaacagtttatctaaagaaattagatgaatttttaactaaatcatatatatgtcataaatatattttagtatcaaactctagtatggaccaaaaattattagaggtatataatcttactccagaagaagaaaatgaattaaaatcatgtgatccattagatttattatttaatattcaaaataacatacctgctatgtattcattatatgatagtatgaacaatgatttacaacatctcttttttgaattatatcaaaaggaaatgatttattatttacataaactaaaagaggaaaatcacatcaaaaaattattagaggagcaaaaacaaataactggaacatcatctacatccagtcctggaaatacaaccgtaaatactgctcaatccgcaactcacagtaattcccaaaaccaacaatcaaatgcatcctctaccaatacccaaaatggtgtagctgtatcatctggtcctgctgtagttgaagaaagtcatgatcccttaacagtattgtctattagtaacgatttgaaaggtattgttagtctcttaaatcttggaaataaaactaaagtacctaatccattaaccatttctacaacagagatggaaaaattttatgagaatattttaaaaaataatgatacctattttaatgatgatatcaaacaattcgtaaaatctaattcaaaagtaattacaggtttgaccgaaacacaaaaaaatgcattaaatgatgaaattaaaaaattaaaagatactttacagttatcatttgatttatataataaatataaattaaaattagatagattatttaataagaaaaaagaacttggccaagacaaaatgcaaattaaaaaacttactttattaaaagaacaattagaatcaaaattgaattcacttaataacccacataatgtattacaaaacttttctgttttctttaacaaaaaaaaagaagctgaaatagcagaaactgaaaacacattagaaaacacaaaaatattattgaaacattataaaggacttgttaaatattataatggtgaatcatctccattaaaaactttaagtgaagtatcaattcaaacagaagataattatgccaatttagaaaaatttagagtattaagtaaaatagatggaaaactcaatgataatttacatttaggaaagaaaaaattatctttcttatcaagtggattacatcatttaattactgaattaaaagaagtaataaaaaataaaaattatacaggtaattctccaagtgaaaataataagaaagttaacgaagctttaaaatcttacgaaaattttctcccagaagcaaaagttacaacagttgtaactccacctcaaccagatgtaactccatctccattatctgtaagggtaagtggtagttcaggatccacaaaagaagaaacacaaataccaacttcaggctctttattaacagaattacaacaagtagtacaattacaaaattatgacgaagaagatgattccttagttgtattacccatttttggagaatccgaagataatgacgaatatttagatcaagtagtaactggagaagcaatatctgtcacaatggataatatcctctcaggatttgaaaatgaatatgatgttatatatttaaaacctttagctggagtatatagaagcttaaaaaaacaaattgaaaaaaacatttttacatttaatttaaatttgaacgatatcttaaattcacgtcttaagaaacgaaaatatttcttagatgtattagaatctgatttaatgcaatttaaacatatatcctcaaatgaatacattattgaagattcatttaaattattgaattcagaacaaaaaaacacacttttaaaaagttacaaatatataaaagaatcagtagaaaatgatattaaatttgcacaggaaggtataagttattatgaaaaggttttagcgaaatataaggatgatttagaatcaattaaaaaagttatcaaagaagaaaaggagaagttcccatcatcaccaccaacaacacctccgtcaccagcaaaaacagacgaacaaaagaaggaaagtaagttccttccatttttaacaaacattgagaccttatacaataacttagttaataaaattgacgattacttaattaacttaaaggcaaagattaacgattgtaatgttgaaaaagatgaagcacatgttaaaataactaaacttagtgatttaaaagcaattgatgacaaaatagatctttttaaaaacccttacgacttcgaagcaattaaaaaattgataaatgatgatacgaaaaaagatatgcttggcaaattacttagtacaggattagttcaaaattttcctaatacaataatatcaaaattaattgaaggaaaattccaagatatgttaaacatttcacaacaccaatgcgtaaaaaaacaatgtccagaaaattctggatgtttcagacatttagatgaaagagaagaatgtaaatgtttattaaattacaaacaagaaggtgataaatgtgttgaaaatccaaatcctacttgtaacgaaaataatggtggatgtgatgcagatgccacatgtaccgaagaagattcaggtagcagcagaaagaaaatcacatgtgaatgtactaaacctga

SEQ ID NO: 216 - protein, 1720 aa, Artificial Sequence
  MSP1
MKIIFFLCSFLFFIINTQCVTHESYQELVKKLEALEDAVLTGYSLFQKEKMVLNEEEITTKGASAQSGASAQSGASAQSGASAQSGASAQSGASAQSGTSGPSGPSGTSPSSRSNTLPRSNTSSGASPPADASDSDAKSYADLKHRVRNYLFTIKELKYPELFDLTNHMLTLCDNIHGFKYLIDGYEEINELLYKLNFYFDLLRAKLNDVCANDYCQIPFNLKIRANELDVLKKLVFGYRKPLDNIKDNVGKMEDYIKKNKTTIANINELIEGSKKTIDQNKNADNEEGKKKLYQAQYDLSIYNKQLEEAHNLISVLEKRIDTLKKNENIKKLLDKINEIKNPPPANSGNTPNTLLDKNKKIEEHEEKIKEIAKTIKFNIDSLFTDPLELEYYLREKNKKVDVTPKSQDPTKSVQIPKVPYPNGIVYPLPLTDIHNSLAADNDKNSYGDLMNPHTKEKINEKIITDNKERKIFINNIKKKIDLEEKNINHTKEQNKKLLEDYEKSKKDYEELLEKFYEMKFNNNFNKDVVDKIFSARYTYNVEKQRYNNKFSSSNNSVYNVQKLKKALSYLEDYSLRKGISEKDFNHYYTLKTGLEADIKKLTEEIKSSENKILEKNFKGLTHSANGSLEVSDIVKLQVQKVLLIKKIEDLRKIELFLKNAQLKDSIHVPNIYKPQNKPEPYYLIVLKKEVDKLKEFIPKVKDMLKKEQAVLSSITQPLVAASETTEDGGHSTHTLSQSGETEVTEETEETEETVGHTTTVTITLPPTQPSPPKEVKVVENSIEHKSNDNSQALTKTVYLKKLDEFLTKSYICHKYILVSNSSMDQKLLEVYNLTPEEENELKSCDPLDLLFNIQNNIPAMYSLYDSMNNDLQHLFFELYQKEMIYYLHKLKEENHIKKLLEEQKQITGTSSTSSPGNTTVNTAQSATHSNSQNQQSNASSTNTQNGVAVSSGPAVVEESHDPLTVLSISNDLKGIVSLLNLGNKTKVPNPLTISTTEMEKFYENILKNNDTYFNDDIKQFVKSNSKVITGLTETQKNALNDEIKKLKDTLQLSFDLYNKYKLKLDRLFNKKKELGQDKMQIKKLTLLKEQLESKLNSLNNPHNVLQNFSVFFNKKKEAEIAETENTLENTKILLKHYKGLVKYYNGESSPLKTLSEVSIQTEDNYANLEKFRVLSKIDGKLNDNLHLGKKKLSFLSSGLHHLITELKEVIKNKNYTGNSPSENNKKVNEALKSYENFLPEAKVTTVVTPPQPDVTPSPLSVRVSGSSGSTKEETQIPTSGSLLTELQQVVQLQNYDEEDDSLVVLPIFGESEDNDEYLDQVVTGEAISVTMDNILSGFENEYDVIYLKPLAGVYRSLKKQIEKNIFTFNLNLNDILNSRLKKRKYFLDVLESDLMQFKHISSNEYIIEDSFKLLNSEQKNTLLKSYKYIKESVENDIKFAQEGISYYEKVLAKYKDDLESIKKVIKEEKEKFPSSPPTTPPSPAKTDEQKKESKFLPFLTNIETLYNNLVNKIDDYLINLKAKINDCNVEKDEAHVKITKLSDLKAIDDKIDLFKNPYDFEAIKKLINDDTKKDMLGKLLSTGLVQNFPNTIISKLIEGKFQDMLNISQHQCVKKQCPENSGCFRHLDEREECKCLLNYKQEGDKCVENPNPTCNENNGGCDADATCTEEDSGSSRKKITCECTKPDSYPLFDGIFCSSSNFLGISFLLILMLILYSFI

SEQ ID NO: 217 - DNA, 1869 bp, Artificial Sequence
  AMA1
atgagaaaattatactgcgtattattattgagcgcctttgagtttacatatatgataaactttggaagaggacagaattattgggaacatccatatcaaaatagtgatgtgtatcgtccaatcaacgaacatagggaacatccaaaagaatacgaatatccattacaccaggaacatacataccaacaagaagattcaggagaagacgaaaatacattacaacacgcatatccaatagaccacgaaggtgccgaacccgcaccacaagaacaaaatttattttcaagcattgaaatagtagaaagaagtaattatatgggtaatccatggacggaatatatggcaaaatatgatattgaagaagttcatggttcaggtataagagtagatttaggagaagatgctgaagtagctggaactcaatatagacttccatcagggaaatgtccagtatttggtaaaggtataattattgagaattcaaatactacttttttaacaccggtagctacgggaaatcaatatttaaaagatggaggttttgcttttcctccaacagaacctcttatgtcaccaatgacattagatgaaatgagacatttttataaagataataaatatgtaaaaaatttagatgaattgactttatgttcaagacatgcaggaaatatgattccagataatgataaaaattcaaattataaatatccagctgtttatgatgacaaagataaaaagtgtcatatattatatattgcagctcaagaaaataatggtcctagatattgtaataaagacgaaagtaaaagaaacagcatgttttgttttagaccagcaaaagatatatcatttcaaaactatacatatttaagtaagaatgtagttgataactgggaaaaagtttgccctagaaagaatttacagaatgcaaaattcggattatgggtcgatggaaattgtgaagatataccacatgtaaatgaatttccagcaattgatctttttgaatgtaataaattagtttttgaattgagtgcttcggatcaacctaaacaatatgaacaacatttaacagattatgaaaaaattaaagaaggtttcaaaaataagaacgctagtatgatcaaaagtgcttttcttcccactggtgcttttaaagcagatagatataaaagtcatggtaagggttataattggggaaattataacacagaaacacaaaaatgtgaaatttttaatgtcaaaccaacatgtttaattaacaattcatcatacattgctactactgctttgtcccatcccatcgaagttgaaaacaattttccatgttcattatataaagatgaaataatgaaagaaatcgaaagagaatcaaaacgaattaaattaaatgataatgatgatgaagggaataaaaaaattatagctccaagaatttttatttcagatgataaagacagtttaaaatgcccatgtgaccctgaaatggtaagtaatagtacatgtcgtttctttgtatgtaaatgtgtagaaagaagggcagaagtaacatcaaataatgaagttgtagttaaagaagaatataaagatgaatatgcagatattcctgaacataaaccaacttatgataaaatgaaaattataattgcatcatcagctgctgtcgctgtattagcaactattttaatggtttatctttataaaagaaaaggaaatgctgaaaaatatgataaaatggatgaaccacaagattatgggaaatcaaattcaagaaatgatgaaatgttagatcctgaggcatctttttggggggaagaaaaaagagcatcacatacaacaccagttctgatggaaaaaccatactattaa

SEQ ID NO: 218 - protein, 622 aa, Artificial Sequence
  AMA1
MRKLYCVLLLSAFEFTYMINFGRGQNYWEHPYQNSDVYRPINEHREHPKEYEYPLHQEHTYQQEDSGEDENTLQHAYPIDHEGAEPAPQEQNLFSSIEIVERSNYMGNPWTEYMAKYDIEEVHGSGIRVDLGEDAEVAGTQYRLPSGKCPVFGKGIIIENSNTTFLTPVATGNQYLKDGGFAFPPTEPLMSPMTLDEMRHFYKDNKYVKNLDELTLCSRHAGNMIPDNDKNSNYKYPAVYDDKDKKCHILYIAAQENNGPRYCNKDESKRNSMFCFRPAKDISFQNYTYLSKNVVDNWEKVCPRKNLQNAKFGLWVDGNCEDIPHVNEFPAIDLFECNKLVFELSASDQPKQYEQHLTDYEKIKEGFKNKNASMIKSAFLPTGAFKADRYKSHGKGYNWGNYNTETQKCEIFNVKPTCLINNSSYIATTALSHPIEVENNFPCSLYKDEIMKEIERESKRIKLNDNDDEGNKKIIAPRIFISDDKDSLKCPCDPEMVSNSTCRFFVCKCVERRAEVTSNNEVVVKEEYKDEYADIPEHKPTYDKMKIIIASSAAVAVLATILMVYLYKRKGNAEKYDKMDEPQDYGKSNSRNDEMLDPEASFWGEEKRASHTTPVLMEKPYY

SEQ ID NO: 219 - DNA, 5742 bp, Artificial Sequence
  LSA-1
atgaaacatattttgtacatatcattttactttatccttgttaatttattgatatttcatataaatggaaagataataaagaattctgaaaaagatgaaatcataaaatctaacttgagaagtggttcttcaaattctaggaatcgaataaatgaggaaaagcacgagaagaaacacgttttatctcataattcatatgagaaaactaaaaataatgaaaataataaatttttcgataaggataaagagttaacgatgtctaatgtaaaaaatgtgtcacaaacaaatttcaaaagtcttttaagaaatcttggtgtttcagagaatatattccttaaagaaaataaattaaataaggaagggaaattaattgaacacataataaatgatgatgacgataaaaaaaaatatattaaagggcaagacgaaaacagacaagaagatcttgaagaaaaagcagctaaagaaacgttacaggggcaacaaagcgatttagaacaagagagacttgctaaagaaaagttgcaagaacaacaaagcgattcagaacaagagagacttgctaaagaaaagttgcaagaacaacaaagcgatttagaacaagagagacttgctaaagaaaagttacaagagcagcaaagcgatttagaacaagagagacttgctaaagaaaagttgcaagagcagcaaagcgatttagaacaagagagacgtgctaaagaaaagttgcaagaacaacaaagcgatttagaacaagagagacgtgctaaagaaaagttgcaagaacaacaaagcgatttagaacaagagagacgtgctaaagaaaagttgcaagaacaacaaagcgatttagaacaagagagacttgctaaagaaaagttgcaagaacaacaaagcgatttagaacaagagagacgtgctaaagaaaagttgcaagaacaacaaagcgatttagaacaagagagacttgctaaagaaaagttacaagagcagcaaagcgatttagaacaagagagacttgctaaagaaaagttgcaagaacaacaaagcgatctagaacaagagagactagctaaagaaaagttacaggggcaacaaagcgatctagaacaagagagacttgctaaagaaaagttgcaagaacaacaaagcgatttagaacaagatagacttgctaaagaaaagttacaagagcaacaaagcgatttagaacaagagagacttgctaaagaaaagttgcaagaacaacaaagcgatttagaacaagagagacgtgctaaagaaaagttgcaagaacaacaaagcgatttagaacaagagagacttgctaaagaaaagttgcaagaacaacaaagcgatttagaacaagagagacgtgctaaagaaaagttgcaagaacaacaaagcgatttagaacaagagagacgtgctaaagaaaagttgcaagaacaacaaagcgatttagaacaagagagacttgctaaagaaaagttacaagagcagcaaagcgatttagaacaagagagacttgctaaagaaaagttgcaagaacaacaaagcgattcagaacaagagagacttgctaaagaaaagttgcaagaacaacaaagcgatttagaacaagagagacttgctaaagaaaagttacaagagcagcaaagcgatttagaacaagagagacttgctaaagaaaagttgcaagaacaacaaagcgatttagaacaagagagacttgctaaagaaaagttgcaagaacaacaaagcgatctagaacaagagagactagctaaagaaaagttacaggggcaacaaagcgatctagaacaagagagactagctaaagaaaagttacaggggcaacaaagcgatttagaacaagagagacttgctaaagaaaagttacaagagcagcaaagcgatttagaacaagagagacttgctaaagaaaagttacaagagcaacaaagcgatttagaacgaacgaaggcatctaaagaaacgttgcaagaacaacaaagcgatttagaacaagagagacttgctaaagaaaagttgcaagaacaacaaagcgatttagaacaagagagacgtgctaaagaaaagttgcaagaacaacaaagcgatttagaacaagagagacgtgctaaagaaaagttgcaagagcaacaaagcgatttagaacaagagcgacgtgctaaagaaaagttgcaagaacaacaaagcgatttagaacaagagagacgtgctaaagaaaagttgcaagaacaacaaagcgatttagaacaagatagacttgctaaagaaaagttacaagagcaacaaagcgatttagaacaagagagacgtgctaaagaaaagttgcaagaacaacaaagcgatttagaacaagatagacttgctaaagaaaagttacaagagcaacaaagcgatttagaacaagagagacgtgctaaagaaaagttgcaagaacaacaaagcgatttagaacaagagagacttgctaaagaaaagttgcaagaacaacaaagcgatttagaacaagagagacgtgctaaagaaaagttgcaagaacaacaaagcgatttagaacaagatagacttgctaaagaaaagttacaagagcaacaaagcgatttagaacaagagagacgtgctaaagaaaagttgcaagaacaacaaagcgatttagaacaagagagacgtgctaaagaaaagttgcaagaacaacaaagcgatttagaacaagagagacttgctaaagaaaagttgcaagagcaacaaagagatttagaacaagagagacgtgctaaagaaaagttgcaagaacaacaaagcgatttagaacaagagagacgtgctaaagaaaagttgcaagaacaacaaagcgatttagaacaagagagacttgctaaagaaaagttacaagagcagcaaagcgatttagaacaagagagacttgctaaagaaaagttgcaagaacaacaaagcgatctagaacaagagagactagctaaagaaaagttacaggggcaacaaagcgatctagaacaagagagactagctaaagaaaagttacaggggcaacaaagcgatctagaacaagagagactagctaaagaaaagttgcaagaacaacaaagcgatttagaacaagagagacttgctaaagaaaagttgcaagaacaacaaagcgatctagaacaagagagactagctaaagaaaagttacaggggcaacaaagcgatctagaacaagagagactagctaaagaaaagttacaggggcaacaaagcgatctagaacaagagagactagctaaagaaaagttacaggggcaacaaagcgatctagaacaagagagactagctaaagaaaagttacaggggcaacaaagcgatctagaacaagagagactagctaaagaaaagttgcaagaacaacaaagcgatttagaacaagagagacttgctaaagaaaagttacaagagcagcaaagcgatttagaacaagagagacgtgctaaagaaaagttacaagagcaacaaagcgatttagaacgaacgaaggcatctaaagaaacgttgcaagaacaacaaagcgatttagaacaagagagacttgctaaagaaaagttgcaagaacaacaaagcgatttagaacaagagagacgtgctaaagaaaagttgcaagaacaacaaagcgatttagaacaagagagactagctaaggaaaagttacaagagcaacaaagcgatttagaacaagagagacgtgctaaagaaaagttgcaagaacaacaaagcgatttagaacaagagcgacgtgctaaagaaaagttgcaagaacaacaaagcgatttagaacaagagagacgtgctaaagaaaagttgcaagaacaacaaagcgatttagaacaagagagacttgctaaagaaaagttgcaagaacaacaaagcgatttagaacaagagagacttgctaaagaaaagttgcaagaacaacaaagcgatttagaacaagagagacgtgctaaagaaaagttgcaagaacaacaaagcgatttagaacaagagagacttgctaaagaaaagttacaagaacaacaaagcgatttagaacaagagagacgtgctaaagaaaagttgcaagaacaacaaagcgatttagaacaagatagacttgctaaagaaaagttacaagagcaacaaagagatttagaacaagagagacgtgctaaagaaaagttgcaagaacaacaaagcgatttagaacaagagagacgtgctaaagaaaagttgcaagaacaacaaagcgatttagaacaagagagacgtgctaaagaaaagttgcaagaacaacaaagcgatttagaacaagagagacgtgctaaagaaaagttgcaagaacaacaaagcgatttagaacaagagagacttgctaaagaaaagttgcaagagcaacaaagagatttagaacaagagagacgtgctaaagaaaagttgcaagaacaacaaagcgatttagaacaagagagacgtgctaaagaaaagttgcaagaacaacaaagcgatttagaacaagagagacttgctaatgaaaagttgcaagagcaacaaagagatttagaacaagagagacgtgctaaagaaaagttgcaagaacaacaaagcgatttagaacaagagagacgtgctaaagaaaagttgcaagaacaacaaagcgatttagaacaagagagacgtgctaaagaaaagttgcaagaacaacaaagcgatttagaacaagagagacttgctaaagaaaagttgcaagaacaacaaagagatttagaacaagagagacttgctaaagaaaagttgcaagagcagcaaagagatttagaacaaaggaaggctgatacgaaaaaaaatttagaaagaaaaaaggaacatggagatgtattagcagaggatttatatggtcgtttagaaataccagctatagaacttccatcagaaaatgaacgtggatattatataccacatcaatcttctttacctcaggacaacagagggaatagtagagattccaaggaaatatctataatagaaaaaacaaatagagaatctattacaacaaatgttgaaggacgaagggatatacataaaggacatcttgaagaaaagaaagatggttcaataaaaccagaacaaaaagaagataaatctgctgacatacaaaatcatacattagagacagtaaatatttctgatgttaatgattttcaaataagtaagtatgaggatgaaataagtgctgaatatgacgattcattaatagatgaagaagaagatgatgaagacttagacgaatttaagcctattgtgcaatatgacaatttccaagatgaagaaaacataggaatttataaagaactagaagatttgatagagaaaaatgaaaatttagatgatttagatgaaggaatagaaaaatcatcagaagaattatctgaagaaaaaataaaaaaaggaaagaaatatgaaaaaacaaaggataataattttaaaccaaatgataaaagtttgtatgatgagcatattaaaaaatataaaaatgataagcaggttaataaggaaaaggaaaaattcataaaatcattgtttcatatatttgacggagacaatgaaattttacagatcgtggatgagttatctgaagatataactaaatattttatgaaactataaaaggttatatat

SEQ ID NO: 220 - protein, 1909 aa, Artificial Sequence
  LSA-1
MKHILYISFYFILVNLLIFHINGKIIKNSEKDEIIKSNLRSGSSNSRNRINEEKHEKKHVLSHNSYEKTKNNENNKFFDKDKELTMSNVKNVSQTNFKSLLRNLGVSENIFLKENKLNKEGKLIEHIINDDDDKKKYIKGQDENRQEDLEEKAAKETLQGQQSDLEQERLAKEKLQEQQSDSEQERLAKEKLQEQQSDLEQERLAKEKLQEQQSDLEQERLAKEKLQEQQSDLEQERRAKEKLQEQQSDLEQERRAKEKLQEQQSDLEQERRAKEKLQEQQSDLEQERLAKEKLQEQQSDLEQERRAKEKLQEQQSDLEQERLAKEKLQEQQSDLEQERLAKEKLQEQQSDLEQERLAKEKLQGQQSDLEQERLAKEKLQEQQSDLEQDRLAKEKLQEQQSDLEQERLAKEKLQEQQSDLEQERRAKEKLQEQQSDLEQERLAKEKLQEQQSDLEQERRAKEKLQEQQSDLEQERRAKEKLQEQQSDLEQERLAKEKLQEQQSDLEQERLAKEKLQEQQSDSEQERLAKEKLQEQQSDLEQERLAKEKLQEQQSDLEQERLAKEKLQEQQSDLEQERLAKEKLQEQQSDLEQERLAKEKLQGQQSDLEQERLAKEKLQGQQSDLEQERLAKEKLQEQQSDLEQERLAKEKLQEQQSDLERTKASKETLQEQQSDLEQERLAKEKLQEQQSDLEQERRAKEKLQEQQSDLEQERRAKEKLQEQQSDLEQERRAKEKLQEQQSDLEQERRAKEKLQEQQSDLEQDRLAKEKLQEQQSDLEQERRAKEKLQEQQSDLEQDRLAKEKLQEQQSDLEQERRAKEKLQEQQSDLEQERLAKEKLQEQQSDLEQERRAKEKLQEQQSDLEQDRLAKEKLQEQQSDLEQERRAKEKLQEQQSDLEQERRAKEKLQEQQSDLEQERLAKEKLQEQQRDLEQERRAKEKLQEQQSDLEQERRAKEKLQEQQSDLEQERLAKEKLQEQQSDLEQERLAKEKLQEQQSDLEQERLAKEKLQGQQSDLEQERLAKEKLQGQQSDLEQERLAKEKLQEQQSDLEQERLAKEKLQEQQSDLEQERLAKEKLQGQQSDLEQERLAKEKLQGQQSDLEQERLAKEKLQGQQSDLEQERLAKEKLQGQQSDLEQERLAKEKLQEQQSDLEQERLAKEKLQEQQSDLEQERRAKEKLQEQQSDLERTKASKETLQEQQSDLEQERLAKEKLQEQQSDLEQERRAKEKLQEQQSDLEQERLAKEKLQEQQSDLEQERRAKEKLQEQQSDLEQERRAKEKLQEQQSDLEQERRAKEKLQEQQSDLEQERLAKEKLQEQQSDLEQERLAKEKLQEQQSDLEQERRAKEKLQEQQSDLEQERLAKEKLQEQQSDLEQERRAKEKLQEQQSDLEQDRLAKEKLQEQQRDLEQERRAKEKLQEQQSDLEQERRAKEKLQEQQSDLEQERRAKEKLQEQQSDLEQERRAKEKLQEQQSDLEQERLAKEKLQEQQRDLEQERRAKEKLQEQQSDLEQERRAKEKLQEQQSDLEQERLANEKLQEQQRDLEQERRAKEKLQEQQSDLEQERRAKEKLQEQQSDLEQERRAKEKLQEQQSDLEQERLAKEKLQEQQRDLEQERLAKEKLQEQQRDLEQRKADTKKNLERKKEHGDVLAEDLYGRLEIPAIELPSENERGYYIPHQSSLPQDNRGNSRDSKEISIIEKTNRESITTNVEGRRDIHKGHLEEKKDGSIKPEQKEDKSADIQNHTLETVNISDVNDFQISKYEDEISAEYDDSLIDEEEDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKL

SEQ ID NO: 221 - DNA, 5256 bp, Artificial Sequence
  MSP1
atgaaggcgctactctttttgttctctttcatttttttcgttaccaaatgtcaatgtgaaacagaaagttataagcagcttgtagccaagctggacaagttagaggcgctcgtggtggacggctacgagctcttccacaaaaaaaagttaggagaaaatgatattaaggtagaaaccaatgctagtgcaaataataataataacaatcaggttagcgttttaacttccaaaataagaaatttcctgagcaagtttttggagctacaaattcctggacataccgacttgctacacctgataagagaattggccgtggaacccaatgggataaaataccttgtggagagctacgaagaattcaatcaactgatgcacgtgatcaacttccactatgatttgttgagggcgaagctccacgacatgtgtgcccatgattattgcaaaataccggagcatctaaaaatctctgacaaagagctggacatgctgaagaaagttgtgctgggttataggaagcccttggacaacataaaggacgatattggaaaattggagaccttcatcactaaaaacaagataacaataaaaaatataagtgatttaattattgcggagaacaagaaaaggagtggccatcccaccaccacgactaatggagccggcacgcaacccgctaatggttcaattgcggcagccagttcggaaactactcaaatttctggttcgtctaactctggttcgagtagcactggttcgtctaactctggttcgagtagcactggttcgagtggcactggttcgactggcactggacaatctcctccagcagctgctgatgcatcttcaacaaatgcaaactacgaagcgaagaaaatcatctaccaagccgtgtacaacaccatattttacacgaaccagctgcaggaagctcaaaagttaatcgcagtcctggaaaagcgcgtgaaagtgctgaaggagcacaaagacattaaggtgctactcgaacaggtcgcaaaagaaaaggaaaagcttcctagtgattatcccaacactacaaatcttacaaatgtacacaaagaagccgaaagcaaaattgccgagctcgagaagaaaatcgaagccatcgccaagactgtgaacttcgacctggacggtctgtttactgacgcagaggagttggagtactatttgagggagaaggcaaagatggccggcacgctaatcatcccagaaagcaccaaatcagcaggcacccctggaaagacagttccaaccctgaaagagacctacccacacggaataagctacgctttagcagaaaacagtatttatgaactgatagaaaaaattggatctgatgaaacatttggtgatttgcaaaatccagatgatggaaagcaaccgaagaagggaatcctcattaatgaaacaaagaggaaagaattgctggaaaaaattatgaataaaattaagatagaagaagacaaattgcccaacctaaaaaaagaatacgaggaaaaatataaggtgtacgaggcaaaggttaatgagttcaaaccagcatttaatcacttttatgaggcaagactggacaacacccttgttgaaaacaaatttgatgattttaagaaaaaaagagaggcatatatggaggagaagaaaaaactagaaagctgctcctacgaacagaacagcaatctgattaacaagctgaaaaaacaactaacatacttggaggactacgttttaagaaaagacatcgccgacgatgaaattaaacacttcagtttcatggagtggaaattaaagagcgaaatttatgatctagcccaggaaatccgaaaaaacgaaaacaagctcaccattgaaaacaaattcgacttctccggggttgtggaattacaagtacaaaaggtattgataatcaaaaaaattgaggctctaaagaatgtccagaatcttcttaagaatgccaaggtgaaggacgacctgtacattccaaaggtgtataagacaagcgagaaacctgagccctactacttgatggtcctcaaaagggaaattgacaagttgaaggacttcatccccaaaatcgagagcatgatcgccactgagaagaacaagccgaccgtggcagcggcagatatagtggcaaagggacaatcgcttagaggagcaagtgaaacagggacaactggcaatacagtcaatgcgcaaacagctgtagtacaaccacaacatcaagtagtaaatgcagtaacggtacagcctggaacaacaggacatcaagcacaaggtggagaagcagaaacacaaacaaattcagtacaagcagcacaagttcaacaaacacctgcaggagcgggcggacaggtagcctcaacacaaacgattagccaagccccagcaccaactcaagcctccccagaaccagcaccagccgccccaccatcgacacctgctgccgcagttgctccagcaccaaccatgtccaaactggaatacctcgaaaagctccttgattttttaaaatccgcttacgcatgtcacaagcacatttttgtaaccaactccaccatgaaaaaggagctactcgatcagtacaaacttaacgctgatgagcaaaacaaaattaacgaaactaaatgcgatgaattggacctcctattcaatgtccagaacaacttgccagccatgtactccatatatgactctatgagcaacgaactgcagaacctttacattgagctgtaccagaaggaaatggtttacaatatatacaagaacaaggacacggacaagaagattaaggctttcctggaaacactcaagagcaaagcggctgctcctgctcagtcagcggcaaaacccagcggtcaagcgggtactactccagtaacgacaactgcgccagtaaccacaacaacagttactccaagtccccaaacatcagttgtaacaagcacacctcctacaccccaagcagaagaaaaccgacgcgtgggaggtaacagcgaggagaaacccgaagccgacactgcgcaagtggaaaagttttacgagaagcacctatcccaaattgacaagtacaacgactatttccagaagttccttgaatcccaaaaagatgaaatcaccaaaatggatgaaacaaagtggaaagcactaggtgcagaaattgaggaactgaagaagaagctacaagtatctctggaccactatggaaagtacaagctcaaattggagaggctcctcaaaaagaagaataaaatctctaacagcaaggatcaaattaaaaagctcaccagtttgaaaaacaaattggagagaagacaaaatctgttgaataacccaacaagtgtgttgaaaaattacaccgcttttttcaacaaaaagagagaaacagaaaagaaggaggtggaaaatacccttaagaataccgagattttgctgaagtactataaggcacgagccaaatattatataggagagcccttccctctgaagaccttaagtgaagaatcaatgcagaaggaggacaactacctcaacttagaaaagtttagagtgctcagcagattggaaggaagattaggaaagaacatcgagttggaaaaggagaacataagctacctgtccagtggactgcaccacgtcttgacagagctgaaggaaattatcaaaaacaagaaatactccggtaacgaccacacgaagaacattgcagctgttaaggaagctttgcaagcctaccaagaattgatccccaaggtgaccactcaggaaggcgcatccacaacagcggcaacattaccagtaacagtaccatcagcagtaccaggaggattacctggagcaggagtaccaggagcagcagcaggactaacaccaccaccaccagcaggatcagtaccagcaacaggaccaggagcagcagcaggatcaacagaagaaaacgtagcagcaaaagcgcaggactacgccgaggactacgacaaagtaatcgcactccctctgttcggcaacaacgatgacgacggggaggaagaccaagtaacaacgggagaggcagaatctgaggcgcctgagatcctcgtgccagcaggaatcagcgattacgatgtggtctacttaaagccattagccggaatgtacaaaacgataaagaagcaattggaaaatcacgtaaacgcatttaacactaacataacggatatgttagactctagactgaagaagagaaactacttcttagaagttctgaactctgatttgaacccatttaagtattcatcatctggtgagtacatcattaaggacccatacaagctgctcgacttggagaagaagaagaagcttataggcagctacaagtacatcggtgcatcgatcgacatggatctggccaccgcgaatgatggcgtgacctactacaacaagatgggggagctctacaagacgcacttggatggagtgaaaacagagattaagaaagtcgaagatgatattaaaaagcaagatgaggaacttaaaaagttaggaaatgttaacagtcaagatagtaaaaagaacgaatttattgccaaaaaggccgagctggagaagtacctcccgttcctgaatagcctccaaaaggagtacgagtccctcgtgagcaaggtgaacacctacacagacaacctaaaaaaagtcatcaacaactgccagctggagaaaaaggaagccgagatcactgtaaagaaattgcaggactacaacaagatggatgagaagttggaggagtacaaaaaatcggagaaaaaaaatgaagtgaagtcttctggtcttctggaaaaattgatgaaatcaaaattgattaaagaaaacgagtccaaggaaatattatcccagctgctaaatgtgcaaactcagttattaactatgagctccgagcacacatgtatagacaccaatgtgcctgataatgcagcctgctataggtacttggacggaacggaagaatggagatgcttgttaacctttaaagaagaaggcggcaagtgtgtgccagcatcgaatgtgacttgtaaggataacaatggtggttgtgcccctgaagctgaatgtaaaatgacggacagcaataaaatcgtctgtaaatgtactaaagaaggttctgagccactctttgagggagttttctgtagctcctccagcttcctaagcttgtccttcttgttgctcatgttgcttttcctcctgtgcatggagctttaa

SEQ ID NO: 222 - protein, 1751 aa, Artificial Sequence
  MSP1
MKALLFLFSFIFFVTKCQCETESYKQLVAKLDKLEALVVDGYELFHKKKLGENDIKVETNASANNNNNNQVSVLTSKIRNFLSKFLELQIPGHTDLLHLIRELAVEPNGIKYLVESYEEFNQLMHVINFHYDLLRAKLHDMCAHDYCKIPEHLKISDKELDMLKKVVLGYRKPLDNIKDDIGKLETFITKNKITIKNISDLIIAENKKRSGHPTTTTNGAGTQPANGSIAAASSETTQISGSSNSGSSSTGSSNSGSSSTGSSGTGSTGTGQSPPAAADASSTNANYEAKKIIYQAVYNTIFYTNQLQEAQKLIAVLEKRVKVLKEHKDIKVLLEQVAKEKEKLPSDYPNTTNLTNVHKEAESKIAELEKKIEAIAKTVNFDLDGLFTDAEELEYYLREKAKMAGTLIIPESTKSAGTPGKTVPTLKETYPHGISYALAENSIYELIEKIGSDETFGDLQNPDDGKQPKKGILINETKRKELLEKIMNKIKIEEDKLPNLKKEYEEKYKVYEAKVNEFKPAFNHFYEARLDNTLVENKFDDFKKKREAYMEEKKKLESCSYEQNSNLINKLKKQLTYLEDYVLRKDIADDEIKHFSFMEWKLKSEIYDLAQEIRKNENKLTIENKFDFSGVVELQVQKVLIIKKIEALKNVQNLLKNAKVKDDLYIPKVYKTSEKPEPYYLMVLKREIDKLKDFIPKIESMIATEKNKPTVAAADIVAKGQSLRGASETGTTGNTVNAQTAVVQPQHQVVNAVTVQPGTTGHQAQGGEAETQTNSVQAAQVQQTPAGAGGQVASTQTISQAPAPTQASPEPAPAAPPSTPAAAVAPAPTMSKLEYLEKLLDFLKSAYACHKHIFVTNSTMKKELLDQYKLNADEQNKINETKCDELDLLFNVQNNLPAMYSIYDSMSNELQNLYIELYQKEMVYNIYKNKDTDKKIKAFLETLKSKAAAPAQSAAKPSGQAGTTPVTTTAPVTTTTVTPSPQTSVVTSTPPTPQAEENRRVGGNSEEKPEADTAQVEKFYEKHLSQIDKYNDYFQKFLESQKDEITKMDETKWKALGAEIEELKKKLQVSLDHYGKYKLKLERLLKKKNKISNSKDQIKKLTSLKNKLERRQNLLNNPTSVLKNYTAFFNKKRETEKKEVENTLKNTEILLKYYKARAKYYIGEPFPLKTLSEESMQKEDNYLNLEKFRVLSRLEGRLGKNIELEKENISYLSSGLHHVLTELKEIIKNKKYSGNDHTKNIAAVKEALQAYQELIPKVTTQEGASTTAATLPVTVPSAVPGGLPGAGVPGAAAGLTPPPPAGSVPATGPGAAAGSTEENVAAKAQDYAEDYDKVIALPLFGNNDDDGEEDQVTTGEAESEAPEILVPAGISDYDVVYLKPLAGMYKTIKKQLENHVNAFNTNITDMLDSRLKKRNYFLEVLNSDLNPFKYSSSGEYIIKDPYKLLDLEKKKKLIGSYKYIGASIDMDLATANDGVTYYNKMGELYKTHLDGVKTEIKKVEDDIKKQDEELKKLGNVNSQDSKKNEFIAKKAELEKYLPFLNSLQKEYESLVSKVNTYTDNLKKVINNCQLEKKEAEITVKKLQDYNKMDEKLEEYKKSEKKNEVKSSGLLEKLMKSKLIKENESKEILSQLLNVQTQLLTMSSEHTCIDTNVPDNAACYRYLDGTEEWRCLLTFKEEGGKCVPASNVTCKDNNGGCAPEAECKMTDSNKIVCKCTKEGSEPLFEGVFCSSSSFLSLSFLLLMLLFLLCMEL

SEQ ID NO: 223 - DNA, 1689 bp, Artificial Sequence
  AMA1
atgaataaaatatactacataatctttttaagcgcccagtgccttgtgcacattgggaagtgcgggcgaaaccagaagccgagcaggctgacccgtagcgccaacaacgttctactggaaaaggggcctaccgttgagagaagcacacgaatgagtaacccctggaaagcgttcatggaaaaatacgacatcgaaagaacacacagttctggggttcgagtggatttaggggaagatgcagaagtggaaaatgcaaagtacagaattccagctggaagatgtcctgtttttggaaagggtatcgttatagagaattctgacgttagcttcttaagacctgtggctacaggagatcagaagctgaaggatggaggtttcgccttccccaatgcgaatgaccatatctccccaatgacattagcgaaccttaaggaaaggtataaagacaatgtagagatgatgaagttaaacgatatagctttgtgcagaacccacgcagctagctttgtcatggcaggggatcaaaattcgtcctacagacacccagctgtatacgacgaaaaggaaaaaacatgccacatgttgtatttatcagcgcaggaaaatatgggtccgaggtactgcagcccagatgcacaaaatagagatgccgtgttctgcttcaagccagataaaaatgaaagctttgaaaacctggtgtatttgagcaaaaatgtgcgtaatgattgggataaaaaatgcccccgtaaaaatttaggaaacgccaagttcggattatgggtggatgggaactgcgaagaaattccatacgttaaagaagtggaggcagaagatctgcgcgaatgcaaccgaatcgttttcggagcgagtgcctcagatcaaccaactcagtatgaagaagaaatgacggattatcaaaaaatacaacaagggtttagacaaaacaaccgagagatgattaaaagtgcctttcttccagtgggtgcattcaactcggataatttcaaaagtaaaggaagaggatttaactgggcaaatttcgattctgtaaaaaagaagtgttacatttttaataccaaaccgacttgcctcattaatgacaaaaattttattgcaacaacggcgttatctcacccacaagaagtagacctggagttcccctgcagcatatataaagacgaaattgaaagagaaattaagaaacaatcgaggaacatgaatctgtacagtgttgatggggaacgcattgtcctgccgaggatatttatctccaacgataaggagagtatcaaatgtccctgcgaacctgagcgcatttccaacagtacctgcaacttttacgtttgtaactgtgtagagaaaagggcggaaattaaggaaaataaccaagttgttataaaggaagaatttagggattattacgaaaatggggaggaaaaatcgaacaagcagatgctactaatcattatcggaataactggtggcgtgtgcgtcgtcgcgctggcctctatggcctacttcaggaagaaggctaacaatgataagtatgacaagatggaccaggcagaggggtacgggaagcccaccaccaggaaggacgagatgctcgaccccgaggcctccttctggggcgaggacaagcgggcctcccacaccacgcccgtgctgatggagaagccatactactaa

SEQ ID NO: 224 - protein, 562 aa, Artificial Sequence
  AMA1
MNKIYYIIFLSAQCLVHIGKCGRNQKPSRLTRSANNVLLEKGPTVERSTRMSNPWKAFMEKYDIERTHSSGVRVDLGEDAEVENAKYRIPAGRCPVFGKGIVIENSDVSFLRPVATGDQKLKDGGFAFPNANDHISPMTLANLKERYKDNVEMMKLNDIALCRTHAASFVMAGDQNSSYRHPAVYDEKEKTCHMLYLSAQENMGPRYCSPDAQNRDAVFCFKPDKNESFENLVYLSKNVRNDWDKKCPRKNLGNAKFGLWVDGNCEEIPYVKEVEAEDLRECNRIVFGASASDQPTQYEEEMTDYQKIQQGFRQNNREMIKSAFLPVGAFNSDNFKSKGRGFNWANFDSVKKKCYIFNTKPTCLINDKNFIATTALSHPQEVDLEFPCSIYKDEIEREIKKQSRNMNLYSVDGERIVLPRIFISNDKESIKCPCEPERISNSTCNFYVCNCVEKRAEIKENNQVVIKEEFRDYYENGEEKSNKQMLLIIIGITGGVCVVALASMAYFRKKANNDKYDKMDQAEGYGKPTTRKDEMLDPEASFWGEDKRASHTTPVLMEKPYY

SEQ ID NO: 225 - protein, 24 aa, Artificial Sequence
  AMA1
NANPNANPNANPNANPNANPNANP

SEQ ID NO: 226 - protein, 20 aa, Artificial Sequence
  T-cell epitope
QYLKKLKNSLSTEWSPCSVT

SEQ ID NO: 227 - protein, 4 aa, Artificial Sequence
  B-cell epitope
NVDP

SEQ ID NO: 228 - protein, 9 aa, Artificial Sequence
  CTL epitope NF54
YLNKIQNSL

SEQ ID NO: 229 - protein, 9 aa, Artificial Sequence
  CTL epitope 7G8
YLKKIKNSL

SEQ ID NO: 230 - protein, 28 aa, Artificial Sequence
  (TIB)4 repeat
DPNANPNVDPNANPNVNANPNANPNANP

(19) United States
(12) Patent Application Publication
(10) Pub. No.: US 2011/0008383 A1
(43) Pub. Date: Jan. 13, 2011

(54) COMPOSITIONS OF TOLL-LIKE RECEPTOR AGONISTS AND MALARIA ANTIGENS AND METHODS OF USE

(76) Inventors: THOMAS J. POWELL, MADISON, CT (US); VALERIAN NAKAAR, HAMDEN, CT (US); WILLIAM F. MCDONALD, MADISON, CT (US); ELIZABETH H. NARDIN, LEONIA, NJ (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/814,945

(22) Filed: Jun. 14, 2010

Related U.S. Application Data
(63) Continuation of application No. PCT/US2008/013713, filed on Dec. 15, 2008.
(60) Provisional application No. 61/008,010, filed on Dec. 18, 2007.
(60) Provisional application No. 61/195,971, filed on Oct. 14, 2008.

(51) Int. Cl. A61K 39/015; A61P 37/04; A61P 33/06

(52) U.S. Cl. 424/192.1

(57) ABSTRACT

Compositions that include at least one fusion protein that includes at least a portion of at least one flagellin and at least a portion of at least one malaria antigen can be employed in methods that stimulate an immune response in a subject, in particular, sterile immunity and a protective immune response in a subject.

### RELATED APPLICATIONS

This application is a continuation of International Application No.: PCT/US2008/013713, which designated the United States and was filed on Dec. 15, 2008, published in English, which claims the benefit of U.S. Provisional Application Nos. 61/008,010, filed on Dec. 18, 2007, and 61/195,971, filed on Oct. 14, 2008. The entire teachings of the above applications are incorporated herein by reference.

### GOVERNMENT SUPPORT

The invention was supported, in whole or in part, by a grant R01 A145138 from The National Institutes of Health (NIAID). The Government has certain rights in the invention.

### BACKGROUND OF THE INVENTION

Every year, hundred of millions of people worldwide are infected by malaria carrying mosquitoes, which results in millions of deaths. Malaria is caused by one-cell protozoan parasites of the genus Plasmodium, such as Plasmodium falciparum, Plasmodiu vivax, Plasmodium ovale and Plasmodium malaria, and is transmitted to humans by female Anopheline mosquitoes. Malaria is diagnosed by clinical symptoms, such as fever, shivering, pain in the joints, headaches, and microscopic examination of a blood sample for the presence of blood stage parasites. Currently, treatment for malaria can include the use of antimalaria drugs, in particular, chloroquine and hydroxychloroquine. However, in certain regions of the world, malaria parasites have developed resistance to these drugs. In endemic regions of the world, where transmission of the malaria parasite is high, humans are continuously infected and can gradually develop immunity to disease consequent to malaria infection. Until immunity is acquired, children are highly susceptible to malaria infection. Thus, there is a need to develop new, improved and effective methods to prevent disease consequent to malaria infection and to prevent the onset of malaria infection.

### SUMMARY OF THE INVENTION

The present invention relates to compositions that include malaria antigens, such as fusion proteins that include malaria antigens and Toll-like Receptor agonists that provide sterile immunity and stimulate protective immunity in a subject.

In an embodiment, the invention is a composition that includes at least one fusion protein comprising at least a portion of at least one Toll-like Receptor 5 agonist and at least a portion of at least one malaria antigen, wherein the malaria antigen is not a Plasmodium vivax merozoite surface protein 1 antigen.

In another embodiment, the invention is a composition that includes at least one fusion protein comprising at least a portion of at least one Toll-like Receptor 5 agonist and at least a portion of at least one malaria antigen.

Another embodiment of this invention is a composition that includes at least one fusion protein comprising at least a portion of at least one Toll-like Receptor 5 agonist, at least a portion of at least one malaria T-cell epitope and at least a portion of at least one malaria antigen B-cell epitope.

In a further embodiment, the invention is a composition that includes at least one fusion protein comprising at least a portion of at least one Toll-like Receptor agonist and at least a portion of at least one malaria antigen, wherein the Toll-like Receptor agonist is not a Pam3Cys.

A further embodiment of the invention is a method of stimulating an immune response in a subject, comprising the step of administering to the subject a composition that includes at least one fusion protein comprising at least a portion of at least one Toll-like Receptor 5 agonist and at least a portion of at least one malaria antigen.

An additional embodiment of the invention is a method of stimulating an immune response in a subject, comprising the step of administering to the subject a composition that includes at least one fusion protein comprising at least a portion of at least one Toll-like Receptor 5 agonist and at least a portion of at least one malaria antigen, wherein the malaria antigen is not a Plasmodium vivax merozoite surface protein 1 antigen.

In another embodiment, the invention is a method of stimulating an immune response in a subject, comprising the step of administering to the subject a composition that includes at least one fusion protein comprising at least a portion of at least one Toll-like Receptor agonist and at least a portion of at least one malaria antigen, wherein the Toll-like Receptor agonist is not a Pam3Cys.

Another embodiment of the invention is a method of stimulating an immune response in a subject, comprising the step of administering to the subject a composition that includes at least one fusion protein comprising at least a portion of at least one Toll-like Receptor 5 agonist, at least a portion of at least one malaria T-cell epitope and at least a portion of at least one malaria antigen B-cell epitope.

In yet another embodiment, the invention is a method of providing sterile immunity in a subject, comprising the step of administering to the subject a composition that includes at least one fusion protein comprising at least a portion of at least one Toll-like Receptor 5 agonist and at least a portion of at least one malaria antigen, wherein the malaria antigen is not a Plasmodium vivax merozoite surface protein 1 antigen.

In an additional embodiment, the invention is a method of providing sterile immunity against a malaria infection in a subject, comprising the step of administering to the subject a composition that includes at least one fusion protein comprising at least a portion of at least one Toll-like Receptor 5 agonist and at least a portion of at least one malaria antigen.

A further embodiment of the invention is a method of providing sterile immunity against a malaria infection in a subject, comprising the step of administering to the subject a composition that includes at least one fusion protein comprising at least a portion of at least one Toll-like Receptor agonist and at least a portion of at least one malaria antigen, wherein the Toll-like Receptor agonist is not a Pam3Cys.

Another embodiment of the invention is a method of providing sterile immunity against a malaria infection in a subject, comprising the step of administering to the subject a composition that includes at least one fusion protein comprising at least a portion of at least one Toll-like Receptor 5 agonist, at least a portion of at least one malaria T-cell epitope and at least a portion of at least one malaria antigen B-cell epitope.

In still another embodiment, the invention is a method of stimulating a protective immune response in a subject, comprising the step of administering to the subject a composition that includes at least one fusion protein comprising at least a portion of at least one Toll-like Receptor 5 agonist and at least a portion of at least one malaria antigen.

An additional embodiment of the invention is a method of stimulating a protective immune response in a subject, comprising the step of administering to the subject a composition that includes at least one fusion protein comprising at least a portion of at least one Toll-like Receptor 5 agonist and at least a portion of at least one malaria antigen, wherein the malaria antigen is not a Plasmodium vivax merozoite surface protein 1 antigen.

Another embodiment of the invention is a method of stimulating a protective immune response in a subject, comprising the step of administering to the subject a composition that includes at least one fusion protein comprising at least a portion of at least one Toll-like Receptor agonist and at least a portion of at least one malaria antigen, wherein the Toll-like Receptor agonist is not a Pam3Cys.

In yet another embodiment, the invention is a method of stimulating a protective immune response in a subject, comprising the step of administering to the subject a composition that includes at least one fusion protein comprising at least a portion of at least one Toll-like Receptor 5 agonist, at least a portion of at least one malaria T-cell epitope and at least a portion of at least one malaria antigen B-cell epitope.

The compositions of the invention can be employed to stimulate an immune response in a subject, in particular sterile immunity and protective immunity consequent to a malaria infection in a subject. Advantages of the claimed invention include, for example, cost effective methods and compositions that can be produced in relatively large quantities for use in the prevention and treatment of disease consequent to malaria infection, thereby avoiding and diminishing illness and death consequent to malaria infection.

### BRIEF DESCRIPTION OF THE FIGURES

FIGS. 1A and 1B depict the strain, GenBank Accession number and amino acid sequence of Plasmodium falciparum circumsporozite proteins (SEQ ID NOs: 25-33). The T-cell epitope T* (EYLNKIQNSLSTEWSPCSVT; SEQ ID NO: 34) is indicated, which is polymorphic and can vary in different Plasmodium falciparum strains. The T1 cell epitope is located in the minor repeat region, located in the 5′ end of the central repeat region and includes alternating NANPNVDP sequences (SEQ ID NO: 35), while the major repeat region include repeats of NANP (SEQ ID NO: 36). The T1 epitope is located in the CS repeat region and functions as both a T helper epitope as well as a B cell epitope. The T1 epitope is DPNANPNVDPNANPNV (SEQ ID NO: 37) is also referred to herein as “(DPNANPNV)₂,” which includes the malaria antigen component of the STF2.T1BT* fusion protein (SEQ ID NO: 9). The minimal B cell epitope is three NAND (SEQ ID NO: 36) repeats, NANPNANPNANP (SEQ ID NO: 38), also referred to herein as “(NANP)₃.”

FIGS. 2A, 2B and 2C depict the strain, GenBank Accession number and nucleic acid sequence of Plasmodium vivax circumsporozite proteins (SEQ ID NOs: 39-54). The T-cell epitope T* (EYLDKVRATVGTEWTPCSVT; SEQ ID NO: 55) is indicated.

FIGS. 3A, 3B and 3C depict the strain, GenBank Accession number and nucleic acid sequence of Plasmodium malariae circumsporozite proteins (SEQ ID NOs: 56-72). The T-cell epitope T* (NYLESIRNSITEEWSPCSVT; SEQ ID NO: 73) is indicated.

FIGS. 4A-4H depict the strain, GenBank Accession number and nucleic acid sequence of Plasmodium falciparum circumsporozite proteins (SEQ ID NOs: 74-81).

FIGS. 5A-5M depict the strain, GenBank Accession number and nucleic acid sequence of Plasmodium vivax circumsporozite proteins (SEQ ID NOs: 82-97).

FIGS. 6A-6J depict the strain, GenBank Accession number and nucleic acid sequence of Plasmodium malariae circumsporozite proteins (SEQ ID NOs: 98-114).

FIG. 7 depicts the activation of an antigen-presenting cell (APC) by Toll-like Receptor (TLR) signaling.

FIG. 8 depicts the D1 domain, D2 domain, TLR5 activation domain and hypervariable (D3 domain) of flagellin.

FIG. 9 depicts the D1 domain, D2 domain, TLR5 activation domain and hypervariable (D3 domain) of flagellin (Yonekura, et al. Nature 424: 643-650 (2003)).

FIG. 10 depicts the amino acid sequence (SEQ ID NO: 118) of a flagellin for use in the compositions of the invention. The hinge region of the flagellin is underlined.

FIG. 11 depicts the nucleic acid sequence encoding a flagellin for use in the compositions of the invention (SEQ ID NO: 119). The nucleic acid sequence encoding the hinge region is underlined.

FIG. 12 depicts the amino acid sequence of a flagellin lacking a hinge region (SEQ ID NO: 120) for use in compositions of the invention and the corresponding nucleic acid sequence (SEQ ID NO: 121).

FIG. 13 depicts the amino acid sequence of a flagellin (SEQ ID NO: 122) for use in the compositions of the invention. The hinge region of the flagellin is underlined.

FIG. 14 depicts a nucleic acid sequence (SEQ ID NO: 123) encoding a flagellin for use in compositions of the invention. The nucleic acid sequence encoding the hinge region of the flagellin is underlined.

FIG. 15 depicts the amino acid sequence (SEQ ID NO: 124) of flagellin for use in the compositions of the invention. The hinge region of the flagellin is underlined.

FIG. 16 depicts a nucleic acid sequence (SEQ ID NO: 125) encoding a flagellin for use in the compositions of the invention. The nucleic acid sequence encoding the hinge region of flagellin is underlined.

FIG. 17 depicts malaria antigen T-cell epitopes for use in the compositions of the invention. EYLNKIQNSLSTEWSPCSVT (SEQ ID NO: 34); KYLKRIKNSISTEWSPCSVT (SEQ ID NO: 133); QYLQTIRNSLSTEWSPCSVT (SEQ ID NO: 134); EYLDKVRATVGTEWTPCSVT (SEQ ID NO: 55); NYLESIRNSITEEWSPCSVT (SEQ ID NO: 73); EFLKQIQNSLSTEWSPCSVT (SEQ ID NO: 135); EFVKQISSQLTEEWSQCNVT (SEQ ID NO: 136); and EFVKQIRDSITEEWSQCSVT (SEQ ID NO: 137).

FIG. 18 depicts malaria antigen B-cell epitopes for use in the compositions of the invention. A P. falciparum B-cell epitope can include NANPNANPNANP (SEQ ID NO: 38, also referred to herein as “(NANP)₃”). P. vivax type 210 (VK210 repeat) epitope includes DRADGQPAG (SEQ ID NO: 138), DRADGQPAGDRADGQPAG (SEQ ID NO: 139; also referred to herein as “(DRADGQPAG)₂”) and DRAAGQPAG (SEQ ID NO: 140) DRAAGQPAGDRAAGQPAG (SEQ ID NO: 141); also referred to herein as “(DRAAGQPAG)₂”); and DRADGQPAGDRAAGQPAG (SEQ ID NO: 142). P. vivax type 247 (VK247 repeat) includes ANGAGNQPGANGAGNQPGANGAGNQPGANGAGNQPG (SEQ ID NO: 143; also referred to herein as “(ANGAGNQPG)₄”). P. malariae includes NAAGNAAGNAAGNAAG (SEQ ID NO: 144; also referred to herein as “(NAAG)₄”). P. berghei includes PPPPNPNDPPPPNPND (SEQ ID NO: 145); also referred to herein as “(PPPPNPND)₂”).

FIG. 19 depicts ELISA IgG-GMT for serum obtained following intranasal administration of fusion proteins (STF2Δ.CS and STF2.T1BT*-4×) and T1BT* peptides.

FIG. 20 depicts antibody elicited by s.c. and intranasal (i.n.) immunization with STF2Δ.CS, STF2Δ and (T1B)₄.

FIG. 21 depicts the amino acid sequences of exemplary P. falciparum CSP (SEQ ID NO: 146); T1BT* (SEQ ID NO: 147); 4×T1BT* (SEQ ID NO: 148); 10×T1BT (SEQ ID NO: 149); 10×TIT* (SEQ ID NO: 150); and 10×BT* (SEQ ID NO: 151) malaria antigens employed in fusion proteins of the invention.

FIG. 22 is a schematic illustration of P. falciparum CS protein showing T1 (SEQ ID NO: 152) and B (SEQ ID NO: 38) epitopes within the central repeat region and the T* epitope (SEQ ID NO: 34) located in the carboxy-terminus of the CSP.

FIGS. 23A and 23B depict anti-repeat and anti-sporozoite IgG antibody titers in C57B1 mice immunized s.c. with T1BT* branched (FIG. 23A) or linear (FIG. 23B) peptide in various adjuvants.

FIG. 24 depicts T cell responses in IFN-γ ELISPOT using spleen cells of C57B1 mice immunized s.c. with branched or linear T1BT* peptide in ISA 720 adjuvant.

FIGS. 25A and 25B depict levels of liver stage parasites following challenge by exposure to the bites of PfPb infected mosquitoes in mice immunized s.c. with T1BT* peptide emulsified in Freunds adjuvant (FIG. 25A) or ISA 720 adjuvant (FIG. 25B).

FIGS. 26A and 26B depict resistance to PfPb sporozoite challenge in T1BT* peptide immunized mice depleted of CD4+ or CD8+ T cells prior to challenge (FIG. 26A) and presence of sporozoite neutralizing antibodies in sera of protected mice immunized s.c. with T1BT* peptide in ISA 720 (FIG. 26B). Each symbol represents an individual mouse.

FIG. 27 is a schematic illustration of flagellin (STF2) modified CS constructs containing P. falciparum CS T1BT* sequences either as one copy (1×) or as four copies (4×), and STF2Δ-CS containing the nearly full length. P. falciparum CS protein conjugated to a truncated flagellin (STF2Δ) moiety. T1 (SEQ ID NO: 37); B (SEQ ID NO: 38) and T*(SEQ ID NO: 34) epitopes are depicted.

FIG. 28 depicts TLR5 signaling by STF2-T1BT*-1× as measured by TNF production by RAW cells transfected with human TLR5.

FIGS. 29A and 29B depict IgG geometric mean titers (GMT) and kinetics of antibody response in BALB/c immunized s.c. with STF2.T1BT*-1× (FIG. 29A) or STF2.T1BT*-4× (FIG. 29B) constructs. Results shown as IgG geometric mean titers (GMT) determined by ELISA using immunogen or CS repeats as antigen.

FIG. 30 depicts IgG antibody in serum of C57Bl mice immunized s.c. with STF2.T1BT*-4× as measured by ELISA using immunogen or CS repeats as antigen. Numbers above each bar indicate number of seropositive mice in each group of mice.

FIGS. 31A and 31B depict STF2Δ.CS antigenicity and functional TLR stimulation. FIG. 31A depicts O.D. obtained in ELISA plate coated with indicated concentrations of STF2Δ.CS protein and reacted with anti-CS antibody (MAb 2A10). FIG. 31B depicts stimulation of hTLR5/RAW cells (closed symbols) or non-transfected RAW cells (open symbols) with varying concentrations of STF2Δ.CS or flagellin control protein.

FIGS. 32A and 32B depict immunogenicity of STF2Δ-CS (also referred to herein as “STF2Δ.CS”) construct administered s.c. to Balb/c (FIG. 32A) or C57Bl (FIG. 32B) mice. Results shown as IgG ELISA GMT using STFΔ-CS, flagellin or CS repeat peptide as antigen.

FIGS. 33A and 33B depict T cell responses measured byTh1-type cytokine IFN-γ ELISPOT in spleens of mice immunized s.c. with STF2Δ.CS (FIG. 33A) or STF2.T1BT*-4× (FIG. 33B). Spleen cells were tested directly ex vivo or following a one week expansion in vitro with malaria peptide T1BT*.

FIGS. 34A and 34B depict T cell responses measured by Th2-type cytokine IL-5 ELISPOT in spleens of mice immunized s.c. with STF2Δ.CS (FIG. 34A) or STF2.T1BT*-4× (FIG. 34B). Spleen cells were tested directly ex vivo or following a one week expansion in vitro with malaria peptide T1BT*.

FIG. 35 depicts kinetics of IgG anti-repeat antibody responses in serum of mice immunized intranasally with 10 μg of STF2Δ.CS or STF2.T1BT*-4× (also referred to herein as “STF2.4×T1BT*”). Results are compared to titers following s.c. immunization with the same immunogens.

FIGS. 36A and 36B depict T cell responses measured by Th2-type cytokine IL-5 ELISPOT in spleens of mice immunized intranasally with STF2Δ.CS (FIG. 36A) or STF2.T1BT*-4× (FIG. 36B). Spleen cells were tested directly ex vivo or following a one week expansion in vitro with malaria peptide T1BT*.

FIG. 37 depicts the level of IL-6 present in supernatant of expanded spleen cell cultures from mice immunized intranasally with STF2Δ.CS, STF2.T1BT*-4× or unmodified linear T1BT* peptide as measured by Cytokine Bead Assay (CBA).

FIG. 38 depicts sporozoite neutralizing activity in serum of mice immunized intranasally with 10 μg of STF2Δ.CS or STF2.T1BT*-4× or unmodified linear peptide T1BT*. Pooled serum of each group of mice, obtained following seven doses of immunogen, were incubated with transgenic sporozoites expressing P. falciparum CS repeats, prior to addition to hepatoma cells. Results shown as the number of copies of parasite 18S rRNA detected in cell cultures at 48 hours, as measured by real-time PCR. The anti-repeat antibody GMT for each group is shown above each bar.

FIGS. 39A, 39B and 39C depict kinetics and fine specificity of IgG antibody elicited following immunization with STF2Δ.CS (50 μg dose) administered either s.c. or intranasally. Results shown as IgG GMT for each group of mice.

FIG. 40 depicts sporozoite neutralizing activity in serum of mice immunized intranasally or s.c. with 50 μg of STF2Δ.CS. Pooled serum of each group of mice, obtained following five doses of immunogen, were incubated with transgenic sporozoites expressing P. falciparum CS repeats, prior to addition to hepatoma cells. Results shown as the number of copies of parasite 18S rRNA detected in cell cultures at 48 hours, as measured by real-time PCR.

FIG. 41 depicts protective efficacy of immunization with STF2Δ.CS administered either intranasally or s.c. Mice were challenged after the fifth dose of immunogen by exposure to the bites of PfPb infected mosquitoes. Levels of parasite 18S rRNA in the livers of challenged mice were determined at 40 hours post challenge by realtime PCR. Each symbol represents an individual mouse with the bar indicating the mean copy number of 18S rRNA for each group.

FIGS. 42A and 42B depict in vitro TLR5 bioactivity of fusion proteins of the invention employing a HEK293 cell assay. FIG. 42A depicts in vitro TLR5 bioactivity of STF2.10×T1BT*His6 (SEQ ID NO: 20); STF2.10×T1T* His6 (SEQ ID NO: 24) and STF2.10×BT* His6 (SEQ ID NO: 22). FIG. 42B depicts in vitro TLR5 bioactivity STF2.T1BT* (SEQ ID NO: 9) and STF2.4×T1BT* (SEQ ID NO: 11).

FIG. 43 depicts in vitro TLR5 bioactivity of STF2Δ.CSP (SEQ ID NO: 13) assayed using the RAW/TLR5 cell assay. Closed circles indicates proteins assayed on RAW/TLR5 cells. Open circles indicates proteins assayed on RAW264.7 cells (negative control).

FIG. 44 depicts Toll-like Receptors (TLR) and TLR ligands.

FIG. 45 depicts T1 epitopes for use in the compositions of the invention (SEQ ID NO: 37).

FIG. 46 depicts B-cell epitopes for use in the compositions of the invention

(SEQ ID NOs: 38, 139, 143 and 144).

FIG. 47 depicts T* epitopes for use in the compositions of the invention (SEQ ID NOs: 34, 170, 226, 55 and 73).

FIGS. 48A and 48B depict direct ELISA of STF2.1×T1BT* (SEQ ID NO: 9) (FIG. 48A) and SFT2.4×T1BT* (SEQ ID NO: 11) (FIG. 48B).

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

The invention is generally directed to compositions that include a fusion protein of a Toll-like Receptor agonist and malaria antigens; and methods of using the compositions to provide sterile and protective immunity in a subject.

In an embodiment, the invention is a composition that includes at least one fusion protein comprising at least a portion of at least one Toll-like Receptor 5 agonist and at least a portion of at least one malaria antigen.

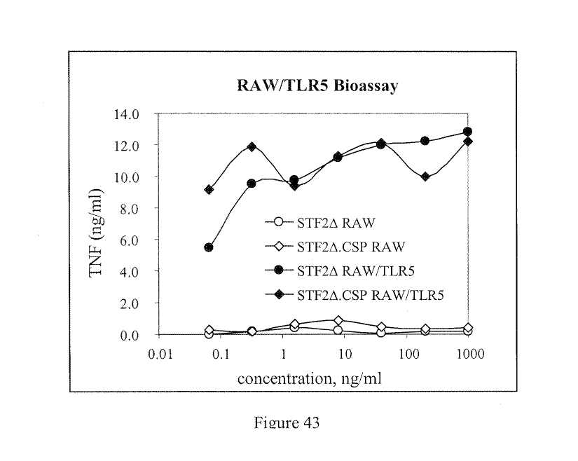

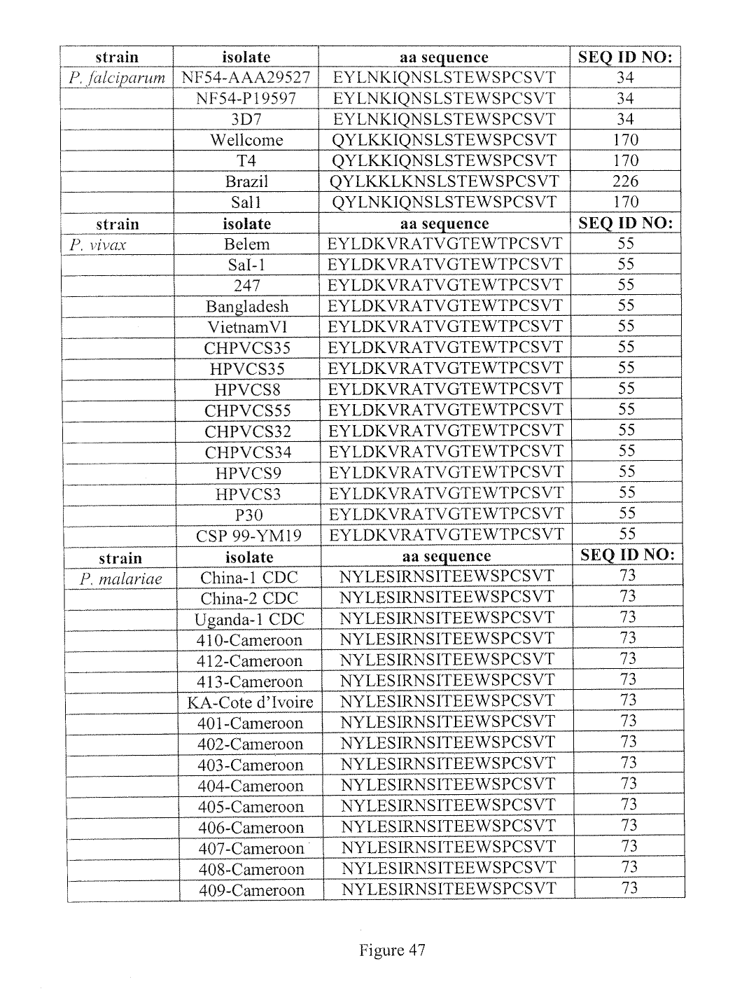

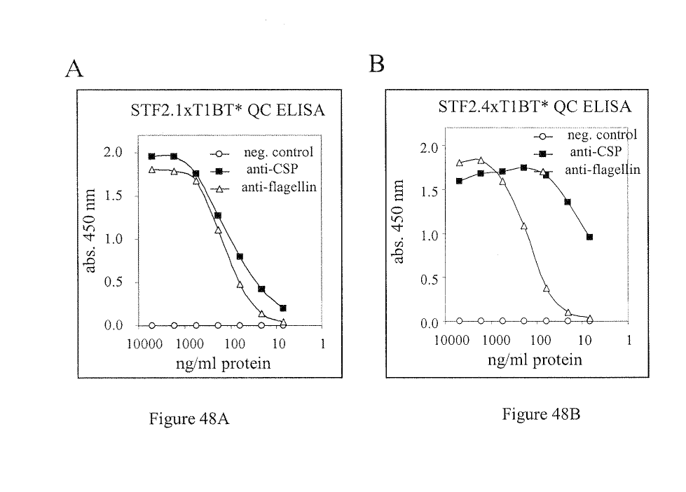

“At least a portion,” as used herein in reference to the malaria antigens of the invention, means any part or the entirety of the malaria antigen. For example, at least a portion of a malaria antigen can include at least one member selected from the group consisting of a T-cell epitope and a B-cell epitope of the malaria antigen, also referred to herein as a “malaria antigen B-cell epitope,” respectively. Exemplary portions of a malaria antigen for use in the compositions of the invention are listed in FIGS. 43, 44, 47, 48, 53, 71-73 and 75-89.

“At least a portion,” as used herein in reference to a Toll-like Receptor agonist, for example, a Toll-like Receptor 5 agonist, such as flagellin (e.g., fljB/STF2, E. coli fliC, S. muenchen fliC), refers to any part of the TLR agonist that can activate a Toll-like Receptor signaling pathway. At least a portion of a flagellin (e.g., motif C; motif N; domain 1, 2, 3) or the entirety of the TLR agonist can initiate or activate an intracellular signal transduction pathway for a Toll-like Receptor 5.

“At least a portion” is also referred to herein as a “fragment.”

Toll-like Receptors (TLRs) were named based on homology to the Drosophila melangogaster Toll protein. Toll-like Receptors are type I transmembrane signaling receptor proteins characterized by an extracellular leucine-rich repeat domain and an intracellular domain homologous to an interleukin 1 receptor. Toll-like Receptors can control innate and adaptive immune responses.

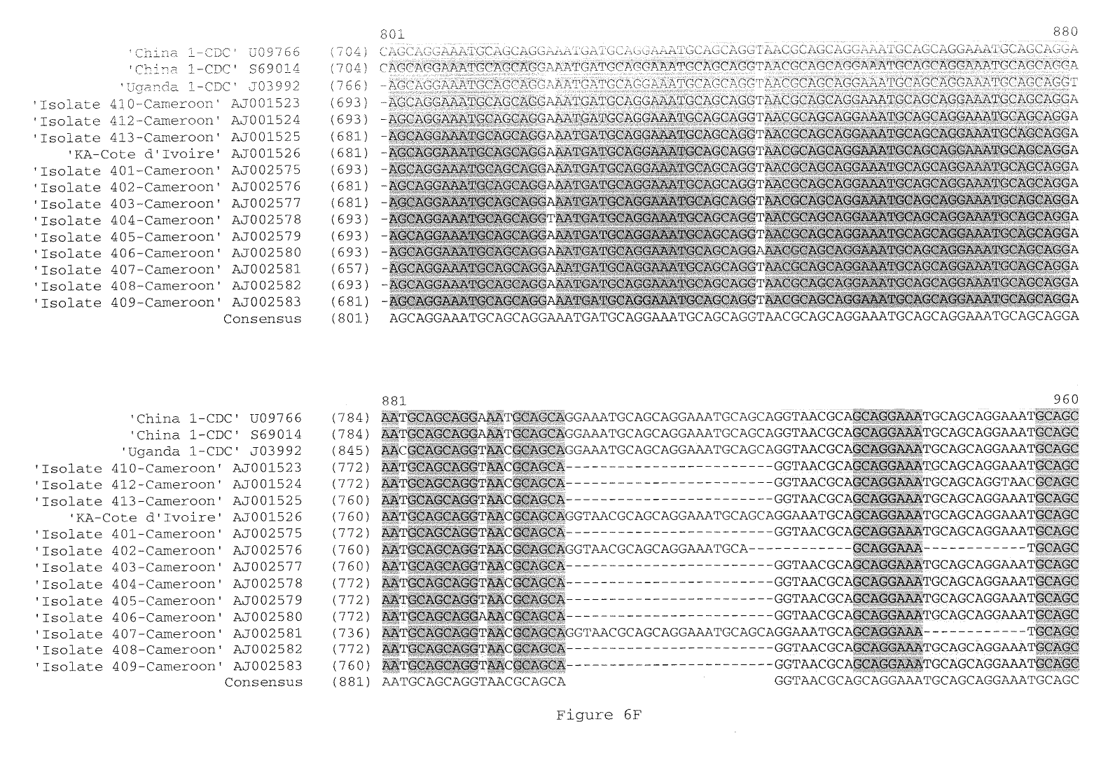

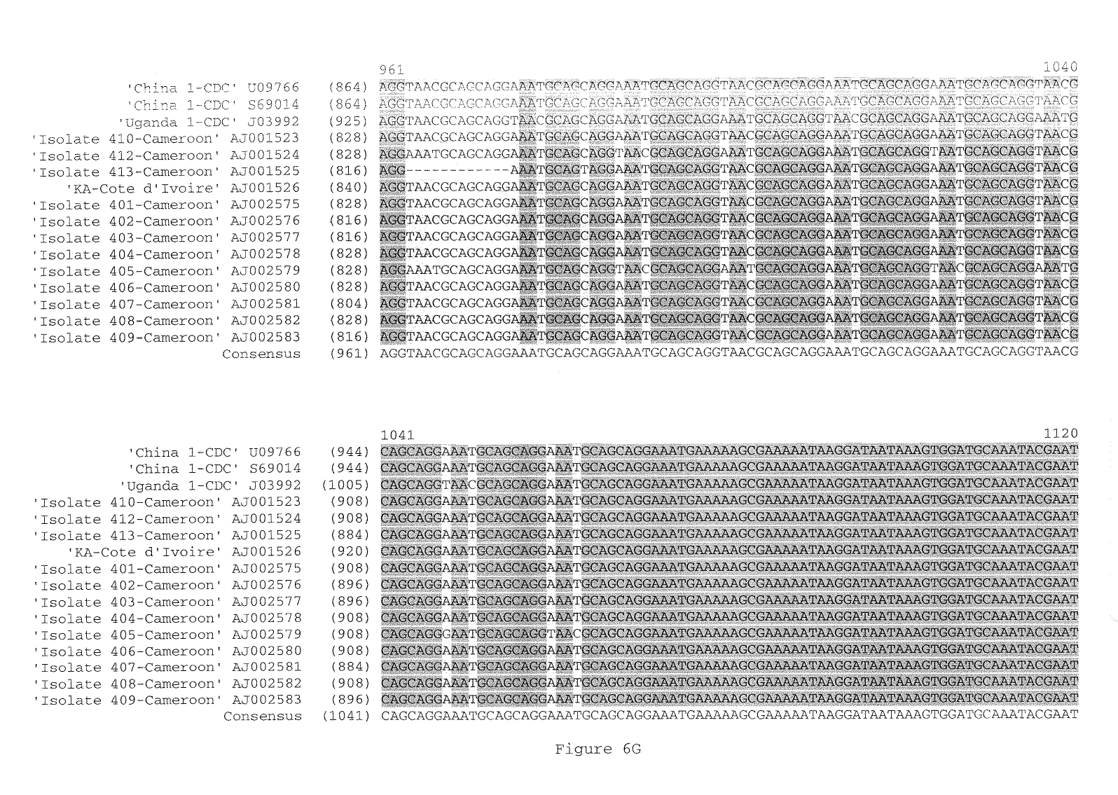

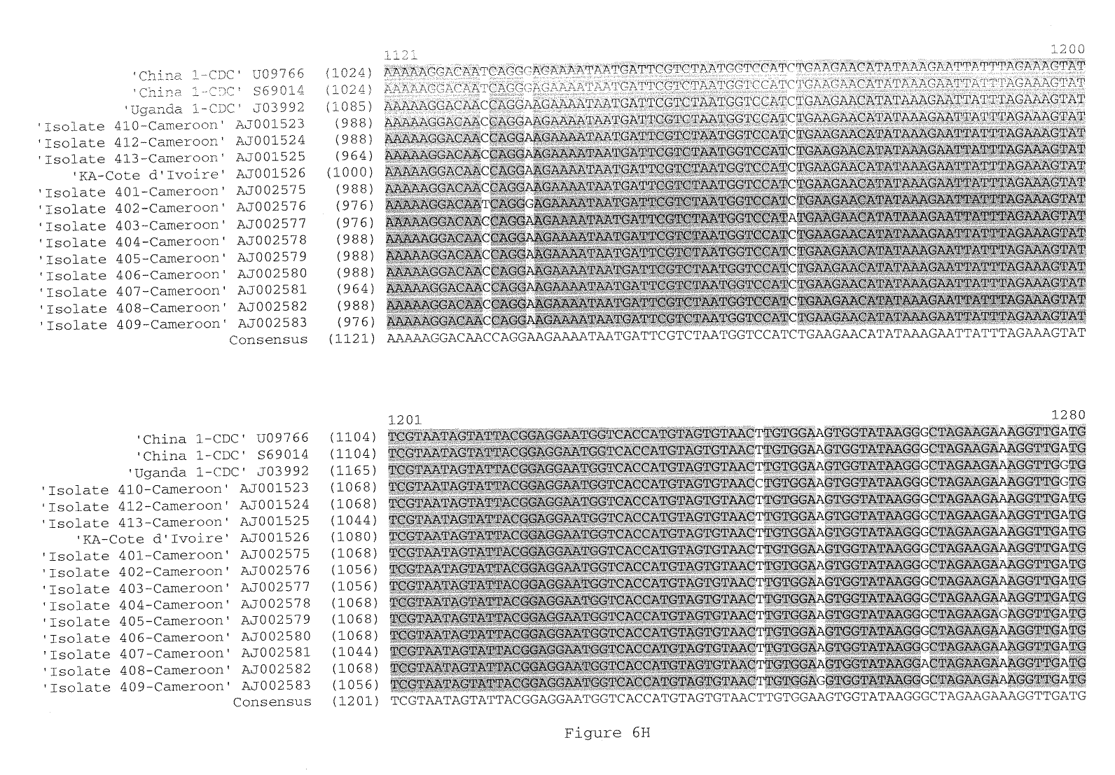

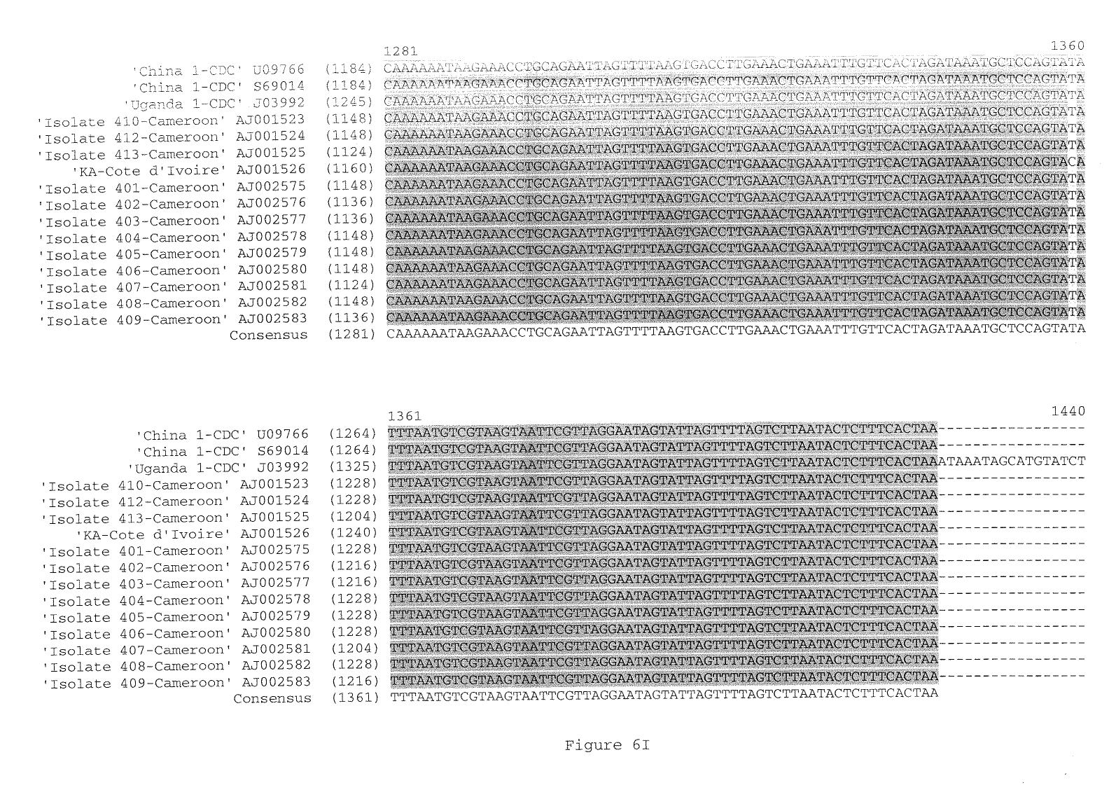

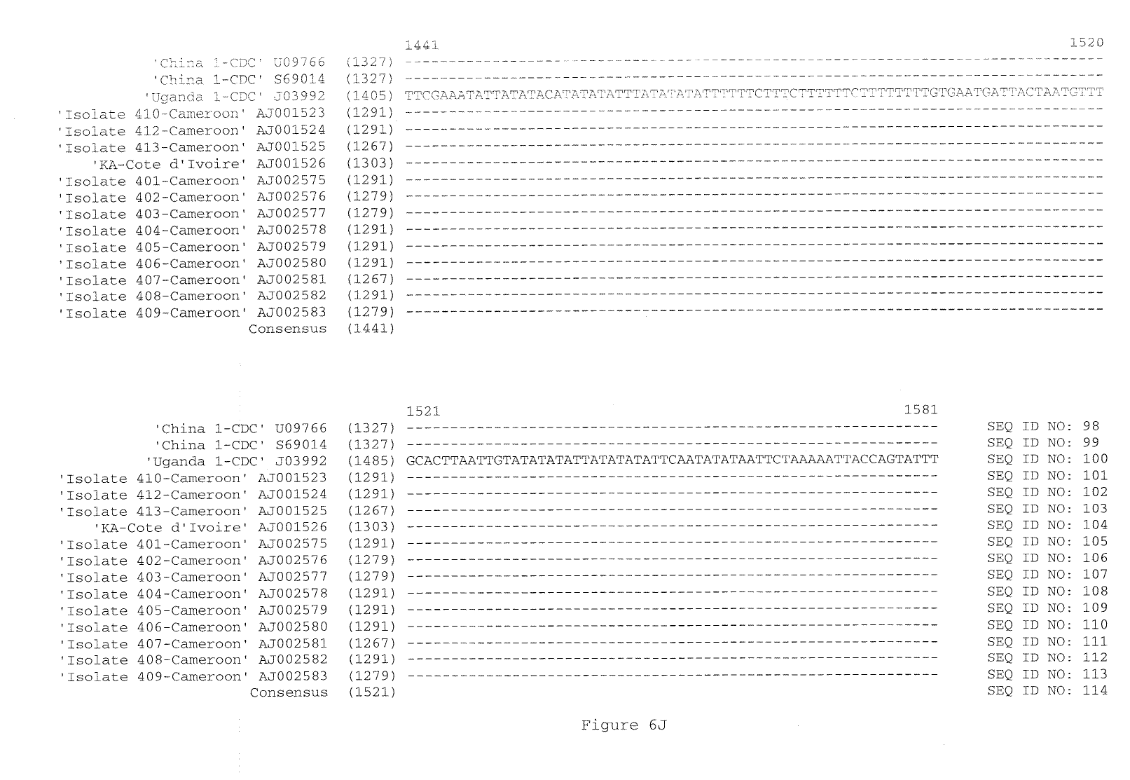

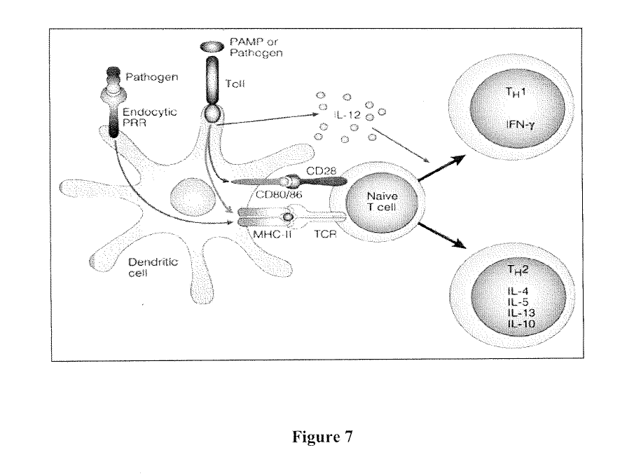

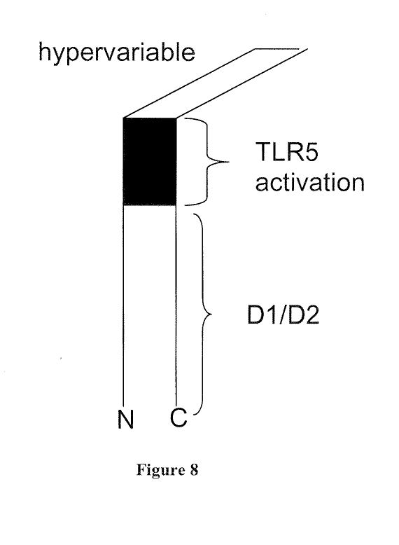

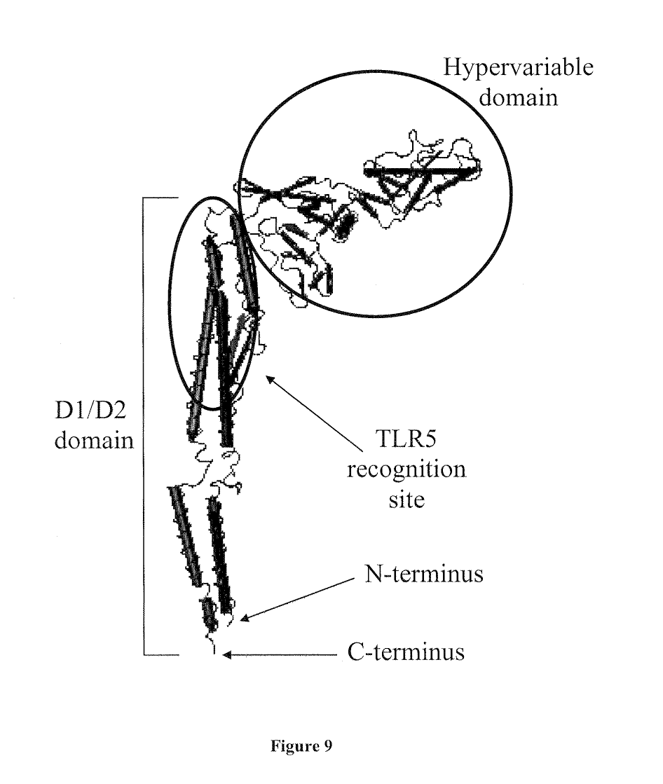

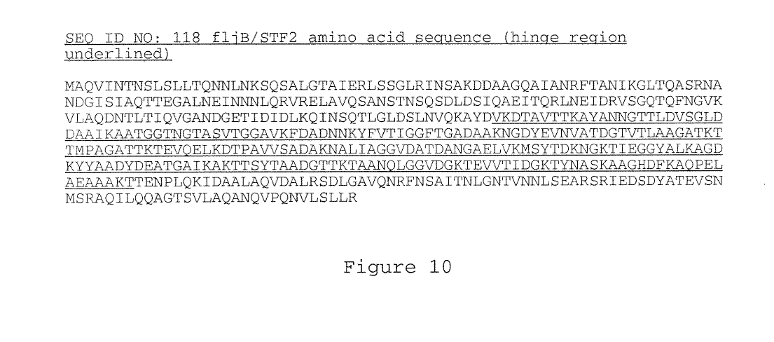

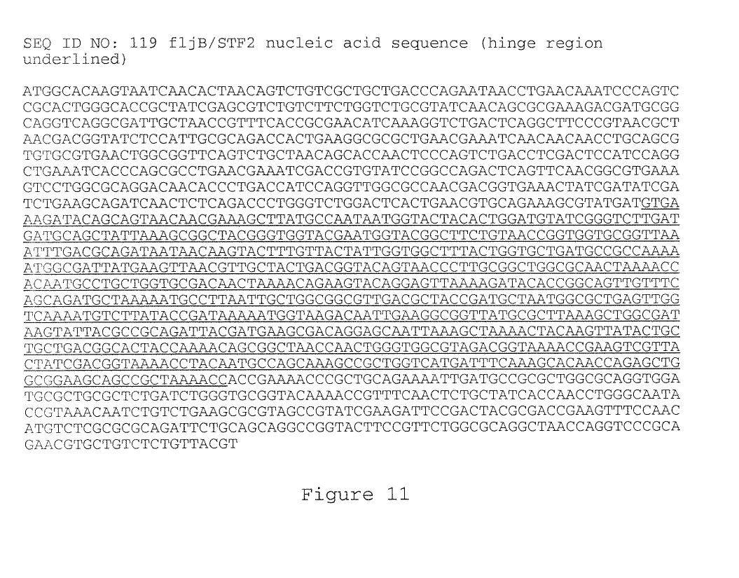

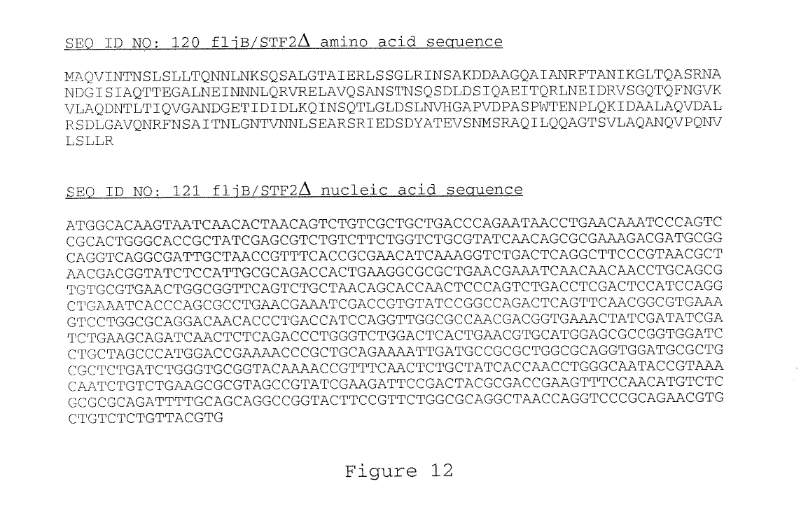

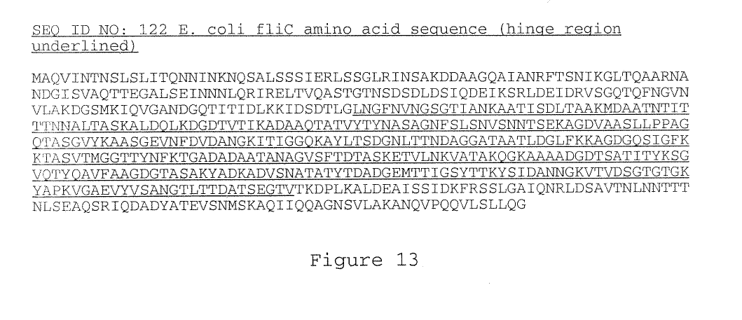

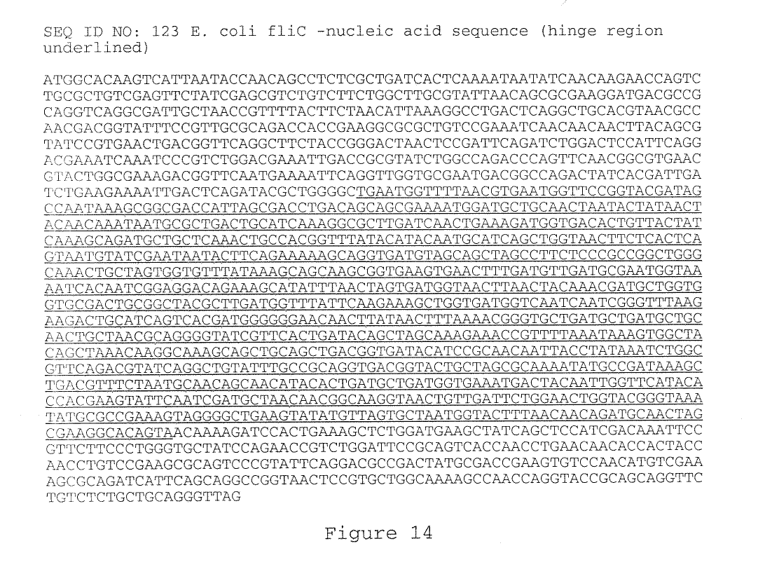

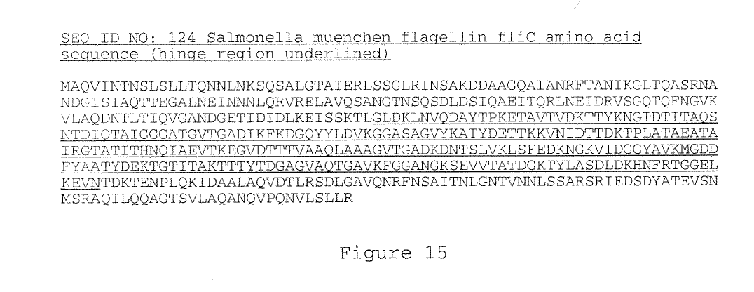

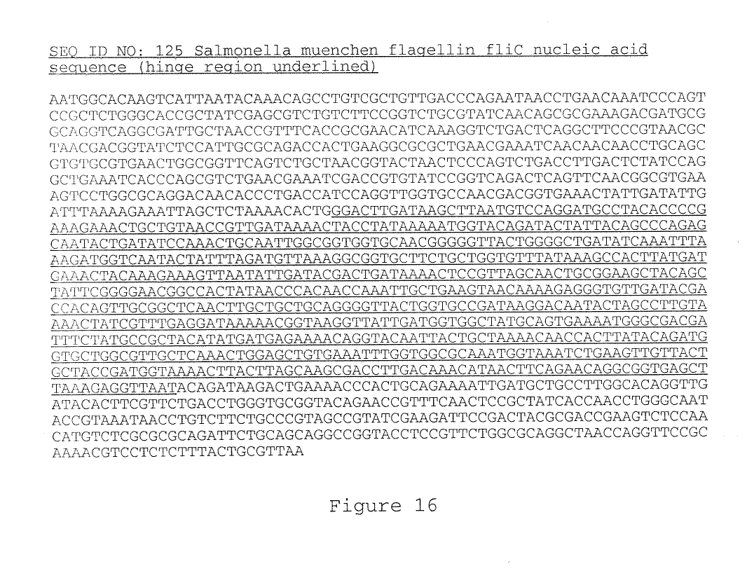

The binding of pathogen-associated molecular patterns (PAMPs) to TLRs can activate innate immune pathways. Target cells can result in the display of co-stimulatory molecules on the cell surface, as well as antigenic peptide in the context of major histocompatibility complex molecules (see FIG. 7). The compositions of the invention include fusion proteins that include Toll-like Receptor 5 (TLR5) that can promote differentiation and maturation of the antigen presenting cells (APC), including production and display of co-stimulatory signals. The fusion proteins of the compositions of the invention can be internalized by interaction with TLR5 and processed through the lysosomal pathway to generate antigenic malaria peptides, which are displayed on the surface in the context of the major histocompatibility complex.

The compositions and proteins of the invention can employ TLR5 agonists (e.g., a flagellin) that trigger cellular events resulting in the expression of costimulatory molecules, secretion of critical cytokines and chemokines; and efficient processing and presentation of antigens to T-cells.

The compositions and fusion proteins of the invention can trigger an immune response to a malaria antigen (e.g., circumsporozite protein (CSP)) and, thus, signal transduction pathways of the innate and adaptive immune system of the subject to thereby stimulate the immune system of a subject to generate antibodies, and provide sterile immunity and protective immunity to malaria. Thus, stimulation of the immune system of the subject may prevent infection by a malaria parasite and thereby treat the subject or prevent the subject from disease, illness and, possibly, death.

“Agonist,” as used herein in referring to a TLR, for example, a TLR5 agonist, means a molecule that activates a TLR signaling pathway. As discussed above, a TLR intracellular signaling pathway is an intracellular signal transduction pathway employed by a particular TLR that can be activated by a TLR ligand or a TLR agonist. Common intracellular pathways are employed by TLRs and include, for example, NF-κB, Jun N-terminal kinase and mitogen-activated protein kinase. Techniques to assess activation of a TLR signaling pathway are known to one of skill in the art. For example, TLR5 activation by a Toll-like Receptor 5 agonist or a fusion protein that includes a TLR5 agonist can be assessed by employing HEK293 cells, which constitutively express TLR5 and secrete several soluble factors, including IL-8, in response to TLR5 signaling. HEK293 cells can be seeded in microplates (about 50,000 cells/well) and TLR5 agonists and/or fusion proteins that include a TLR5 agonist can be added. After about 24 hours of culture, the conditioned medium can be harvested and assayed for the presence of IL-8 in a sandwich ELISA using an anti-human IL-8 matched antibody pair (Pierce; Rockland, Ill.) #M801E and M802B) following the manufacturer's instructions. Optical density can be measured using a microplate spectrophotometer (FARCyte, GE Healthcare; Piscataway, N.J.). The presence of IL-8 signals is indicative of TLR5 agonist activity and activation of a Toll-like Receptor 5, The flagellin for use in the fusion proteins of the invention can include at least one member selected from the group consisting of a Salmonella typhimurium flagellin (e.g., SEQ ID NO: 1), an E. coli flagellin, a S. muenchen flagellin, a Yersinia flagellin, a P. aeruginosa flagellin and a L. monocytogenes flagellin. Portions of flagellin for use in the methods of the invention can include portions of flagellin described in PCT/US2009/002428 (WO 2009/128950), filed Apr. 17, 2009, the entire teachings of which are hereby incorporated by reference in its entirety.

In an embodiment, the flagellin in the compositions and methods described herein can be at least a portion of a S. typhimurium flagellin (GenBank Accession Number AF045151); at least a portion of the S. typhimurium flagellin selected from the group consisting of SEQ ID NO: 1, SEQ ID NO: 118, SEQ ID NO: 130, SEQ ID NO: 124 and SEQ ID NO: 115; at least a portion of an S. muenchen flagellin (GenBank Accession Number AB028476) that includes at least a portion of SEQ ID NO: 124 and SEQ ID NO: 127; at least a portion of P. aeruginosa flagellin that includes at least a portion of SEQ ID NO: 129; at least a portion of a Listeria monocytogenes flagellin that includes at least a portion of SEQ ID NO: 131; at least a portion of an E. coli flagellin that includes at least a portion of SEQ ID NO: 122 and SEQ ID NO: 128; at least a portion of a Yersinia flagellin; and at least a portion of a Campylobacter flagellin. Exemplary flagellin constructs for use in the invention are described, for example, in U.S. application Ser. Nos. 11/820,148; 11/714,873 and 11/714,684, the teachings of all of which are hereby incorporated by reference in their entirety.

The flagellin employed in the compositions and method of the invention can lack at least a portion of a hinge region. Hinge regions are the hypervariable regions of a flagellin. Hinge regions of a flagellin are also referred to herein as “D3 domain or region,” “propeller domain or region,” “hypervariable domain or region” and “variable domain or region.” “Lack” of a hinge region of a flagellin, means that at least one amino acid or at least one nucleic acid codon encoding at least one amino acid that comprises the hinge region of a flagellin is absent in the flagellin. Examples of hinge regions include amino acids 176-415 of SEQ ID NO: 118, which are encoded by nucleic acids 528-1245 of SEQ ID NO: 119; amino acids 174-422 of SEQ ID NO: 122, which are encoded by nucleic acids 522-1266 of SEQ ID NO: 123; or amino acids 173-464 of SEQ ID NO: 124, which are encoded by nucleic acids 519-1392 of SEQ ID NO: 125. Thus, if amino acids 176-415 were absent from the flagellin of SEQ ID NO: 118, the flagellin would lack a hinge region. A flagellin that lacks at least a portion of a hinge region can include SEQ ID NO: 116. A flagellin lacking at least a portion of a hinge region is also referred to herein as a “truncated version” of a flagellin.

“At least a portion of a hinge region,” as used herein, refers to any part of the hinge region of the flagellin, or the entirety of the hinge region. “At least a portion of a hinge region” is also referred to herein as a “fragment of a hinge region.” At least a portion of the hinge region of fljB/STF2 can be, for example, amino acids 200-300 of SEQ ID NO: 118. Thus, if amino acids 200-300 were absent from SEQ ID NO: 118, the resulting amino acid sequence of STF2 would lack at least a portion of a hinge region.

Alternatively, at least a portion of a naturally occurring flagellin can be replaced with at least a portion of an artificial hinge region. “Naturally occurring,” in reference to a flagellin amino acid sequence, means the amino acid sequence present in the native flagellin (e.g., S. typhimurium flagellin, S. muenchin flagellin, E. coli flagellin). The naturally occurring hinge region is the hinge region that is present in the native flagellin. For example, amino acids 176-415 of SEQ ID NO: 118, amino acids 174-422 of SEQ ID NO: 122 and amino acids 173-464 of SEQ ID NO: 124, are the amino acids corresponding to the natural hinge region of STF2, E. coli fliC and S. muenchen flagellins, fliC, respectively. “Artificial,” as used herein in reference to a hinge region of a flagellin, means a hinge region that is inserted in the native flagellin in any region of the flagellin that contains or contained the native hinge region.

The hinge region of a flagellin can be deleted and replaced with at least a portion of a malaria antigen (e.g., CSP of SEQ ID NOs: 25-33, 39-54 and 56-72) or combinations of malaria antigens, such as SEQ ID NOs: 146-151. Exemplary malaria antigens for use in the invention in a CS protein or at least a portion of a CS protein, such as Plasmodium knowlesi CS protein H (GenBank Accession No: K00772; SEQ ID NOs: 201, 202), Plasmodium knowlesi CS protein MKEL3 (GenBank Accession No: EU687467; SEQ ID NOs: 203, 204), Plasmodium knowlesi CS protein MPHG38 (GenBank Accession No: EU687468; SEQ ID NO: 205, 206), Plasmodium knowlesi CS protein MPHG38 (GenBank Accession No: EU687468; SEQ ID NOs: 207, 208), Plasmodium knowlesi CS protein MPRK13 (GenBank Accession No: EU687469; SEQ ID NOs: 209, 210), Plasmodium knowlesi CS protein MSEL26 (GenBank Accession No: EU687470; SEQ ID NOs: 211, 212) and Plasmodium knowlesi CS protein NUR1 (GenBank Accession No: M11031; SEQ ID NOs: 213, 214); a merozite surface protein 1 (MSP1) or at least a portion of a merozite surface protein 1, such as (SEQ ID NO: 215) of Plasmodium falciparum 3D7 merozoite surface protein 1 (MSP1) (GenBank Accession No: XM_{—}001352134; SEQ ID NOs: 215, 216), Plasmodium vivax merozoite surface protein 1 (MSP1) (GenBank Accession No: XM_{—}001614792; SEQ ID NOs: 221, 222); a liver stage antigen 1 (LSA1) or at least a portion of a liver stage antigen, such as Plasmodium falciparum liver stage antigen 1 (LSA1) (GenBank Accession No: X56203; SEQ ID NOs: 219, 220); an apical membrane antigen 1 (AMA1) or at least a portion of an apical membrane antigen 1, such as Plasmodium falciparum 3D7 apical membrane antigen 1 (AMA1) (GenBank Accession No: XM_{—}001347979; SEQ ID NO: 217) and Plasmodium vivax apical membrane antigen 1 (AMA1) (GenBank Accession No: AF063138; SEQ ID NOs: 223, 224). An artificial hinge region may be employed in a flagellin that lacks at least a portion of a hinge region, which may facilitate interaction of the carboxy- and amino-terminus of the flagellin for binding to TLR5 and, thus, activation of the TLR5 innate signal transduction pathway. A flagellin lacking at least a portion of a hinge region is designated by the name of the flagellin followed by a “Δ.” For example, an STF2 (e.g., SEQ ID NO: 113) that lacks at least a portion of a hinge region is referenced to as “STF2Δ” or “fljB/STF2Δ” (e.g., SEQ ID NO: 3).

The flagellin for use in the methods and compositions of the invention can be a at least a portion of a flagellin, wherein the flagellin includes at least one cysteine residue that is not present in the naturally occurring flagellin and the flagellin component activates a Toll-like Receptor 5; a flagellin component that is at least a portion of a flagellin, wherein at least one lysine of the flagellin component has been substituted with at least one arginine and the flagellin component activates a Toll-like Receptor 5; a flagellin component that is at least a portion of a flagellin, wherein at least one lysine of the flagellin component has been substituted with at least one serine residue and the flagellin component activates a Toll-like Receptor 5; a flagellin component that is at least a portion of a flagellin, wherein at least one lysine of the flagellin component has been substituted with at least one histidine residue and the flagellin component activates a Toll-like Receptor 5.

“Fusion proteins,” as used herein, refers to the joining of two components (also referred to herein as “fused” or linked”) (e.g., a Toll-like Receptor agonist and at least a portion of a malaria antigen, such as at least a portion of a CSP). The portion of the CSP protein can include at least one T-cell epitope (e.g., SEQ ID NOs: 34-38, 55, 73, 133-137, 170 and 226) and a B-cell epitope e.g., (SEQ ID NO: 38, 138-145). Fusion proteins of the invention can be generated from at least two similar or distinct malaria antigens. For example, a fusion protein of the invention can include two malaria antigen T-cell epitopes of SEQ ID NO: 34 (two similar antigens); two malaria antigen B-cell epitopes of SEQ ID NO: 139 (two similar antigens); a malaria antigen B-cell epitope of SEQ ID NO: 139 and a T-cell epitope of SEQ ID NO: 34 (two distinct antigens); or any combination thereof.

Fusion proteins of the invention can be generated by recombinant DNA technologies or by chemical conjugation of the components (e.g., Toll-like Receptor agonist and malaria antigen) of the fusion protein. Recombinant DNA technologies and chemical conjugation techniques are well established procedures and known to one of skill in the art. Exemplary techniques to generate fusion proteins that include Toll-like Receptor agonists are described herein and in U.S. application Ser. Nos. 11/714,684 and 11/714,873, the teachings of both of which are hereby incorporated by reference in their entirety.

The fusion proteins of the invention can activate a Toll-like Receptor. In particular, the fusion proteins of the invention that include a Toll-like Receptor 5 and at least a portion of a malaria antigen can activate a Toll-like Receptor 5.

“Activates,” when referring to a TLR, means that the Toll-like Receptor δ agonist (e.g., a flagellin) or the fusion protein of the invention stimulates a response associated with a TLR. For example, bacterial flagellin activates TLR5 and host inflammatory responses (Smith, K. D., et al., Nature Immunology 4:1247-1253 (2003)).

In an embodiment, a carboxy-terminus of the malaria antigen is fused (also referred to herein as “linked”) to an amino terminus of the flagellin component of the protein. In another embodiment, an amino-terminus of the malaria antigen is fused to a carboxy-terminus of the flagellin component of the protein.

Fusion proteins of the invention can be designated by the components of the fusion proteins separated by a “.”. For example, “STF2.CSP” refers to a fusion protein comprising one flagellin, Salmonella typhimurium flagellin (STF2) and one CSP protein; and “STF2Δ.CSP” refers to a fusion protein comprising one flagellin, Salmonella typhimurium flagellin (STF2) protein without the hinge region (STF2A, also referred to herein as “STF2 delta”) and a CSP protein. Exemplary fusion proteins of the invention include SEQ ID NOs: 7, 9, 11, 13, 15, 17, 20, 22 and 24).

Proteins of the invention can include, for example, two, three, four, five, six, seven, eight, nine or ten or more Toll-like Receptor agonists (e.g., flagellin) and two, three, four, five, six, seven, eight, nine, ten or more malaria antigen proteins. When two or more TLR agonists and/or two or more malaria antigen proteins comprise fusion proteins of the invention, they are also referred to as “multimers.” For example, a multimer of a CSP protein can be four CSP sequences, which is referred to herein as 4×CSP. Likewise, a multimer of at least a portion of a malaria antigen that includes a T-cell epitope and a B-cell epitope can be four or ten T-cell and B-cell epitopes each alone (e.g., 4×T1) or in any combination (e.g., 4×T1BT* (also referred to herein as “T1BT*-4×”), 10×T1BT* (also referred to herein as “T1BT*-10×”), 4× T1T*, 10×T1T*, 4×T1B, 10×T1B, 4×BT*, 10×BT*).

The proteins of the invention can further include a linker between at least one component of the protein (e.g., a malaria antigen) and at least one other component of the protein (e.g., flagellin) of fusion proteins of the composition, a linker (e.g., an amino acid linker) between at least two of similar components of the protein (e.g., a malaria antigen and a Toll-like Receptor 5 agonist) or any combination thereof. The linker can be between the Toll-like Receptor agonist and malaria antigen of a fusion protein. “Linker,” as used herein in reference to a protein of the invention, refers to a connector between components of the protein in a manner that the components of the protein are not directly joined. For example, one part of the protein (e.g., flagellin component) can be linked to a distinct part (e.g., a malaria antigen) of the protein. Likewise, at least two or more similar or like components of the protein can be linked (e.g., two flagellin components can further include a linker between each flagellin component) or two malaria antigens (e.g., CSP, such as SEQ ID NOs: 25-33, 39-54 and 56-72; T-cell epitopes, such as SEQ ID NOs: 34-39, 55, 73 and 133-137 and B-cell epitopes, such as SEQ ID NO: 138-145) components can further include a linker between each malaria antigen.

Additionally, or alternatively, the proteins of the invention can include a combination of a linker between distinct components of the protein and similar or like components of the protein. For example, a protein can comprise at least two TLR agonists that further includes a linker between, for example, two or more flagellin; at least two malaria antigens that further include a linker between them; a linker between one component of the protein (e.g., flagellin) and another distinct component of the protein (e.g., a malaria antigen), or any combination thereof.

The linker can be an amino acid linker. The amino acid linker can include synthetic or naturally occurring amino acid residues. The amino acid linker employed in the proteins of the invention can include at least one member selected from the group consisting of a lysine residue, a glutamic acid residue, a serine residue and an arginine residue.

The Toll-like Receptor agonist can be fused to a carboxy-terminus, the amino-terminus or both the carboxy- and amino-terminus of the malaria antigen.

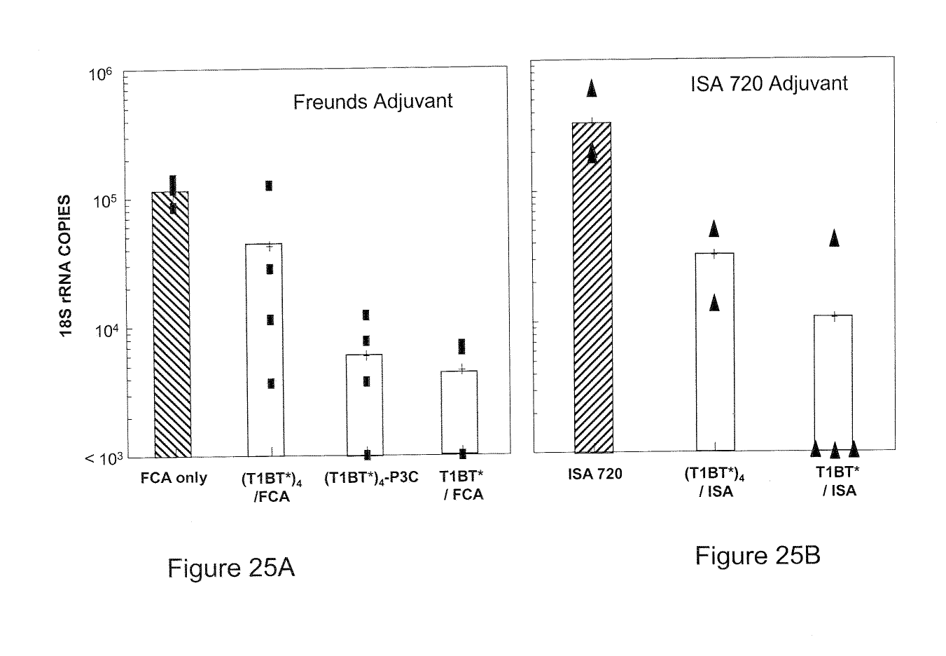

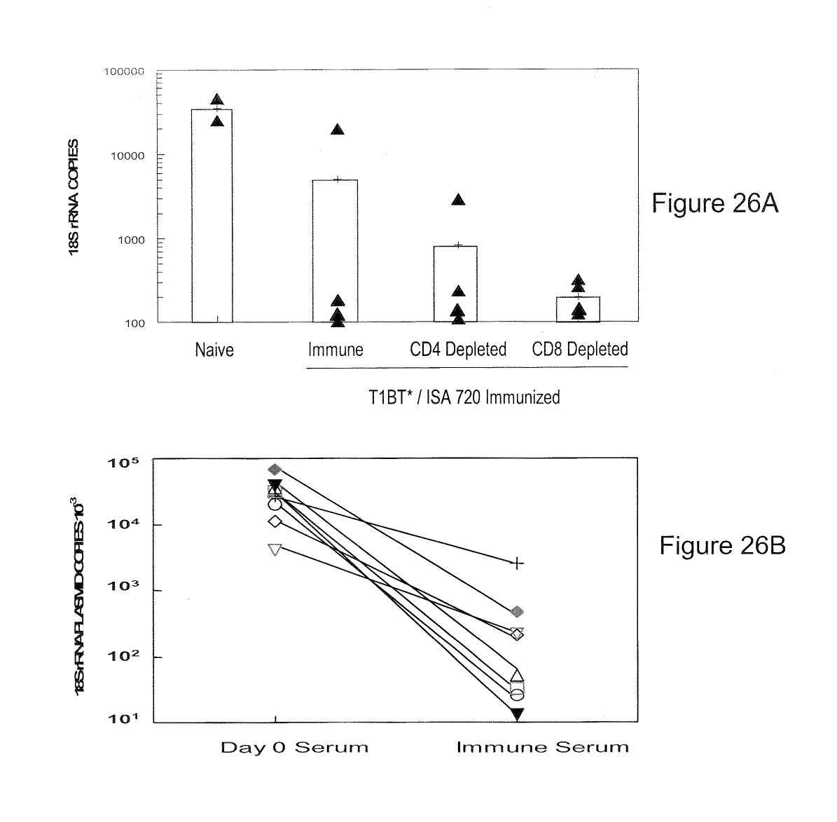

Proteins can be generated by fusing the malaria antigen to at least one of four regions (Regions 1, 2, 3 and 4) of flagellin, which have been identified based on the crystal structure of flagellin (PDB:1UCU) (see, for example, FIGS. 25 and 26). Region 1 is also referred to as Domain O or DO. Region 2 is also referred to as Domain 1 or D1. Region 3 is also referred to as D2. Region 4 is also referred to as D3.

Region 1 is TIAL (SEQ ID NO: 153) . . . - . . . GLG (194-211 of SEQ ID NO: 126). The corresponding residues for Salmonella typhimurium fljB construct are TTLD (SEQ ID NO: 154) . . . - . . . GTN (196-216 of SEQ ID NO: 132). This region is an extended peptide sitting in a groove of two beta strands (GTDQKID (SEQ ID NO: 155) and NGEVTL (SEQ ID NO: 156) of (SEQ ID NO: 126).

Region 2 of the Salmonella flagellin is a small loop GTG (238-240 of SEQ ID NO: 126) in 1UCU structure (see, for example, FIGS. 25 and 26). The corresponding loop in Salmonella fljB is GADAA (SEQ ID NO: 157) (244-248 of SEQ ID NO: 132).

Region 3 is a bigger loop that resides on the opposite side of the Region 1 peptide (see, for example, FIGS. 25 and 26). This loop can be simultaneously substituted together with region 1 to create a double copy of the malaria antigen. The loop starts from ALGA (SEQ ID NO: 158) and ends at PATA (SEQ ID NO: 159) (259-274 of SEQ ID NO: 126). The corresponding Salmonella fljB sequence is AAGA (SEQ ID NO: 160 . . . - . . . ATTK (SEQ ID NO: 161) (266-281 of SEQ ID NO: 132). The sequence AGATKTTMPAGA (SEQ ID NO: 162) (267-278 of SEQ ID NO: 132) can be replaced with a malaria antigen.

Region 4 is the loop (GVTGT (SEQ ID NO: 163)) connecting a short α-helix (TEAKAALTAA (SEQ ID NO: 164)) and a β-strand (ASVVKMSYTDN (SEQ ID NO: 165) SEQ ID NO: 126. The corresponding loop in Salmonella fljB is a longer loop VDATDANGA (SEQ ID NO: 166 (307-315 of SEQ ID NO: 132). At least a portion of a malaria antigen, including a CSP, such as SEQ ID NOs: 25-33, 39-54 and 56-72 and/or SEQ ID NOs: 34-39, 55, 73 and 133-137, can be inserted into or replace this region.

Fusion proteins of at least a portion of at least one Toll-like Receptor agonist (e.g., TLR5) and at least a portion of at least one malaria antigen can be generated by recombinant DNA technologies or chemical conjugation techniques. Fusion of the TLR to a malaria antigen would result in a fusion protein that can activate a Toll-like Receptor. Methods to generate fusion proteins of the invention are known in the art and are described herein.

Fusion proteins of the invention can include Toll-like Receptor agonists that include cysteine residues that are substituted for at least one amino acid residue in a naturally occurring Toll-like Receptor agonist remote to the Toll-like Receptor recognition or binding site that binds the respective Toll-like Receptor. For example, a cysteine residue can be substituted for a naturally occurring amino acid in a flagellin for use in the fusion proteins of the invention remote to the TLR5 binding or recognition site.

For example, flagellin from Salmonella typhimurium STF1 (FliC) is depicted in SEQ ID NO: 126 (Accession No: P06179). The TLR5 recognition site is amino acid about 79 to about 117 and about 408 to about 439 of SEQ ID NO: 126. Cysteine residues can substitute for or be included in combination with amino acid about 408 to about 439 of SEQ ID NO: 126; amino acids about 1 and about 495 of SEQ ID NO: 126; amino acids about 237 to about 241 of SEQ ID NO: 126; and/or amino acids about 79 to about 117 and about 408 to about 439 of SEQ ID NO: 126.

Salmonella typhimurium flagellin STF2 (F1jB) is depicted in SEQ ID NO: 118. The TLR5 recognition site is amino acids about 80 to about 118 and about 420 to about 451 of SEQ ID NO: 118. Cysteine residues can substitute for or be included in combination with amino acids about 1 and about 505 of SEQ ID NO: 118; amino acids about 240 to about 244 of SEQ ID NO: 118; amino acids about 79 to about 117 and/or about 419 to about 450 of SEQ ID NO: 118.

Salmonella muenchen flagellin is depicted in SEQ ID NO: 124 (Accession No: #P06179). The TLR5 recognition site is amino acids about 79 to about 117 and about 418 to about 449 of SEQ ID NO: 124. Cysteine residues can substitute for or be included in combination with amino acids about 1 and about 504 of SEQ ID NO: 124; about 237 to about 241 of SEQ ID NO: 124; about 79 to about 117; and/or about 418 to about 449 of SEQ ID NO: 124.

Escherichia coli flagellin is depicted in SEQ ID NO: 122 (Accession No: P04949). The TLR5 recognition site is amino acids about 79 to about 117 and about 410 to about 441 of SEQ ID NO: 122. Cysteine residues can substitute for or be included in combination with amino acids about 1 and about 497 of SEQ ID NO: 122; about 238 to about 243 of SEQ ID NO: 122; about 79 to about 117; and/or about 410 to about 441 of SEQ ID NO: 122.

Pseudomonas auruginosa flagellin is depicted in SEQ ID NO: 129. The TLR5 recognition site is amino acids about 79 to about 114 and about 308 to about 338 of SEQ ID NO: 129. Cysteine residues can substitute for or be included in combination with amino acids about 1 and about 393 of SEQ ID NO: 129; about 211 to about 213 of SEQ ID NO: 129; about 79 to about 114; and/or about 308 to about 338 of SEQ ID NO: 129.

Listeria monocytogenes flagellin is depicted in SEQ ID NO: 131. The TLR5 recognition site is amino acids about 78 to about 116 and about 200 to about 231 of SEQ ID NO: 131. Cysteine residues can substitute for or be included in combination with amino acids about 1 and about 287 of SEQ ID NO: 131; about 151 to about 154 of SEQ ID NO: 131; about 78 to about 116; and/or about 200 to about 231 of SEQ ID NO: 131.

The malaria antigen can be chemically conjugated (or fused) to at least a portion of a Toll-like Receptor agonist, such as a flagellin Chemical conjugation (also referred to herein as “chemical coupling”) can include conjugation by a reactive group, such as a thiol group (e.g., a cysteine residue) or by derivatization of a primary (e.g., a amino-terminal) or secondary (e.g., lysine) group. Different crosslinkers can be used to chemically conjugate TLR ligands (e.g., TLR agonists) to a malaria antigen. Exemplary cross linking agents are commerically available, for example, from Pierce (Rockland, Ill.). Methods to chemically conjugate the malaria antigen to the Toll-like Receptor agonist, such as a flagellin, are well-known and include the use of commercially available cross-linkers, such as those described herein.

For example, conjugation of a malaria antigen to at least a portion of a flagellin can be through at least one cysteine residue of the flagellin component or the Toll-like Receptor component and at least one cysteine residue of a malaria antigen employing established techniques. The malaria antigen can be derivatized with a homobifunctional, sulfhydryl-specific crosslinker; desalted to remove the unreacted crosslinker; and then the partner added and conjugated via at least one cysteine residue cysteine. Exemplary reagents for use in the conjugation methods can be purchased commercially from Pierce (Rockland, Ill.), for example, BMB (Catalog No: 22331), BMDB (Catalog No: 22332), BMH (Catalog No: 22330), BMOE (Catalog No: 22323), BM[PEO]₃ (Catalog No: 22336), BM[PEO]₄ (Catalog No: 22337), DPDPB (Catalog No: 21702), DTME (Catalog No: 22335), HBVS (Catalog No: 22334).

Alternatively, the malaria antigen can be conjugated to lysine residues on flagellin or Toll-like Receptor agonists. A malaria antigen or Toll-like Receptor agonist containing no cysteine residues is derivatized with a heterobifunctional amine and sulfhydryl-specific crosslinker. After desalting, the cysteine-containing partner is added and conjugated. Exemplary reagents for use in the conjugation methods can be purchased from Pierce (Rockland, Ill.), for example, AMAS (Catalog No: 22295), BMPA (Catalog No. 22296), BMPS (Catalog No: 22298), EMCA (Catalog No: 22306), EMCS (Catalog No: 22308), GMBS (Catalog No: 22309), KMUA (Catalog No: 22211), LC-SMCC (Catalog No: 22362), LC-SPDP (Catalog No: 21651), MBS (Catalog No: 22311), SATA (Catalog No: 26102), SATP (Catalog No: 26100), SBAP (Catalog No: 22339), SIA (Catalog No: 22349), STAB (Catalog No: 22329), SMCC (Catalog No: 22360), SMPB (Catalog No: 22416), SMPH (Catalog No. 22363), SMPT (Catalog No: 21558), SPDP (Catalog No: 21857), Sulfo-EMCS (Catalog No: 22307), Sulfo-GMBS (Catalog No: 22324), Sulfo-KMUS (Catalog No: 21111), Sulfo-LC-SPDP (Catalog No: 21650), Sulfo-MBS (Catalog No: 22312), Sulfo-SIAB (Catalog No: 22327), Sulfo-SMCC (Catalog No: 22322), Sulfo-SMPB (Catalog No: 22317), Sulfo-LC-SMPT (Catalog No.: 21568).

The malaria antigen for use in the compositions of the invention can include at least a portion of at least one member selected from the group consisting of a Plasmodium malariae malaria antigen, a Plasmodium reichenowi malaria antigen, a Plasmodium yoelii malaria antigen, a Plasmodium berghei malaria antigen, a Plasmodium vivax malaria antigen, a Plasmodium ovale malaria antigen and a Plasmodium knowlesi malaria antigen. In an embodiment, the malaria antigen includes a Plasmodium falciparum malaria antigen.

The malaria parasite life cycle involves two hosts, a mosquito and a human. During a blood meal, a malaria-infected female Anopheles mosquito inoculates sporozoites into the human host. Sporozoites infect liver cells of the human host and mature into schizonts, which rupture and release merozoites. In Plasmodium vivax and Plasmodium ovale parasite species, a dormant stage of the parasite (i.e., hypnozoites) can persist in the human liver and cause relapses by invading the bloodstream weeks or even years later after the initial infection. Following initial replication in the liver (exo-erythrocytic schizogony), the parasites undergo asexual reproduction in erythrocytes (erythrocytic schizogony) of the human host. Merozoites then infect red blood cells of the human host. The ring stage trophozoites of the parasite mature into schizonts, which rupture releasing merozoites. Some parasites differentiate into sexual erythrocytic stages (gametocytes). Blood stage parasites are responsible for the clinical manifestations of malaria disease in a human.

The gametocytes of the malaria parasite, male (microgametocytes) and female (macrogametocytes), are ingested by an Anopheles mosquito during a blood meal on a human. Replication of the parasite in the mosquito is known as the sporogonic cycle. In the stomach of the mosquito, the microgametes penetrate the macrogametes generating zygotes. The zygotes in turn become motile and elongated ookinetes, which invade the midgut wall of the mosquito where they develop into oocysts. The oocysts grow, rupture and release sporozoites, which make their way to the salivary glands of a mosquitoes. Inoculation of the sporozoites into a new human host perpetuates the malaria life cycle.

Plasmodium male and female gametocytes are produced during the blood stage infection. These sexual stages are taken up by the mosquito when it takes a blood meal from a human host. The gametes fuse in the mosquito and form a motile zygote (ookinete) which migrates through the gut wall. The parasite then forms an oocyst in which the haploid sporozoites are formed by schizogony. Sporozoites rupture from the oocyst, migrate to the salivary glands and are then are injected in the next blood meal to transmit the parasite.

Malaria antigens suitable for use in the compositions of the invention can be antigens that are present in the malaria parasite at one or more stages of its life, including the asexual blood stage and the sexual stage of the parasite; pre-erythrocytic stages (sporozoite, liver exo-erythrocytic forms) in blood stages (MSP-1, AMA-1) (see, for example, Vekeman, J. et al., Expert Rev. Vaccines 7:223-240 (2008)). Exemplary malaria antigens for use in the compositions and methods of the invention can include pre-erythrocytic and blood stage antigens, such as sporozite antigens (e.g., circumsporozoite protein (CSP)), Merozoite Surface Proteins (MSP), Duffy-binding protein-1, apical membrane antigen-1 (AMA-1), reticulocyte-binding protein and a liver stage antigen-1 (LSA-1)). Exemplary malaria antigens and nucleic acid sequences encoding the antigens for use in the compositions of the invention are depicted in FIGS. 75-89.

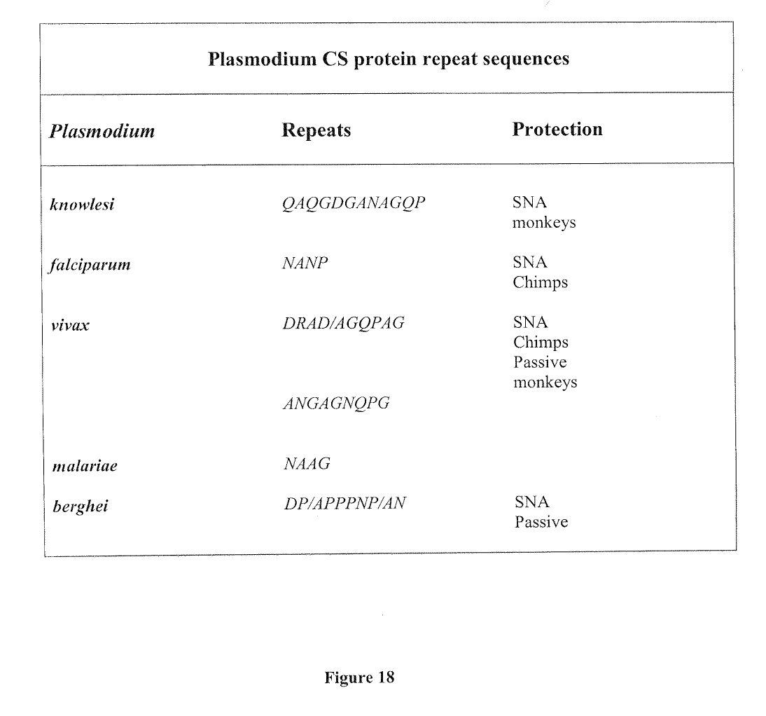

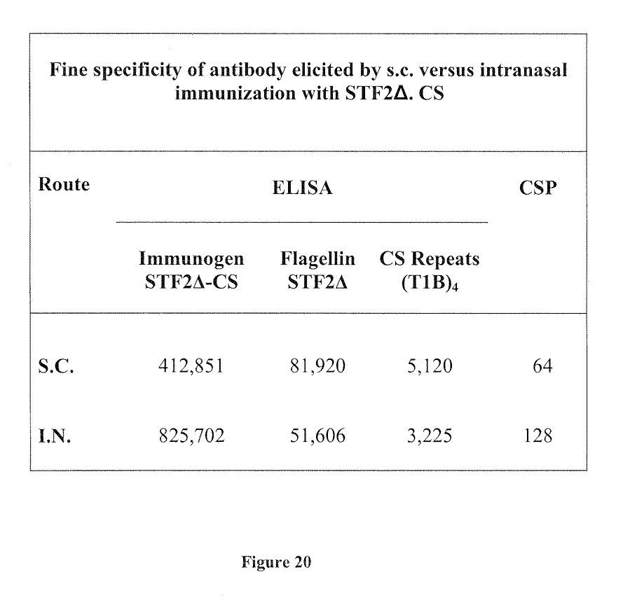

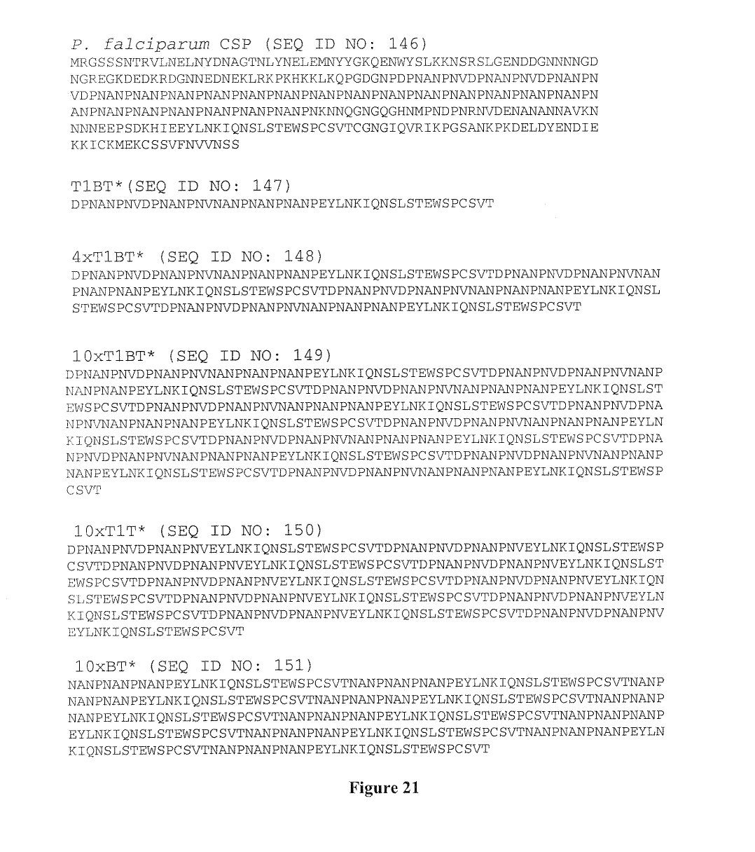

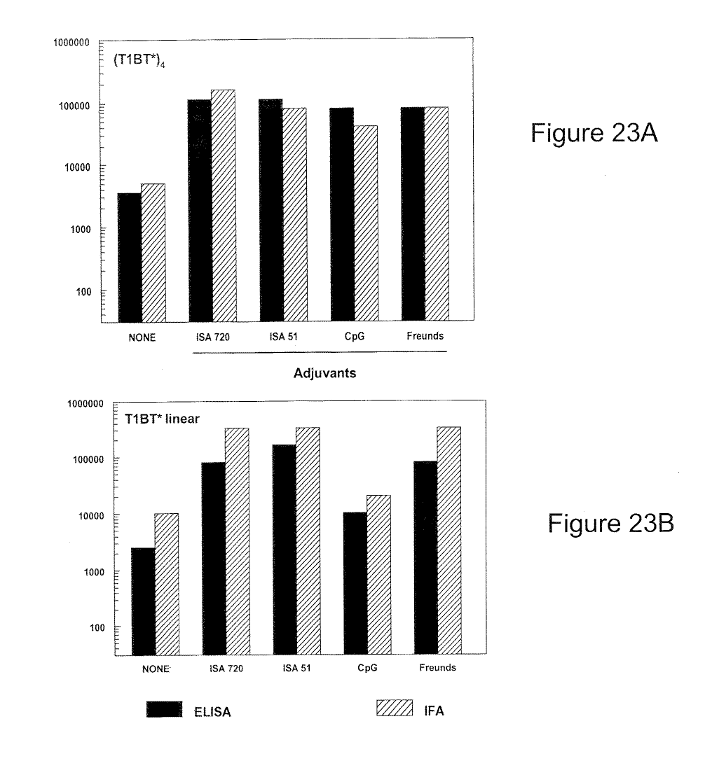

The CSP is present in both the sporozoite and liver stages of the parasite. Exemplary CSP antigens for use in the fusion proteins of the invention are described in U.S. Pat. No. 6,669,945, the entire teachings of which are hereby incorporated by reference in its entirety. The circumsporozite protein antigen for use in the compositions of the invention can include at least a portion of at least one member selected from the group consisting of SEQ ID NOs: 25-33, 39-54 and 56-72 (See FIGS. 18-23).

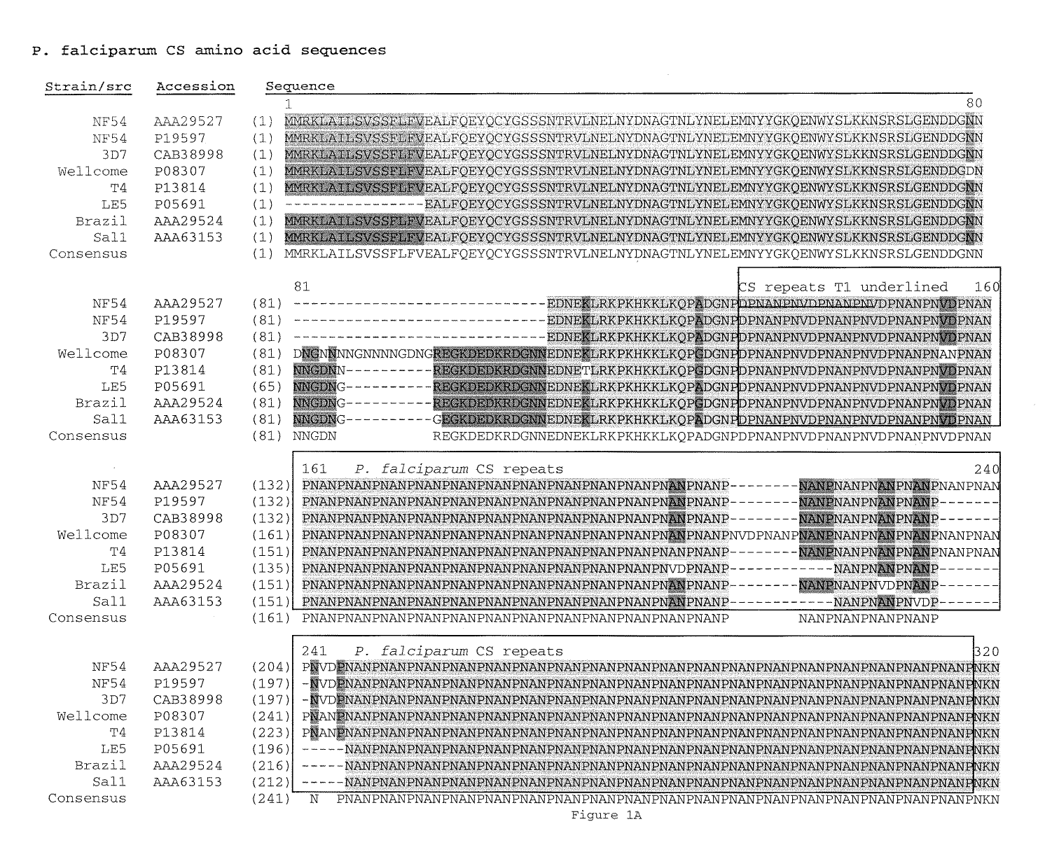

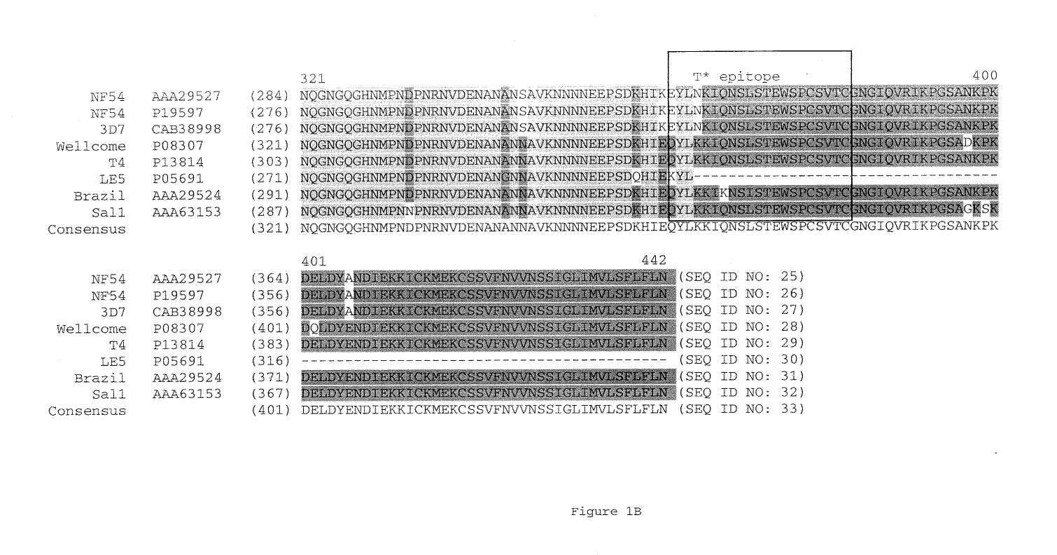

Exemplary Plasmodium falciparum CS proteins for use in the invention are shown in FIG. 1. The T-cell epitope T* (EYLNKIQNSLSTEWSPCSVT; SEQ ID NO: 34) of the Plasmodium falciparum CS protein is indicated, which is polymorphic and can vary in different Plasmodium falciparum strains. The T1 cell epitope is located in the minor repeat region, located in the 5′ end of the central repeat region and includes alternating NANPNVDP sequences (SEQ ID NO: 35), while the major repeat region include repeats of NANP (SEQ ID NO: 36). The T1 epitope is located in the CS repeat region and functions as both a T helper epitope as well as a B cell epitope. The T1 epitope is DPNANPNVDPNANPNV (SEQ ID NO: 37) is also referred to herein as “(DPNANPNV)₂”), which is the malaria antigen component of the STF2.T1BT* fusion protein (SEQ ID NO: 9). The minimal B cell epitope is three NANP (SEQ ID NO: 30) repeats, NANPNANPNANP (SEQ ID NO: 38), also referred to herein as “(NANP)₃”.

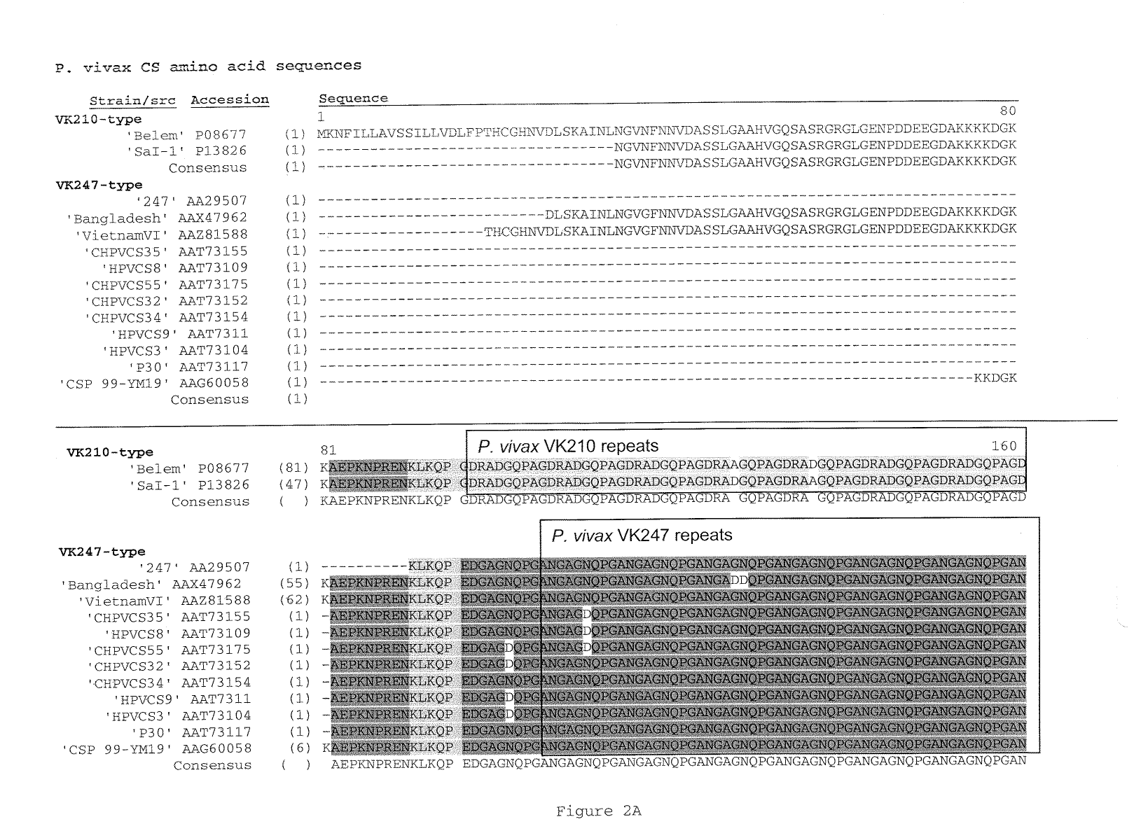

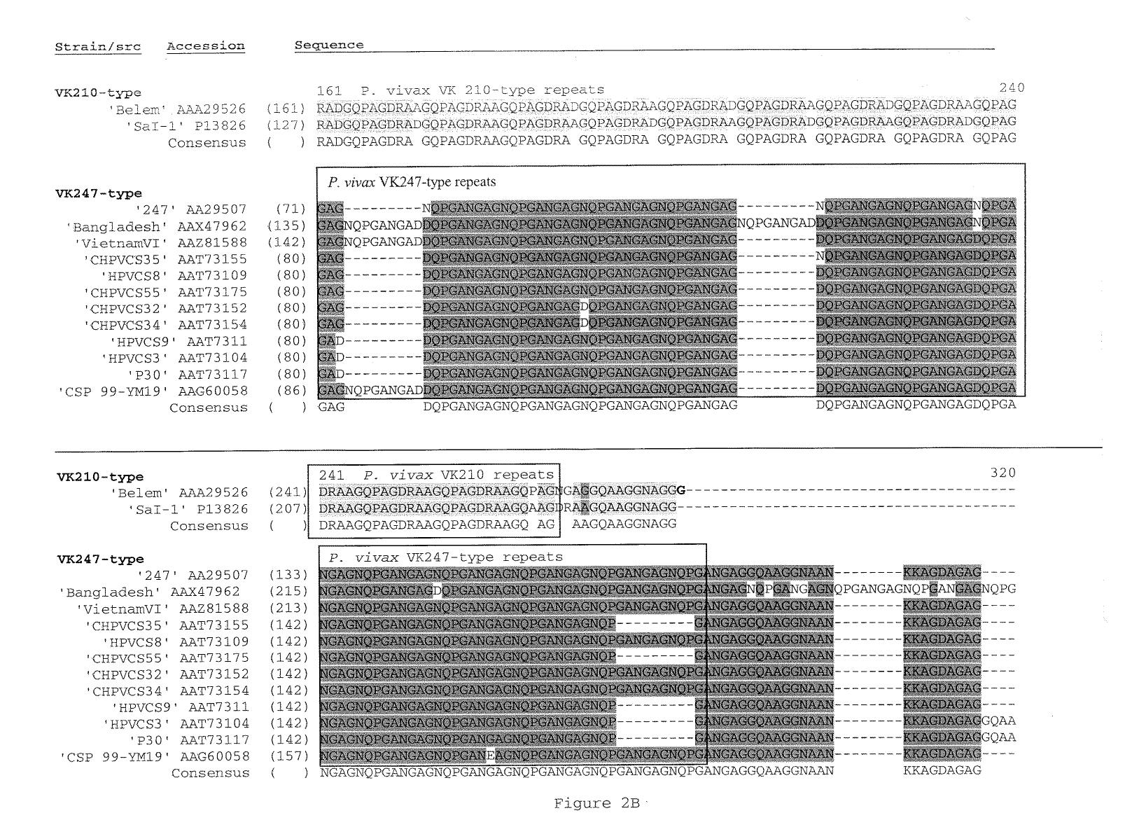

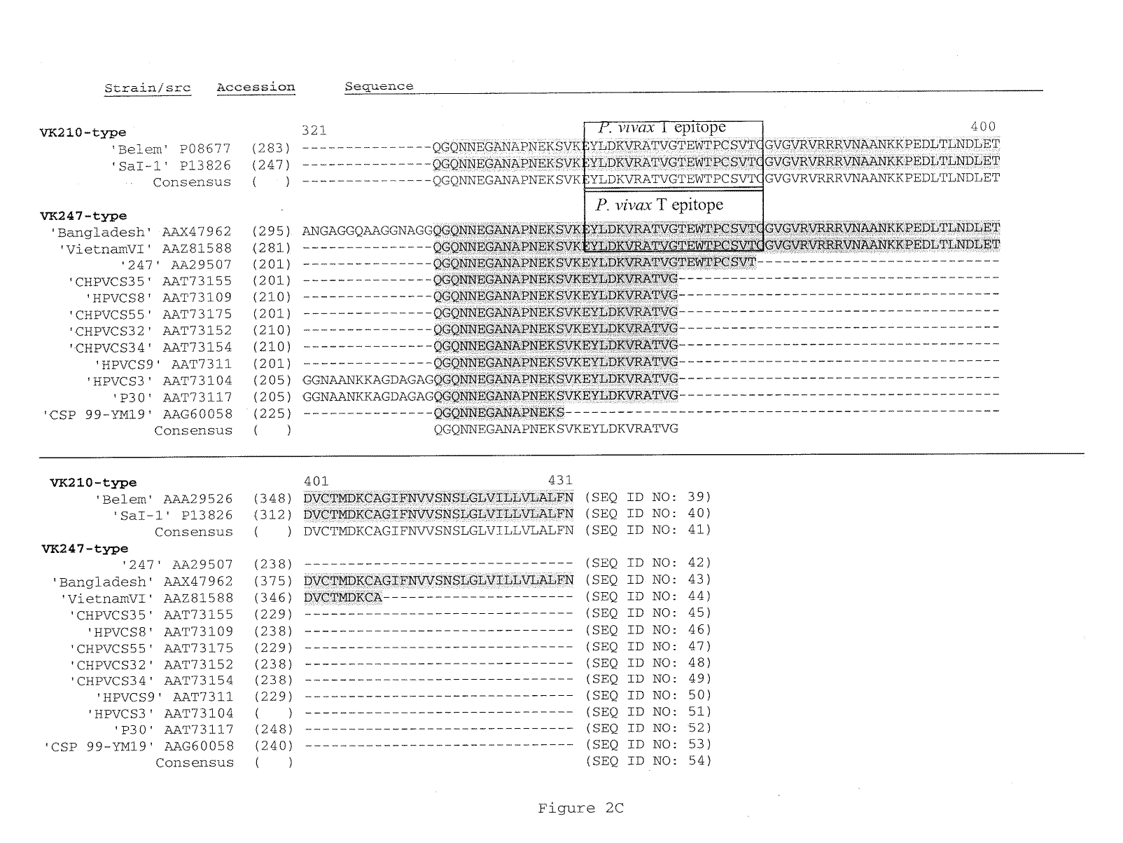

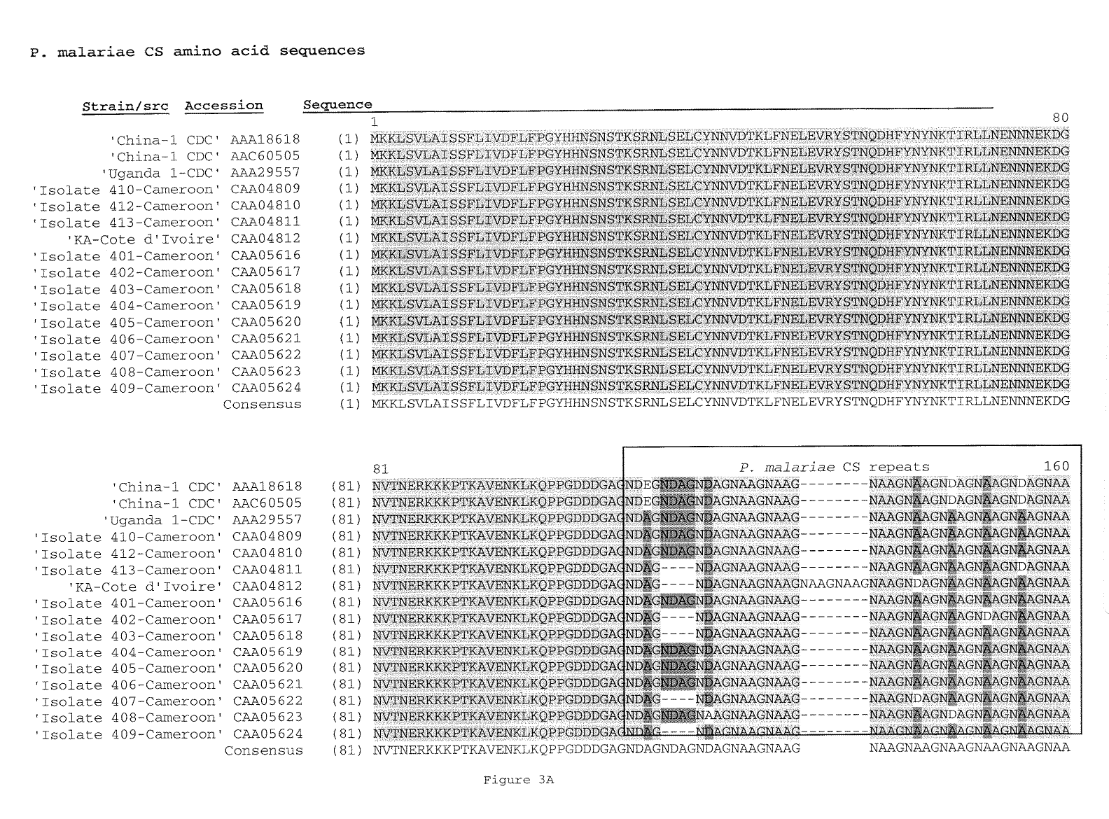

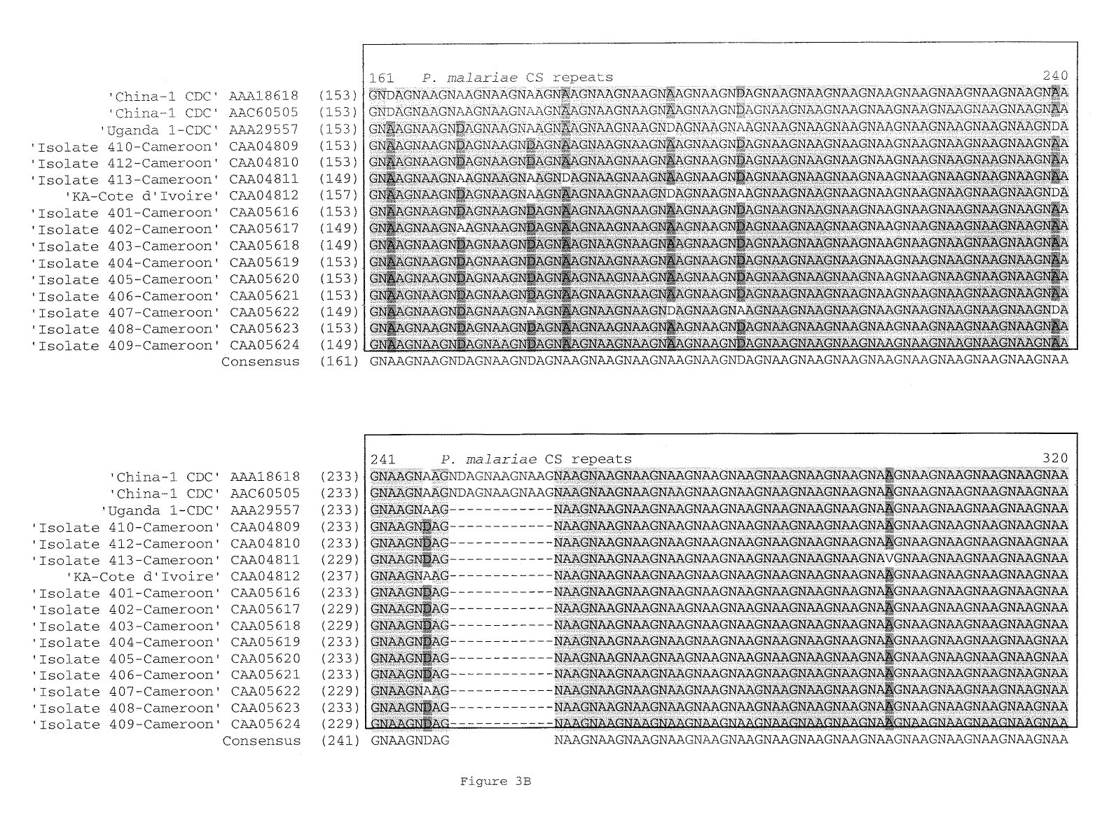

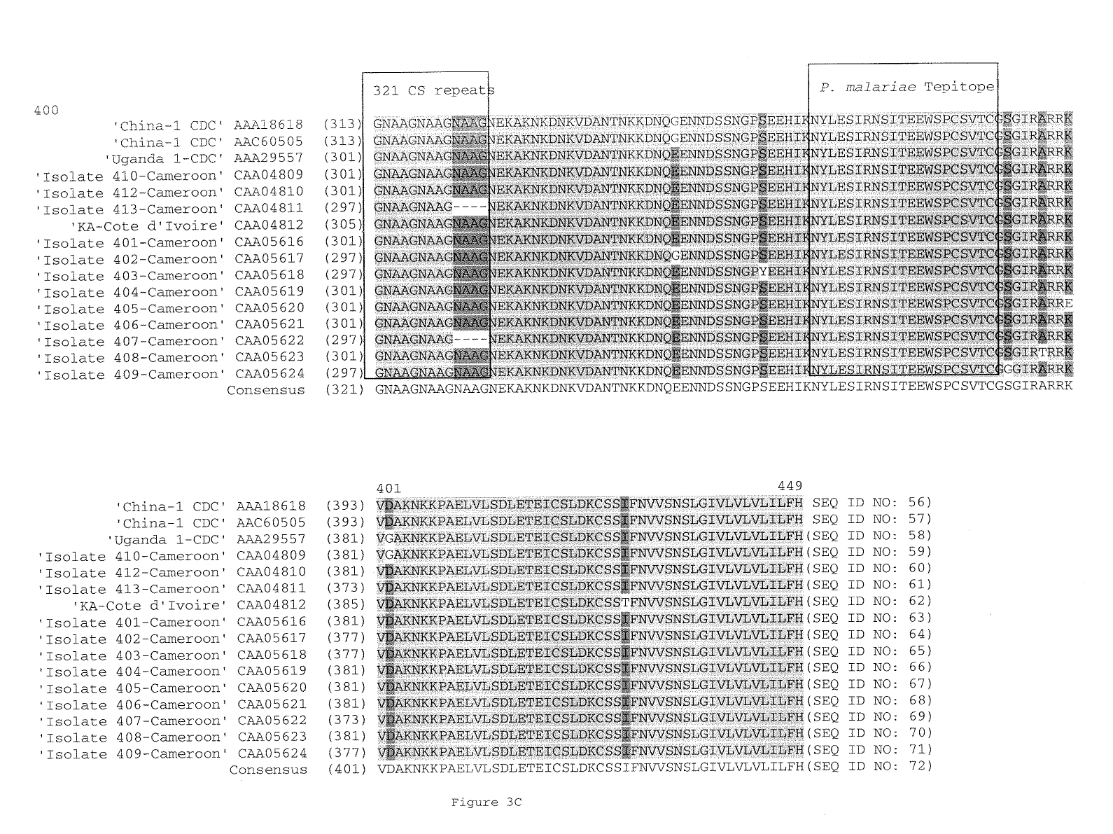

Exemplary Plasmodium vivax CS proteins for use in the invention are shown in FIG. 2. The P. vivax CS protein has two types of repeats, referred to herein as “VK210” (also referred to herein as “type210”) and “VK247” (also referred to herein as “type247”). The VK210 and VK247 repeats are antigenically distinct. The initial P. vivax CS cloned (type210) encoded a 9 mer repeat sequence of at least one member selected from the group consisting of DRADGQPAG (SEQ ID NO: 138) and DRAAGQPAG (SEQ ID NO: 140). The minimal epitope recognized by protective monoclonal antibodies is two tandem repeats that include at least one member selected from the group consisting of DRADGQPAGDRADGQPAG (SEQ ID NO: 139; also referred to herein as “(DRADGQPAG)₂”), DRAAGQPAGDRAAGQPAG (SEQ ID NO: 141; also referred to herein as “(DRAAGQPAG)₂”); and DRADGQPAGDRAAGQPAG (SEQ ID NO: 139). Subsequent studies cloned a second type of P. vivax CS protein (type 247) that contained a different 9 mer repeat sequence ANGAGNQPG (SEQ ID NO: 167). The minimal epitope for protective monoclonal antibody is four repeats ANGAGNQPGANGAGNQPGANGAGNQPGANGAGNQPG (SEQ ID NO: 168; also referred to herein as “(ANGAGNQPG)₄”. Antibodies to the VK210 repeats do not cross react with VK247 repeats. Likewise, antibodies to VK247 repeats do not cross react with VK210. The non-repeat regions of the type 210 and type 247 CS proteins are similar and the T* sequences are similar. When a CSP is employed in a fusion protein with a TLR5 agonist, the composition can include at least one additional malaria antigen, such as a MSP1 antigen, a AMA-1 antigen and a LSA1 antigen (see, for example, FIGS. 82-89).

In an embodiment, the circumsporozite antigen includes at least a portion of at least one T-cell epitope. “T-cell epitope,” as used herein in reference to a malaria antigen, refers to a portion of a malaria antigen that activates T-cells in a manner that is specific for malaria parasite. The T-cell epitopes of the malaria antigens for use in the invention can bind to several MHC class II molecules in a manner that activates T cell function in a class II- or class I-restricted manner. The activated T-cells may be helper cells (CD4+) and/or cytotoxic cells (class II-restricted CD4+ and/or class I-restricted CD8+). The T-cell epitope can include at least one member selected from the group consisting of EYLNKIQNSLSTEWSPCSVT (SEQ ID NO: 34); DPNANPNVDPNANPNV (SEQ ID NO: 37); DPNANPNVDPNANPNVDPNANPNVDP (SEQ ID NO: 169; EYLDKVRATVGTEWTPCSVT (SEQ ID NO: 55); NYLESIRNSITEEWSPCSVT (SEQ ID NO: 73); QYLKKIQNSLSTEWSPCSVT (SEQ ID NO: 170); QYLKKIKNSISTEWSPCSVT (SEQ ID NO: 171); EYLNKIQNSLSTEWSPCSVT (SEQ ID NO: 34); KYLKRIKNSISTEWSPCSVT (SEQ ID NO: 133); QYLQTIRNSLSTEWSPCSVT (SEQ ID NO: 134); EYLDKVRATVGTEWTPCSVT (SEQ ID NO: 55); NYLESIRNSITEEWSPCSVT (SEQ ID NO: 73); EFLKQIQNSLSTEWSPCSVT (SEQ ID NO: 135); EFVKQISSQLTEEWSQCNVT (SEQ ID NO: 136); and EFVKQIRDSITEEWSQCSVT (SEQ ID NO: 137).

SEQ ID NOs: 37 and 152, for example, are also referred to herein as a “T1” epitope. “T1,” as used herein in reference to a T-cell epitope of a malaria antigen, refers to an initial T-cell epitope that was identified in CD4+T-cell clones derived from humans immunized by repeated exposure to the bites of irradiated Plasmodium falciparum malaria infected mosquitoes and who developed protection against infection as shown by the absence of blood stage infection (see, U.S. Pat. No. 6,669,945, the teachings of all of which are hereby incorporated by reference in its entirety) and its related sequence in other Plasmodium strains. The T1 epitope in the CS repeat region (see, for example, FIG. 1) is a T-cell and B-cell epitope. The T-cell epitope DPNANPNVDPNANPNV (SEQ ID NO: 37) is a T-cell and B-cell epitope. Exemplary T1 epitopes are described in U.S. application Ser. No. 11/200,723, the teachings of which are hereby incorporated by reference in their entirety.

SEQ ID NO: 34, for example, are also referred to herein as a “T*” epitope. “T*,” as used herein in reference to a T-cell epitope of a malaria antigen, refers to a T-cell epitope that was identified in CD4+ T-cell clones derived from humans immunized by repeated exposure to the bites of irradiated Plasmodium falciparum malaria infected mosquitoes and who developed protection against infection as shown by the absence of blood stage infection (see, U.S. Pat. No. 6,669,945, the teachings of all of which are hereby incorporated by reference in its entirety) and its related sequence in other Plasmodium strains.

The malaria antigen for use in the compositions of the invention can further include at least a portion of at least one B-cell epitope for use alone or in combination with at least one T-cell epitope. A “B-cell epitope,” as used herein, refers to at least a portion of a malaria antigen that elicits the production of specific antibodies (i.e., antibodies that recognize the parasite and the portion of the malaria antigen) in a mammalian host.

The B-cell epitope can include at least one amino acid sequence as set forth in the amino acid sequence NANP (SEQ ID NO: 36), such as NANPNANPNANP (SEQ ID NO: 38; also referred to herein as “(NANP)₃”). The B-cell epitope (NANP)₃ (SEQ ID NO: 38), for example, can be employed in the compositions of the invention. (NANP)₃ and (NANP)₄ can also be employed, wherein the subscript denotes the number of NANP units employed. Exemplary B-cell epitopes for use in the compositions of the invention, can be two (NANPNANP(NANP)₂; SEQ ID NO: 172), three (NANPNANPNANP(NANP)₃; SEQ ID NO: 38), four (NANPNANPNANPNANP(NANP)₄; SEQ ID NO: 173), five (NANPNANPNANPNANPNANP(NANP)₅; SEQ ID NO: 174) or six (NANPNANPNANPNANPNANPNANP(NANP)₆; SEQ ID NO: 225) sequences in tandem, as set forth in SEQ ID NO: 36. The B-cell epitope NANPNANPNANP (SEQ ID NO: 38) is also a T-cell epitope.

Malaria antigen B-cell epitopes can be characterized by repeats of amino acid sequences, which can be distinct for each malaria parasite species. The NANP (SEQ ID NO: 36) tetramer repeats (SEQ ID NOs: 38 and 172-174) and NVDP (SEQ ID NO: 227) repeats characterize P. falciparum CSP repeats. The B-cell epitope repeats are highly conserved in P. falciparum isolates. In all Plasmodium species, the repeats are enriched for amino acids aspargine (N), alanine (H), proline (P), glycine (G) and glutamine (Q). These malaria antigen B-cell epitopes are also referred to herein as “Repeats.”

In another embodiment, the invention is a composition that includes at least one fusion protein comprising at least a portion of at least one Toll-like Receptor 5 agonist, at least a portion of at least one malaria antigen T-cell epitope and at least a portion of at least one malaria antigen B-cell epitope. The malaria T-cell antigen can include a Plasmodium falciparum malaria T-cell antigen. The T-cell epitope can include, for example, at least one member selected from the group consisting of SEQ ID NOs: 34, 55 and 133-137. The malaria B-cell epitope can include a Plasmodium falciparum malaria B-cell epitope. The B-cell epitope can include at least three amino acid sequence repeats as set forth in SEQ ID NO: 36.

The compositions that include a fusion protein of a Toll-like Receptor 5 agonist and a malaria antigen can further include at least a portion of at least one member selected from the group consisting of a Toll-like Receptor 1 agonist, Toll-like Receptor 2 agonist (e.g., Pam3Cys, Pam2Cys, bacterial lipoprotein), a Toll-like Receptor 3 agonist (e.g., dsRNA), a Toll-like Receptor 5 agonist (e.g., bacterial lipopolysaccharide), a Toll-like Receptor 5 agonist, a Toll-like Receptor 5 agonist, a Toll-like Receptor 5 agonist, a Toll-like Receptor 5 agonist (e.g., unmethylated DNA motifs) and a Toll-like Receptor 10 agonist. Exemplary suitable Toll-like Receptor agonist components for use in the invention are described, for example, in U.S. applicatio Nos. 11/820,148; 11/879,695; 11/714,873; 11/714,684; PCT/US 2006/002906/WO 2006/083706; PCT/US 2006/003285/WO 2006/083792; PCT/US 2006/041865; and PCT/US 2006/042051, the entire teachings of all of which are hereby incorporated by reference in their entirety.

TLR4 agonists for use in the compositions and methods of the invention can include at least one member selected from the group consisting of:

| | |
|---|---|
| GGKSGRTG | SEQ ID NO: 175 |
| KGYDWLVVG | SEQ ID NO: 176 |
| EDMVYRIGVP | SEQ ID NO: 177 |
| VKLSGS | SEQ ID NO: 178 |
| GMLSLALF | SEQ ID NO: 179 |
| CVVGSVR | SEQ ID NO: 180 |
| IVRGCLGW | SEQ ID NO: 181 |

TLR2 agonists for use in the compositions and methods of the invention can also include at least one member selected from the group consisting of (see, PCT/US 2006/002906/WO 2006/083706; PCT/US 2006/003285/WO 2006/083792; PCT/US 2006/041865; PCT/US 2006/042051):

| | |
|---|---|
| NPPTT | SEQ ID NO: 182 |
| MRRIL | SEQ ID NO: 183 |
| MISS | SEQ ID NO: 184 |
| RGGSK | SEQ ID NO: 185 |
| RGGF | SEQ ID NO: 186 |

In another embodiment, the invention is a composition that includes at least one fusion protein comprising at least a portion of at least one Toll-like Receptor agonist and at least a portion of at least one malaria antigen, wherein the Toll-like Receptor agonist is not a Pam3Cys.

The TLR2 agonist can also include at least a portion of at least one member selected from the group consisting of flagellin modification protein F1 mB of Caulobacter crescentus; Bacterial Type III secretion system protein; invasin protein of Salmonella; Type 4 fimbrial biogenesis protein (PilX) of Pseudomonas; Salmonella SciJ protein; putative integral membrane protein of Streptomyces; membrane protein of Pseudomonas; adhesin of Bordetella pertusis; peptidase B of Vibrio cholerae; virulence sensor protein of Bordetella; putative integral membrane protein of Neisseria meningitidis; fusion of flagellar biosynthesis proteins FliR and FlhB of Clostridium; outer membrane protein (porin) of Acinetobacter; flagellar biosynthesis protein FlhF of Helicobacter; ompA related protein of Xanthomonas; omp2a porin of Brucella; putative porin/fimbrial assembly protein (LHrE) of Salmonella; wbdk of Salmonella; Glycosyltransferase involved in LPS biosynthesis; Salmonella putative permease.

The TLR2 agonist can include at least a portion of at least one member selected from the group consisting of lipoprotein/lipopeptides (a variety of pathogens); peptidoglycan (Gram-positive bacteria); lipoteichoic acid (Gram-positive bacteria); lipoarabinomannan (mycobacteria); a phenol-soluble modulin (Staphylococcus epidermidis); glycoinositolphospholipids (Trypanosoma Cruzi); glycolipids (Treponema maltophilum); porins (Neisseria); zymosan (fungi) and atypical LPS (Leptospira interrogans and Porphyromonas gingivalis).

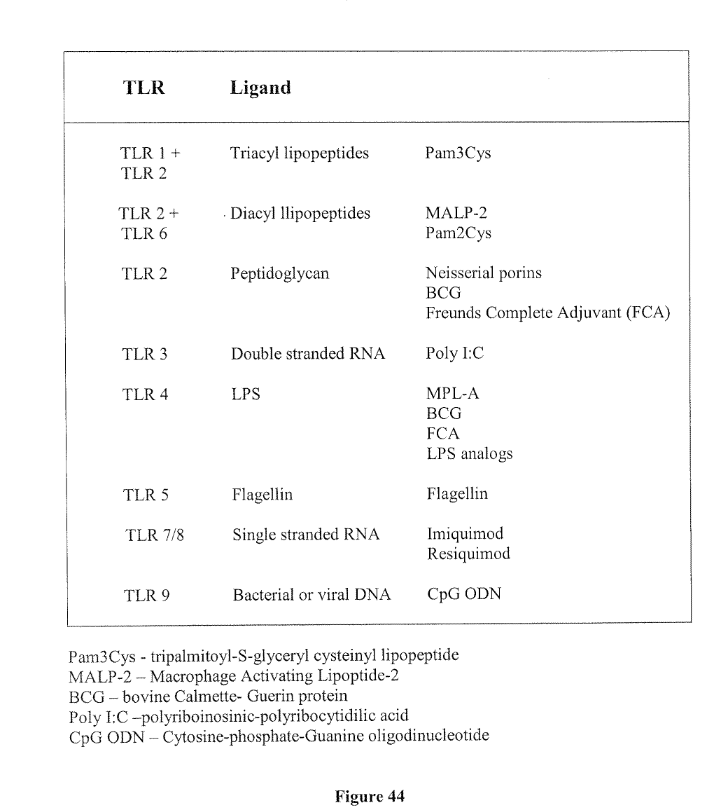

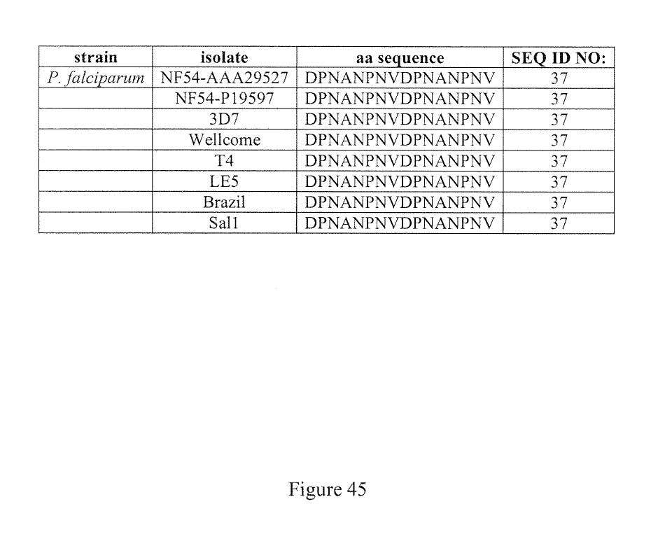

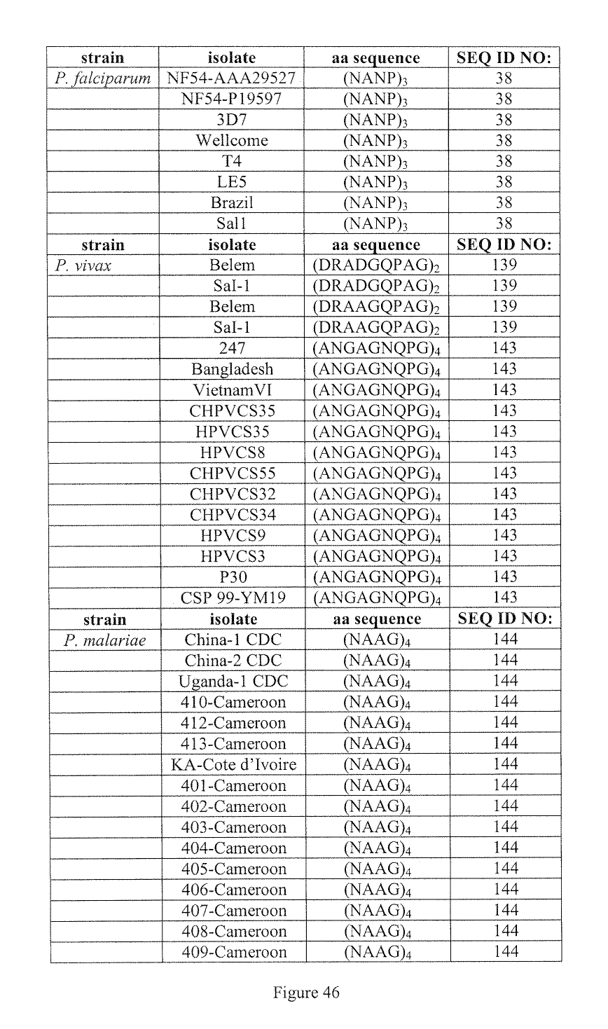

Compositions of the invention that include a fusion protein that includes at least a portion of a Toll-like Receptor 5 agonist and at least a portion of a malaria antigen and other Toll-like Receptor agonists can activate one or more TLR pathways. For example, bacterial lipopeptide activates TLR1; Pam3Cys, Pam2Cys activate TLR2; dsRNA activates TLR3; LBS (LPS-binding protein) and LPS (lipopolysaccharide) activate TLR4; imidazoquinolines (anti-viral compounds and ssRNA) activate TLR7; and bacterial DNA (CpG DNA) activates TLR9. TLR1 and TLR6 require heterodimerization with TLR2 to recognize ligands (e.g., TLR agonists, TLR antagonists). TLR1/2 are activated by triacyl lipoprotein (or a lipopeptide, such as Pam3Cys), whereas TLR6/2 are activated by diacyl lipoproteins (e.g., Pam2Cys), although there may be some cross-recognition. In addition to the natural ligands, synthetic small molecules including the imidazoquinolines, with subclasses that are specific for TLR7 or TLR8 can activate both TLR7 and TLR8. There are also synthetic analogs of LPS that activate TLR4, such as monophosphoryl lipid A [MPL]. Exemplary TLR agonists (also referred to herein as “ligands”) are depicted in FIG. 44.

TLR activation can result in signaling through MyD88 and NF-κB. There is some evidence that different TLRs induce different immune outcomes. For example, Hirschfeld, et al. Infect Immun 69:1477-1482 (2001)) and Re, et al. J Biol Chem 276:37692-37699 (2001) demonstrated that TLR2 and TLR4 activate different gene expression patterns in dendritic cells. Pulendran, et al J Immunol 167: 5067-5076 (2001)) demonstrated that these divergent gene expression patterns were recapitulated at the protein level in an antigen-specific response, when lipopolysaccharides that signal through TLR2 or TLR4 were used to guide the response (TLR4 favored a Th1-like response with abundant IFNγ secretion, while TLR2 favored a Th2-line response with abundant IL-5, IL-10, and IL-13 with lower IFNγ levels).

Activation of TLRs can result in increased effector cell activity that can be detected, for example, by measuring IFNγ-secreting CD8+ cells (e.g., cytotoxic T-cell activity flow cytometry); increased antibody responses that can be detected by, for example, ELISA (Schnare, M., et al., Nat Immunol 2:947 (2001); Alexopoulou, L., et al., Nat Med 8:878 (2002); Pasare, C., et al., Science 299:1033 (2003); Napolitani, G., et al., Nat Immunol 6:769 (2005); and Applequist, S. E., et al. J Immunol 175:3882 (2005)).

In a further embodiment, the invention is a composition that includes at least one fusion protein comprising at least a portion of at least one Toll-like Receptor agonist and at least a portion of at least one malaria antigen, wherein the Toll-like Receptor agonist is not a Pam3Cys. The malaria antigen for use in the compositions of the invention can include at least one T-cell epitope, such as SEQ ID NOs: 34-35, 55, 73 and 133-137 and at least one B-cell epitope, such as SEQ ID NOs: 38 and 138-145. The malaria antigen for use in the invention can include an antigen expressed by a malaria parasite at any stage of its development, such as a CSP protein.

A further embodiment of the invention is a method of stimulating an immune response in a subject, comprising the step of administering to the subject a composition that includes at least one fusion protein (e.g., SEQ ID NOs: 7, 9, 11, 13, 15, 17, 20, 22 and 24) comprising at least a portion of at least one Toll-like Receptor 5 agonist, such as a flagellin, and at least a portion of at least one malaria antigen. The flagellin can lack at least a portion of a hinge region.

In an embodiment, the composition of the invention administered to the subject provides sterile immunity against a malaria infection in the subject. In another embodiment, the composition of the invention administered to the subject provides protective immunity against an infection consequent to exposure of the subject to a source of the malaria antigen.

In an additional embodiment, the invention is a method of stimulating an immune response in a subject, comprising the step of administering to the subject a composition that includes at least one fusion protein comprising at least a portion of at least one Toll-like Receptor 5 agonist and at least a portion of at least one malaria antigen, wherein the malaria antigen is not a Plasmodium vivax merozoite surface protein 1 antigen.

“Stimulating an immune response,” as used herein, refers to the generation of antibodies and/or T-cells to at least a portion of the protein, the malaria antigen component of the fusion proteins described herein. The antibodies and/or T-cells can be generated to at least a portion of a malaria antigen, such as CSP (e.g., SEQ ID NOS: 25-33, 39-54 and 56-72), T-cell epitopes of malaria antigens (e.g., SEQ ID NOS: 34-38, 55, 73 and 133-137) and B-cell epitopes of malaria antigens (e.g., SEQ ID NOS: 38 and 138-145).

Stimulating an immune response in a subject can include the production of humoral and/or cellular immune responses that are reactive against the malaria antigen.

The compositions of the invention for use in methods to stimulate immune responses in subjects, can be evaluated for the ability to stimulate an immune response in a subject using well-established methods. Exemplary methods to determine whether the compositions of the invention stimulate an immune response in a subject, include measuring the production of antibodies specific to the antigen (e.g., IgG antibodies) by a suitable technique such as, ELISA assays; assessment of cellular immune responses, such as the production of cytokines (e.g., IFNγ); and the ability to generate serum antibodies in non-human models (e.g., mice, rabbits, monkeys) (Putnak, et al., Vaccine 23:4442-4452 (2005)).

“Stimulates a protective immune response,” as used herein, means administration of the compositions of the invention results in production of antibodies to the malaria protein mitigates disease consequent to malaria infection.

Protective immunity can be assessed by measuring the levels of parasitemia in the blood and cumulative blood stage parasite burden, determined using Giemsa stained blood smears; or the absence of clinical symptoms of malaria disease, such as fever and anemia in the presence of parasite.

For protection against pre-erythrocytic stages, the levels of parasites in the liver following sporozoite challenge can be determined measured by real-time PCR in rodents as described herein.

Protective immunity can also be assessed by determining whether a subject survives challenge by an otherwise lethal dose of malaria. Techniques to determine a lethal dose of a parasite are known to one of skill in the art. Exemplary techniques for determining a lethal dose can include administration of varying doses of the malaria parasite or varying stages of the malaria parasite and a determination of the percent of subjects that survive following administration of the dose of the parasite (e.g., LD₁₀, LD₂₀, LD₄₀, LD₅₀, LD₆₀, LD₇₀, LD₈₀, LD₉₀). For example, a lethal dose of a parasite that results in the death of 50% of a population of subjects is referred to as an “LD₅₀”; a lethal dose of a parasite that results in the death of 80% of a population of subjects is referred to herein as “LD₈₀”; a lethal dose of a parasite that results in death of 90% of a population of subjects is referred to herein as “LD₉₀.”

“Sterile immunity,” as used herein, refers to the absence of blood stage parasite in subjects following challenge by exposure to bites by parasite infected mosquitoes. Techniques to assess sterile immunity can include exposure of a subject such as a rodent to intravenous challenge with sporozoites, or of human volunteers to the bites of malaria infected mosquitoes, preceded by administration of the compositions of the invention and assessment of parasites in a blood sample.

Sterile immunity can be measured by taking daily blood smears after challenge and determining whether the subject develops a patent blood stage infection. The pre-patent period (the time to appearance of first parasites in the blood), is also measured to determine if there is a delayed pre-patent period. A one-two day delay in appearance of parasites in the blood usually reflects destruction of about greater than 90% of the liver stage parasites, either through the action of inhibitory antibodies that block hepatocyte invasion and/or the direct targeting of infected hepatocytes by induction of NO stimulated by inhibitory cytokines (IFNγ) secreted by T cells. Direct cytotoxicity by CTL against liver stage infected cells may also decrease the number of EEF and result in a prolonged pre-patent period. In more recent studies, PCR has been used to monitor blood stage infection to increase the sensitivity of determining time to patent infection.

Fusion proteins described herein can be made in a prokaryotic host cell or a eukaryotic host cell. The prokaryotic host cell can be at least one member selected from the group consisting of an E. coli prokaryotic host cell, a Pseudomonas prokaryotic host cell, a Bacillus prokaryotic host cell, a Salmonella prokaryotic host cell and a P. fluorescens prokaryotic host cell. The eukaryotic host cell can include a Saccharomyces eukaryotic host cell, an insect eukaryotic host cell (e.g., at least one member selected from the group consisting of a Baculovirus infected insect cell, such as Spodoptera frugiperda (Sf9) or Trichhoplusia ni (Highs) cells; and a Drosophila insect cell, such as Dme12 cells), a fungal eukaryotic host cell, a parasite eukaryotic host cell (e.g., a Leishmania tarentolae eukaryotic host cell), CHO cells, yeast cells (e.g., Pichia) and a Kluyveronmyces lactis lactic host cell.

Suitable eukaryotic host cells to make the fusion proteins described herein and vectors can also include plant cells (e.g., tomato; chloroplast; mono- and dicotyledonous plant cells; Arabidopsis thaliana; Hordeum vulgare; Zea mays; potato, such as Solanum tuberosum; carrot, such as Daucus carota L.; and tobacco, such as Nicotiana tabacum, Nicotiana benthamiana (Gils, M., et al., Plant Biotechnol J. 3:613-20 (2005); He, D. M., et al., Colloids Surf B Biointerfaces, (2006); Huang, Z., et al., Vaccine 19:2163-71 (2001); Khandelwal, A., et al., Virology. 308:207-15 (2003); Marquet-Blouin, E., et al., Plant Mol Biol 51:459-69 (2003); Sudarshana, M. R., et al. Plant Biotechnol J. 4:551-9 (2006); Varsani, A., et al., Virus Res, 120:91-6 (2006); Kamarajugadda S., et al., Expert Rev Vaccines 5:839-49 (2006); Koya V, et al., Infect Immun. 73:8266-74 (2005); Zhang, X., et al., Plant Biotechnol J 4:419-32 (2006)). The fusion proteins of the invention can be made by well-established methods and can be purified and characterized employing well-known methods (e.g., gel chromatography, cation exchange chromatography, SDS-PAGE), as described herein.

In an embodiment, the methods of making a protein of the invention, in particular, a fusion protein, can include a step of deleting a signal sequence of the fusion protein or component of the fusion protein in the nucleic acid sequence encoding the fusion protein or component of the fusion protein to thereby prevent secretion of the protein in the host cell, which results in accumulation of the protein in the cell. The accumulated protein can be purified from the cell.

In another embodiment, the methods of making a protein of the invention, in particular, a fusion protein, can include a step of deleting at least one putative glycosulation site (e.g., an N-glycosylation site NXST (SEQ ID NO: 187)) in the nucleic acid sequence encoding the fusion protein or component of the fusion protein (e.g., at least a portion of a flagellin).

A “subject,” as used herein, can be a mammal, such as a primate or rodent (e.g., rat, mouse). In a particular embodiment, the subject is a human.

An “effective amount,” when referring to the amount of a composition and fusion protein of the invention, refers to that amount or dose of the composition and fusion protein, that, when administered to the subject is an amount sufficient for therapeutic efficacy (e.g., an amount sufficient to stimulate an immune response in the subject, provide protective immunity for the subject, provide sterile immunity for the subject). The compositions and fusion proteins of the invention can be administered in a single dose or in multiple doses.

The methods of the present invention can be accomplished by the administration of the compositions and fusion proteins of the invention by enteral or parenteral means. Specifically, the route of administration is by oral ingestion (e.g., drink, tablet, capsule form) or intramuscular injection of the composition and fusion protein. Other routes of administration as also encompassed by the present invention including intravenous, intradermal, intraarterial, intraperitoneal, or subcutaneous routes and intranasal administration. Suppositories or transdermal patches can also be employed.

The compositions and proteins of the invention can be administered alone or can be coadministered to the patient. Coadministration is meant to include simultaneous or sequential administration of the composition, protein or polypeptide of the invention individually or in combination. Where the composition and protein are administered individually, the mode of administration can be conducted sufficiently close in time to each other (for example, administration of the composition close in time to administration of the fusion protein) so that the effects on stimulating an immune response in a subject are maximal. It is also envisioned that multiple routes of administration (e.g., intramuscular, oral, transdermal) can be used to administer the compositions and proteins of the invention.

The compositions and proteins of the invention can be administered alone or as admixtures with conventional excipients, for example, pharmaceutically, or physiologically, acceptable organic, or inorganic carrier substances suitable for enteral or parenteral application which do not deleteriously react with the extract. Suitable pharmaceutically acceptable carriers include water, salt solutions (such as Ringer's solution), alcohols, oils, gelatins and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which do not deleteriously react with the compositions, proteins or polypeptides of the invention. The preparations can also be combined, when desired, with other active substances to reduce metabolic degradation. The compositions and proteins of the invention can be administered by is oral administration, such as a drink, intramuscular or intraperitoneal injection or intranasal delivery. The compositions and proteins alone, or when combined with an admixture, can be administered in a single or in more than one dose over a period of time to confer the desired effect (e.g., alleviate or prevent malaria infection, to alleviate symptoms of malaria infection).

When parenteral application is needed or desired, particularly suitable admixtures for the compositions and proteins are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compositions, proteins or polypeptides can also be incorporated into liposomes or administered via transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309 the teachings of which are hereby incorporated by reference.

The compositions and proteins of the invention can be administered to a subject on a support that presents the compositions, proteins and polypeptides of the invention to the immune system of the subject to generate an immune response in the subject. The presentation of the compositions, proteins and polypeptides of the invention would preferably include exposure of antigenic portions of the malaria parasite to generate antibodies. The components (e.g., fusion proteins, TLR agonists) of the compositions, proteins and polypeptides of the invention are in close physical proximity to one another on the support. The compositions and proteins of the invention can be attached to the support by covalent or noncovalent attachment. Preferably, the support is biocompatible. “Biocompatible,” as used herein, means that the support does not generate an immune response in the subject (e.g., the production of antibodies). The support can be a biodegradable substrate carrier, such as a polymer bead or a liposome. The support can further include alum or other suitable adjuvants. The support can be a virus (e.g., adenovirus, poxvirus, alphavirus), bacteria (e.g., Salmonella) or a nucleic acid (e.g., plasmid DNA).

The dosage and frequency (single or multiple doses) administered to a subject can vary depending upon a variety of factors, including prior exposure to a malaria parasite, the duration of malaria infection, prior treatment of the malaria infection, the route of administration of the composition, protein or polypeptide; size, age, sex, health, body weight, body mass index, and diet of the subject; nature and extent of symptoms of parasite exposure, parasite infection and the particular parasite responsible for the malaria infection, kind of concurrent treatment, complications from parasite exposure, parasite infection or exposure or other health-related problems. Other therapeutic regimens or agents can be used in conjunction with the methods and compositions, proteins or polypeptides of the present invention. For example, the administration of the compositions and proteins can be accompanied by other malaria therapeutics or use of agents to treat the symptoms of a condition associated with or consequent to exposure to the malaria parasite, or malaria parasite infection, for example. Adjustment and manipulation of established dosages (e.g., frequency and duration) are well within the ability of those skilled in the art.

Subjects can be administered the compositions, fusion proteins or nucleic acids encoding the fusion proteins employing a heterologous prime/boost schedule. The heterologous prime/boost schedule can include priming (e.g., initial administration) the subject by administering the fusion protein or nucleic acid encoding a fusion protein and then boosting (e.g., second or subsequent administration) the subject with the fusion protein or nucleic acid encoding a fusion protein in a vector (e.g., recombinant adenovirus vector). For example, the subject can be primed with a fusion protein of the invention and then boosted with a viral vector that includes a nucleic acid encoding the fusion protein. Likewise, the subject can be primed with a viral vector that includes a nucleic acid encoding a fusion protein and boosted with a fusion protein.

The composition and/or dose of the fusion proteins can be administered to the human in a single dose or in multiple doses, such as at least two doses. When multiple doses are administered to the subject, a second or dose in addition to the initial dose can be administered days (e.g., 1, 2, 3, 4, 5, 6 or 7), weeks (e.g., 1, 2, 3, 4, 5, 6, 7, 8, 9, 10), months (e.g., 1, 2, 3, 4, 5, 6, 7, 8, 9, 10) or years (e.g., 1, 2, 3, 4, 5, 6, 7, 8, 9, 10) after the initial dose. For example, a second dose of the composition can be administered about 7 days, about 14 days or about 28 days following administration of a first dose.

The compositions and methods of employing the compositions of the invention can further include a carrier protein. The carrier protein can be at least one member selected from the group consisting of a tetanus toxoid, a Vibrio cholerae toxoid, a diphtheria toxoid, a cross-reactive mutant of diphtheria toxoid, a E. coli B subunit of a heat labile enterotoxin, a tobacco mosaic virus coat protein, a rabies virus envelope protein, a rabies virus envelope glycoprotein, a thyroglobulin, a heat shock protein 60, a keyhole limpet hemocyanin and an early secreted antigen tuberculosis-6.

“Carrier,” as used herein, refers to a molecule (e.g., protein, peptide) that can enhance stimulation of a protective immune response. Carriers can be physically attached (e.g., linked by recombinant technology, peptide synthesis, chemical conjugation or chemical reaction) to a composition or admixed with the composition.

Carriers for use in the methods and compositions described herein can include, for example, at least one member selected from the group consisting of Tetanus toxoid (TT), Vibrio cholerae toxoid, Diphtheria toxoid (DT), a cross-reactive mutant (CRM) of diphtheria toxoid, E. coli enterotoxin, E. coli B subunit of heat labile enterotoxin (LTB), Tobacco mosaic virus (TMV) coat protein, protein Rabies virus (RV) envelope protein (glycoprotein), thyroglobulin (Thy), heat shock protein HSP 60 Kda, Keyhole limpet hemocyamin (KLH), an early secreted antigen tuberculosis-6 (ESAT-6), exotoxin A, choleragenoid, hepatitis B core antigen, and the outer membrane protein complex of N. meningiditis (OMPC) (see, for example, Schneerson, R., et al., Prog Clin Biol Res 47:77-94 (1980); Schneerson, R., et at, J Exp Med 152:361-76 (1980); Chu, C., et al., Infect Immun 40: 245-56 (1983); Anderson, P., Infect Immun 39:233-238 (1983); Anderson, P., et al., J Clin Invest 76:52-59 (1985); Fenwick, B. W., et al., 54:583-586 (1986); Que, J. U., et al. Infect Immun 56:2645-9 (1988); Que, J. U., et al. Infect Immun 56:2645-9 (1988); (Que, J. U., et al. Infect Immun 56:2645-9 (1988); Murray, K., et al., Biol Chem 380:277-283 (1999); Fingerut, E., et a, Vet Immunol Immunopathol 112:253-263 (2006); and Granoff, D. M., et al., Vaccine 11: Suppl 1:S46-51 (1993)).

Exemplary carrier proteins for use in the methods and compositions described herein can include at least one member selected from the group consisting of: Cross-reactive mutant (CRM) of diphtheria toxin (e.g., SEQ ID NO: 188), Coat protein of Tobacco mosaic virus (TMV) coat protein (e.g., SEQ ID. NO: 189), Coat protein of alfalfa mosaic virus (AMV) (e.g., SEQ ID NO: 190), Coat protein of Potato virus X (e.g., SEQ ID NO: 191), Porins from Neisseria sp (e.g., SEQ ID NO: 192), Major fimbrial subunit protein type I (Fimbrillin) (e.g., SEQ ID NO: 193), Mycoplasma fermentans macrophage activating lipopeptide (MALP-2) (e.g., SEQ ID NO: 194) and p19 protein of Mycobacterium tuberculosis (e.g., SEQ ID NO: 195).

The compositions of the invention can further include at least one adjuvant. Adjuvants contain agents that can enhance the immune response against substances that are poorly immunogenic on their own (see, for example, Immunology Methods Manual, vol. 2, I. Lefkovits, ed., Academic Press, San Diego, Calif., 1997, ch. 13). Immunology Methods Manual is available as a four volume set, (Product Code Z37, 435-0); on CD-ROM, (Product Code Z37, 436-9); or both, (Product Code Z37, 437-7). Adjuvants can be, for example, mixtures of natural or synthetic compounds that, when administered with compositions of the invention, such as proteins that stimulate a protective immune response made by the methods described herein, further enhance the immune response to the protein. Compositions that further include adjuvants may further increase the protective immune response stimulated by compositions of the invention by, for example, stimulating a cellular and/or a humoral response (i.e., protection from disease versus antibody production). Adjuvants can act by enhancing protein uptake and localization, extend or prolong protein release, macrophage activation, and T and B cell stimulation. Adjuvants for use in the methods and compositions described herein can be mineral salts, oil emulsions, mycobacterial products, saponins, synthetic products and cytokines. Adjuvants can be physically attached (e.g., linked by recombinant technology, by peptide synthesis or chemical reaction) to a composition described herein or admixed with the compositions described herein.

In an additional embodiment, the invention includes a protein, peptide or polypeptide having at least about 70%, at least about 75%, at least about 80%, at least about 85%, at least about 90%, at least about 95%, at least about 98% and at least about 99% sequence identity to the fusion proteins, malaria antigens and Toll-like Receptor agonists employed in the compositions and methods of the invention.

The percent identity of two amino acid sequences (or two nucleic acid sequences) can be determined by aligning the sequences for optimal comparison purposes (e.g., gaps can be introduced in the sequence of a first sequence). The amino acid sequence or nucleic acid sequences at corresponding positions are then compared, and the percent identity between the two sequences is a function of the number of identical positions shared by the sequences (i.e., % identity=# of identical positions/total # of positions×100). The length of the protein or nucleic acid encoding can be aligned for comparison purposes is at least 30%, preferably, at least 40%, more preferably, at least 60%, and even more preferably, at least 70%, 75%, 80%, 85%, 90%, 95%, 98%, 99% or 100%, of the length of the reference sequence, for example, the nucleic acid sequence of malaria antigens (e.g., SEQ ID NOS: 74-114), Toll-like Receptor 5 agonists (e.g., SEQ ID NOs: 2, 117, 119, 123 and 125) or fusion proteins (e.g., SEQ ID NOs: 7, 9, 11, 13, 15, 17, 20, 22 and 24) of the invention.

The actual comparison of the two sequences can be accomplished by well-known methods, for example, using a mathematical algorithm. A preferred, non-limiting example of such a mathematical algorithm is described in Karlin et al. (Proc. Natl. Acad. Sci. USA, 90:5873-5877 (1993), the teachings of which are hereby incorporated by reference in its entirety). Such an algorithm is incorporated into the BLASTN and BLASTX programs (version 2.2) as described in Schaffer et al. (Nucleic Acids Res., 29:2994-3005 (2001), the teachings of which are hereby incorporated by reference in its entirety). When utilizing BLAST and Gapped BLAST programs, the default parameters of the respective programs (e.g., BLASTN; available at the Internet site for the National Center for Biotechnology Information) can be used. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Another mathematical algorithm employed for the comparison of sequences is the algorithm of Myers and Miller, CABIOS (1989), the teachings of which are hereby incorporated by reference in its entirety. Such an algorithm is incorporated into the ALIGN program (version 2.0), which is part of the GCG (Accelrys, San Diego, Calif.) sequence alignment software package. When utilizing the ALIGN program for comparing amino acid sequences, a PAM120 weight residue table, a gap length penalty of 12, and a gap penalty of 4 is used. Additional algorithms for sequence analysis are known in the art and include ADVANCE and ADAM as described in Torellis and Robotti (Comput. Appl. Biosci., 10: 3-5 (1994), the teachings of which are hereby incorporated by reference in its entirety); and FASTA described in Pearson and Lipman (Proc. Natl. Acad. Sci. USA, 85: 2444-2448 (1988), the teachings of which are hereby incorporated by reference in its entirety).

The percent identity between two amino acid sequences can also be accomplished using the GAP program in the GCG software package (Accelrys, San Diego, Calif.) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package (Accelrys, San Diego, Calif.), using a gap weight of 50 and a length weight of 3.

The nucleic acid sequence encoding a malaria antigen, a flagellin or a fusion proteins of the invention can include nucleic acid sequences that hybridize to nucleic acid sequences or complements of nucleic acid sequences of the invention and nucleic acid sequences that encode amino acid sequences and fusion proteins of the invention under selective hybridization conditions (e.g., highly stringent hybridization conditions). As used herein, the terms “hybridizes under low stringency,” “hybridizes under medium stringency,” “hybridizes under high stringency,” or “hybridizes under very high stringency conditions,” describe conditions for hybridization and washing of the nucleic acid sequences. Guidance for performing hybridization reactions, which can include aqueous and nonaqueous methods, can be found in Aubusel, F. M., et al., Current Protocols in Molecular Biology, John Wiley & Sons, N.Y. (2001), the teachings of which are hereby incorporated herein in its entirety.

For applications that require high selectivity, relatively high stringency conditions to form hybrids can be employed. In solutions used for some membrane based hybridizations, addition of an organic solvent, such as formamide, allows the reaction to occur at a lower temperature. High stringency conditions are, for example, relatively low salt and/or high temperature conditions. High stringency are provided by about 0.02 M to about 0.10 M NaCl at temperatures of about 50° C. to about 70° C. High stringency conditions allow for limited numbers of mismatches between the two sequences. In order to achieve less stringent conditions, the salt concentration may be increased and/or the temperature may be decreased. Medium stringency conditions are achieved at a salt concentration of about 0.1 to 0.25 M NaCl and a temperature of about 37° C. to about 55° C., while low stringency conditions are achieved at a salt concentration of about 0.15 M to about 0.9 M NaCl, and a temperature ranging from about 20° C. to about 55° C. Selection of components and conditions for hybridization are well known to those skilled in the art and are reviewed in Ausubel et al. (1997, Short Protocols in Molecular Biology, John Wiley & Sons, New York N.Y., Units 2.8-2.11, 3.18-3.19 and 4-64.9).

Therapeutic compositions designed to treat pre-existing malaria infections or to prevent illness due to exposure of a malaria parasite are not available. The compositions described herein may have several advantages, such as, reducing or eliminating blood stage malaria parasites in subjects exposed to or consequent to exposure to the malaria parasite.

The teachings of all patents, published applications and references cited herein are incorporated by reference in their entirety.

A description of example embodiments of the invention follows.

### EXEMPLIFICATION

### Example 1

### Cloning and Expression of Flagellin-Malaria Antigen Fusion Proteins

### DNA Cloning and Protein Expression

### Methods:

DNA cloning: Synthetic genes encoding the malaria antigens were codon optimized for expression in E. coli and synthesized by a commercial vendor (DNA 2.0; Menlo Park, Calif.). To facilitate cloning in fusion with the STF2 (flagellin) (SEQ ID NO: 1) or a flagellin lacking a hinge region (STF2Δ) (SEQ ID NO: 3), the malaria antigen genes (SEQ ID NOS: 147-151) were designed to incorporate flanking BlpI sites on both the 5′ and 3′ ends. The gene fragments were excised from the respective plasmids with BlpI and cloned by compatible ends into either the STF2.blp or STF2Δ.blp vector cassette which had been treated with BlpI and alkaline phosphatase. Fusion proteins listed in Table 1 were generated.

TABLE 1
Malaria antigen DNA constructs for expression in E. coli

| | | Predicted protein |
|---|---|---|
| | Fusion Protein | molecular weight |
| SEQ ID NO: | Construct | (Da) |
| 10 | STF2.T1BT* | 57,565 |
| 16 | STF2Δ.T1BT* | 34,571 |
| 12 | STF2.4xT1BT* | 72,908 |
| 18 | STF2Δ.4xT1BT* | 49,915 |
| 8 | STF2.CSP | 86,856 |
| 14 | STF2Δ.CSP | 63,862 |
| 19 | STF2.10xT1BT*His₆ | 104,695 |
| 23 | STF2.10xT1T*His₆ | 92,805 |
| 21 | STF2.10xBT*His₆ | 88,262 |

In each case, the constructed plasmids were used to transform competent E. coli TOP10 cells and putative recombinants were identified by PCR screening and restriction mapping analysis. The integrity of the constructs was verified by DNA sequencing and used to transform the expression host, BLR(DE3) (Novagen, San Diego, Calif.; Cat #69053). Transformants were selected on plates containing kanamycin (50 μg/mL), tetracycline (5 μg/mL) and glucose (0.5%). Colonies were picked and inoculated into 2 mL of LB medium supplemented with 25 μg/mL kanamycin, 12.5 μg/mL tetracycline and 0.5% glucose and grown overnight. Aliquots of these cultures were used to innoculate fresh cultures in the same medium formulation, cultured until an optical density (OD₆₀₀ₙₘ)=0.6 was reached, at which time protein expression was induced by the addition of 1 mM IPTG and cultured for 3 hours at 37° C. The cells were then harvested and analyzed for protein expression.

SDS-PAGE and Western blot: Protein expression and identity were determined by gel electrophoresis and immunoblot analysis. Cells were harvested by centrifugation and lysed in Laemmli buffer. An aliquot of 10 μl of each lysate was diluted in SDS-PAGE sample buffer with or without 100 mM dithiothreitol (DTT) as a reductant. The samples were boiled for 5 minutes, loaded onto a 10% SDS polyacrylamide gel and electrophoresed by SDS-PAGE. The gel was stained with Coomassie R-250 (Bio-Rad; Hercules, Calif.) to visualize protein bands. For Western blots, 0.5 ml/lane of cell lysate was electrophoresed and electrotransferred onto a PVDF membrane and blocked with 5% (w/v) dry milk.

The membrane was then probed with an anti-flagellin antibody (Inotek; Beverly, Mass.) or mouse anti-Plasmodium flaciparum (Pf) CSP monoclonal antibody. After probing with alkaline phosphatase-conjugated secondary antibody (Pierce; Rockland, Ill.), protein bands were visualized with an alkaline phosphatase chromogenic substrate (Promega, Madison, Wis.). Bacterial clones which yielded protein bands of the correct molecular weight and reactive with the appropriate antibodies were selected for production of protein for use in biological assays and animal immunogenicity experiments.

### Results:

As assayed by Coomassie blue staining of the SDS-PAGE gel, all the IPTG-induced flagellin-malaria antigen clones displayed a band that migrated at the expected molecular weight. The absence of this band in the control culture (without IPTG) indicates that it is specifically induced by IPTG. Western blotting with antibodies specific for flagellin and the Pf CSP protein confirmed that this induced species is the flagellin-malaria antigen fusion protein and that both parts of the fusion protein were expressed intact.

### Example 2

### Purification of Flagellin-Malaria Antigen Fusion Proteins

### Methods:

Bacterial growth and cell lysis: Flagellin-malaria antigen fusion constructs were expressed in the E. coli host strain BLR (DE3). E. coli cells carrying a plasmid encoding one of the constructs in Table 1 were cultured and harvested as described above. Individual strains were retrieved from glycerol stocks and grown in shake flasks to a final volume of 12 liters. Cells were grown in LB medium containing 50 μg/mL kanamycin/12.5 μg/mL tetracycline/0.5% dextrose to OD₆₀₀=0.6 and induced by the addition of 1 mM IPTG for 3 hours at 37° C. The cells were harvested by centrifugation (7000 rpm×7 minutes in a Sorvall RC5C centrifuge) and resuspended in 1×PBS, 1% glycerol, 1 μg/mL DNAse I, 1 mM PMSF, protease inhibitor cocktail and 1 mg/mL lysozyme. The cells were then lysed by two passes through a microfluidizer at 15,000 psi. The lysate was then centrifuged at 45,000×g for one hour to separate soluble and insoluble fractions.

Purification of STF2Δ.CSP (SEQ ID NO: 13) from E. coli. The insoluble (inclusion body) fraction was resuspended in buffer A (50 mM Tris, pH8+0.5% (w/v) Triton X-100 and homogenized with a glass-ball Dounce homogenizer. The homogenate was then centrifuged for 10 minutes at 45,000×g to pellet the insoluble material. This process was repeated two more times. The inclusion body protein was then washed once with Buffer B (50 mM Tris, pH 8). Finally, the insoluble protein was dissolved in Buffer C (20 mM citric acid, pH 3.5+8M urea). The urea-denatured protein was then fractionated on a Source S cation exchange column (GE Healthcare; Piscataway, N.J.), eluting the column with a 5 column-volume gradient of 0-1M NaCl in Buffer C. Eluate fractions were assayed for protein content by SDS-PAGE followed by Coomassie staining and Western blotting. Peak fractions were pooled, the pH was adjusted to >6.0, and the protein was refolded by ten-fold dilution in Buffer B. The refolded protein was then fractionated on a Source Q anion exchange column (GE Healthcare, Piscataway, N.J.). The bound protein was eluted in a 5 column-volume linear gradient 0-0.5M NaCl in buffer B. Eluate fractions were assayed by SDS-PAGE followed by Coomassie staining and Western blotting. Peak fractions were pooled and fractionated on a Superdex 200 size exclusion (SEC) column equilibrated in Buffer D (50 mM Tris-Cl, pH 8.0, 0.1M NaCl, 0.5% (w/v) sodium deoxycholate). Peak fractions were pooled, dialyzed against 1× Tris-buffered saline (TBS), pH 8.0, sterile-filtered and stored at −80° C.

Purification of STF2.1× T1BT* (SEQ ID NO: 9) and STF2.4×T1BT* (SEQ ID NO: 11) from E. coli: Following cell lysis and centrifugation, the supernatant (soluble) fraction was collected and supplemented with 50 mM Tris, pH 8 and solid urea to a final concentration of 8M to denature the proteins. The solution was then applied to a Q Sepharose Fast Flow anion exchange column (GE Healthcare: Piscataway, N.J.) equilibrated in Buffer E (50 mM Tris, pH 8.0+8M urea) and eluted in a linear gradient of 0-1M NaCl in Buffer E. Eluate fractions were assayed by SDS-PAGE with Coomassie staining and Western blotting. Peak fractions were pooled and dialyzed overnight to Buffer C (20 mM citric acid, pH 3.5+8M urea) and applied to a Source S cation exchange column equilibrated in Buffer C. After eluting with a 5 column-volume linear gradient of 0-1M NaCl in Buffer C, eluate fractions were assayed by SDS-PAGE with Coomassie staining and Western blotting. Peak fractions were pooled and dialyzed overnight with buffer B (50 mM Tris, pH 8.0+8M urea). The denatured protein was then refolded by ten-fold dilution in Buffer B (50 mM Tris, pH 8.0). The refolded protein was then applied to a Source Q anion exchange column (GE Healthcare; Piscataway, N.J.) equilibrated in Buffer B and eluted with a 5 column-volume linear gradient 0-1M NaCl in Buffer B. Eluate fractions were assayed by SDS-PAGE followed by Coomassie staining and Western blotting. Peak fractions were pooled and fractionated by size-exclusion chromatography (SEC) on a Superdex 200 column (GE Healthcare; Piscataway, N.J.). Peak fractions were pooled, sterile-filtered and stored at −80° C.

Purification of STF2.10×T1BT*His₆ (SEQ ID NO:20), STF2.10T1T* His₆ (SEQ ID NO: 24) and STF2.10×BT* His₆ (SEQ ID NO:22): Following cell lysis and centrifugation, as described above, the supernatant (soluble) fraction was collected, supplemented with Buffer F (1× phosphate-buffered saline (PBS)+20 mM imidazole) and applied to a nickel-NTA column (GE Healthcare; Piscataway, N.J.). After washing with Buffer F, the column was eluted with a 5 column-volume linear gradient 0-0.5M imidazole in Buffer F. Eluate fractions were assayed by SDS-PAGE followed by Coomassie staining or Western blotting. Peak fractions were pooled and extracted three times with Triton X-114 to reduce endotoxin, according to the following protocol. Triton X-114 was added to a final concentration of 1% (w/v) and the sample was incubated for 30 minutes on ice. The sample was then transferred to a 37° C. bath for five minutes to cause detergent clouding. The sample was then centrifuged for ten minutes at 16,000×g to separate the detergent and aqueous phases. The aqueous (upper) phase was then collected and the process repeated. Following detergent extraction, the sample was applied to a Superdex 200 gel filtration column equilibrated in 1× Tris-buffered saline, pH 8.0. Peak fractions were pooled, sterile-filtered and stored at −80° C.

SDS-PAGE and Western blot analysis: Protein identity and purity of all constructs was determined by SDS-PAGE. An aliquot of 5 μg of each sample was diluted in SDS-PAGE sample buffer with or without 100 mM DTT as a reductant. The samples were boiled for 5 minutes and loaded onto a 10% polyacrylamide gel (LifeGels; French's Forest, New South Wales, AUS) and electrophoresed. The gel was stained with Coomassie R250 (Bio-Rad; Hercules, Calif.) to visualize protein bands. For Western blot, 0.5 μg/lane total protein was electrophoresed as described above and the gels were then electro-transferred to a PVDF membrane and blocked with 5% (w/v) non-fat dry milk before probing with anti-flagellin antibody (Inotek; Beverly, Mass.) or anti-CSP monoclonal antibody. After probing with alkaline phosphatase-conjugated secondary antibodies (Pierce; Rockland, Ill.), protein bands were visualized with an alkaline phosphatase chromogenic substrate (Promega; Madison, Wis.).

Protein assay: Total protein concentration for all proteins was determined using the Micro BCA (bicinchonic acid) Assay (Pierce; Rockland, Ill.) in the microplate format, using bovine serum albumin as a standard, according to the manufacturer's instructions.

Endotoxin assay: Endotoxin levels for all proteins were determined using the QCL-1000 Quantitative Chromogenic LAL test kit (Cambrex; E. Rutherford, N.J.), following the manufacturer's instructions for the microplate method.

TLR bioactivity assay: HEK293 cells constitutively express TLR5, and secrete several soluble factors, including IL-8, in response to TLR5 signaling. Cells were seeded in 96-well microplates (50,000 cells/well), and the following test proteins were added and incubated overnight: STF2.T1BT* (SEQ ID NO: 9); STF2.4×T1BT* (SEQ ID NO: 11); and STF2Δ.CSP (SEQ ID NO: 13); STF2.10×T1BT*His₆ (SEQ ID NO: 20); STF2.10×T1T* His₆ (SEQ ID NO: 24) and STF2.10×BT* His₆ (SEQ ID NO: 22). The next day, the conditioned medium was harvested, transferred to a clean 96-well microplate and frozen at −20° C. After thawing, the conditioned medium was assayed for the presence of IL-8 in a sandwich ELISA using an anti-human IL-8 matched antibody pair (Pierce; Rockland, Ill.) #M801E and M802B) following the manufacturer's instructions, Optical density was measured using a microplate spectrophotometer (FARCyte, GE Healthcare; Piscataway, N.J.).

TLR5 bioactivity of STF2Δ.CSP (SEQ ID NO: 13) was assayed using the RAW264.7 cell line (ATCC; Rockville, Md.), which expresses TLR2 and TLR4, but not TLR5. TLR5-specific activity of flagellin fusion proteins, RAW cells was assessed by transfection with a plasmid encoding human TLR5 (Invivogen; San Diego, Calif.) to generate the RAW/TLR5 cell line. TLR5 activation was evaluated based on NF-κB dependent induction of TNFα. RAW264.7 and RAW/TLR5 cells were cultured in 96-well microtiter plates at a seeding density of 5×10⁴ cells in 100 μl/well in DMEM medium supplemented with 10% FCS and antibiotics. The next day, cells were treated for 5 hours with serial dilutions of STF2Δ.CSP (SEQ ID NO: 13). Secretion of TNFα was then evaluated by ELISA (Invitrogen; Carlsbad, Calif.). As shown in FIGS. 68A, 68B and 69, the fusion proteins of the invention activated a TLR5.

Protein antigenicity ELISA: To determine whether the recombinant fusion proteins correctly presented epitopes of malaria antigens, the antigenicity of individual fusion proteins was evaluated by ELISA. ELISA plates (96-well) were coated overnight at 4° C. with serial dilutions in PBS (100 μL/well) of each target protein starting at 5 μg/ml. Plates were blocked with 200 ml/well of Assay Diluent Buffer (ADB; BD Pharmingen) for on hour at room temperature, then washed three times in PBS-T. To assay CSP reactivity, 100 μL/well of a 1:10,000 dilution of anti-CSP mouse immune serum was added. For ELISA of flagellin, monoclonal antibody against flagellin (Inotek; Beverly, Mass.) was added at 1 μg/ml in ADB (100 μL/well) and the plates were incubated for 1 hour at room temperature or overnight at 4° C. The plates were then washed three times with PBS-T. HRP-labeled goat anti-mouse IgG antibodies (Jackson Immunochemical; West Grove, Pa.) diluted in ADB were added (100 μL/well) and the plates were incubated at room temperature for 1 hour. The plates were then washed three times with PBS-T. After adding TMB Ultra substrate (Pierce; Rockland, Ill.) and monitoring color development, absorbance at 450 nm was measured on a microplate spectrophotometer (FARCyte, GE Healthcare; Piscataway, N.J.).

### Results and Discussion:

Protein yield and purity: Results for the purification of recombinant flagellin-malaria antigen fusion proteins are shown in Table 2. All proteins were produced in high yield, with estimated purity exceeding 90% and endotoxin well below the standard acceptable level of 0.1 EU/μg. The fusion proteins demonstrated high in vitro TLR5 bioactivity (see FIGS. 68A, 68B and 69).

TABLE 2
Purification results for flagellin-malaria antigen fusion proteins

| | SEQ ID | Yield | purity est. | Endotoxin | TLR 5 |
|---|---|---|---|---|---|
| Protein | NO: | (mg) | (%) | (EU/μg) | activity |
| STF2.T1BT* | 9 | 8 | >90 | <0.01 | active |
| STF2.4xT1BT* | 11 | 5 | >95 | <0.01 | active |
| STF2Δ.CSP | 13 | 15 | >95 | <0.01 | active |
| STF2.10T1BT*His₆ | 20 | 5.75 | >90 | <0.01 | active |
| STF2.10T1T*His₆ | 24 | 7.5 | >90 | <0.01 | active |
| STF2.10BT*His₆ | 22 | 7.2 | >90 | <0.01 | active |

Antigenicity of malaria antigens fused to flagellin: STF2Δ.CSP (SEQ ID NO:13), STF2.1×T1BT* (SEQ ID NO:9) and STF2.4×T1BT* (SEQ ID NO:11) were analyzed by Western blotting with antibody against STF2 (Inotek; Beverly, Mass.) and anti-CSP mouse immune serum. STF2.T1BT* (SEQ ID NO: 9) and SFT2.4×T1BT* (SEQ ID NO: 11) were also shown by ELISA to react with antibodies directed against both flagellin and Plasmodium flaciparum CSP (FIGS. 74A and 74B). The fusion proteins appeared to react comparably with anti-flagellin antibody and anti-CSP antibody. This result suggests that these fusion proteins are intact with regard to the flagellin component and the malaria antigen component.

### Example 3

### Characterization of Fusion Proteins

### Introduction

Over one-third of the world's population is at risk of Plasmodium infection, which causes about 250 million cases of malaria and about 1 million deaths each year. Attenuated P. falciparum sporozoites can induce protective sterile immunity in humans (Nussenzweig, Vanderberg et al. 1967; Nussenzweig and Nussenzweig 1989; Clyde 1990). Although promising results in reducing risk of clinical disease in African children (Stoute, Kester et al. 1998; Aponte, Aide et al. 2007) have been obtained with a CS subunit virus like particle vaccine, there is currently no commercial vaccine available that elicits high levels of sterile immunity against the Plasmodium parasite, such as P. falciparum, which is the most lethal of the four malaria species. Vaccines based on attenuated sporozoites face enormous challenges for commercial production, as sporozoites cannot be produced in vitro and must be dissected from the salivary glands of malaria infected mosquitoes that have fed on gametocyte cultures that require human blood products (Hoffman, Goh et al. 2002; Luke and Hoffman 2003; Ballou 2007).

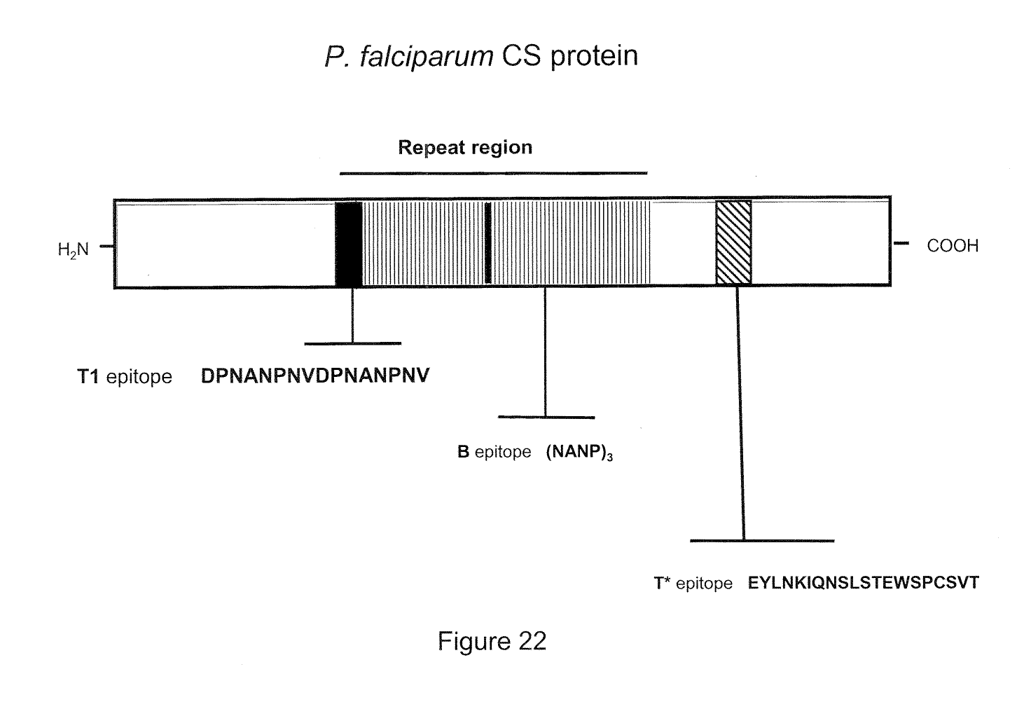

Sporozoite antigens can be employed in compositions to provide protective and sterile immunity. The P. falciparum circumsporozoite (CS) protein is depicted in FIG. 22.

Protective B cell epitopes have been identified within the central repeat region of the CS protein (FIG. 22) (Nussenzweig and Nussenzweig 1989). Numerous functional CD4+ and CD8+ T cell epitopes have been identified primarily in the carboxy-(C)-terminus of the CS protein based on studies in rodent malaria models and sporozoite immunized and naturally infected individuals (Nardin and Nussenzweig 1993; Sinnis and Nardin 2002).

Compositions described herein include epitopes of the P. falciparum CS protein defined using sera and CD4+ T cell clones derived from volunteers immunized with irradiated P. falciparum sporozoites (Nardin, Herrington et al. 1989; Moreno, Clavijo et al. 1991; Moreno, Clavijo et al. 1993). These epitopes include the repeat B cell epitope containing multiple tandem copies of the major repeat NANP (SEQ ID NO: 36), such as NANPNANPNANP (SEQ ID NO: 38; also referred to herein as “(NANP)₃”), or of the minor repeats that include NANPNVDP (SEQ ID NO: 35) and DPNANPNVDPNANPNV (SEQ ID NO: 37; also referred to herein as “(DPNANPNV)₂”), which is conserved in isolates of P. falciparum. The immunodominant repeat region of malaria CS protein is distinct for each malaria species as shown in FIG. 18. In addition, two CD4+ T cell epitopes, T1 and T* (FIG. 22), identified using CD4+ T cell clones from the protected volunteers immunized with irradiated P. falciparum sporozoites, were also employed in the compositions described herein.

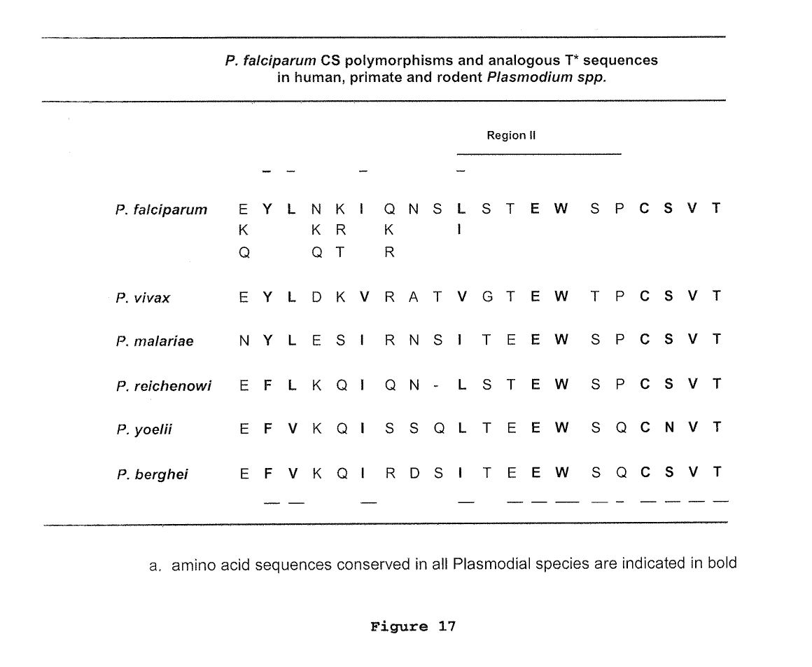

The T1 epitope is contained within the conserved repeat region and is restricted by a limited number of class II molecules (Nardin, Herrington et al. 1989; Munesinghe, Clavijo et al. 1991; Nardin, Oliveira et al. 2000). In contrast the T* epitope is located within a polymorphic region of the CS protein and is recognized by murine and human CD4+ T cells in the context of a broad range of class II molecules and is thus considered a “universal” T cell epitope (Moreno, Clavijo et al. 1993; Calvo-Calle, Hammer et al. 1997; Nardin, Calvo-Calle et al. 2001; Calvo-Calle, Oliveira et al. 2005). The universal T* epitope also contains a class I restricted CD8+ T cell epitope that is recognized by cells of naturally infected individuals living in malaria endemic areas (Blum-Tirouvanziam, Servis et al. 1995). The analogous region of other Plasmodium species also contain CD4+ T cell epitopes that can bind to multiple class II molecules (Nardin, Clavijo et al. 1991) (FIG. 17).

The T* epitope is unique in that it overlaps both a highly variable, as well as a highly conserved region (R11), of the P. falciparum CS protein (FIG. 17). However, only a limited subset of amino acid residues are found at each polymorphic position, while other amino acid positions within this region, such as Y₃₂₇ and L₃₂₈, are highly conserved. Analysis of large numbers of P. falciparum isolates from Africa, Asia and South America indicate that the repertoire of amino acid residues found at each variant position is limited (Yoshida, Di Santi et al. 1990; Doolan, Saul et al. 1992), which may indicate structural constraints in the tertiary structure of this region of the protein that restrict variation (Nussenzweig and Sinnis). In vitro binding studies demonstrated that the naturally occurring substitutions found in the T* epitope in different strains of P. falciparum did not abrogate binding to soluble class II molecules (Moreno, Clavijo et al. 1993).

In all species of malaria, the CS proteins exhibit a pattern of conserved amino acids in the region analogous to P. falciparum T* universal epitope (FIG. 17). An analogous P. vivax CS sequence, EYLDKVRATVGTEWTPCSVT (SEQ ID NO: 55), is recognized by CD4+ T cells derived from a P. vivax sporozoite-immunized chimpanzee (Hardin, Clavijo et al. 1991). The ability to detect T cells specific for this region in humans or chimps immunized by multiple exposures to the bites of malaria infected mosquitoes indicates that the CS universal T cell epitope is a natural peptide produced by processing of native CS following exposure to sporozoites of various plasmodial species.

Aromatic and aliphatic amino acid residues, which can function as critical P1 anchors for binding to DR molecules, are conserved in this region of all CS proteins (FIG. 17). The presence of these conserved residues may indicate that these analogous regions may also be capable of binding to multiple class II molecules and thus be potential immunodominant T cell epitopes. P. vivax and the P. yoelii peptides bound to soluble DR 4 molecules in the peptide competition assay with IC₅₀ 3 μM, as compared to 0.8 μM for P. falciparum indicating that T cell epitopes contained in this region of the CS protein of all Plasmodial species can bind to class II molecules. In addition, binding to DR 13 molecules, which are expressed in higher frequency than the DR 4 in African and in some South American populations, was also demonstrated for all three malarial peptides with an IC₅₀ of 1.8 μM for the P. falciparum peptide and 4-5 μM for P. vivax and P. yoelii peptide. These findings indicate that the 17 amino acid that differ between the DR 4 and DR13 molecules do not affect binding of the T cell epitopes and provide further support for incorporation of epitopes from this region of the malaria CS protein in malaria vaccines.

Peptide binding to the class II molecule is a requirement for, but not a guarantee of, T cell mediated immune responses of the desired specificity and function. TCR interaction with peptide/MHC complexes can elicit a total (agonist), partial or no response (antagonists) in the T cell (Evavold and Allen, 1993; Jameson and Bevan. 1995). In addition, peptide/MHC/TCR affinity may modulate the subset of T helper cells that predominate in an immune response (Kumar et al., 1995). The corresponding “universal T cell epitopes” of rodent malaria CS proteins have also been shown to elicit sporozoite specific T cell responses that are functional in vivo. The P. berghei CS sequence analogous to the P. falciparum T* epitope (FIG. 18), when synthesized in tandem with P. berghei CS repeats, elicited high levels of protective antibodies in A/J mice (Tam, Clavijo et al. 1990). Similarly, a peptide containing the homologous P. yoelii CS sequence, which shares about 12/20 amino acids with the P. falciparum universal T* sequence elicited protective CD4+ T cell responses in Balb/c mice (Takita-Sonoda, Tsuji et al. 1996)

A branched peptide containing only the epitopes from the P. falciparum CS repeat epitopes, T1 and B, stimulated high levels of antibody and T cell responses in mice and humans expressing a limited number of MHC class II genotypes (Munesinghe, Clavijo et al. 1991; Nardin, Oliveira et al. 2000). Additional studies demonstrated that the HLA restriction of the anti-CS repeat response could be overcome by including the malaria universal T* epitope in the vaccine (Nardin, Calvo-Calle et al. 1998; Nardin, Calvo-Calle et al. 2001). In Phase I trials, a tetrabranched peptide (T1BT*)₄, containing the CS protein B and T1 repeats linked to the universal T* epitope, was shown to elicit antibody and T cell responses specific for CS in human volunteers of diverse genetic backgrounds (Nardin, Calvo-Calle et al. 2001). In the human volunteers, the malaria specific antibody and CD4+ T cell responses induced by the tri-epitope peptide were similar to that stimulated by irradiated sporozoites (Herrington, Davis et al. 1991; Moreno, Clavijo et al. 1993; Calvo-Calle, Oliveira et al. 2005). However, the difficulty of synthesis of multibranched peptides and their low yields prevented development of commercial malaria vaccines based on this delivery platform.

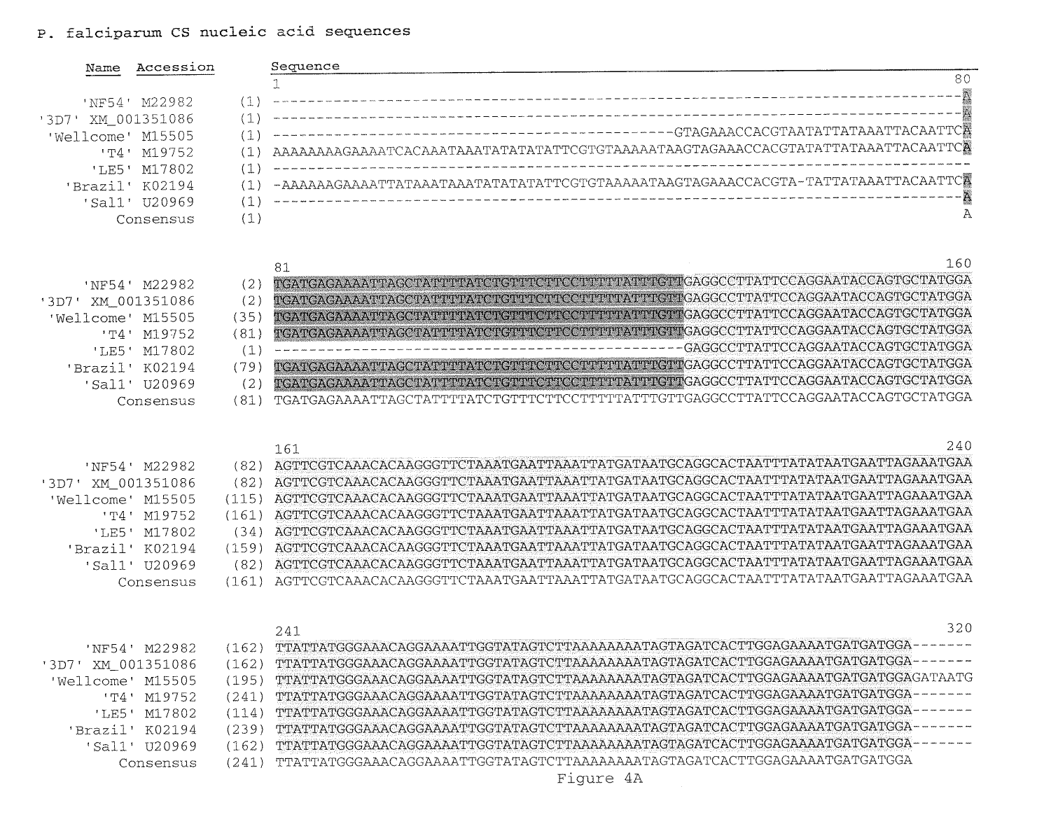

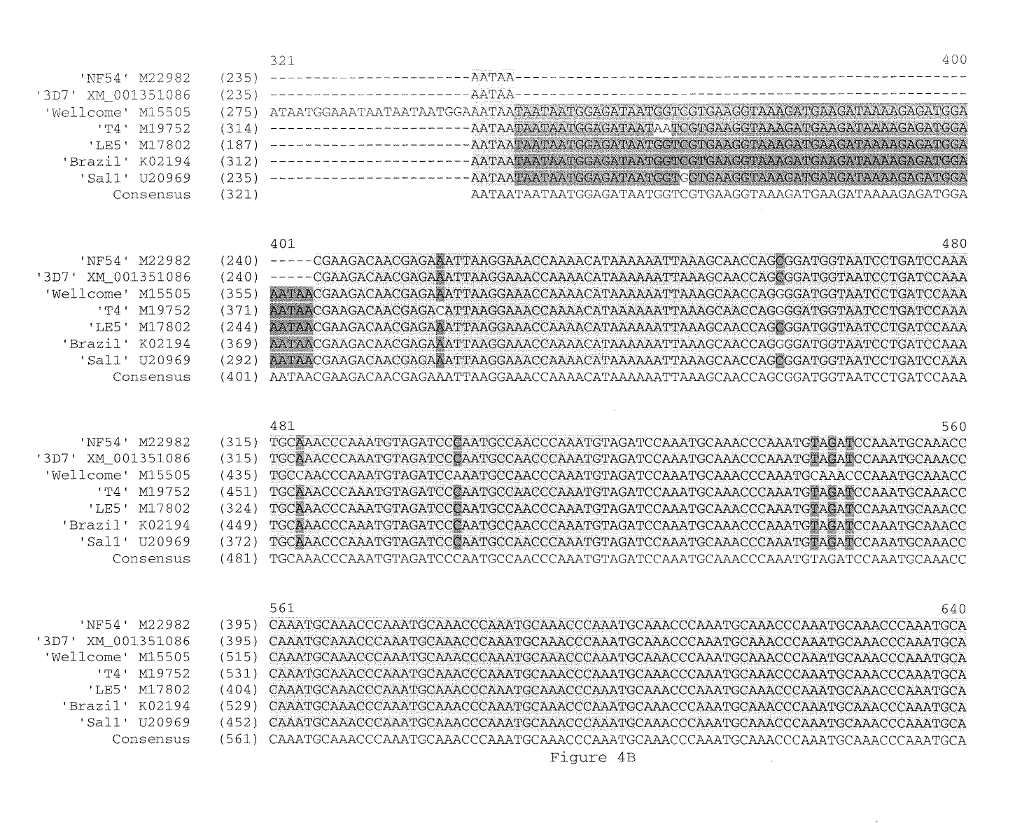

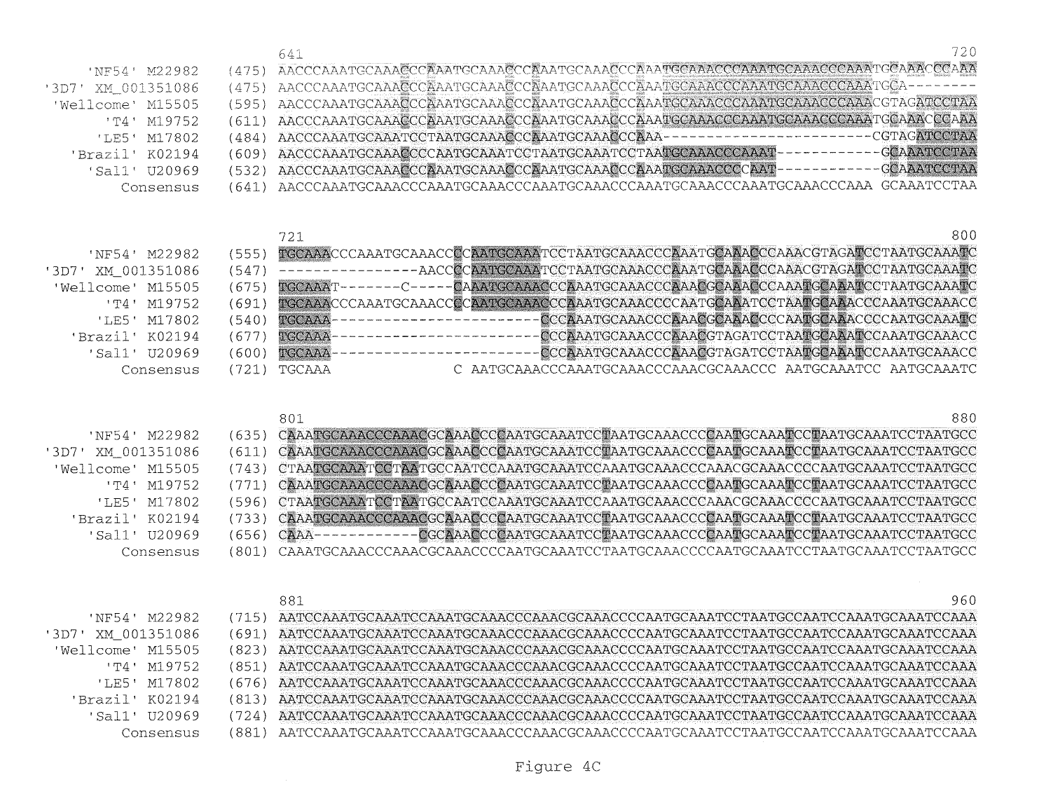

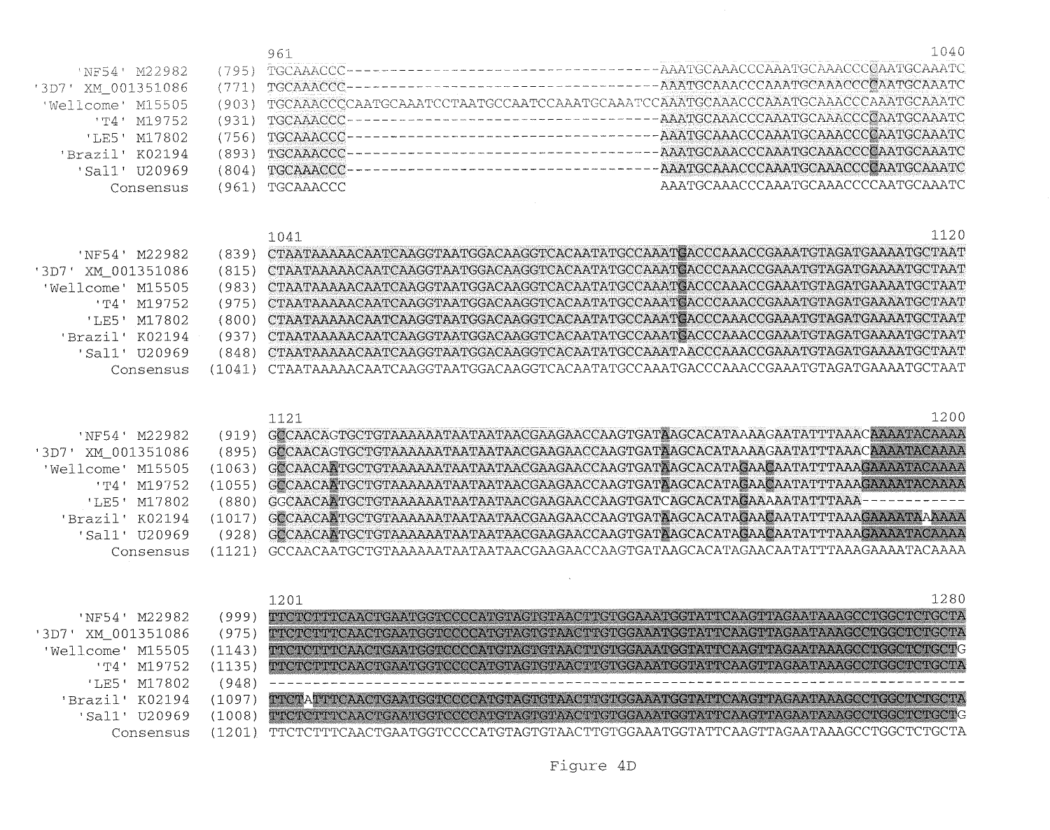

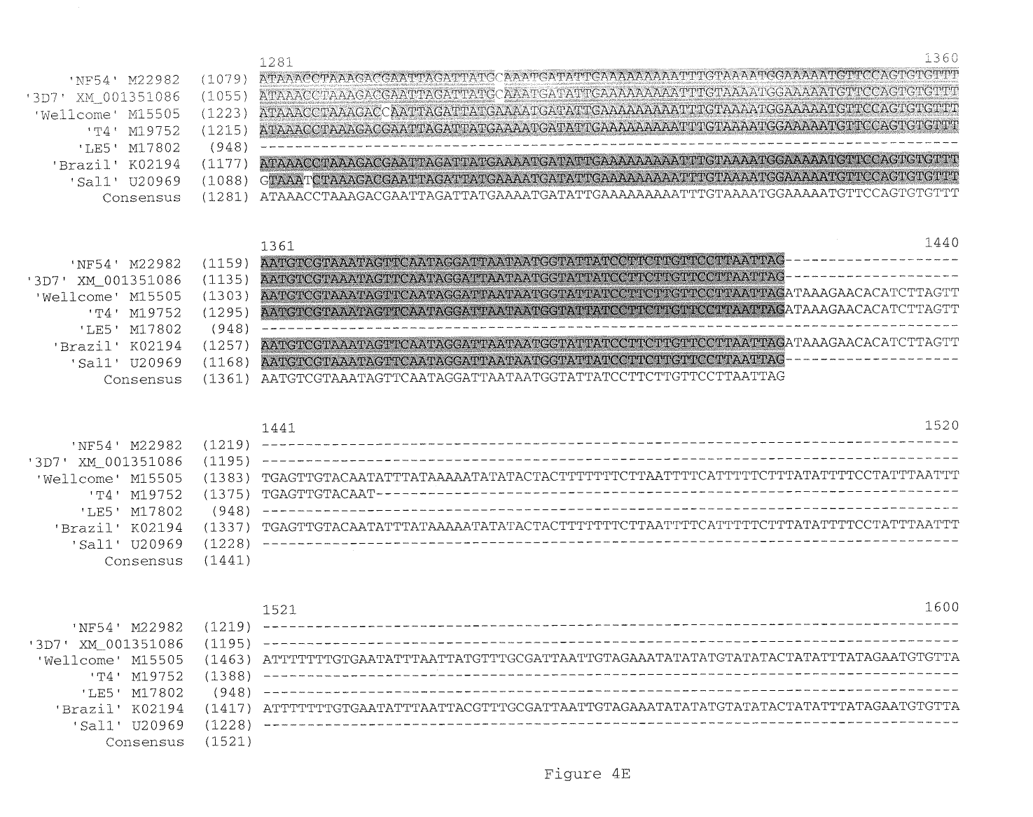

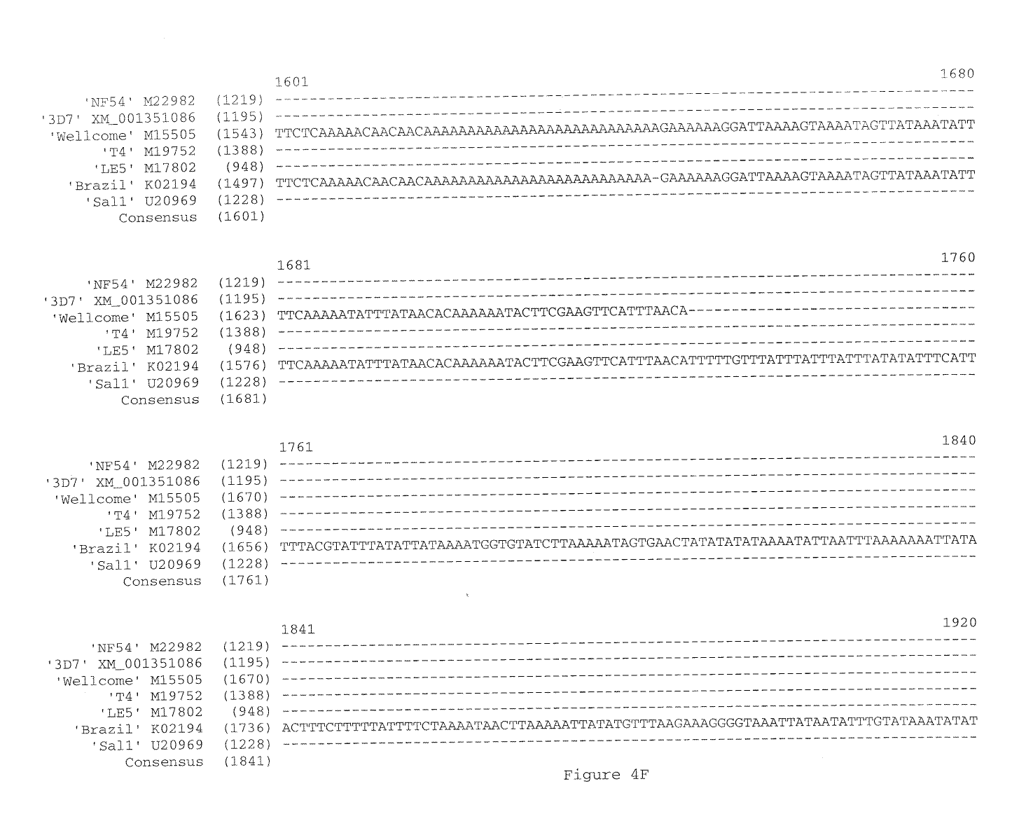

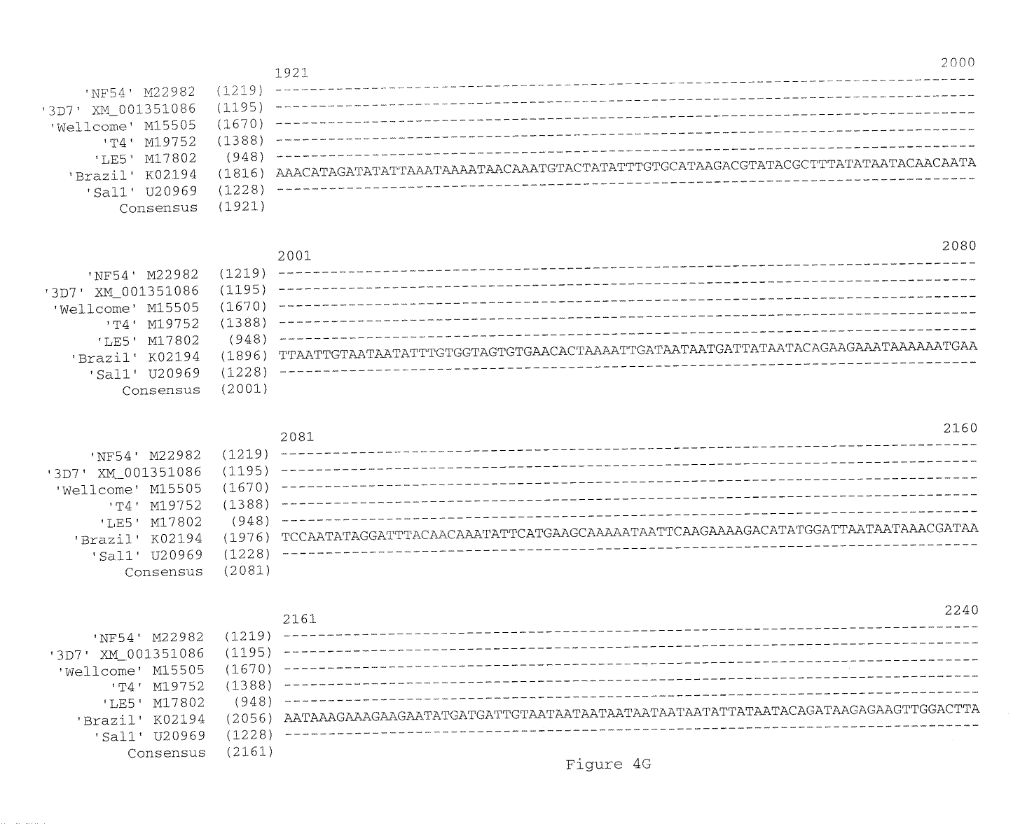

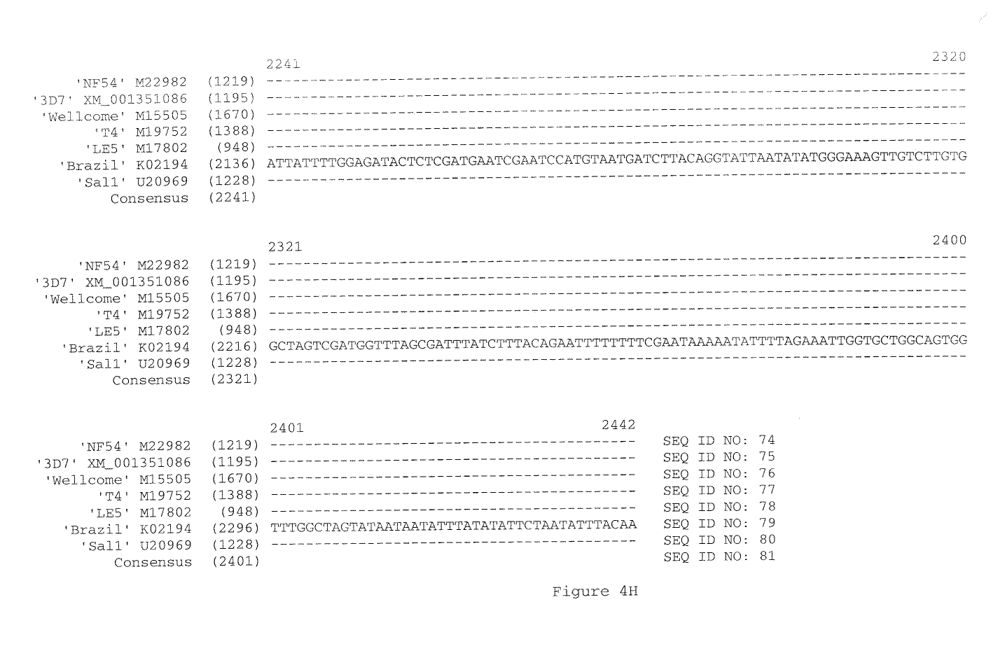

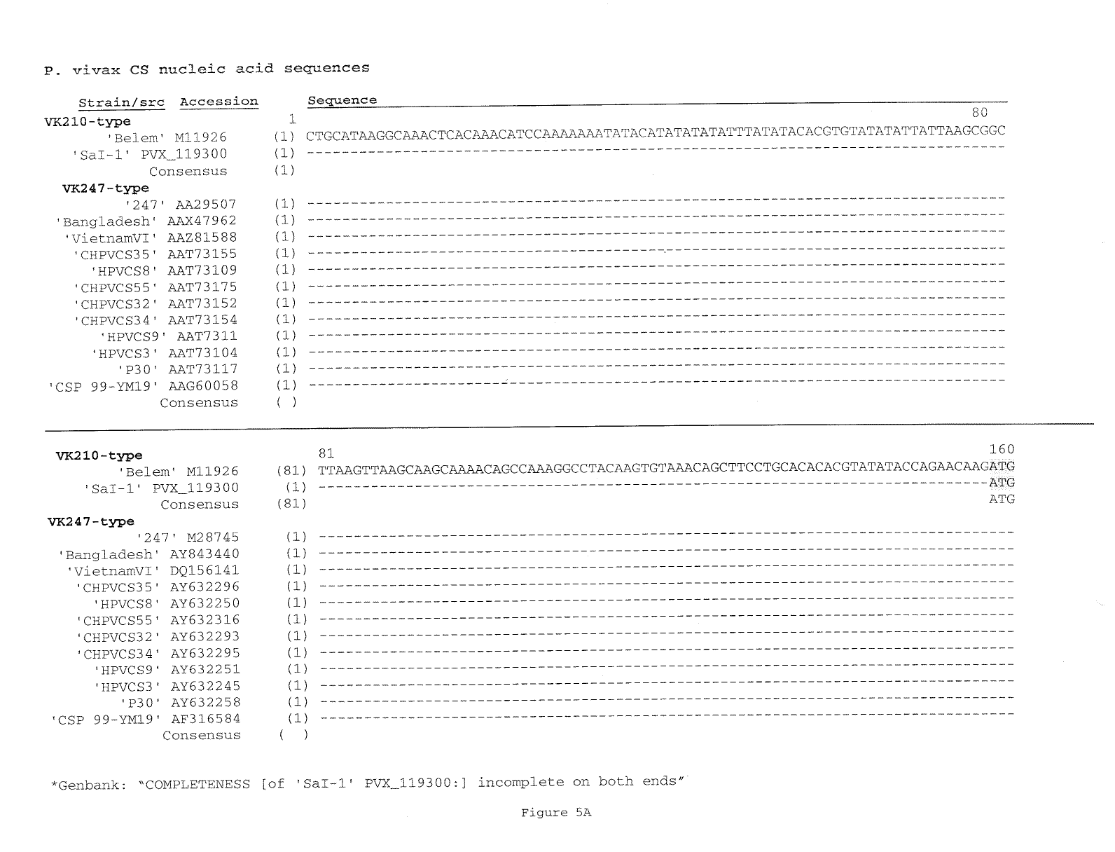

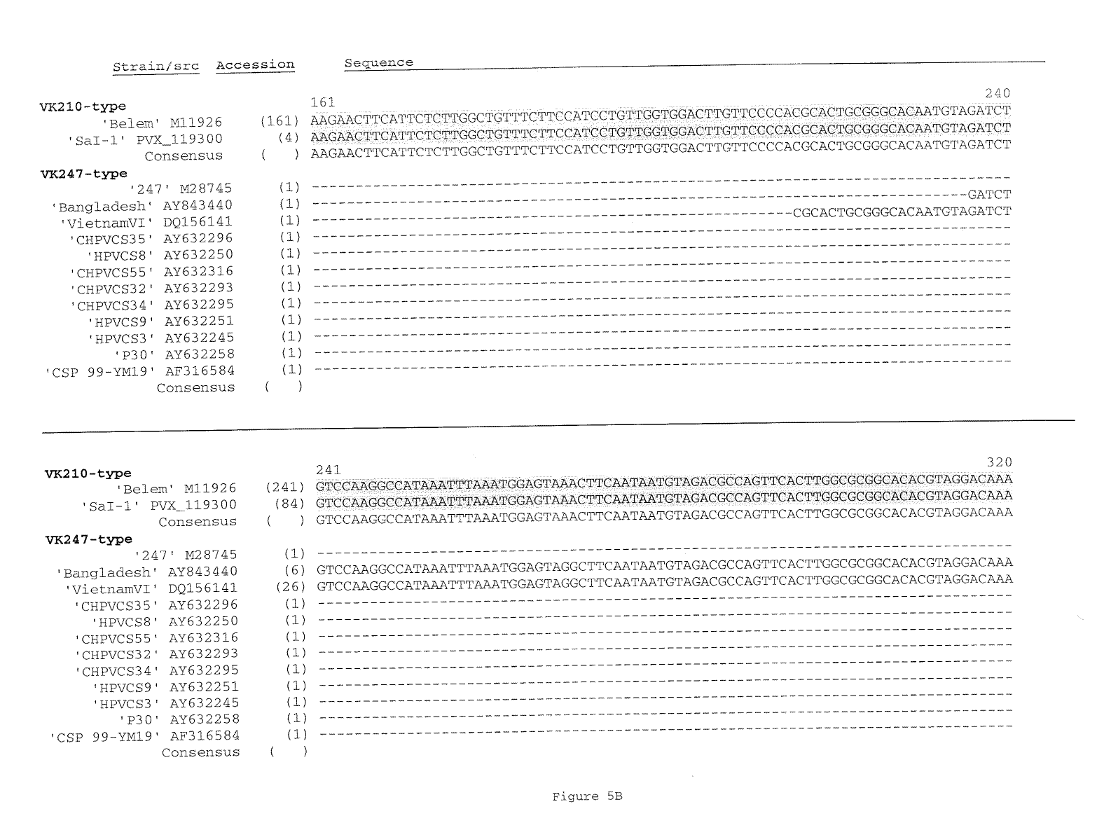

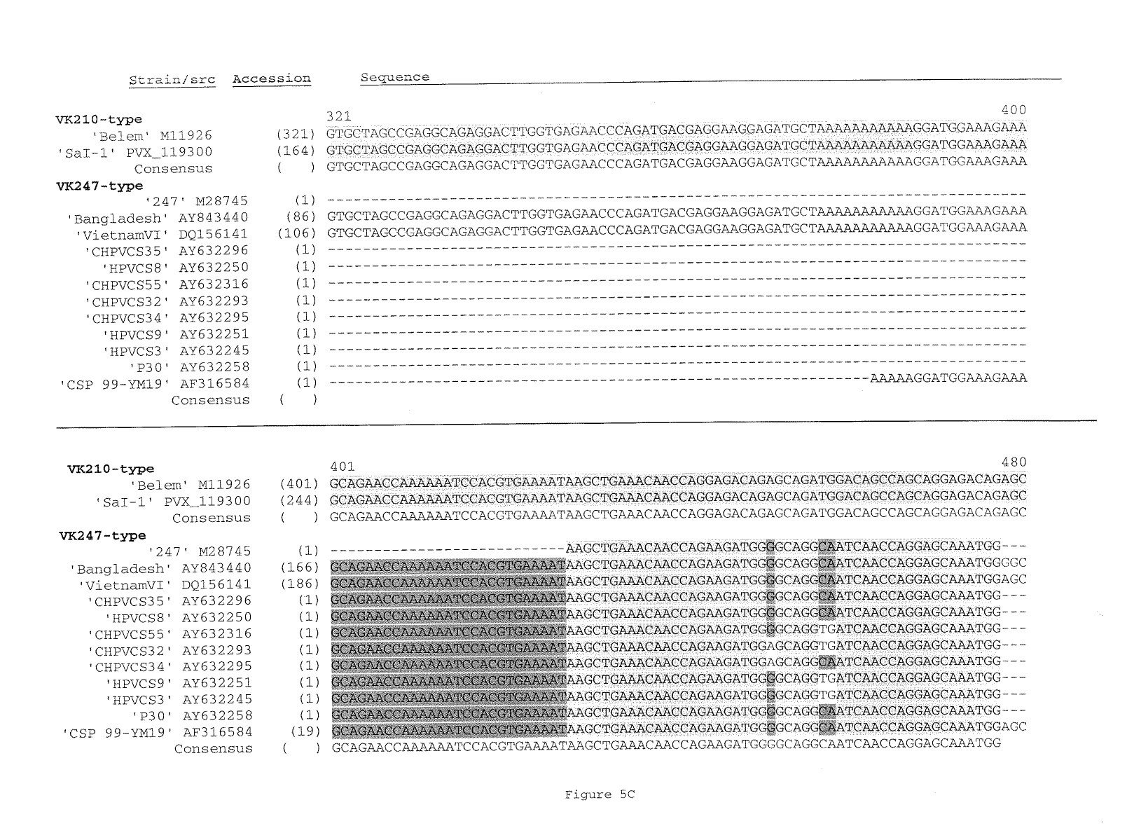

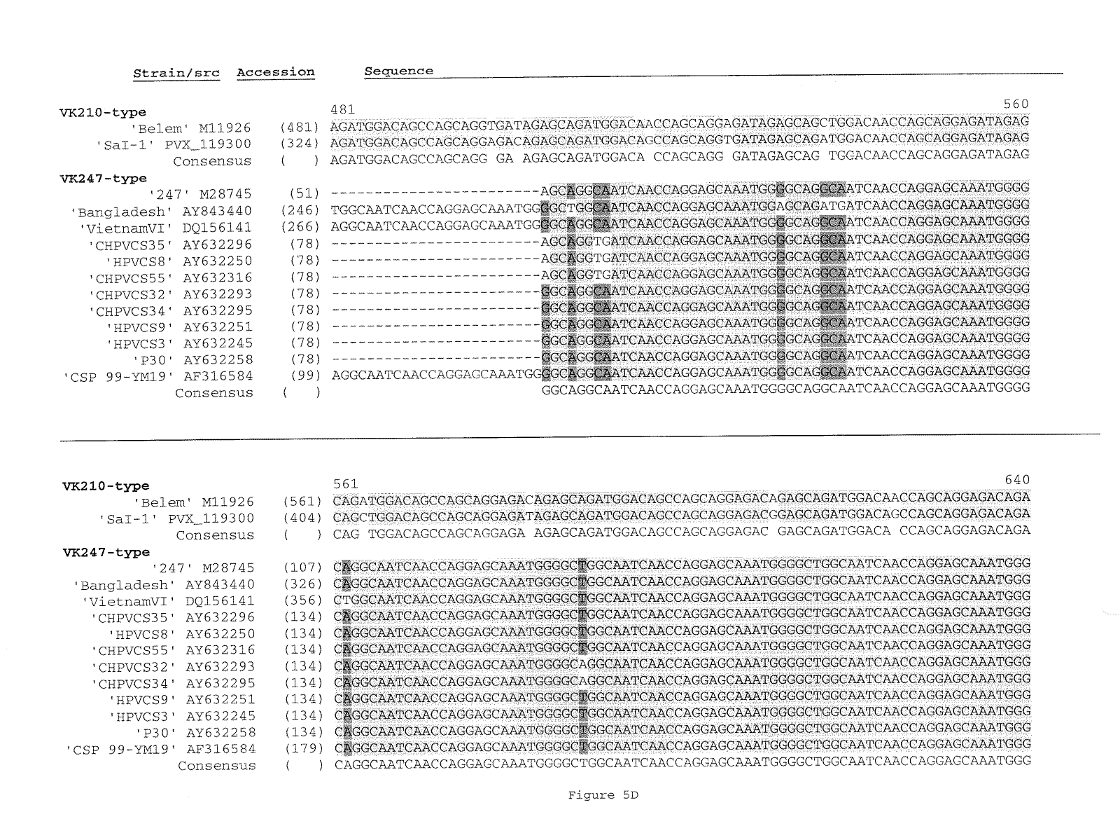

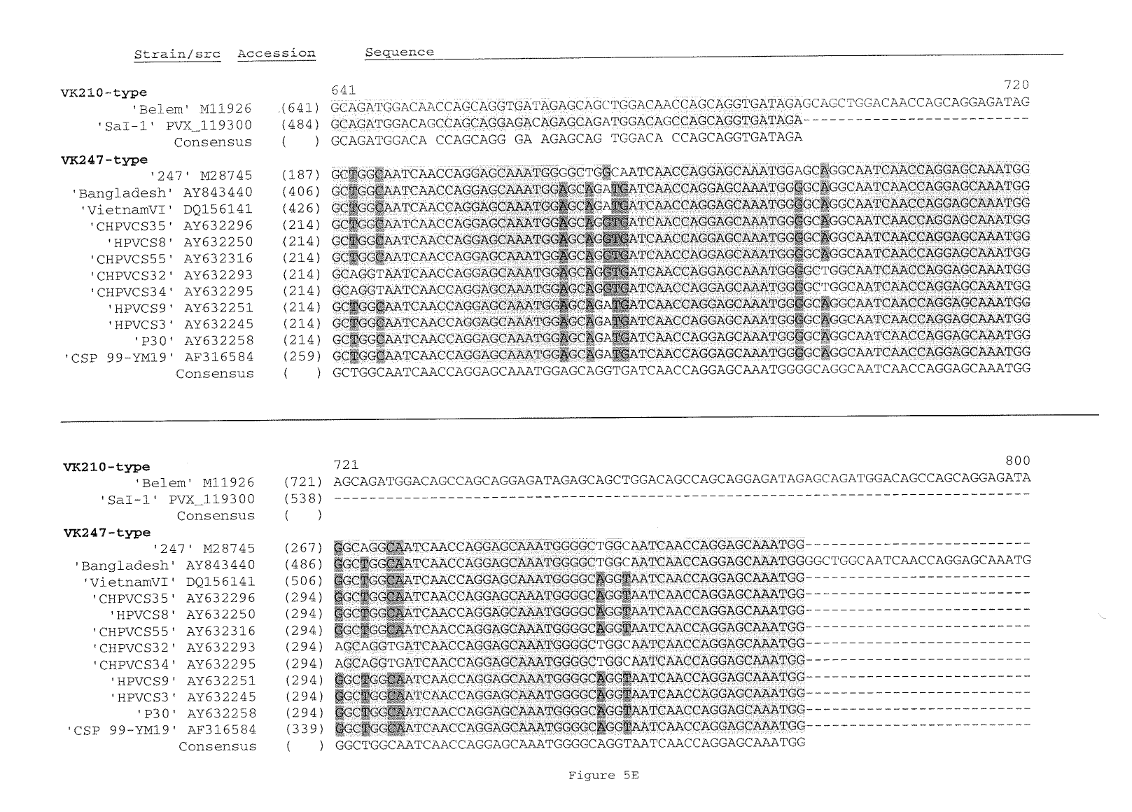

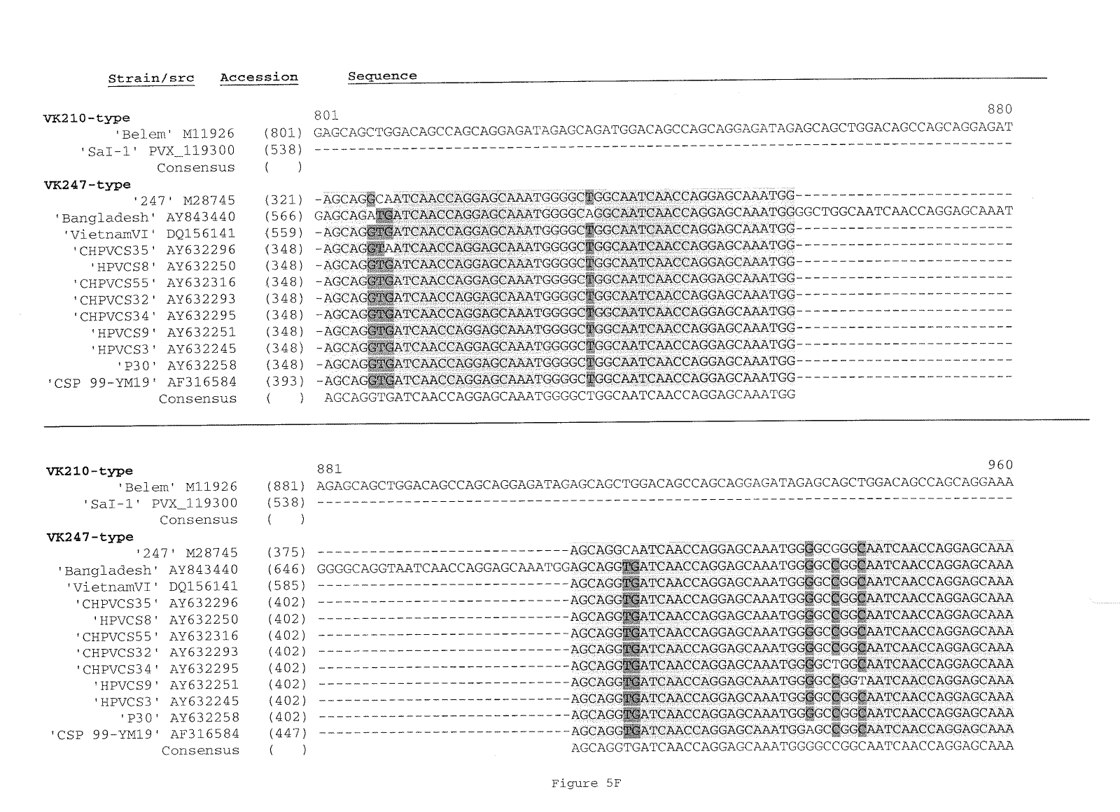

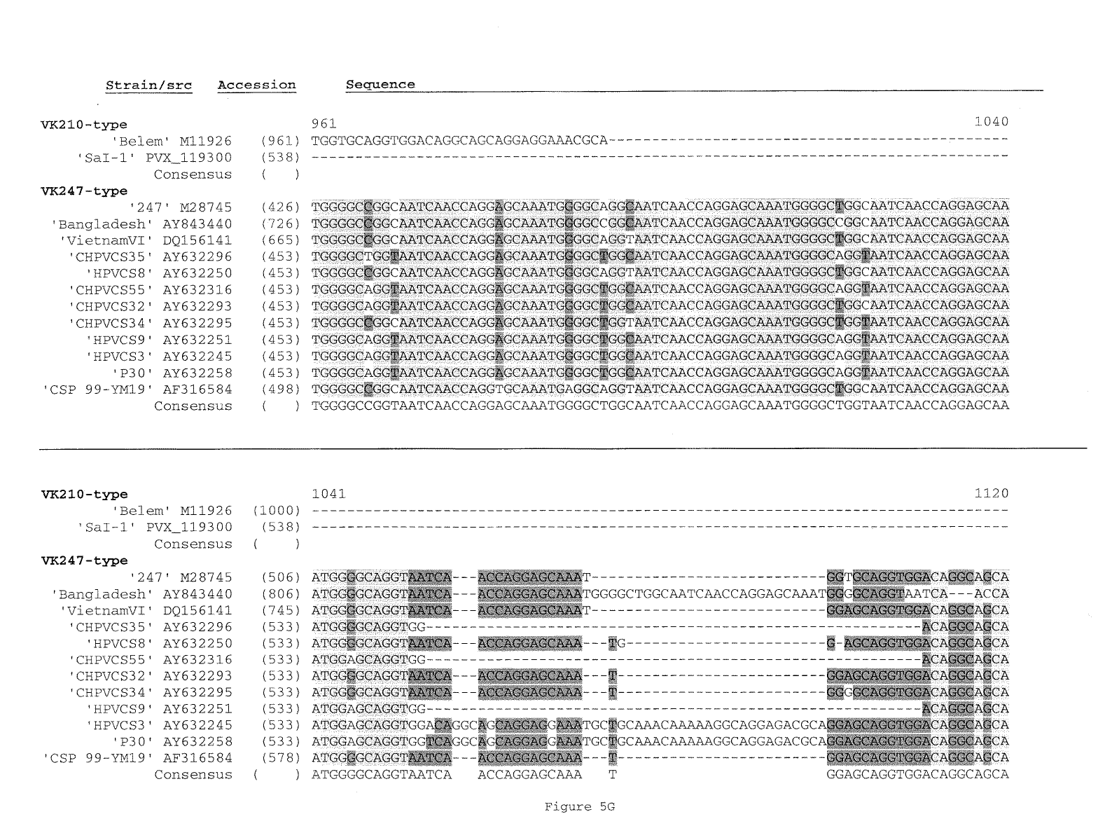

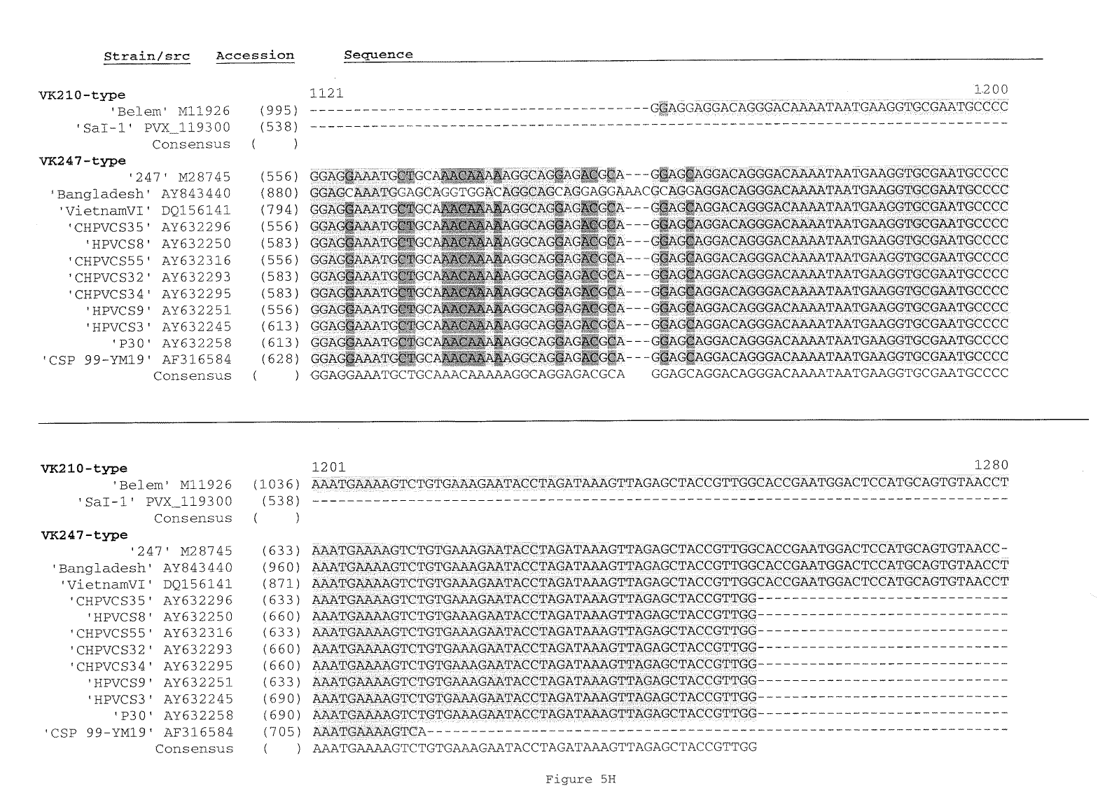

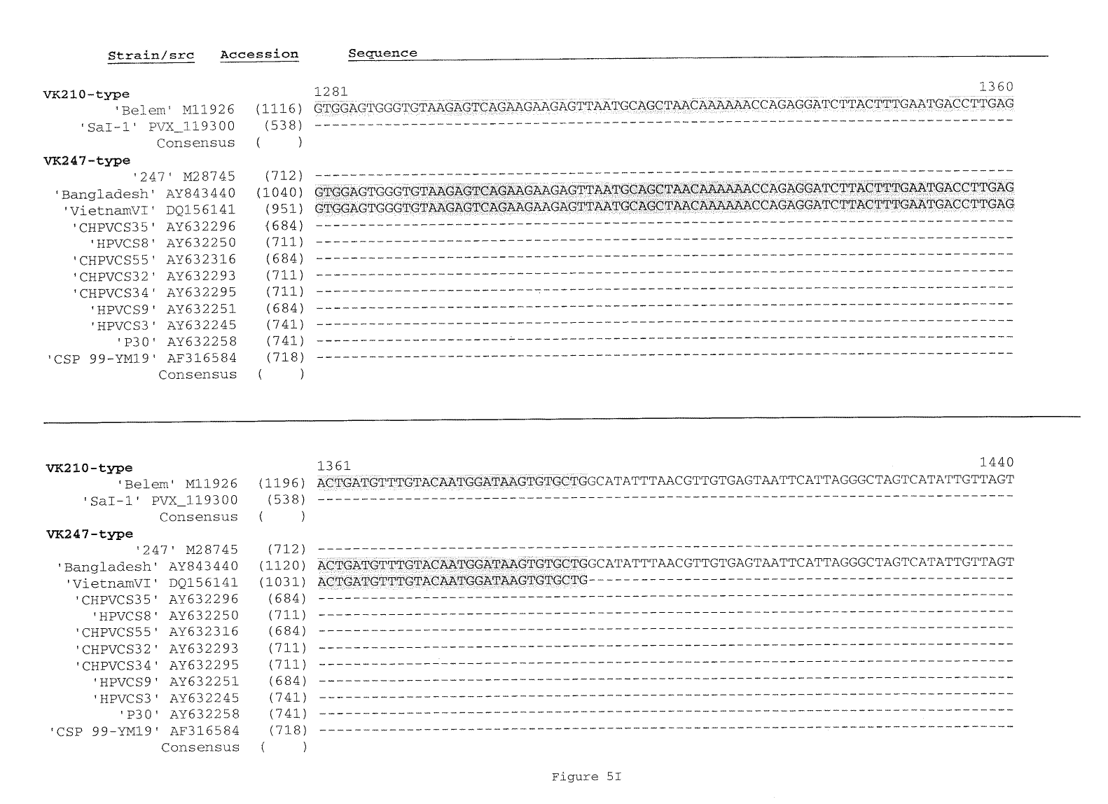

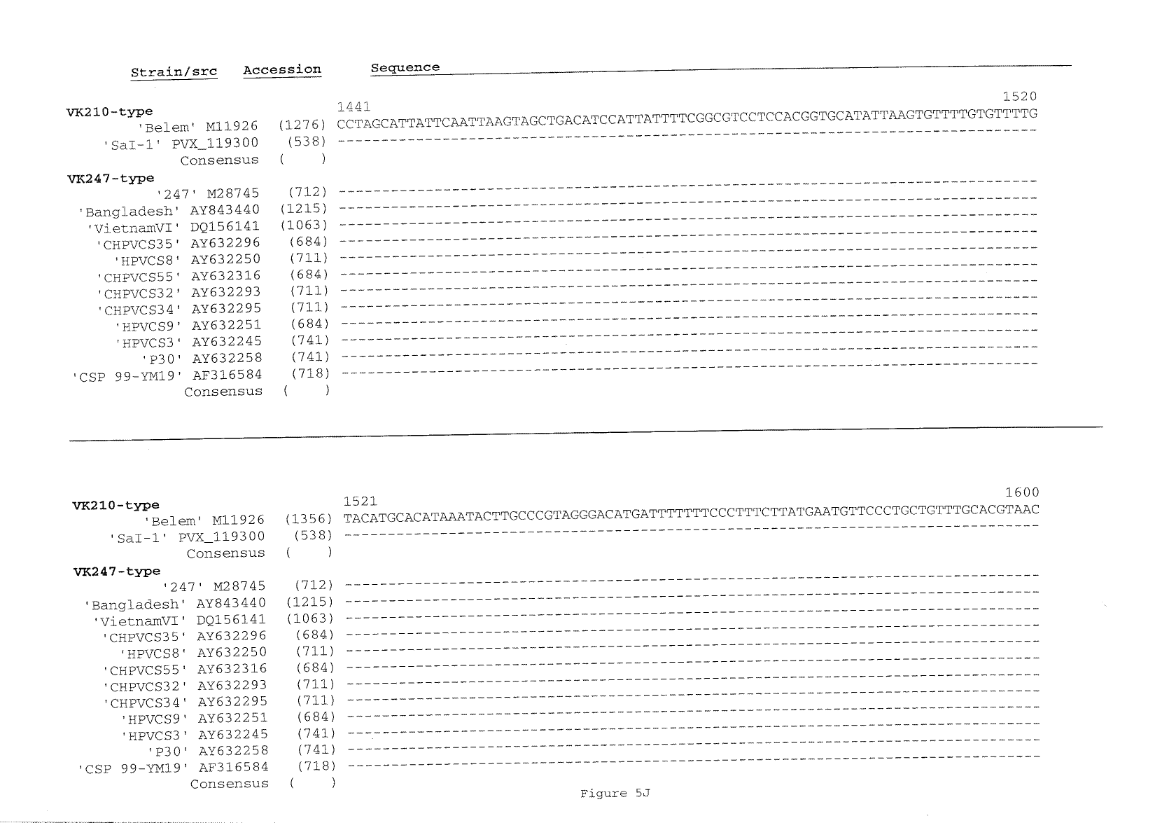

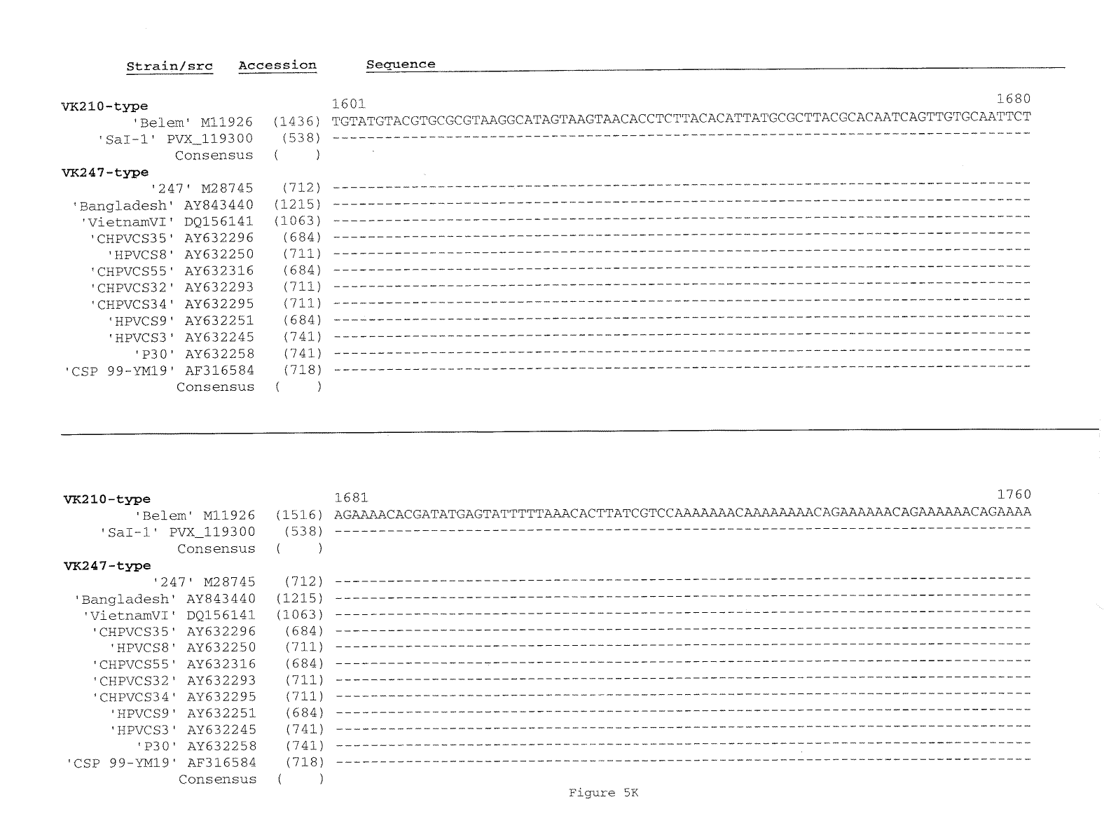

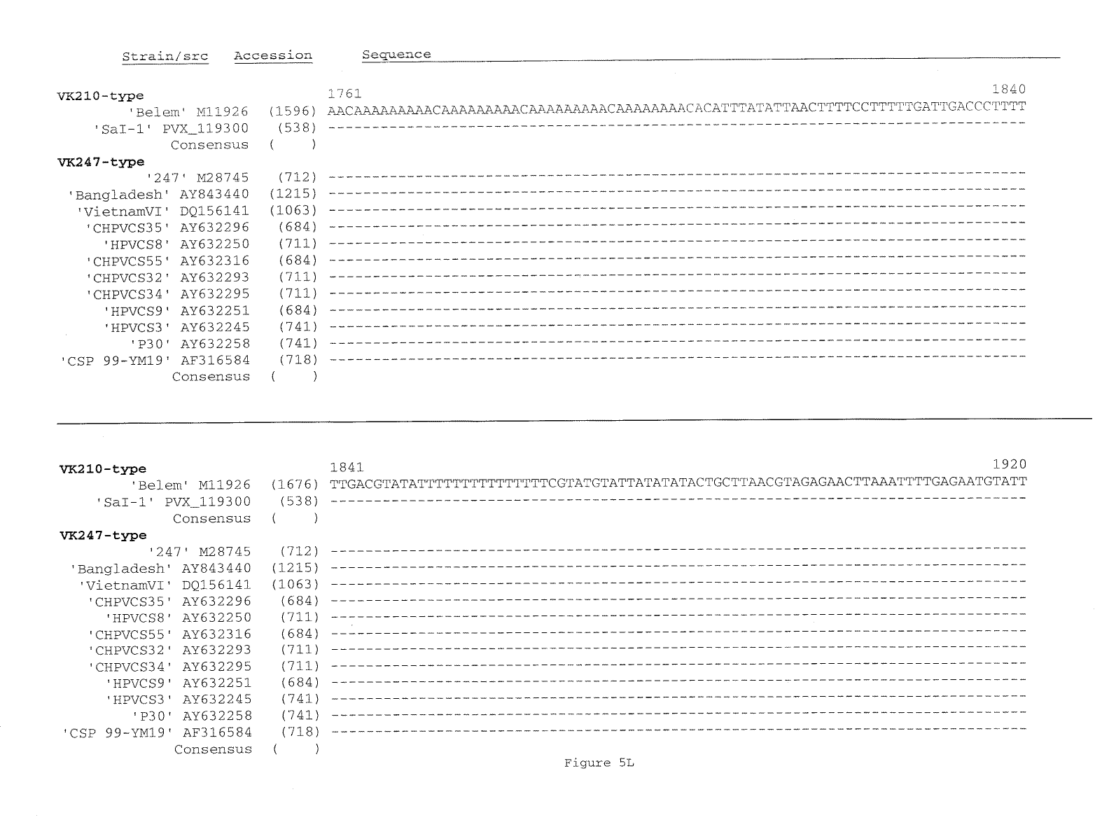

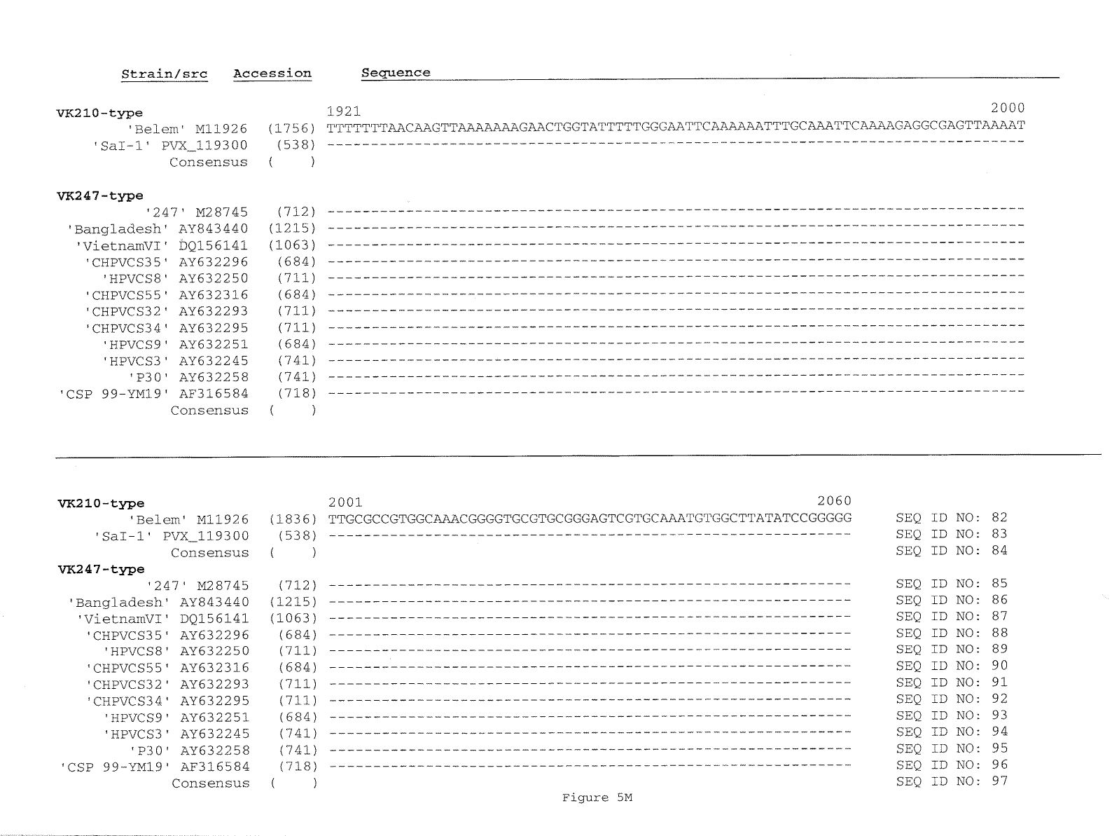

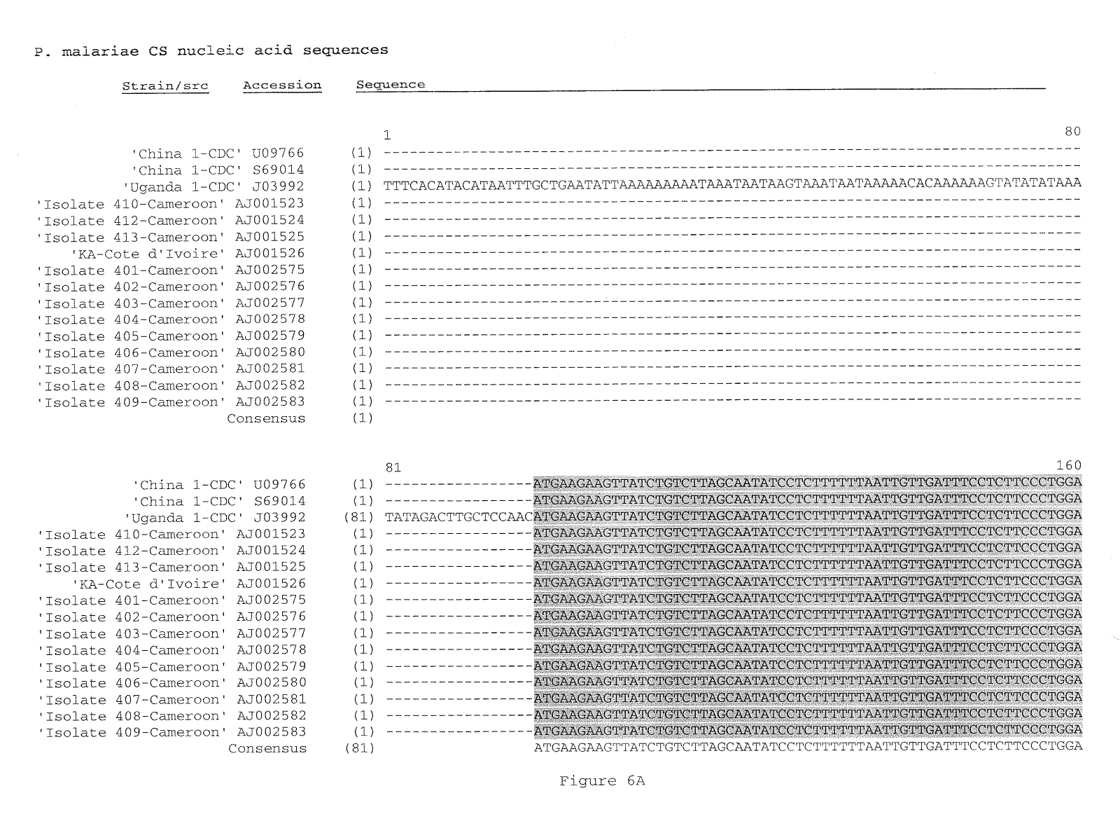

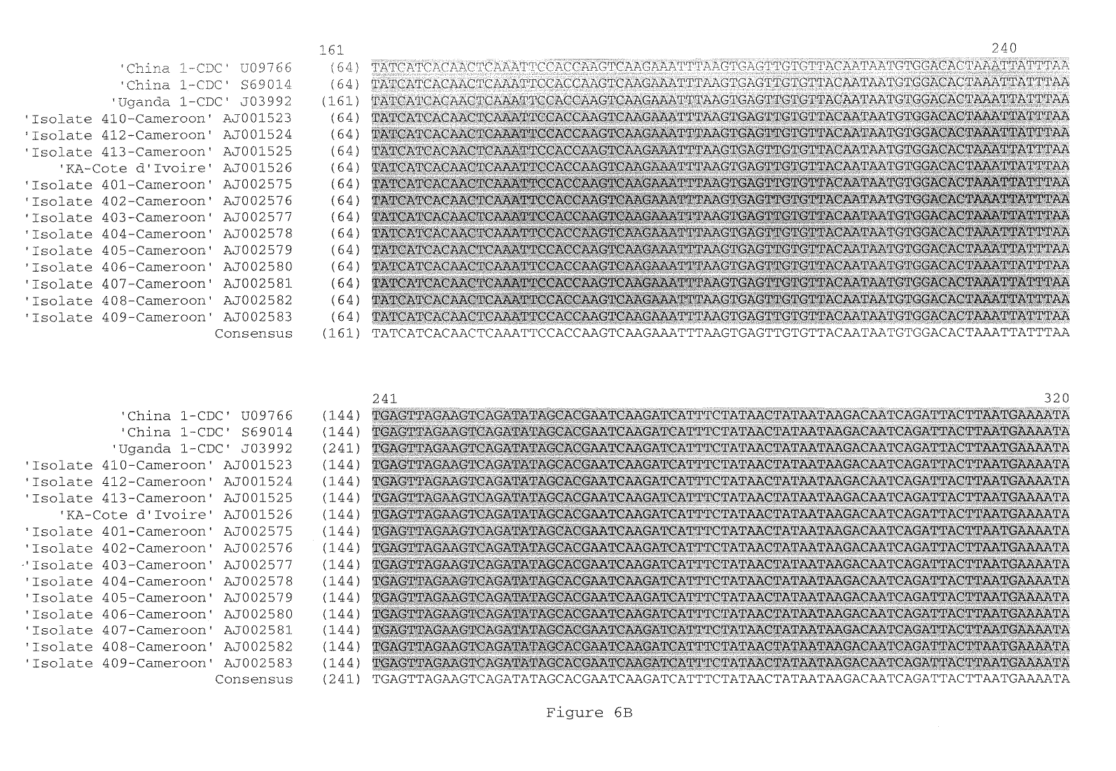

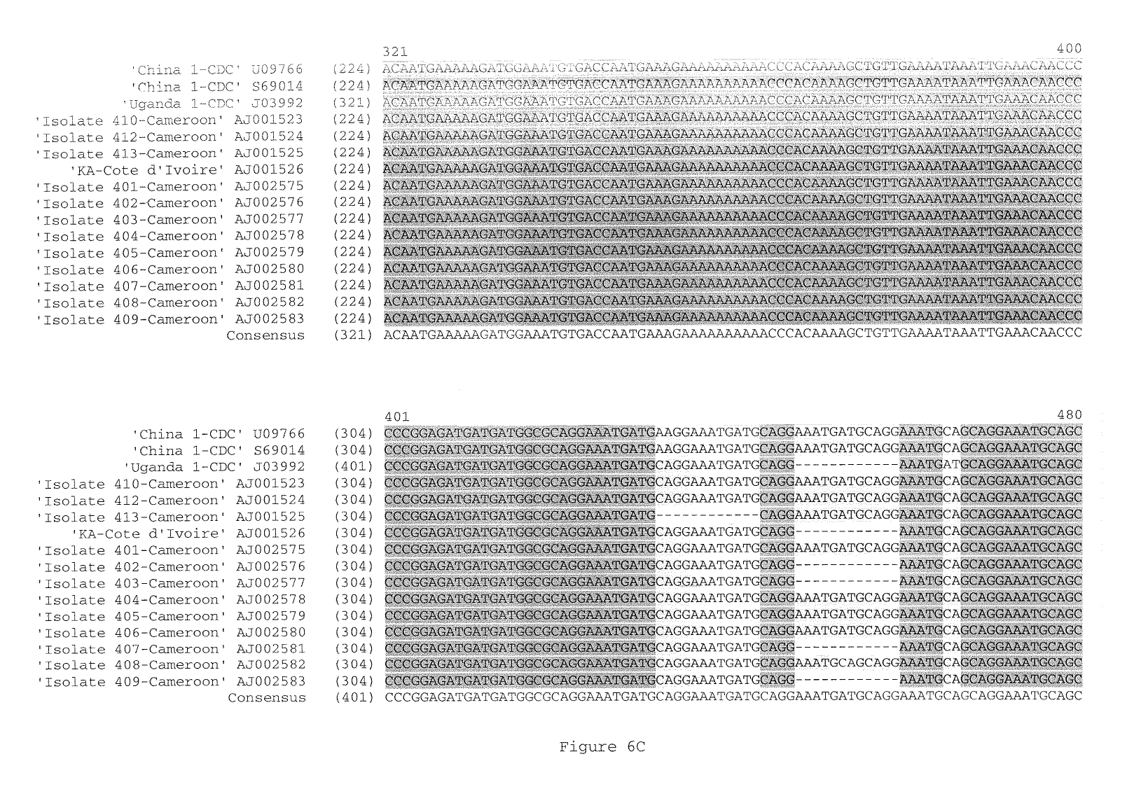

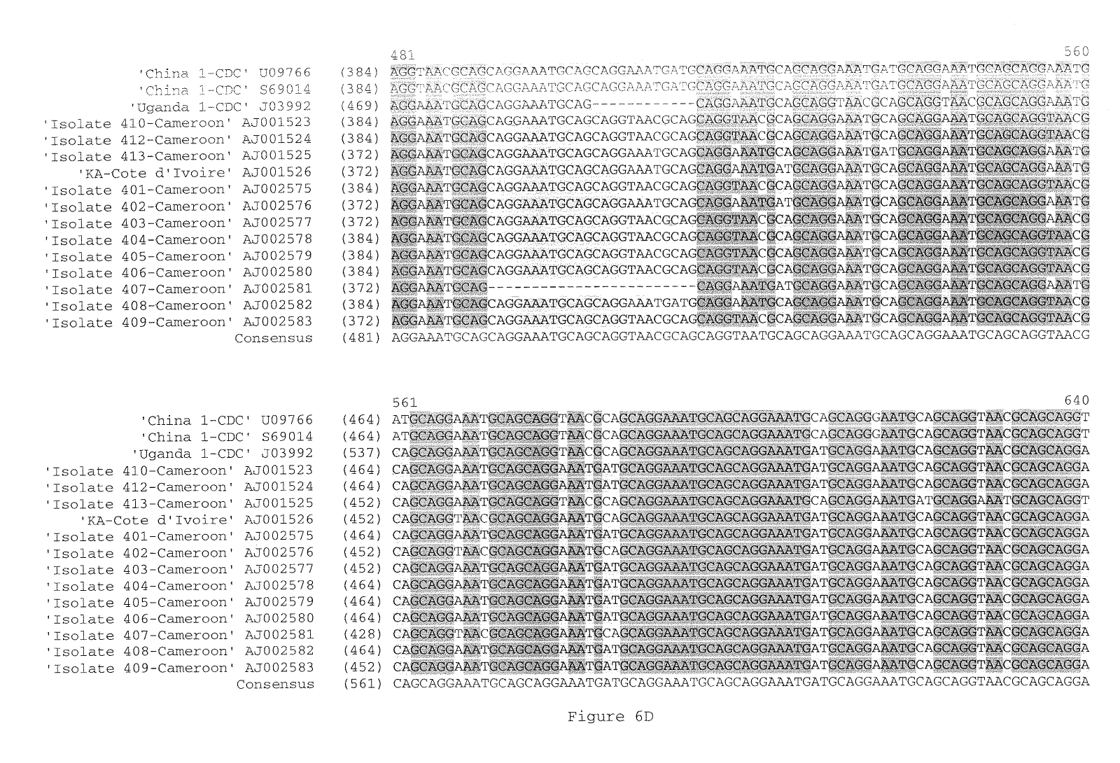

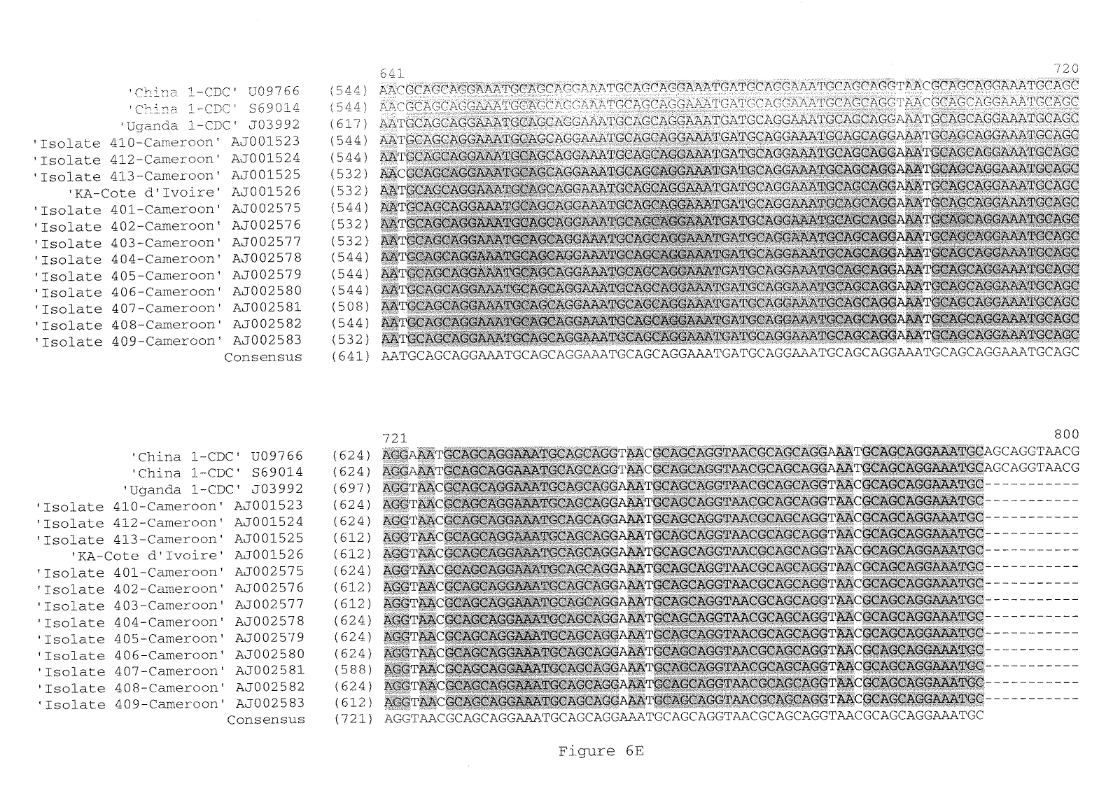

More recent murine studies have demonstrated that the branched peptide configuration is not required for immunogenicity of the malaria T1BT* sequence (Calvo-Calle, Oliveira et al. 2006). A linear 48 mer peptide containing the T1BT* sequence was as immunogenic as the more complex tetrabranched construct when tested in C57BL mice using water-in-oil adjuvants, Montanide ISA 720, ISA 51 or Freunds Adjuvant (FIGS. 49A and 4B). ELISA anti-repeat titers (black bars) correlated with anti-parasite titers, measured by indirect immunofluorescence (IFA) using P. falciparum sporozoites (hatched bars), indicating that the T1BT* linear peptide induced antibodies that effectively react with native CS on the sporozoite surface, as observed for the branched construct. Similar immune responses to the T1BT* sequence were observed in Balb/c mice indicating that immunogenicity of the linear T1BT* peptide was not genetically restricted (Calvo-Calle, Oliveira et al. 2006).

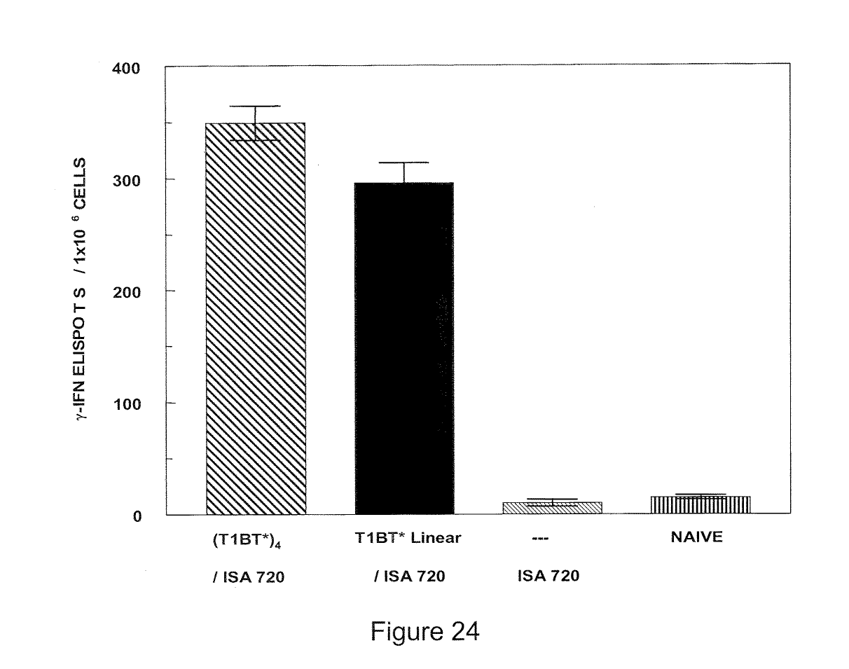

In addition to eliciting anti-repeat antibodies, the T1BT* sequence also elicited CS-specific IFNγ producing T cells (FIG. 24). The positive IFNγ ELISPOT reflected the presence of malaria-specific immune cells, as spleen cells of naïve mice, or mice receiving adjuvant only, had negligible numbers of SFC. Cytokine profiles measured by Cytokine Bead Assay (CBA) in supernatants of peptide-stimulated spleen cell cultures were consistent with the results of IFNγ ELISPOT assays. A dose dependent increase in levels of IFNγ was obtained, with no detectable IL-4.

A critical issue in vaccine development is whether immunization with P. falciparum vaccines can protect against sporozoite challenge. Since humans are the only host that is highly susceptible to P. falciparum sporozoites, studies of vaccine efficacy have required costly and labor intensive Phase II clinical trials to assess ability of vaccine induced responses to protect against sporozoite challenge. To address this limitation, a transgenic P. berghei rodent malaria parasite that expresses P. falciparum CS repeats, termed PfPb, which allows direct measurement of the biological activity of immune responses elicited by vaccines containing P. falciparum CS repeats, termed PfPb, has been described, which allow direct measurement (Persson, Oliveira et al. 2002). In addition to providing a small animal model for measuring protection in vivo, the rodent model allows direct measurement of liver stages and the dissection of the immunological mechanisms functioning in immune resistance to P. falciparum CS repeats, studies that cannot be carried out in human volunteers.

Using the PfPb transgenic sporozoites, it has been demonstrated that mice immunized with the T1BT* minimal epitopes, synthesized as either a linear or a branched peptide and formulated in ISA 720 adjuvant, were protected against challenge by the bite of infected mosquitoes (FIGS. 25A and 25B). Resistance to sporozoite challenge was malaria specific, as mice receiving only adjuvant, either Freunds or ISA 720 (hatched bars), remained susceptible to sporozoite challenge.

Depletion of T cells from the peptide immunized mice, by treatment with MAB specific for murine CD4 or CD8 prior to sporozoite challenge, did not abrogate immune resistance to sporozoite challenge (FIG. 26A). − Levels of parasite 18S rRNA in the livers of immunized mice depleted of CD4+ or CD8+ T cells were as low as those observed in the untreated immunized mice. Therefore, repeat specific T cells did not appear to play a significant role in resistance to viable sporozoite challenge, suggesting protection was mediated by high levels of anti-repeat antibodies.

To analyze the role of sporozoite neutralizing antibodies, sera of the peptide immunized mice were tested for the ability to block sporozoite invasion of human hepatoma cells in vitro (Kumar, Oliveira et al. 2004). Immune sera obtained from protected mice inhibited 80-90% of sporozoite invasion, when compared to levels of parasite 18sRNA in cultures receiving parasites incubated with pre-immune sera (FIG. 26B). Sporozoite neutralizing activity directly correlated with high levels of anti-repeat antibodies in all of the immune sera (GMT about 54,613; range about 20,480 to 163,840). These studies demonstrate that strong anti-repeat antibody responses induced by immunization with the minimal T1BT* sequence can function in protective immunity against sporozoites.

In clinical studies, numerous CS subunit vaccines, comprised of peptides, recombinant proteins, viral vectors and virus-like particles (VLP), were of suboptimal immunogenicity due to the lack of strong adjuvants. Many of the oil-in-water adjuvants that give high levels of immunogenicity in murine studies were too reactogenic for human use. These limitations were noted in studies of a malaria VLP vaccine based on hepatitis B core antigen containing the P. falciparum T1BT* epitopes (Birkett, Lyons et al. 2002; Nardin, Oliveira et al. 2004; Oliveira, Wetzel et al. 2005; Gregson et al. 2007). Phase I testing demonstrated that these VLP were safe and immunogenic when formulated with alum. While anti-repeat antibodies and malaria specific CD4+ Th1-type T cells producing IFNγ were elicited in the volunteers immunized with the VLP adsorbed to alum, the responses were low in the majority of the vacinees (Nardin, Oliveira et al. 2004; Gregson et al. 2007). However, efforts to use the more potent water-in-oil adjuvant ISA 720 were limited by reactogenicity (Langermans, Schmidt et al. 2005; Oliveira, Wetzel et al. 2005), as has been reported for other malaria and HIV vaccine candidates formulated in ISA adjuvants (Saul, Lawrence et al. 1999; Saul, Lawrence et al. 2005). Due to potential reactogenicity, only a single dose immunization with the T1BT* VLP/ISA 720 formulation was tested in humans. In Phase I/II trials, this single dose immunization elicited suboptimal antibody and T cell responses that did not protect against sporozoite challenge (Walther, Dunachie et al. 2005). Thus, there is a need to development more potent and less reactogenic compositions for use in preventing malaria disease, for example, in formulations for efficacious malaria vaccines.

The limitations of complex adjuvant formulations were also confronted during development of the CS subunit vaccine, which is currently in Phase III trials in Africa. The formulation is a VLP comprised of a hepatitis B virus surface antigen fused with the repeats and C terminus of P. falciparum CS protein. In malaria naïve volunteers, the composition stimulated high levels of anti-CS antibodies, CD4+Th1 cells and sterile immunity only when administered in a multicomponent adjuvant formulation (Gordon, McGovern et al. 1995; Stoute, Slaoui et al. 1997; Kester, McKinney et al. 2001). The composition includes MPL, a monophohoryl lipid A derived from bacterial LPS, and QS21, a purified fraction of saponin, mixed in a proprietary oil-in-water emulsion. Early clinical studies demonstrated this potent adjuvant/VLP combination was reactogenic (Stoute, Slaoui et al. 1997; Kester, McKinney et al. 2001) and unstable on storage (Bojang, Milligan et al. 2001), requiring point-of-use formulation, a critical limitation for vaccines that will be administered predominantly in underdeveloped countries. In clinical trials in Africa, vaccine efficacy was about 34% in adults (Bojang, Milligan et al. 2001) and about 56% of immunized children were protected against severe clinical disease (Alonso, Sacarlal et al. 2004). Sterile immunity was transient in adults, however, lasting only weeks to months (Stoute, Kester et al. 1998; Bojang, Milligan et al. 2001).

The clinical trials of pre-erythrocytic malaria vaccines demonstrate that irradiated sporozoite and CS based subunit vaccines can elicit protection against P. falciparum in humans. These studies also demonstrate that malaria vaccines require potent adjuvants that are simple to produce and stable on storage and that can elicit optimal immune responses without reactogenicity.

TLRs are Pattern Recognition Receptors (PRR) expressed on antigen-presenting cells (APC) that act as initiators of the innate immune response required for potent adaptive immunity (Medzhitov and Janeway 1997; Kopp and Medzhitov 1999; Barton and Medzhitov 2002; Bendelac and Medzhitov 2002; Pasare and Medzhitov 2004). Engagement of PRRs by their cognate ligands, Pathogen-Associated Molecular Patterns (PAMPs), trigger important cellular mechanisms which lead to the expression of costimulatory molecules, secretion of critical cytokines and chemokines, and efficient processing and presentation of antigens to T cells. To date, a total of 13 TLRs (TLR1-13) have been discovered and the corresponding PAMPs for some of these receptors have been identified, as shown in FIG. 22. Some well characterized PAMPs include bacterial cell wall components (e.g. lipoproteins and lipopolysaccharides) that function as TLR2/TLR4 agonists, while bacterial DNA sequences that contain unmethylated CpG residues function as TLR9 agonists, and bacterial flagellin as a potent TLR5 agonist. Compositions that include TLR agonists and malaria antigens are described herein.

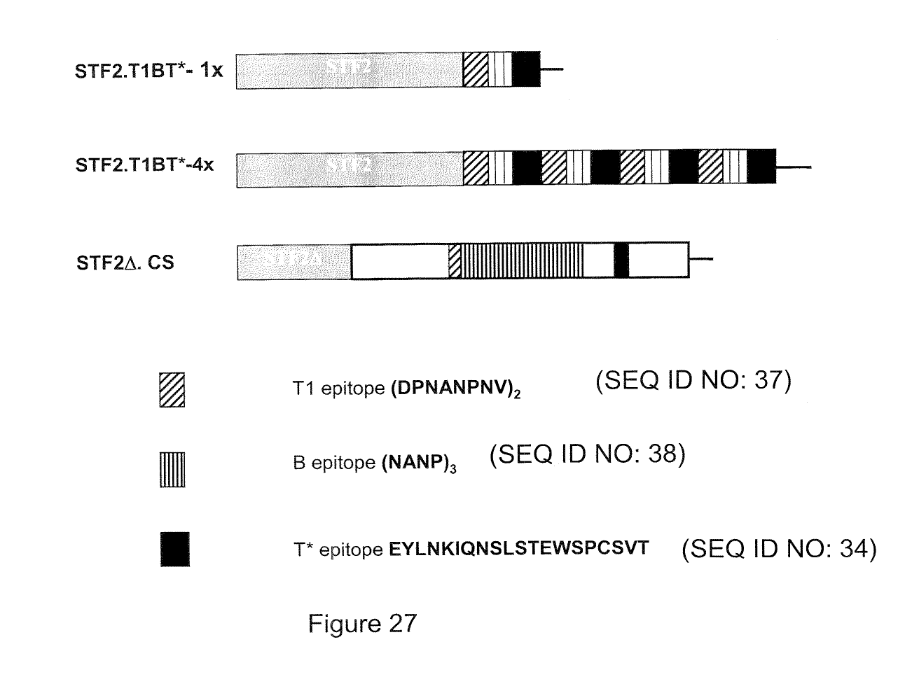

Compositions that include TLR agonists described herein may elicit high levels of sporozoite neutralizing antibodies to reduce the number of parasites that enter hepatocytes, as well as cellular responses that can target the residual intracellular stages that develop from sporozoites that escape these antibodies. It is believed that an advantageous method to generate a potent malaria vaccine is to target the protective CS protein directly to Toll-like receptors (TLRs), such as flagellin and malaria antigens of P. falciparum CS (3D7) protein (FIG. 27). Due to low manufacturing costs and high yields, expression in E. coli has been the most attractive approach to protein production. As described herein, fusion proteins that include flagellin (STF2) and minimal T1BT* epitopes of the CS protein, either as a single copy (STF2.T1BT*-1×) or multiple copies (STF2.T1Bt*-4×), as well as a fusion protein comprised of a truncated flagellin (STF2Δ) conjugated to nearly full length P. falciparum CS protein (STF2Δ-CS) have expressed, purified and immunogenicity assessed (FIG. 27). Immune responses elicited by these constructs have been compared in Balb/c and C57Bl mice, representing genetic backgrounds known to be responder and non-responder to the CS repeats, respectively.

### Construction and Immunogenicity of Flagellin Fusion Protein Containing Minimal T1BT* Epitopes of P. Falciparum CS

One or four copies of the P. falciparum CS protein minimal epitopes T1BT* to the C-terminus of flagellin (STF2; SEQ ID NO: 2) to yield STF2.T1BT*-1× (SEQ ID NO: 10) or STF2.T1BT*-4× (SEQ ID NO: 12) constructs (FIG. 27). The plasmids for each construct was transformed into E. coli BLR DE3, expressed in shake flask cultures, and purified under denaturing conditions using ion exchange chromatographic techniques, as previously developed for other flagellin fusion proteins. Protein was present in soluble as well as insoluble fractions, and was purified from the soluble fraction. The supernatant was denatured prior to purification to prevent degradation. The lysate from the soluble fraction was applied to Q Sepharose and peak fractions were pooled and dialyzed against low pH buffer. Following application onto Source S column, peak fractions were pooled and refolded by rapid dilution. Refolded protein was again applied on Source Q column for further purification and concentration of the protein. This pool was finally applied onto SEC to obtain a pure product. Peak fractions were pooled, sterile filtered, aliquoted and frozen at −80° C. Test for endotoxin was negative (<0.01 EU/ug).

SDS-PAGE and Coomassie staining demonstrated that both the purified fusion protein was monomeric and reacted with monoclonal antibody specific for P. falciparum CS repeats (MAB 2A10) in Western blot. Both STF2-T1BT*-1× (SEQ ID NO: 9) and STF2-T1BT*-4× (SEQ ID NO: 11) constructs reacted with antibodies to flagellin and to CSP when used as antigen in ELISA.

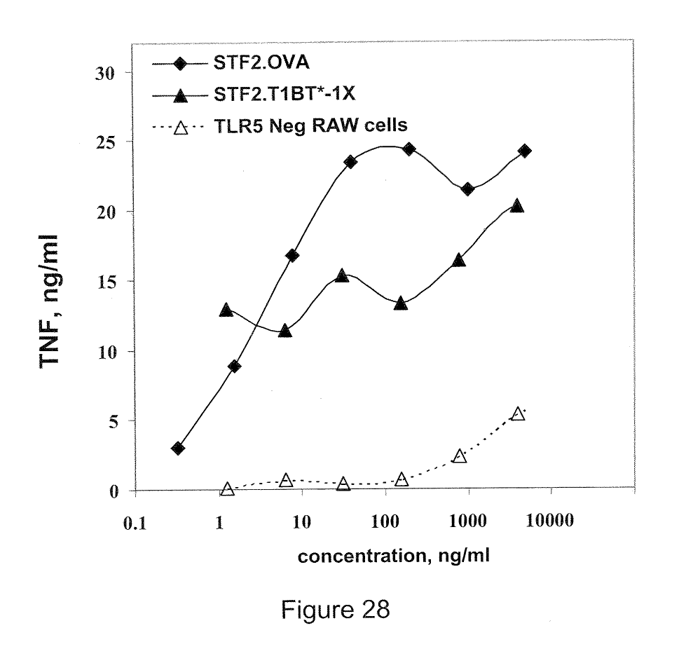

The purified flagellin modified STF2-T1BT*-1× (SEQ ID NO: 9) construct displayed potent TLR5 activity, as measured by production of TNF by RAW cells transfected with human TLR5 (FIG. 28). When stimulated with STF2.T1BT*-1× (SEQ ID NO: 9), the levels of TNFα produced by the hTLR5 transfected cells (closed symbols) were comparable to those elicited by purified STF2.OVA from previous studies (Huleatt, Jacobs et al. 2007). Cytokine production was specific for TLR5 as significant TNFα production was not obtained with STF2.T1BT*-1× (SEQ ID NO: 9) stimulation of untransfected RAW cells (open symbols). Similar results were obtained with STF2. T1BT*-4× (SEQ ID NO: 11). Since RAW cells also express TLR2 and TLR4, the lack of stimulation of untransfected RAW by the purified STF2.T1BT*-1× (SEQ ID NO: 9) or STF2.T1BT*-4× (SEQ ID NO: 11) confirms the absence of LPS contaminants, consistent with endotoxin about <0.1 EU/ug as measured by LAL assay, in the flagellin modified CS constructs.

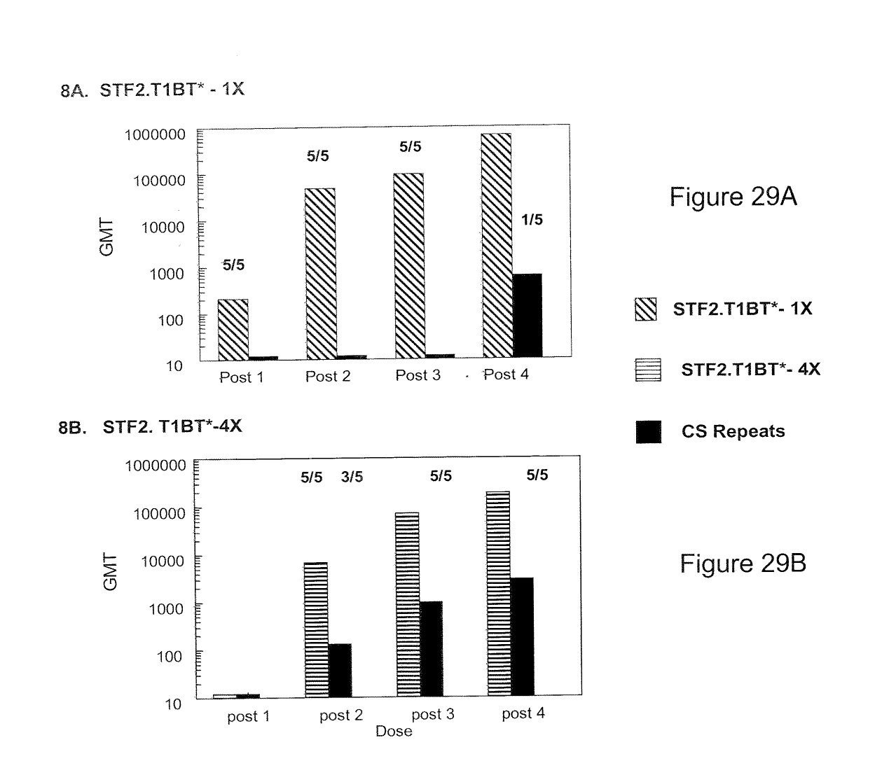

To assess immunogenicity, Balb/c mice were immunized s.c. with four doses of 50 μg STF2.T1BT*-1× (SEQ ID NO: 9) protein. Serum was obtained at 14 days post each immunization and IgG antibody titers to the malaria epitope and the immunogen was determined in individual serum by ELISA (FIG. 29A). Antibody reactive with the STF2.T1BT*-1× (SEQ ID NO: 9) immunogen could be detected after a single dose, with 5/5 mice developing IgG antibody (GMT 211), levels increased with booster immunization, reaching peak IgG titers of 655,360 after the fourth dose. However, antibody was predominantly against the flagellin moiety, as only 1/5 mice had a positive antibody response to the malaria CS repeats (titer 640) following four doses of STF2.T1BT*-1× (SEQ ID NO: 9). Consistent with the absence of anti-CS antibody responses, no malaria specific T cells were detected by IFNγ ELISPOT in spleen cells of mice immunized with STF2.T1BT*-1× (SEQ ID NO: 9).

A second fusion protein containing four copies of the malaria T1BT* epitopes linked to flagellin, STF2.T1BT*-4× (SEQ ID NO: 11), was constructed and immunogenicity tested in a similar manner. Significantly enhanced immunogenicity was observed in BALB/c mice immunized with STF2.T1BT*-4× (SEQ ID NO: 11), as compared to the −1× (SEQ ID NO: 9) construct (FIG. 29B). While only a single BALB/c mouse seroconverted to CS repeats following four immunizations with −1× construct (SEQ ID NO: 9), positive anti-repeat antibodies were observed in 40% (2/5) of the mice after two doses of the STF2.T1BT*-4× construct (SEQ ID NO: 11). A third immunization elicited anti-repeat antibodies in all of the mice (5/5). Peak anti-repeat antibodies GMT 2,941 (range about 1280 to about 20480) were obtained following the fourth dose of STF2-T1BT*-4× (SEQ ID NO: 11). High antibody titers against the immunogen were also obtained in all of the mice (GMT 188,203). A fifth immunization did not significantly increase anti-repeat or anti-immunogen antibody responses.

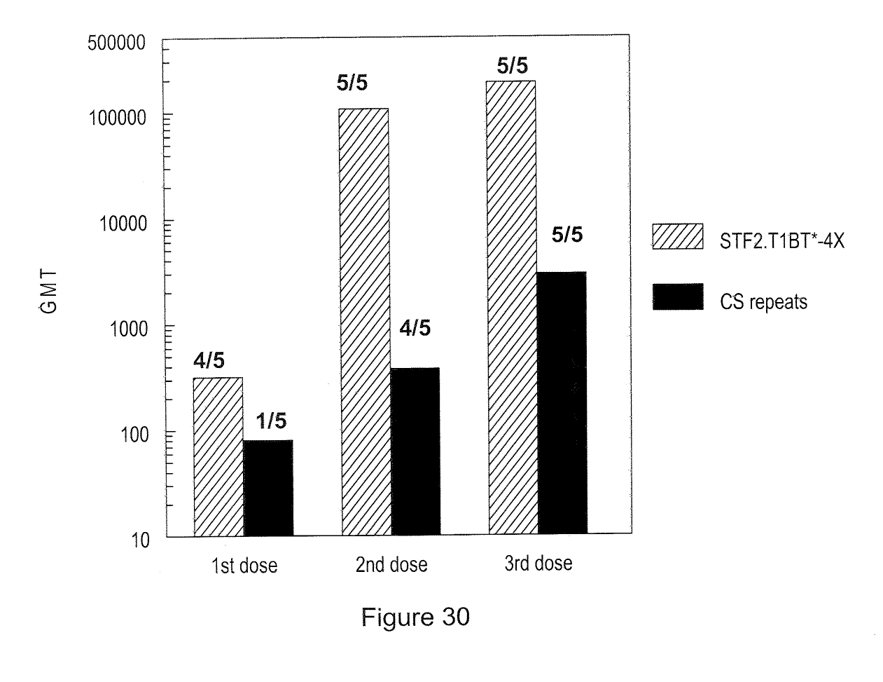

The response to the flagellin modified constructs was not genetically restricted. In C57B1 mice immunized with STF2.T1BT*-4× (SEQ ID NO: 11), the majority (4/5) seroconverted to the immunogen following a single dose of STF2.T1BT*-4× (SEQ ID NO: 11) (FIG. 30). A booster immunization significantly increased response, with anti-immunogen titers increasing to GMT 108,094, with the majority of C57B1 mice (4/5) also having detectable anti-repeat antibodies. A third immunization elicited anti-repeat antibodies in all mice, with GMT 2,941 (range about 1,280 to about 10,240). As found in BALB/c mice, additional immunizations did not increase anti-repeat antibody responses.

Similar levels of anti-repeat antibody responses (GMT 10³) were also observed in C3H/HeJ mice. The results in the C3H/HeJ mice, which lack TLR4, indicate that LPS contaminants are not contributing to immunogenicity of the flagellin constructs, consistent with the low levels of endotoxin detectable in the purified flagellin modified constructs and their inability to stimulate cytokine secretion from RAW cells that express TLR4 and TLR2.

### Construction and Immunogenicity of Flagellin Modified P. Falciparum CS Protein (STF2Δ.CS)

Previous studies of alum adsorbed recombinant CS proteins, expressed in bacteria or yeast, were poorly immunogenic in human volunteers, indicating the need for more potent compositions (Ballou, Hoffman et al. 1987; Herrington, Nardin et al. 1991; Herrington, Losonsky et al. 1992). To determine if increased antibody responses could be obtained by the presence of additional CS repeats and Th epitopes, flagellin-modified fusion protein that contains nearly full length P. falciparum CS protein, STF2Δ.CS (SEQ ID NO: 13) was constructed, expressed and purified. The protein contained the entire repeat region, comprised of 42 repeats of NANP (SEQ ID NO: 36) and 4 NVDP (SEQ ID NO: 227) (NVDPNVDPNVDPNVDP; SEQ ID NO: 196, also referred to herein as “(NVDP)₄”), and lacks only the amino-terminal 13 amino acids containing a putative signal sequence and 23 amino acids of the putative GPI linked carboxy-terminus (Sinnis and Nardin 2002). Multiple CD4+ and CD8+ T cell epitopes have been identified in the C-terminus of the P. falciparum CS protein using cells of naturally infected individuals, rodent malaria models, and predictive algorithms for binding to class I and class II molecules (Sinigaglia, Guttinger et al. 1988; Nardin and Nussenzweig 1993; Doolan, Hoffman et al. 1997; Doolan, Southwood et al. 2000; Reece, Pinder et al. 2004). In naturally infected individuals, protection has been correlated with IFNγ producing CD4+ T cells specific for a highly conserved region of the CS that flanks the C—C pair located proximal to the putative CS transmembrane region (Reece, Pinder et al. 2004). This region contains a second universal T cell epitope identified by predictive algorithm for peptides that bind to multiple class II molecules (Sinigaglia, Guttinger et al. 1988). Alternatively, NVDPNVDPNVDPNVDP (SEQ ID NO: 196; also referred to herein as “(NVDP)₄”) can be employed or NVDPNANP (SEQ ID NO: 197) can be employed. Three of these 8 mer repeats NVDPNANPNVDPNANPNVDPNANP (SEQ ID NO: 198; also referred to herein as “(NVDPNANP)₃”) and is in the 5′ repeat region. NVDPNVDPNVDPNVDP (SEQ ID NO: 199; also referred to herein as “(NVDP)₄”) is not be found in the native CS protein.

To minimize the size of the recombinant fusion protein and to increase protein production yields, the hyper-variable (hinge) region of flagellin (amino acid residues 170-415 of SEQ ID NO: 1) was deleted to generate a flagellin that lacks a hinge region (STF2Δ; SEQ ID NO: 3).

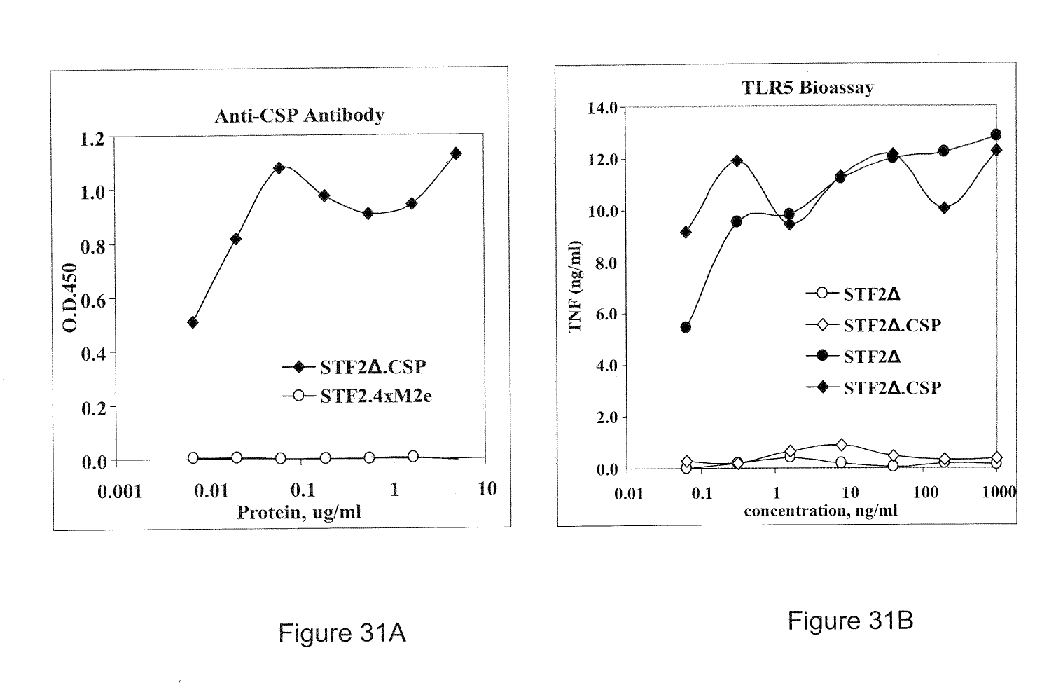

The STF2Δ.CS (SEQ ID NO: 14) construct was expressed in E. coli as inclusion bodies which simplified the purification process. Following extraction of inclusion bodies, column chromatography yield a recombinant STF2Δ.CSP (SEQ ID NO: 13) that was about 95% pure as determined by Western blot. The antigenicity of the malaria epitopes contained in the fusion protein was confirmed by reactivity in ELISA with MAB 2A10, a monoclonal antibody specific for P. falciparum CS repeats (FIG. 31A). Reactivity was specific for the malaria epitope.

Removal of the hinge region did not alter ability of the STF2Δ.CS (SEQ ID NO: 13) to interact with TLR 5 on transfected RAW cells (FIG. 31B). Both STF2Δ.CS (SEQ ID NO: 13), and STF2Δ (SEQ ID NO: 3) without the CS moiety, induced high levels of TNFα when incubated with hTLR5-transfected RAW cells. Cytokine production was specific for TLR5 as no stimulation of untransfected RAW cells was observed. These results demonstrate that nearly full length CS of 233 amino acids can be modified with flagellin without affecting the ability of the agonist to interact with TLR5.

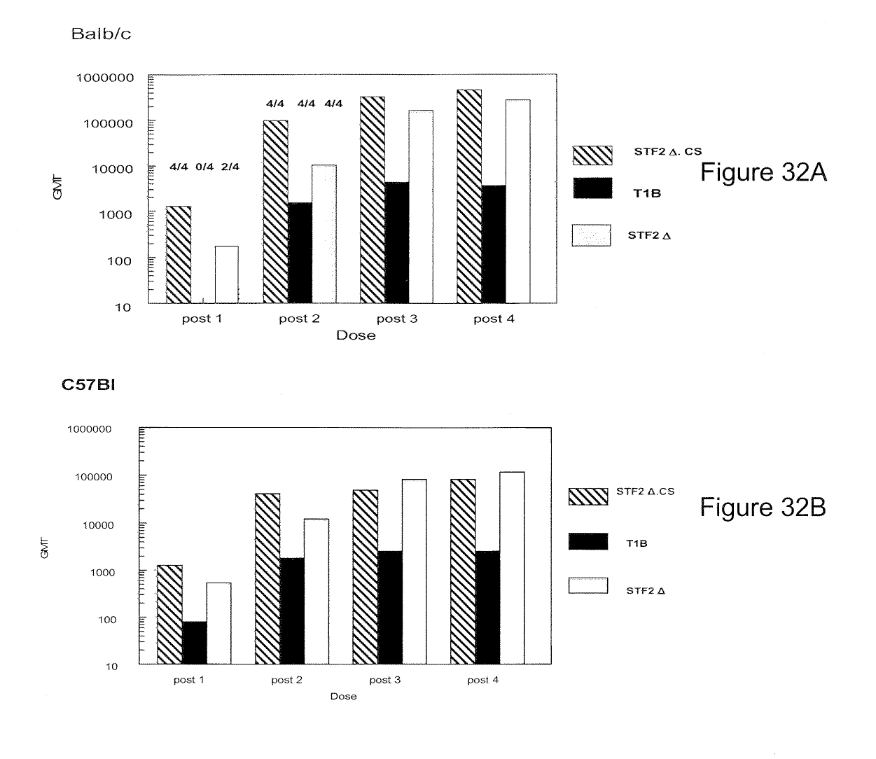

To investigate the impact of inclusion of these additional T and B cell epitopes on the immunogenicity of the flagellin-modified vaccine, C57B1 and Balb/c mice were immunized s.c with STF2Δ.CS (SEQ ID NO: 13) and kinetics of IgG antibody responses determined by ELISA. The flagellin-modified full length CS was found to be of comparable immunogenicity as STF2.T1BT*-4× (SEQ ID NO: 11), with more rapid antibody kinetics following priming. BALB/c mice (4/4) immunized with STF2Δ.CS (SEQ ID NO: 13), developed antibodies specific for the immunogen after a single dose, with GMT=about 1280 (FIG. 32A). Enhanced immunogenicity was also noted in the kinetics of the anti-repeat antibody response. After booster immunization with STFΔ.CSP (SEQ ID NO: 13), all of the BALB/c had positive antibody responses to CS repeats. In contrast only about 60% (3/5) of mice immunized with two doses of STF2. T1BT*-4× (SEQ ID NO: 11) had positive anti-repeat antibody titers. Following a third immunization with STFΔ.CS (SEQ ID NO: 13), the peak GMT for anti-repeat antibodies was about 4,035, with no significant increase following a fourth dose of vaccine.

STFΔ.CS (SEQ ID NO: 13) displayed similar immunogenicity in C57B1 mice, with all of the mice (4/4) developing anti-immunogen antibodies and about 50% (2/4) developing anti-repeat antibodies following a single immunization (FIG. 32B). Booster immunization increased anti-repeat antibody titers to about 2,560, and seroconversion rate to 100% (4/4). As noted also in BALB/c, additional booster immunization did not increase anti-repeat antibody titers further. These data indicate that although more rapid responses could be elicited with the flagellin modified full length CS, the magnitude of the peak anti-repeat antibody responses was comparable to peak titers elicited by the STF2.T1BT*-4× construct (SEQ ID NO: 11) containing minimal T and B cell epitopes.

### Antibodies Elicited by TLR Agonist Malaria Antigen Fusion Protein

A critical determinant of vaccine efficacy is the ability of antibodies elicited by CS subunit vaccines to react with native protein on the viable sporozoite. Serum from the C57Bl mice immunized with STF2.T1BT*-4× (SEQ ID NO: 11) was assessed to determine whether it could recognize native CS protein expressed on viable sporozoites. For these assays, the PfPb sporozoites that express P. falciparum CS repeats in the context of the P. berghei CS protein (Persson, Oliveira et al. 2002) in the that express P. falciparum CS repeats in the context of the P. berghei CS protein (Persson, Oliveira et. Al 2002) were employed in the circumsporozoite precipitin (CSP) assay were employed. The CSP reaction forms on viable sporozoites as a result of antibody cross-linking of CS protein and the shedding of these Ab/Ag complexes by the parasite (Vanderberg, Nussenzweig et al. 1969; Cochrane, Aikawa et al. 1976). CSP reactivity is dependent on the presence of anti-repeat antibody that effectively binds and cross-links the native CS protein. Binding of high concentrations of anti-repeat antibody can immobilize the sporozoite and neutralize infectivity by blocking egress from the skin into the blood capillaries for transit to the liver and/or invasion of host hepatocytes (Stewart, Nawrot et al. 1986; Vanderberg and Frevert 2004).

For the CSP assays, two-fold dilutions of pooled serum obtained prior to and 14 days after each immunization were incubated with PfPb sporozoites for about 45 min at about 37° C. The presence of a terminal CSP reaction on a total of about 20 sporozoites was determined by phase microscopy with the endpoint titer as the final dilution of serum greater than about ≧2+/20 CSP reactions.

The antibodies elicited by immunization with STF2.T1BT*-4× (SEQ ID NO: 11) reacted with CS protein on viable PfPb sporozoites which express P. falciparum CS repeats. Serum obtained after three immunizations with STF2.T1BT*-4× gave a CSP endpoint titer of about 1:16. The response was dependent on dose, as sera obtained following priming or a single booster immunization with STF2.T1BT*-4×, did not give positive CSP reactions. CSP reactivity correlated with anti-repeat antibody titers as measured by ELISA. CSP positive serum obtained post the third immunization had ELISA GMT 2,941, while CSP negative serum obtained following two doses of STF2.T1BT*-4× had GMT 381. Reactions were specific for P. falciparum CS repeats expressed on the transgenic PfPb sporozoites as no reactivity was observed with WT sporozoites expressing P. berghei CS repeats.

Cellular Responses in Mice Immunized s.c. with STF2Δ.CS (SEQ ID NO: 13) or STF2.T1BT*-4× (SEQ ID NO: 11)

The cellular responses in spleen cells of the mice immunized with the flagellin modified CS constructs was examined using ELISPOT assays specific for Th1-type (IFN-γ) or Th2-type (IL-5) cytokines. Cells were analyzed directly ex vivo or following a one week in vitro expansion with malaria peptide, T1BT*. The ex vivo ELISPOT is believed to measure the presence of effector cells, while the in vitro expanded ELISPOT measures memory T cells.

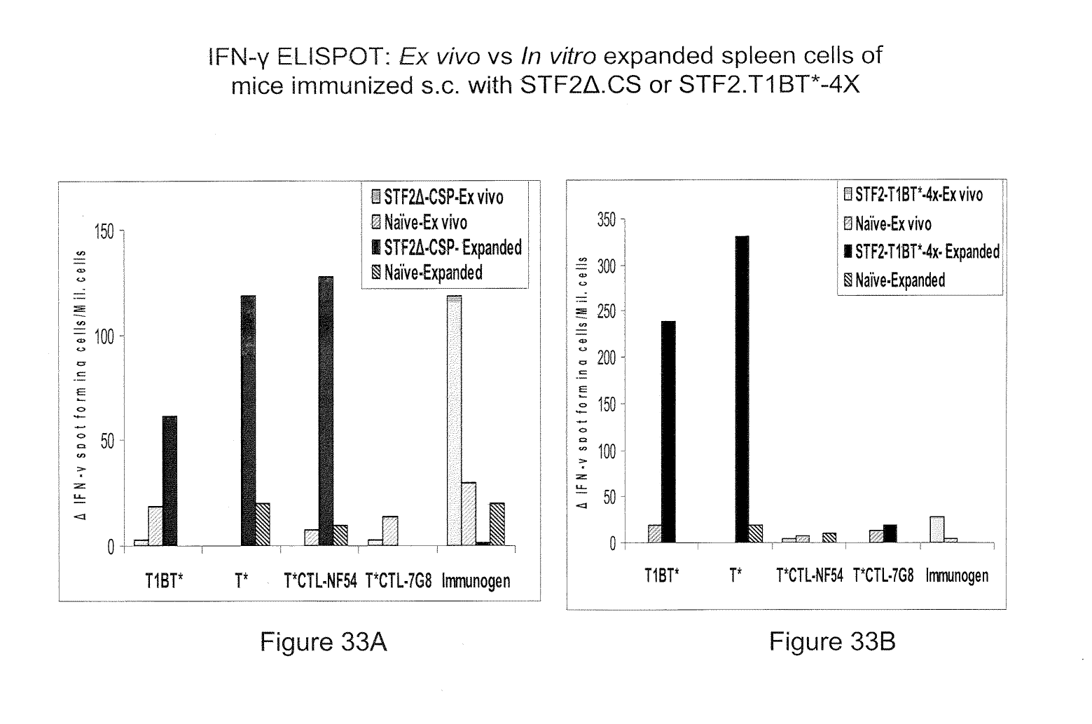

In the IFN-γ ELISPOT, spleen cells from mice immunized with either STF2Δ.CS (SEQ ID NO: 13) (FIG. 33A) or STF2.T1BT*-4× (SEQ ID NO: 11) (FIG. 33B) when tested directly ex vivo revealed predominantly immunogen specific T cell responses. Positive IFN-γ SFC were detected following stimulation with immunogen or flagellin (light bars) with minimal responses to the malaria peptides T1BT* (SEQ ID NO: 147), T* (SEQ ID NO: 34) or the 9 mer CTL epitope from either the NF54 (YLNKIQNSL (SEQ ID NO: 228)) or 7G8 (YLKKIKNSL (SEQ ID NO: 229)) strain. However, following 7 days expansion in vitro with the malaria T1BT* peptide, positive malaria-specific responses could be detected in cells of mice immunized with either the STF2Δ.CS (SEQ ID NO: 13) (FIG. 33A) or STF2.T1BT*-4× (SEQ ID NO: 11) (FIG. 33B) (dark bars). The magnitude and fine specificity of IFN-γ producing T cell responses varied depending on the immunogen. Mice immunized with STF2Δ.CS (SEQ ID NO: 13) had cells specific for T1BT*, T* and the 9 mer T*-CTL peptide, while mice immunized with STF2.T1BT*-4× (SEQ ID NO: 11) had higher levels of SFC to T1BT* and T* but no response to the T*-CTL peptide. Responses were malaria-specific as minimal IFN-γ SFC were detected in spleen cells from naïve mice (hatched bars).

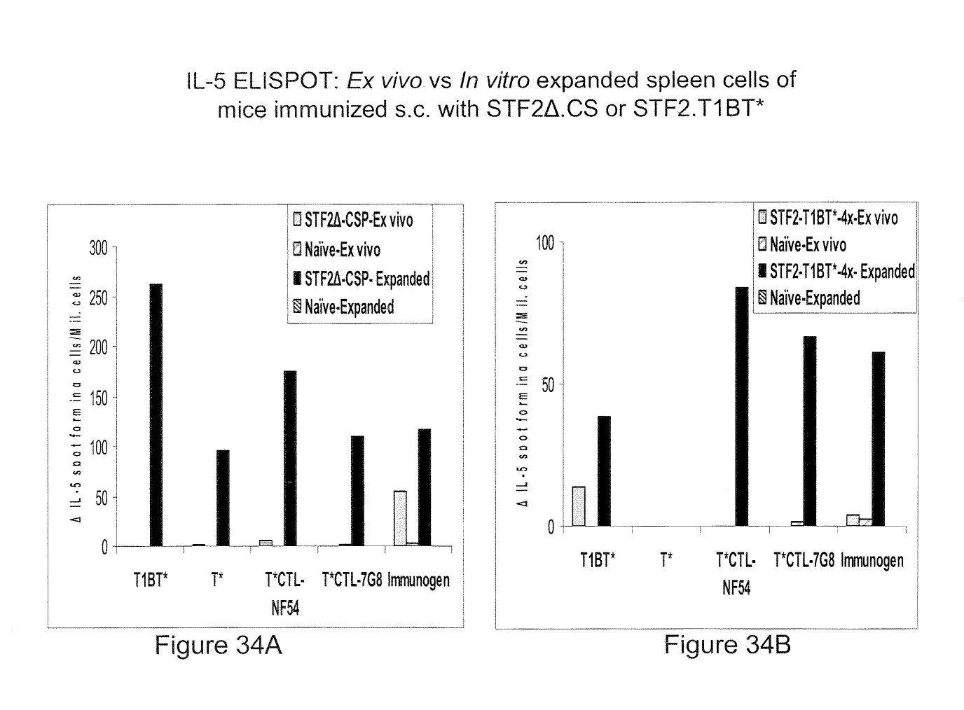

Similar results were obtained when spleen cells were analyzed in IL-5 ELISPOT assay (FIGS. 60A and 60B). As found with IFN-γ production, malaria specific IL-5 SFC were detected only in the in vitro expanded ELISPOT. The T cells in both the STF2Δ.CS (SEQ ID NO: 13) (FIG. 34A) and the STF2.T1BT*-4× (SEQ ID NO: 11) (FIG. 34B) immunized mice recognized primarily the T1BT* and T*CTL epitope, with lower levels of IL-5 SFC elicited by stimulation with the T* peptide. The number of IL-5 SFC in mice immunized with the STF2Δ.CS (SEQ ID NO: 13) was higher than in mice immunized with STF2-T1BT*-4× (SEQ ID NO: 11). In contrast, in the IFN-γ ELISPOT, higher numbers of IFN-γ SFC were obtained with the STF2-T1BT*-4×fusion protein (also referred to herein as “construct”) (SEQ ID NO: 11).

The results of the T cell cytokine assays are consistent with the IgG subtypes detected in the serum of mice immunized with the flagellin modified CS constructs. In all strains of mice tested (C57Bl, Balb/c, C3H), the predominant IgG subtype was IgG1, consistent with the IL-5 Th2-type cytokine responses detected in the ELISPOT. The flagellin modified constructs also elicited IgG2 antibodies, although at lower levels, consistent with the mixed Th1/Th2 cytokine responses measured in the ELISPOT assay.

### Intranasal Immunization

Vaccines that can be administered without injection, such as by oral, nasal or skin applications, can have advantages, such as increased patient compliance, an important factor in the pediatric population that is the target of malaria vaccines.

Mucosal and systemic immune systems are interconnected and oral or intranasal immunization can protect against a number of non-mucosal pathogens (Levine 2003). The potential of mucosal immunity for protection against malaria sporozoites was first shown following oral immunization with a recombinant Salmonella typhi vaccines expressing P. berghei CS protein which elicited CD8+ T cell mediated cellular protection in mice (Sadoff, Ballou et al. 1988; Aggarwal, Kumar et al. 1990).

In contrast to mucosal adjuvants based on ADP-ribosylating exotoxins, flagellin targets a TLR receptor on APCs that has evolved to detect bacterial PAMP and initiate immune responses (Medzhitov 2001; Means, Hayashi et al. 2003). The TLR5 agonist flagellin employed in the fusion proteins described herein can be derived from Salmonella typhmurium, a mucosal pathogen that targets intestinal cells. The innate immune system has evolved to respond to PAMP of pathogenic bacteria such as Salmonella through specific recognition by TLR5 expressed on mucosal cells.

While malaria is a blood-borne pathogen, the potential of mucosally administered malaria vaccines to protect against sporozoites challenge has been demonstrated in previous murine studies using an oral vaccine comprised of attenuated S. typhi (Ty21A) engineered to express CS antigens (Sadoff, Ballou et al. 1988). Mice immunized orally with these chimeric bacteria developed CD8+ T cell mediated protective immunity against sporozoite challenge (Aggarwal, Kumar et al. 1990). However, in Phase I clinical trials, two oral doses of Salmonella typhi expressing P. falciparum CS was poorly immunogenic in humans, with anti-sporozoite antibody or CS specific CD8+ CTL detectable in only 10% of the volunteers (Gonzalez, Hone et al. 1994; Sztein, Wasserman et al. 1994).

Intranasal Immunization with Flagellin/Malaria Antigen Fusion Proteins

Low Dose (10 μg) Intranasal Immunization

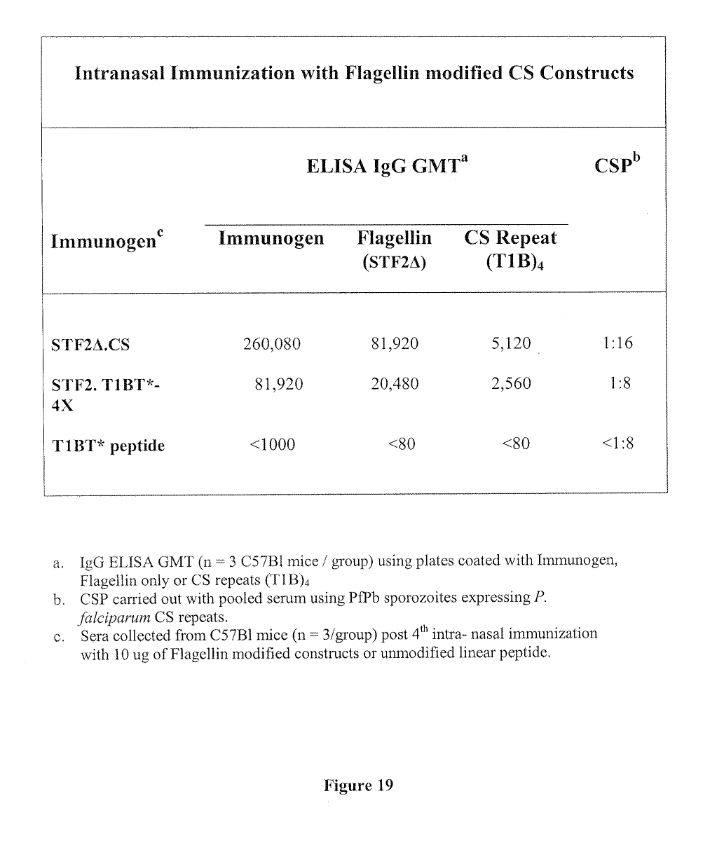

To explore immunogenicity of flagellin-modified CS constructs as a needle-free composition for use in methods of preventing or treating malaria (e.g., vaccines), C57BI mice were immunized intranasally with 10 μg of STF2.T1BT*-4× (SEQ ID NO: 11) or STFΔ.CS (SEQ ID NO: 13). As control, mice were immunized intranasally with unmodified T1BT* (SEQ ID NO: 147) peptide without flagellin, in PBS. The kinetics of antibody response was delayed in intranasal immunized mice when compared to mice immunized s.c., however following the fourth dose of either STF2.T1BT*-4×(SEQ ID NO: 11) or STFΔ.CS (SEQ ID NO: 13), anti-repeat IgG in the mice immunized intranasally reached titers comparable to those observed in mice immunized subcutaneously (FIG. 19). Importantly, these antibodies were also reactive with viable sporozoites expressing P. falciparum CS repeats, as demonstrated by the positive CSP reactions (titers about 1:8 to about 1:16) in the sera of the intranasally immunized mice. As noted also with parenterally immunized mice, titers to immunogen and flagellin were about 1 to about 2 logs higher than those to CS epitope. Consistent with induction of mucosal immunity, sera from the intranasally immunized mice, also had detectable IgA antibodies to the immunogen.

The induction of responses in the intranasally immunized mice was dependent on the presence of the flagellin TLR5 agonist. Mice immunized intranasally with the T1BT* peptide alone did not develop detectable IgG antibodies to CS repeats.

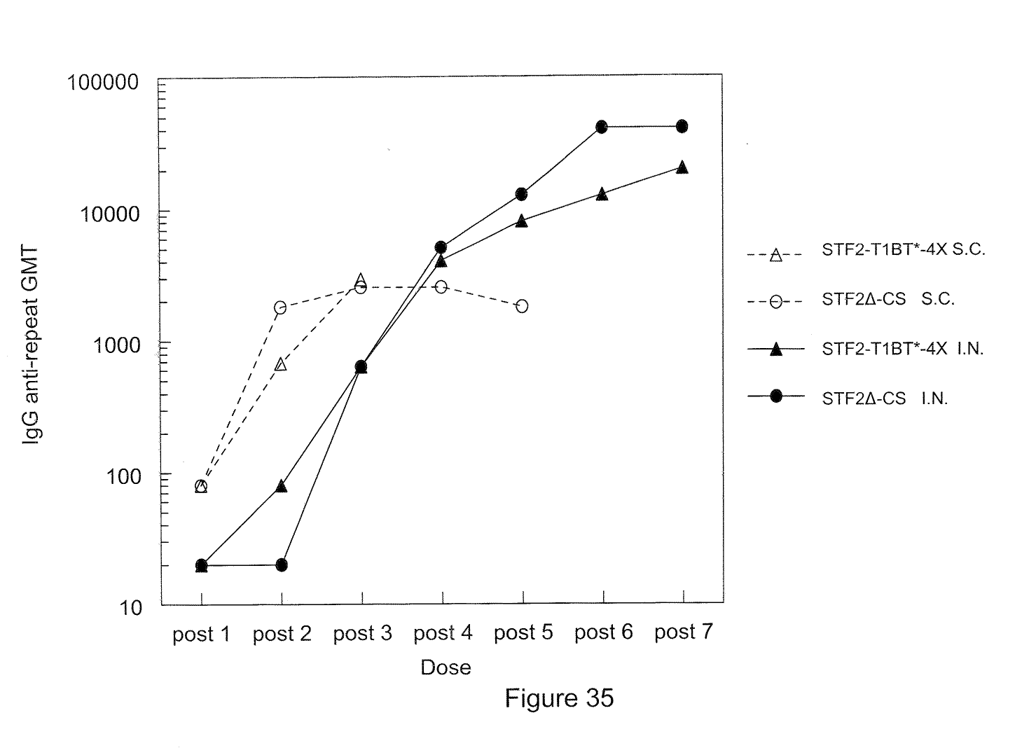

With additional intranasal booster immunizations, the titers of anti-repeat antibodies continued to increase, reaching a peak of 10⁴ GMT following seven doses of either STF2.T1BT*-4× (SEQ ID NO: 11) or STFΔ.CS (SEQ ID NO: 13) (FIG. 35). The IgG subtypes of the anti-repeat antibodies in the serum of the intranasally immunized mice were consistent with those observed following s.c immunization. There was a predominance of IgG1 antibodies, with lower levels of IgG2, in both groups of immunized mice.

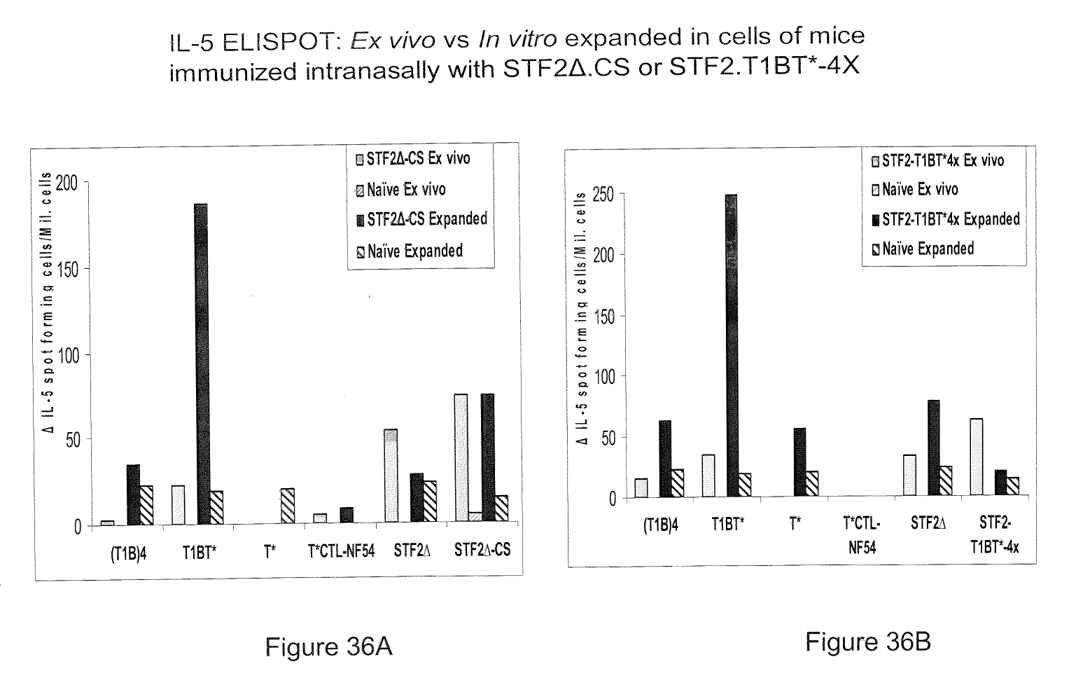

Consistent with results obtained with s.c. immunization, the mice immunized intranasally with either STFΔ.CS (SEQ ID NO: 13) (FIG. 36A) or STF2.T1BT*-4×(SEQ ID NO: 11) (FIG. 36B) had detectable malaria specific IL-5 positive T cell responses in the expanded IL-5 ELISPOT. The predominant response in both groups of mice was to the T1BT* peptide (SEQ ID NO: 147). In contrast to s.c. immunization (FIGS. 60A and 60B), there were no detectable IL-5 SFC following stimulation with the T*-CTL peptide of the spleen cells from the mice immunized intranasally (FIGS. 62A and 62B).

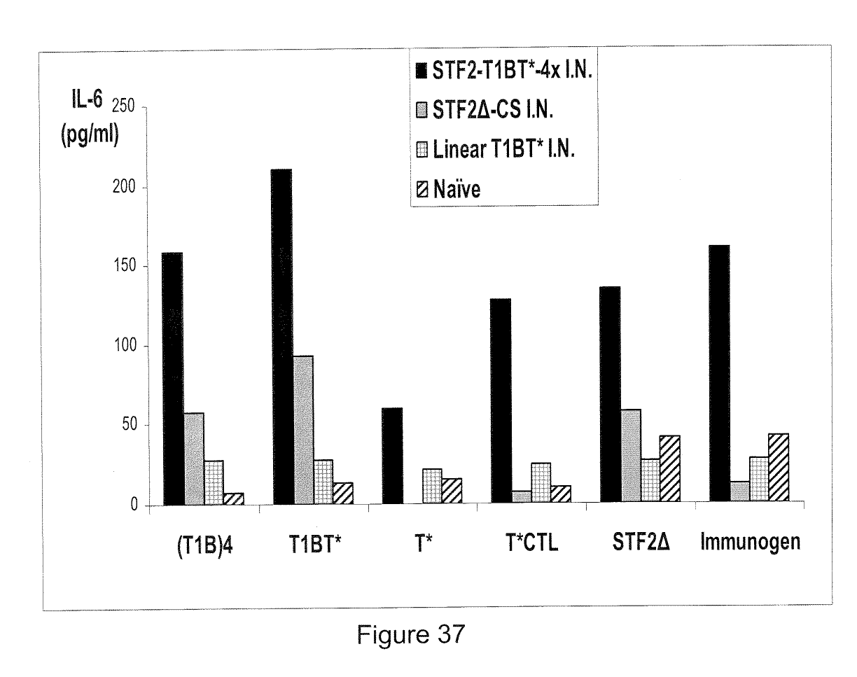

Measurement of Th2 cytokines in the supernatant of these cells was carried out using the Cytokine Bead Assay (BD) and flow cytometry. Consistent with the presence of Th2-type IL-5 SFC, supernatants of the expanded cell cultures also had detectable levels of IL-6. The highest levels of IL-6 were obtained following stimulation with the malaria peptides, as well as flagellin, in spleen cells from the mice immunized intranasally with STF2-T1BT*-4× (SEQ ID NO: 11) (FIG. 37). Cells of mice immunized intranasally with STFΔ.CS (SEQ ID NO: 13) produced IL-6 when stimulated only with CS repeats and T1BT* peptides (SEQ ID NO: 147). Consistent with the absence of antibody responses, mice immunized intranasally with unmodified linear T1BT* peptide (SEQ ID NO: 147) had low levels of IL-6 comparable to naïve mice (hatched bars).

To determine if the enhanced antibody responses elicited by intranasal immunization had sporozoite neutralizing activity, in vitro Transgenic sporozoite Neutralization Assay (TSNA) using the transgenic PfPb sporozoites that express P. falciparum CS repeats (Kumar, Oliveira et al. 2004) was performed. For this assay, immune or normal serum (1:5 dilution) was incubated with 5×10⁴ PfPb sporozoites for about 45 minutes prior to addition to confluent cultures of human (HepG2) hepatoma cells (Kumar, Oliveira et al. 2004; Calvo-Calle, Oliveira et al. 2006). After about 48 hours incubation at about 37° C., the number of intracellular liver stage parasites was determined by lysing the wells and measuring levels of parasite 18S ribosomal RNA by realtime-PCR, as previously described (Kumar, Oliveira et al. 2004). Percent inhibition was measured based on the number of rRNA copies in cultures receiving sporozoites pre-incubated in immune serum as compared to cultures receiving normal serum, with about >90% inhibition considered significant.

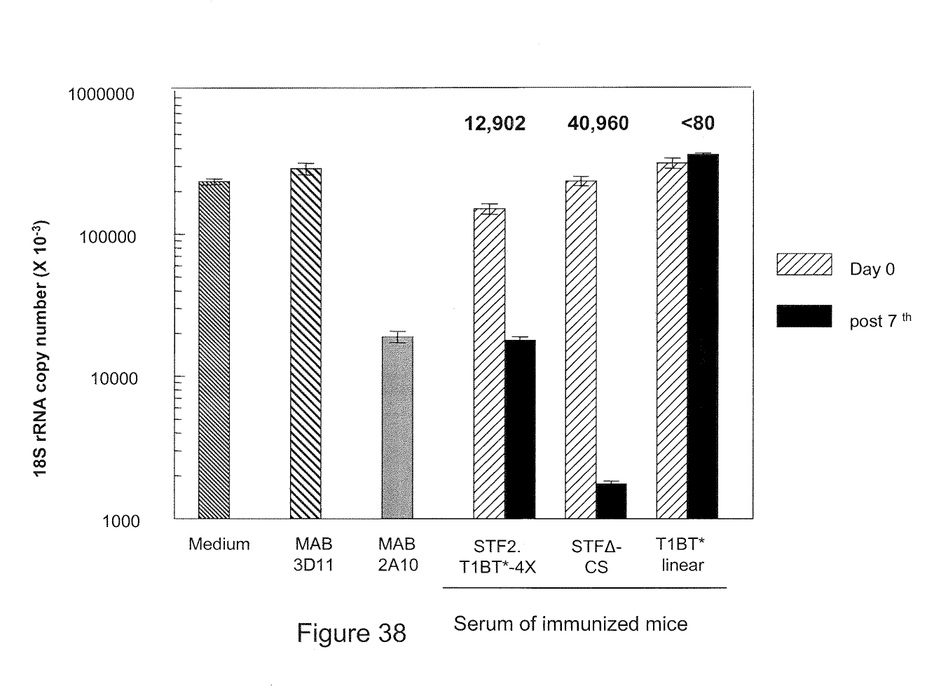

Sera was obtained prior to immunization (Day 0) and following immunization with seven i.n doses of either STF2.T1BT*-4× (SEQ ID NO: 11), STFΔ.CS (SEQ ID NO: 13) or unmodified T1BT* peptide (SEQ ID NO: 147) without flagellin Inhibitory activity was compared with that obtained with about 25 μg of MAB 2A10, a protective antibody specific for P. falciparum CS repeats. Negative control included equal amount of MAB 3D11, specific for P. berghei CS repeats. Significant sporozoite neutralizing activity was observed in the immune serum as compared to pre-immune serum (FIG. 38). Greater than about 90% of sporozoites were inhibited by serum from the mice immunized with either STF2.T1BT*-4× (SEQ ID NO: 11) or STFΔ.CS (SEQ ID NO: 13), when compared with the parasite 18S rRNA levels in cultures receiving sporozoites in pre-immune serum or without serum (medium control). The level of inhibition was comparable to that obtained with about 25 μg of MAB 2A10. Inhibition was specific for P. falciparum CS repeats, as MAB 3D11 specific for P. berghei repeats was not inhibitory. Sporozoite neutralizing activity correlated with anti-repeat antibody titer, as serum of mice immunized i.n. with the unmodified T1BT* peptide (SEQ ID NO: 147) did not have detectable anti-repeat antibodies and did not have any sporozoite neutralizing activity.

High Dose (50 μg) Intranasal Immunization

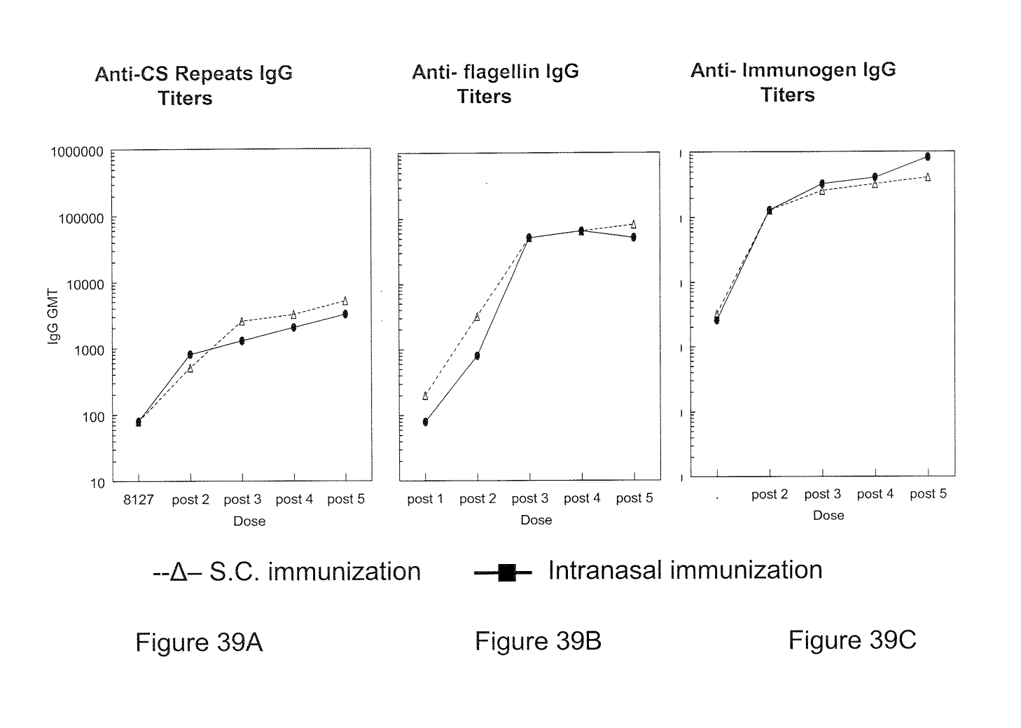

To determine if immunogenicity of intranasal immunization could be increased using higher doses, mice were immunized intranasally (IN) with about 50 μg of STFΔ.CS (SEQ ID NO: 13) and antibody responses compared with the same dose administered subcutaneously. While intranasal immunization with low dose (about 10 μg) required at least two booster immunizations to obtain anti-immunogen antibodies, a single dose of about 50 μg STFΔ.CS (SEQ ID NO: 13) elicited positive responses to the immunogen in all of the mice. Malaria specific antibodies were detected in all of the intranasally immunized mice following a booster immunization, as found also with s.c. immunization. The kinetics of the IgG antibody response to the malaria CS repeats (FIG. 39A), to flagellin (FIG. 39B) and to the immunogen STFΔ.CS (SEQ ID NO: 13) (FIG. 39C) were similar in mice immunized intranasally or s.c. with about 50 μg dose. Post the fifth dose, peak antibodies to CS repeats were about 10³ (range about 5,120 to about 3,225), while the anti-flagellin responses were a log higher (about 5 to about 8×10⁴). The antibody response to the immunogen was highest, with peak antibody titers of about 10⁵ (about 4 to about 8×10⁵).

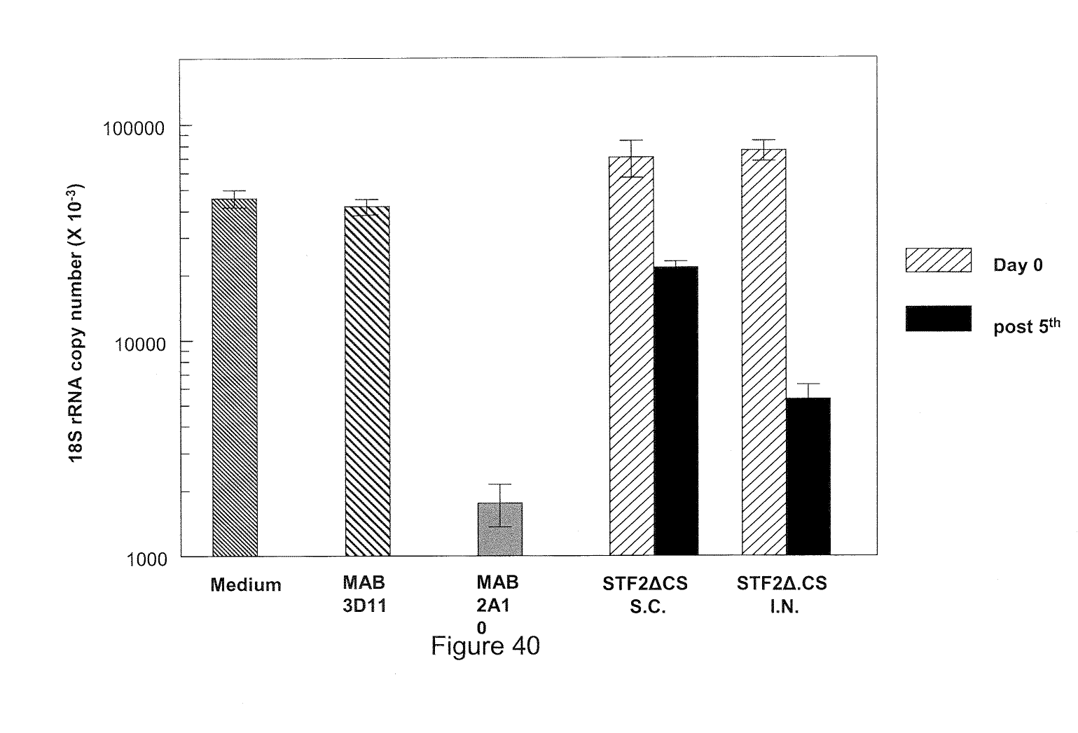

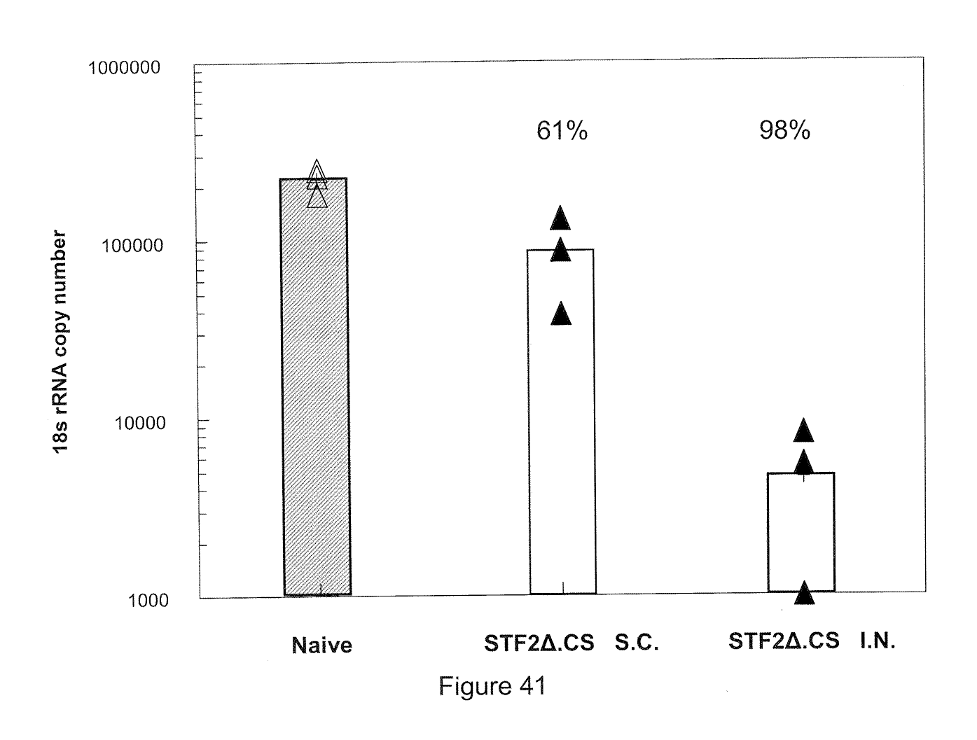

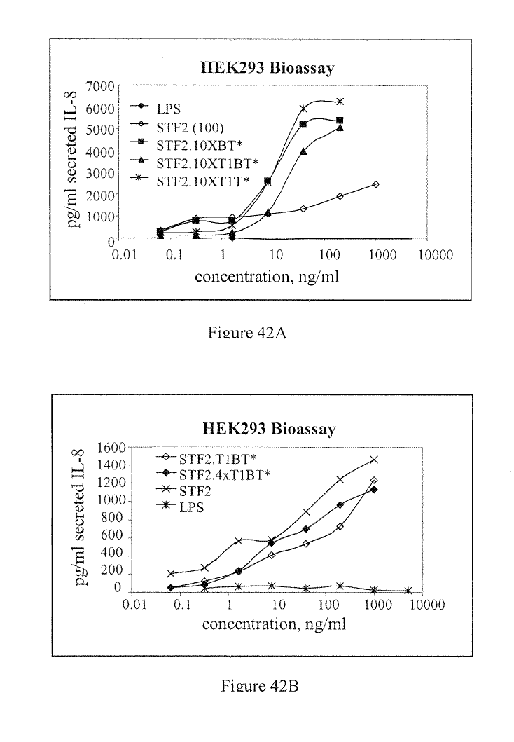

Significant sporozoite neutralizing activity was demonstrated in the sera of the mice immunized intranasally with about 50 μg STFΔ-CS (SEQ ID NO: 13) (FIG. 40). The level of parasite rRNA was reduced about 93% in hepatoma cells inoculated with PfPb sporozoites incubated in serum from mice immunized intranasally when compared to levels in cultures receiving sporozoites in pre-immune serum. The levels of inhibition in cultures receiving immune serum from mice immunized s.c. with about 50 μg STFΔ-CS (SEQ ID NO: 13) had lower levels of inhibition (about 69%). The levels of inhibition obtained with the intranasal immune serum was comparable to that obtained with about 25 μg of monoclonal antibody 2A10 specific for P. falciparum CS (about 96%). Inhibition was specific for P. falciparum CS repeats, as MAB 3D11, which is specific for P. berghei CS repeats, did not inhibit sporozoite infectivity. To demonstrate the relevance of the in vitro sporozoite neutralizing activity to in vivo protection, the mice immunized i.n. or s.c. with about 50 μg STFΔ-CS (SEQ ID NO: 13) were challenged by exposure to the bites of PfPb infected mosquitoes (FIG. 41). There was significant protection against liver stage parasites in the mice immunized intranasally with about 50 μg STFΔ-CS (SEQ ID NO: 13). Levels of hepatic stage parasites in these mice were reduced about 98% when compared to naïve mice (hatched bar). The mice immunized s.c had lower levels of protection, with liver stage burden reduced only about 61% when compared to naives, consistent with the lower levels of protection noted in vitro. These findings indicate that intranasal immunization can provide a new route for induction of protective immunity against sporozoites.

### CONCLUSION

Fusion proteins that include TLR agonists, such as flagellin, and malaria antigens, such as portion of a CSP (e.g., T-cell epitopes and B-cell epitopes) were immunogenic when administered either s.c. or i.n. The anti-P. falciparum CS repeat antibodies elicited by STF2-T1BT*-4× (SEQ ID NO: 11) and STFΔ.CS (SEQ ID NO: 13) reacted with viable transgenic sporozoites expressing P. falciparum CS repeats and with air dried P. falciparum sporozoites by indirect immunofluorescence, indicating that the antibodies recognize the protective repeat epitope in the context of native CS protein on the sporozoite surface. In addition, the mice immunized with the flagellin-modified constructs developed malaria-specific T cells secreting Th1 and Th2 type cytokines, consistent with the mixed IgG1 and IgG2 subtypes of anti-repeat antibodies detected in the serum. The intranasally administered fusion protein of the invention elicited systemic IgG malaria responses comparable to those obtained following subcutaneous immunization. The immune sera elicited by intranasal immunization with flagellin modified CS constructs was biologically functional and neutralized sporozoite infectivity in vitro. In addition, the in vitro sporozoite neutralizing activity of serum from the intranasally immunized mice directly correlated with resistance to sporozoite challenge in vivo, supporting the potential of fusion proteins of the invention as a composition to prevent or treat malaria in, for example, needle-free malaria vaccines.

### Materials and Methods

Assays of Malaria Specific Antibody Responses

Individual mice were bled after immunization and sera stored at about −70° C. until used for serologic assays. Antibody titers, fine specificity and biological function against viable sporozoites expressing P. falciparum CS repeats (PfPb transgenic parasites) were measured as defined below

Measurement of Antibody Kinetics and Fine Specificity

The presence of IgG antibodies against the immunogen, the CS repeat peptide, and STF2 flagellin was measured by ELISA and results expressed as geometric mean titer (GMT). The endpoint cutoff was an OD greater than the mean±3 SD obtained with day 0 sera. Reactivity of antibodies with P. falciparum sporozoites was assayed by indirect immunofluorescence (IFA) using air dried P. falciparum sporozoites. Anti-repeat ELISA titers strongly correlate with IFA titers (Herrington, Clyde et al. 1990; Nardin, Oliveira et al. 2000).

Flagellin is known to stimulate proinflammatory cytokines and Th1 responses through interaction with TLR5 on antigen presenting cells. Th1 T cells can provide γ-IFNγ which functions as a Th factor for differentiation of B cells for IgG2a antibody, as well as functioning as an inhibitory cytokine for intracellular liver stage parasites. Serum obtained following final immunization with STF2 modified CS constructs was assayed for IgG1, IgG2a/c, IgG2b, IgG3 subtypes (Southern Biotech) using ELISA plates coated with (T1B)₄ peptide.

CSP Reactivity with Viable Sporozoites

The ability of antibodies raised by immunization with flagellin modified CS to cross react with CS protein expressed on the surface of viable sporozoites was tested by CSP reaction using PfPb that express P. falciparum CS repeats. Sporozoites freshly dissected from salivary glands of PfPb infected mosquitoes were reacted with two fold dilutions of normal or immune serum. After incubation at about 37° C. for about 45 min, the number of CSP positive sporozoites was determined by phase microscopy, counting a total of twenty sporozoites for each sample dilution. Endpoint titer was the final dilution giving positive CSP on a minimum of about 2/20 sporozoites.

Sporozoite Neutralizing Assay

P. falciparum sporozoites are highly infectious only for humans, and invade but fail to develop within HepG2 cell lines in vitro. The transgenic PfPb rodent parasite expressing P. falciparum CS repeats is fully infective to hepatoma cells in vitro and to mice in vivo (Persson, Oliveira et al. 2002). However, the PfPb are antigenically P. falciparum, since they express the immunodominant P. falciparum repeat region. Thus, they provide a small rodent model to measure the inhibitory activity of vaccine induced anti-P. falciparum CS repeat specific responses. The PfPb sporozoites were used to assess the in vitro neutralizing activity of antibodies elicited by the flagellin modified CS vaccine constructs.

The Transgeneic Sporozoite Neutralization Assays (T-SNA) was carried out as described in (Kumar, Oliveira et al. 2004). For this assay, immune or normal serum (about 1:5 dilution) was incubated with about 5×10⁴ PfPb sporozoites for about 45 minutes prior to addition to confluent cultures of human (HepG2) hepatoma cells (Kumar, Oliveira et al. 2004; Calvo-Calle, Oliveira et al. 2006). Controls include sporozoites incubated with species specific anti-P. falciparum MAB 2A10 and, as negative controls, sporozoites incubated with anti-P. berghei MAB 3D11 or normal pre-immune sera. After about 48 hours incubation at 37° C., the number of EEF was determined by lysing the wells and measuring levels of parasite 18S ribosomal RNA by realtime-PCR, as previously described (Kumar, Oliveira et al. 2004). Total RNA (about 1 μg) from cultures was reverse-transcribed to cDNA using a PTC-100 Programmable Themal Controller (MJ Research Inc). An aliquot was used for real-time PCR amplification using a Rotor-Gene RG-3000 (Corbett Research Inc.) and primers specific for P. berghei 18S rRNA (Chomczynski and Sacchi 1987; Bruna-Romero, Gonzalez-Aseguinolaza et al. 2001). The product generated by PCR was detected using dsDNA-specific dye SYBR Green, using SYBR Green, dNTPs and Amplitaq Gold DNA polymerase mixture prepared per manufacturer's instructions (PE Applied Biosystems). Results were expressed as number of copies of parasite rRNA based on an 18S rRNA plasmid reference standard. Percent inhibition was measured based on the number of rRNA copies in cultures receiving sporozoites pre-incubated in immune serum as compared to cultures receiving normal serum. Serum giving about >90% inhibition of parasite infectivity was considered to have significant sporozoite neutralizing activity.

### Assays of Malaria Specific CD4+ and CD8+ Cellular Responses

About seven to about ten days following the final immunization with fusion proteins that includes at least a portion of a TLR agonist (flagellin) and a malaria antigen (e.g., CSP, such as T1, T*), mice were sacrificed and spleen cells collected for cellular assays. Whole spleen cells and CD4+ and CD8+ T cell populations, isolated by negative selection using magnetic beads coated with anti-CD4 or anti-CD8 antibodies (MACS; Miltenyi Biotec, CA), were tested to determine the role of T cell populations in the immune response.

ELISPOT

Malaria-specific T cells were quantified using IL5- or IFNγ-ELISPOT kits (R&D Biosciences, San Jose, Calif.) as described in our prior studies (Calvo-Calle, Oliveira et al. 2006). Whole spleen, or purified CD4+ or CD8+ T cell subpopulations, were immediately tested in the ELISPOT assay (ex vivo ELISPOT assay) and additional cells were expanded for seven days in vitro in the presence of the T1BT* peptide (SEQ ID NO: 147) (about 10 μg/ml) for the in vitro expanded ELISPOT assay.

For the ELISPOT assay, about 4×10⁵ cells were co-incubated with APCs pulsed with flagellin modified CS proteins, flagellin only or malaria peptides. The malaria peptides tested included T1BT* (SEQ ID NO: 147), (T1B)₄ repeat peptide, DPNANPNVDPNANPNVNANPNANPNANP (SEQ ID NO: 230) the 20 mer peptide representing the universal T* epitope (SEQ ID NO: 34) and a 9 mer CTL epitope contained therein from the NF54 strain (SEQ ID NO: 228) or the 7G8 strain (SEQ ID NO: 229) equivalent. Cells were plated in triplicate wells of a 96-well nitrocellulose plate (Millipore) coated with anti-IFNγ or anti-IL-5 antibody. Cells stimulated with ionomycin+PMA were included as positive controls. After about 16-24 hrs, plates were washed and incubated overnight with biotinylated anti-IFNγ or anti-IL-5 MAB followed by incubation with streptavidin conjugated alkaline phosphatase, per the manufacturers protocol (R&D Biosciences, San Jose, Calif.). The presence of cytokine-secreting cells was revealed by adding BCIP/NBT as substrate. The number of spot-forming-cells (SFC) in triplicate wells were counted by an ImmunoSpot Analyzer (CTL Cleveland, Ohio) and results expressed as mean number of SPC/10⁶ cells+/−SEM

Th1/Th2 Cytokine Assays

Flagellin interaction with TLR5 is known to stimulate Th2 responses as well as proinflammatory cytokine production by APCs that enhance Th1 responses. Th1-type CD4⁺ T cells, as well as CD8+ T cells, can secrete IFNγ which is a potent inhibitor of hepatic stage parasites (Ferreira, Schofield et al. 1986; Schofield, Ferreira et al. 1987). Spleen cells and purified CD4+ and CD8+ T cells (Miltenyi Biotec, CA) were incubated with target cells pulsed with ten-fold dilutions of flagellin, recombinant CS protein or malaria peptides, as above. The Th1-type (IL-2, IFN-γ, TNFα) and Th2-type (IL-5, IL-6, IL-10) cytokine profiles were measured in cell culture supernatants using Cytokine Bead Assay (CBA) kits (Becton-Dickenson) and flow cytometry, as previously described (Calvo-Calle, Oliveira et al. 2005). Controls included splenocytes from age-matched naive mice and mice immunized with peptide or protein without TLR agonist as negative controls.

Protective Efficacy Against Sporozoite Challenge

Flagellin modified CS constructs that elicit high levels of anti-repeat antibodies that neutralize sporozoite infectivity in vitro, were tested for protective efficacy in vivo by exposing immunized mice to the bites of mosquitoes infected with PfPb transgenic rodent malaria sporozoites (Zavala, Gwadz et al. 1982; Persson, Oliveira et al. 2002; Calvo-Calle, Oliveira et al. 2006). Prior to challenge, the level of sporozoite infection in the mosquito salivary gland was determined using a two-site assay based on MAB to P. falciparum CS repeats for PfPb (Nardin, 1982; Zavala et al. 1982) or by microscopy, and the number of mosquitoes adjusted to ensure that all mice receive 5-15 infected bites. Protection was determined by the measurement of liver stages at about 40 hrs post challenge by real-time PCR, as described above. This assay provides a rapid, sensitive and quantitative measurement of parasite levels in the liver.

In future studies, vaccine formulations that elicit immunity that results in about >90% inhibition of hepatic stages following sporozoite challenge, as measured by RT-PCR, will be tested for ability to elicit sterile immunity, that is the complete absence of blood stage parasites following challenge. Giemsa stained blood smears will be taken day 3-14 post challenge. Sterile immunity will be defined as total absence of parasitemia at about day 14. The prepatent period will also be determined in mice that become infected to assay whether there is a significantly delayed time to patent infection as compared to naïve mice. While sterile immunity is the more rigorous challenge, it is not quantitative and unless 100% of the infectious sporozoite inoculum is totally neutralized a patent infection will develop. Therefore, only those constructs that elicit significant (about 90%) inhibition, as measured by real-time PCR of liver stages following challenge, will be tested in additional cohorts to determine if sterile immunity is elicited.

The mechanisms of immune resistance in the mice immunized with flagellin-modified CS vaccine will be determined by depleting CD4+ or CD8+ T cells prior to challenge with PfPb infected mosquitoes. Mice will be treated by i.p injection of 200 μg of MAB GK1.5 (ATCC) or MAB 2.43 (ATCC), respectively, for three consecutive days prior to challenge, as in our previous studies (Calvo-Calle, Oliveira et al. 2006). Depletion of the T cell population will be confirmed by FACS analysis using a FACSCalibur™/CELLQuest™-(Becton Dickinson).

To confirm the role of antibodies in protection of the immunized mice, passive transfer experiments will be carried out using sera of protected mice. A total of about 0.4 ml of pooled serum from protected mice, or from naïve animals or adjuvant (flagellin only) controls, will be injected into naïve mice one hour prior to exposure to the bites of PfPb infected mosquitoes. The levels of parasite rRNA in the liver at 40 hours post infection will be measured by RT-PCR, as above. These studies will allow the determination of the functional activity of anti-repeat antibodies elicited by the different flagellin modified vaccine constructs correlation. The correlation of anti-repeat antibodies measured by IFA and CSP with in vitro and in viva SNA and will protection in vivo will be determined.

### REFERENCES

Aggarwal, A., S. Kumar, et al. (1990). “Oral Salmonella: malaria circumsporozoite recombinants induce specific CD8+ cytotoxic T cells.” J Exp Med 172(4): 1083-90.
Alonso, P. L., J. Sacarlal, et al. (2004). “Efficacy of the RTS,S/AS02A vaccine against Plasmodium falciparum infection and disease in young African children: randomised controlled trial.” Lancet 364(9443): 1411-20.
Aponte, J. J., P. Aide, et al. (2007). “Safety of the RTS,S/AS02D candidate malaria vaccine in infants living in a highly endemic area of Mozambique: a double blind randomised controlled phase I/IIb trial.” Lancet 370(9598): 1543-51.
Arakawa, T., A. Komesu, et al. (2005). “Nasal immunization with a malaria transmission-blocking vaccine candidate, Pfs25, induces complete protective immunity in mice against field isolates of Plasmodium falciparum.” Infect Immun 73(11): 7375-80.
Arakawa, T., T. Tsuboi, et al. (2003). “Serum antibodies induced by intranasal immunization of mice with Plasmodium vivax Pvs25 co-administered with cholera toxin completely block parasite transmission to mosquitoes.” Vaccine 21(23): 3143-8.
Ballou, R. W. (2007). “Obstacles to the development of a safe and effective attenuated pre-erythrocytic stage malaria vaccine.” Microbes Infect 9(6): 761-6.
Ballou, W. R., M. Arevalo-Herrera, et al. (2004). “Update on the clinical development of candidate malaria vaccines.” Am J Trop Med Hyg 71(2 Suppl): 239-47.
Ballou, W. R., S. L. Hoffman, et al. (1987). “Safety and efficacy of a recombinant DNA Plasmodium falciparum sporozoite vaccine.” Lancet 1(8545): 1277-81.
Barton, G. M. and R. Medzhitov (2002). “Control of adaptive immune responses by Toll-like receptors.” Curr Opin Immunol 14(3): 380-3.
Bendelac, A. and R. Medzhitov (2002). “Adjuvants of immunity: harnessing innate immunity to promote adaptive immunity.” J Exp Med 195(5): F19-23.
Birkett, A., K. Lyons, et al. (2002). “A modified hepatitis B virus core particle containing multiple epitopes of the Plasmodium falciparum circumsporozoite protein provides a highly immunogenic malaria vaccine in preclinical analyses in rodent and primate hosts.” Infect Immun 70(12): 6860-70.
Blander, J. M. and R. Medzhitov (2006). “Toll-dependent selection of microbial antigens for presentation by dendritic cells.” Nature 440(7085): 808-12.
Blum-Tirouvanziam, U., C. Servis, et al. (1995). “Localization of HLA-A2.1-restricted T cell epitopes in the circumsporozoite protein of Plasmodium falciparum.” J Immunol 154(8): 3922-31.
Bojang, K. A., P. J. Milligan, et al. (2001). “Efficacy of RTS,S/AS02 malaria vaccine against Plasmodium falciparum infection in semi-immune adult men in The Gambia: a randomised trial.” Lancet 358(9297): 1927-1934.
Bruna-Romero, 0., G. Gonzalez-Aseguinolaza, et al. (2001). “Complete, long-lasting protection against malaria of mice primed and boosted with two distinct viral vectors expressing the same plasmodial antigen.” Proc Natl Acad Sci USA 98(20): 11491-6.
Calvo-Calle, J. M., J. Hammer, et al. (1997). “Binding of malaria T cell epitopes to DR and DQ molecules in vitro correlates with immunogenicity in vivo: identification of a universal T cell epitope in the Plasmodium falciparum circumsporozoite protein.” J Immunol 159(3): 1362-73.
Calvo-Calle, J. M., G. A. Oliveira, et al. (2005). “Human CD4+ T cells induced by synthetic peptide malaria vaccine are comparable to cells elicited by attenuated Plasmodium falciparum sporozoites.” J Immunol 175(11): 7575-85.
Calvo-Calle, J. M., G. A. Oliveira, et al. (2006). “A linear peptide containing minimal T- and B-cell epitopes of Plasmodium falciparum circumsporozoite protein elicits protection against transgenic sporozoite challenge.” Infect Immun 74(12): 6929-39.
Chomczynski, P. and N. Sacchi (1987). “Single-step method of RNA isolation by acid guanidinium thiocyanate-phenol-chloroform extraction.” Anal Biochem 162(1): 156-9.
Clyde, D. F. (1990). “Immunity to falciparum and vivax malaria induced by irradiated sporozoites: a review of the University of Maryland studies, 1971-75.” Bull World Health Organ 68(Suppl): 9-12.
Cochrane, A. H., M. Aikawa, et al. (1976). “Antibody-induced ultrastructural changes of malarial sporozoites.” J Immunol 116(3): 859-67.
Doolan, D. L., S. L. Hoffman, et al. (1997). “Degenerate cytotoxic T cell epitopes from P. falciparum restricted by multiple HLA-A and HLA-B supertype alleles.” Immunity 7(1): 97-112.
Doolan, D. L., A. J. Saul, et al. (1992). “Geographically restricted heterogeneity of the Plasmodium falciparum circumsporozoite protein: relevance for vaccine development.” Infect Immun 60(2): 675-82.
Doolan, D. L., S. Southwood, et al. (2000). “HLA-DR-promiscuous T cell epitopes from Plasmodium falciparum pre-erythrocytic-stage antigens restricted by multiple HLA class II alleles.” J Immunol 165(2): 1123-37.
Ferreira, A., L. Schofield, et al. (1986). “Inhibition of development of exoerythrocytic forms of malaria parasites by gamma-interferon.” Science 232(4752): 881-4.
Fremond, C. M., V. Yeremeev, et al. (2004). “Fatal Mycobacterium tuberculosis infection despite adaptive immune response in the absence of MyD88.” J Clin Invest 114(12): 1790-9.
Fujihashi, K., T. Koga, et al. (2002). “A dilemma for mucosal vaccination: efficacy versus toxicity using enterotoxin-based adjuvants.” Vaccine 20(19-20): 2431-8.
Gonzalez, C., D. Hone, et al. (1994). “Salmonella typhi vaccine strain CVD 908 expressing the circumsporozoite protein of Plasmodium falciparum: strain construction and safety and immunogenicity in humans.” J Infect Dis 169(4): 927-31.
Gordon, D. M., T. W. McGovern, et al. (1995). “Safety, immunogenicity, and efficacy of a recombinantly produced Plasmodium falciparum circumsporozoite protein-hepatitis B surface antigen subunit vaccine.” J Infect Dis 171(6): 1576-85.
Gregson, A., Oliveira, G., Othoro, C, Calvo-Calle, J. M., Thorton, G. B., Nardin, E. and Edelman, R. (2007). “Phase I trial of an alhydrogel adjuvanted hepatitis B core virus-like particle containing epitopes of the Plasmodium falciparum Circumsporozoite Protein.” PLoS Biol.
Herrington, D., J. Davis, et al. (1991). “Successful immunization of humans with irradiated malaria sporozoites: humoral and cellular responses of the protected individuals.” Am J Trop Med Hyg 45(5): 539-47.
Herrington, D. A., D. F. Clyde, et al. (1990). “Human studies with synthetic peptide sporozoite vaccine (NANP)3-TT and immunization with irradiated sporozoites.” Bull World Health Organ 68 Suppl: 33-7.
Herrington, D. A., G. A. Losonsky, et al. (1992). “Safety and immunogenicity in volunteers of a recombinant Plasmodium falciparum circumsporozoite protein malaria vaccine produced in Lepidopteran cells.” Vaccine 10(12): 841-6.
Herrington, D. A., E. H. Nardin, et al. (1991). “Safety and immunogenicity of a recombinant sporozoite malaria vaccine against Plasmodium vivax.” Am J Trop Med Hyg 45(6): 695-701.
Hirunpetcharat, C., D. Stanisic, et al. (1998). “Intranasal immunization with yeast-expressed 19 kD carboxyl-terminal fragment of Plasmodium yoelii merozoite surface protein-1 (yMSP119) induces protective immunity to blood stage malaria infection in mice.” Parasite Immunol 20(9): 413-20.
Hoffman, S. L., L. M. Goh, et al. (2002). “Protection of humans against malaria by immunization with radiation-attenuated Plasmodium falciparum sporozoites.” J Infect Dis 185(8): 1155-64.
Huleatt, J. W., A. R. Jacobs, et al. (2007). “Vaccination with recombinant fusion proteins incorporating Toll-like receptor ligands induces rapid cellular and humoral immunity.” Vaccine 25(4): 763-75.
Huleatt, J. W., V. Nakaar, et al. (2008). “Potent immunogenicity and efficacy of a universal influenza vaccine candidate comprising a recombinant fusion protein linking influenza M2e to the TLR5 ligand flagellin.” Vaccine 26(2): 201-14.
Kester, K. E., D. A. McKinney, et al. (2001). “Efficacy of recombinant circumsporozoite protein vaccine regimens against experimental Plasmodium falciparum malaria.” J Infect Dis 183(4): 640-7.
Kopp, E. B. and R. Medzhitov (1999). “The Toll-receptor family and control of innate immunity.” Curr Opin Immunol 11(1): 13-8.
Kumar, K. A., G. A. Oliveira, et al. (2004). “Quantitative Plasmodium sporozoite neutralization assay (TSNA).” J Immunol Methods 292(1-2): 157-64.
Kumar, K. A., G. Sano, et al. (2006). “The circumsporozoite protein is an immunodominant protective antigen in irradiated sporozoites.” Nature 444(7121): 937-40.
Langennans, J. A., A. Schmidt, et al. (2005). “Effect of adjuvant on reactogenicity and long-term immunogenicity of the malaria Vaccine ICC-1132 in macaques.” Vaccine 23(41): 4935-43.
Latz, E., J. Franko, et al. (2004). “Haemophilus influenzae type b-outer membrane protein complex glycoconjugate vaccine induces cytokine production by engaging human Toll-like Receptor 2 (TLR2) and requires the presence of TLR2 for optimal immunogenicity.” J Immunol 172(4): 2431-8.
Levine, M. M. (2003). “Can needle-free administration of vaccines become the norm in global immunization?” Nat Med 9(1): 99-103.
Luke, T. C. and S. L. Hoffman (2003). “Rationale and plans for developing a non-replicating, metabolically active, radiation-attenuated Plasmodium falciparum sporozoite vaccine.” J Exp Biol 206(Pt 21): 3803-8.
McDonald, W. F., J. W. Huleatt, et al. (2007). “A West Nile virus recombinant protein vaccine that coactivates innate and adaptive immunity.” J Infect Dis 195(11): 1607-17.
Means, T. K., F. Hayashi, et al. (2003). “The 5 stimulus bacterial flagellin induces maturation and chemokine production in human dendritic cells.” J Immunol 170(10): 5165-75.
Medzhitov, R. (2001). “Toll-like Receptors and innate immunity.” Nat Rev Immunol 1(2): 135-45.
Medzhitov, R. and C. A. Janeway, Jr. (1997). “Innate immunity: impact on the adaptive immune response.” Curr Opin Immunol 9(1): 4-9.
Moreno, A., P. Clavijo, et al. (1991). “Cytotoxic CD4+ T cells from a sporozoite-immunized volunteer recognize the Plasmodium falciparum CS protein.” Int Immunol 3(10): 997-1003.
Moreno, A., P. Clavijo, et al. (1993). “CD4+ T cell clones obtained from Plasmodium falciparum sporozoite-immunized volunteers recognize polymorphic sequences of the circumsporozoite protein.” J Immunol 151(1): 489-99.
Munesinghe, D. Y., P. Clavijo, et al. (1991). “Immunogenicity of multiple antigen peptides (MAP) containing T and B cell epitopes of the repeat region of the P. falciparum circumsporozoite protein.” Eur J Immunol 21(12): 3015-20.
Mutsch, M., W. Zhou, et al. (2004). “Use of the inactivated intranasal influenza vaccine and the risk of Bell's palsy in Switzerland.” N Engl J Med 350(9): 896-903.
Nardin, E., P. Clavijo, et al. (1991). “T cell epitopes of the circumsporozoite protein of Plasmodium vivax. Recognition by lymphocytes of a sporozoite-immunized chimpanzee.” J Immunol 146(5): 1674-8.
Nardin, E. H., J. M. Calvo-Calle, et al. (1998). “Plasmodium falciparum polyoximes: highly immunogenic synthetic vaccines constructed by chemoselective ligation of repeat B-cell epitopes and a universal T-cell epitope of CS protein.” Vaccine 16(6): 590-600.
Nardin, E. H., J. M. Calvo-Calle, et al. (2001). “A totally synthetic polyoxime malaria vaccine containing Plasmodium falciparum B cell and universal T cell epitopes elicits immune responses in volunteers of diverse HLA types.” J Immunol 166(1): 481-9.
Nardin, E. H., D. A. Herrington, et al. (1989). “Conserved repetitive epitope recognized by CD4+ clones from a malaria-immunized volunteer.” Science 246(4937): 1603-6.
Nardin, E. H. and R. S, Nussenzweig (1993). “T cell responses to pre-erythrocytic stages of malaria: role in protection and vaccine development against pre-erythrocytic stages.” Annu Rev Immunol 11: 687-727.
Nardin, E. H., G. A. Oliveira, et al. (2000). “Synthetic peptide malaria vaccine elicits high levels of antibodies in vaccinees of defined HLA genotypes.” J Infect Dis 182(5): 1486-96.
Nardin, E. H., G. A. Oliveira, et al. (2004). “Phase I testing of a malaria vaccine composed of hepatitis B virus core particles expressing Plasmodium falciparum circumsporozoite epitopes.” Infect Immun 72(11): 6519-27.
Nussenzweig, R. S., J. Vanderberg, et al. (1967). “Protective immunity produced by the injection of x-irradiated sporozoites of plasmodium berghei.” Nature 216(111): 160-2.
Nussenzweig, V. and R. S, Nussenzweig (1989). “Rationale for the development of an engineered sporozoite malaria vaccine.” Adv Immunol 45: 283-334.
Oliveira, G. A., K. Wetzel, et al. (2005). “Safety and enhanced immunogenicity of a hepatitis B core particle Plasmodium falciparum malaria vaccine formulated in adjuvant Montanide ISA 720 in a phase I trial.” Infect Immun 73(6): 3587-97.
Pasare, C. and R. Medzhitov (2004). “Toll-like receptors and acquired immunity.” Semin Immunol 16(1): 23-6.
Persson, C., G. A. Oliveira, et al. (2002). “Cutting edge: a new tool to evaluate human pre-erythrocytic malaria vaccines: rodent parasites bearing a hybrid Plasmodium falciparum circumsporozoite protein.” J Immunol 169(12): 6681-5.
Reece, W. H., M. Pinder, et al. (2004). “A CD4(+) T-cell immune response to a conserved epitope in the circumsporozoite protein correlates with protection from natural Plasmodium falciparum infection and disease.” Nat Med 10(4): 406-10.
Sadoff, J. C., W. R. Ballou, et al. (1988). “Oral Salmonella typhimurium vaccine expressing circumsporozoite protein protects against malaria.” Science 240(4850): 336-8.
Saul, A., G. Lawrence, et al. (2005). “A human phase 1 vaccine clinical trial of the Plasmodium falciparum malaria vaccine candidate apical membrane antigen 1 in Montanide ISA720 adjuvant.” Vaccine 23(23): 3076-83.
Saul, A., G. Lawrence, et al. (1999). “Human phase I vaccine trials of 3 recombinant asexual stage malaria antigens with Montanide ISA720 adjuvant.” Vaccine 17(23-24): 3145-59.
Schofield, L., A. Ferreira, et al. (1987). “Interferon-gamma inhibits the intrahepatocytic development of malaria parasites in vitro.” J Immunol 139(6): 2020-5.
Sinigaglia, F., M. Guttinger, et al. (1988). “A malaria T cell epitope recognized in association with most mouse and human MHC class II molecules.” Nature 336: 778-781.
Sinnis, P. and E. Nardin (2002). “Sporozoite antigens: biology and immunology of the circumsporozoite protein and thrombospondin-related anonymous protein.” Chem Immunol 80: 70-96.
Song, L., V. Nakaar, et al. (2008). “Efficacious recombinant influenza vaccines produced by high yield bacterial expression: a solution to global pandemic and seasonal needs.” PLoS ONE 3(5): e2257.
Stewart, M. J., R. J. Nawrot, et al. (1986). “Plasmodium berghei sporozoite invasion is blocked in vitro by sporozoite-immobilizing antibodies.” Infect Immun 51(3): 859-64.
Stoute, J. A., K. E. Kester, et al. (1998). “Long-term efficacy and immune responses following immunization with the RTS,S malaria vaccine.” J Infect Dis 178(4): 1139-44.
Stoute, J. A., M. Slaoui, et al. (1997). “A preliminary evaluation of a recombinant circumsporozoite protein vaccine against Plasmodium falciparum malaria. RTS,S Malaria Vaccine Evaluation Group [see comments].” N Engl J Med 336(2): 86-91.
Sztein, M. B., S. S. Wasserman, et al. (1994). “Cytokine production patterns and lymphoproliferative responses in volunteers orally immunized with attenuated vaccine strains of Salmonella typhi.” J Infect Dis 170(6): 1508-17.
Takita-Sonoda, Y., M. Tsuji, et al. (1996). “Plasmodium yoelii: peptide immunization induces protective CD4+ T cells against a previously unrecognized cryptic epitope of the circumsporozoite protein.” Exp Parasitol 84(2): 223-30.
Tam, J. P., P. Clavijo, et al. (1990). “Incorporation of T and B epitopes of the circumsporozoite protein in a chemically defined synthetic vaccine against malaria.” J Exp Med 171(1): 299-306.
van Duin, D., R. Medzhitov, et al. (2006). “Triggering TLR signaling in vaccination.” Trends Immunol 27(1): 49-55.
Vanderberg, J., R. Nussenzweig, et al. (1969). “Protective immunity produced by the injection of x-irradiated sporozoites of Plasmodium berghei. V. In vitro effects of immune serum on sporozoites.” Mil Med 134(10): 1183-90.
Vanderberg, J. P. and U. Frevert (2004). “Intravital microscopy demonstrating antibody-mediated immobilisation of Plasmodium berghei sporozoites injected into skin by mosquitoes.” Int J Parasitol 34(9): 991-6.
Walther, M., S. Dunachie, et al. (2005). “Safety, immunogenicity and efficacy of a pre-erythrocytic malaria candidate vaccine, ICC-1132 formulated in Seppic ISA 720.” Vaccine 23(7): 857-64.
Yoshida, N., S. M. Di Santi, et al. (1990). “Plasmodium falciparum: restricted polymorphism of T cell epitopes of the circumsporozoite protein in Brazil.” Experimental Parasitology 71: 386-392.
Zavala, F. and S. Chai (1990). “Protective anti-sporozoite antibodies induced by a chemically defined synthetic vaccine.” Immunol Lett 25(1-3): 271-4.
Zavala, F., R. W. Gwadz, et al. (1982). “Monoclonal antibodies to circumsporozoite proteins identify the species of malaria parasite in infected mosquitoes.” Nature 299(5885): 737-8.

The teachings of all of the above references are hereby incorporated by reference in their entirety.

### EQUIVALENTS

While this invention has been particularly shown and described with references to example embodiments thereof, it will be understood by those skilled in the art that various changes in form and details may be made therein without departing from the scope of the invention encompassed by the appended claims.

What is claimed is:

1. A composition that includes at least one fusion protein comprising at least a portion of at least one flagellin and at least a portion of at least one malaria antigen, wherein the fusion protein activates a Toll-like Receptor 5.

2. The composition of claim 1, wherein the flagellin includes at least one member selected from the group consisting of a Salmonella typhimurium flagellin, an E. coli flagellin, a S. muenchen flagellin, a Yersinia flagellin, a P. aeruginosa flagellin and a L. monocytogenes flagellin.

3. The composition of claim 1, wherein the flagellin lacks at least a portion of a hinge region.

4. The composition of claim 1, wherein the malaria antigen includes at least one member selected from the group consisting of a Plasmodium malaria antigen, a Plasmodium reichenowi antigen, a Plasmodium yoelii antigen, a Plasmodium berghei antigen, a Plasmodium vivax antigen, a Plasmodium ovale antigen and a Plasmodium knowleis antigen.

5. The composition of claim 1, wherein the malaria antigen includes a Plasmodium falciparum malaria antigen.

6. The composition of claim 5, wherein the Plasmodium falciparum malaria antigen includes a sporozite stage malaria antigen.

7. The composition of claim 6, wherein the sporozite stage malaria antigen includes a circumsporozite protein antigen.

8. The composition of claim 7, wherein the circumsporozite antigen includes at least a portion of at least one T-cell epitope.

9. The composition of claim 8, further including at least a portion of at least one B-cell epitope.

10. A composition that includes at least one fusion protein comprising at least a portion of at least one flagellin, at least a portion of at least one malaria antigen T-cell epitope and at least a portion of at least one malaria antigen B-cell epitope, wherein the fusion protein activates a Toll-like Receptor 5.

11. The composition of claim 10, wherein the malaria T-cell antigen includes a Plasmodium falciparum malaria T-cell antigen.

12. The composition of claim 10, wherein the malaria B-cell epitope includes a Plasmodium falciparum malaria B-cell epitope.

13. A composition that includes at least one fusion protein comprising at least a portion of at least one flagellin and at least a portion of at least one Plasmodium falciparum circumsporozite protein antigen, wherein the flagellin activates a Toll-like Receptor 5.

14. The composition of claim 13, further including at least one additional malaria antigen.

15. The composition of claim 14, wherein the additional malaria antigen is at least one member selected from the group consisting of a merozoite surface protein antigen, a Duffy-binding protein-1 antigen, an apical membrane antigen-1 antigen, a reticulocyte-binding protein antigen and a liver stage antigen.

16. A method of stimulating an immune response in a subject, comprising the step of administering to the subject a composition that includes at least one fusion protein comprising at least a portion of at least one flagellin and at least a portion of at least one malaria antigen, wherein the fusion protein activates a Toll-like Receptor 5.

17. The method of claim 16, wherein the flagellin lacks at least a portion of a hinge region.

18. The method of claim 16, wherein the composition provides sterile immunity against a malaria infection in the subject.

19. The method of claim 16, wherein administration of the composition to the subject provides protective immunity against an infection consequent to exposure of the subject to a source of the malaria antigen.

20. A method of stimulating an immune response in a subject, comprising the step of administering to the subject a composition that includes at least one fusion protein comprising at least a portion of at least one flagellin and at least a portion of at least one Plasmodium falciparum circumsporozite protein antigen, wherein the fusion protein activates a Toll-like Receptor 5.

21. The method of claim 20, wherein the composition administered to the subject further includes at least one additional malaria antigen.

22. The method of claim 21, wherein the additional malaria antigen is at least one member selected from the group consisting of a merozoite surface protein antigen, a Duffy-binding protein-1 antigen, an apical membrane antigen-1 antigen, a reticulocyte-binding protein antigen and a liver stage antigen.
